(19) United States
(12) Patent Application Publication
(10) Pub. No.: US 2001/0044445 A1
(43) Pub. Date: Nov. 22, 2001

(54) AZOLE INHIBITORS OF CYTOKINE PRODUCTION

(76) Inventors: NWE Y. BAMAUNG, NILES, IL (US); ANWER BASHA, LAKE FOREST, IL (US); STEVAN W. DJURIC, LIBERTYVILLE, IL (US); EARL J. GUBBINS, LIBERTYVILLE, IL (US); JAY R. LULY, WELLESLEY, MA (US); NOAH P. TU, GURNEE, IL (US); DAVID J. MADAR, GRAYSLAKE, IL (US); USHA WARRIOR, GREEN OAKS, IL (US); PAUL E. WIEDEMAN, LIBERTYVILLE, IL (US); XUN ZHOU, PARK CITY, IL (US); RICHARD J. SCIOTTI, GURNEE, IL (US); FRANK L. WAGENAAR, GURNEE, IL (US)

Correspondence Address: ABBOTT LABORATORIES, DEPT. 377 - AP6D-2, 100 ABBOTT PARK ROAD, ABBOTT PARK, IL, 60064-6050, US

(21) Appl. No.: 09/289,155

(22) Filed: Apr. 8, 1999

(51) Int. Cl. C07D 401/00; C07D 231/02; C07D 231/04; C07D 231/06; A61K 31/4439; A61K 31/415

(52) U.S. Cl. 514/277; 548/356.1; 548/364.1; 548/366.1; 546/274.7; 514/336; 514/341; 514/385

(57) ABSTRACT

Compounds having the formula

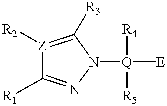

are useful for treating diseases that are prevented by or ameliorated with Interleukin-2, Interleukin-4, or Interleukin-5 production inhibitors.

### TECHNICAL FIELD

The present invention relates to organic compounds and compositions that are cytokine synthesis inhibitors, processes for making such compounds, synthetic intermediates employed in these processes, and methods for inhibiting cytokine production in a mammal.

### BACKGROUND OF THE INVENTION

Therapeutic control of the immune system is the goal of many approaches toward the treatment of autoimmune diseases that differ in organ specific involvement, pathogenic cofactors, response to treatment and prognosis. They range from diseases with “spontaneous” onset such as rheumatoid arthritis to rejection reactions after allograft organ transplantation.

Interleukin 2 (IL-2), a lymphokine produced by activated T-cells, is a key regulator of immune and inflammatory responses. It promotes T cell proliferation in vitro and differentiation of B cells, activated macrophages, NK cells and LAK cells. The central importance of IL-2 in initiating adaptive immune responses such as the rejection of tissue grafts is well-illustrated by drugs that are most commonly used to suppress undesirable effects such as the rejection of tissue grafts. The drugs cyclosporin A and FK506 inhibit IL-2 production by disrupting signalling initiated through the T-cell receptor. The drug rapamycin also inhibits signalling through the T cell receptor. Cyclosporin A and rapamycin act synergistically to inhibit immune responses by preventing the IL-2 driven clonal expansion of T cells (Brazelton and Morris, Current Opinion in Immunology 8, 710 (1996)).

Compounds of this invention, due to their ability to inhibit IL-2 production, can be anticipated to demonstrate therapeutic efficacy in disease states where IL-2 is a key orchestrator of the immune response such as rheumatoid arthritis, atopic dermatitis, psoriasis and the rejection of tissue grafts.

Increased local elaboration of the Th2-type cytokines Interleukin-5 (IL-5) and Interleukin-4 (IL-4) has clearly been implicated in the pathogenesis of atopic asthma (Am. J.Respir. Crit. Care Med. 154 1497 (1996)). IL-5 has selective biologic effects on eosinophils and their precursors and may regulate selective accumulation of these cells in the asthmatic bronchial mucosa. IL-4 is an essential co-factor for IgE switching in B-lymphocytes and is therefore likely to be involved in situations where there is inappropriate IgE synthesis. Compounds of this invention inhibit the production of both IL-4 and IL-5 and can be expected to exhibit efficacy in atopic diseases where the aforementioned cytokines play a prominent role in disease pathophysiology.

### SUMMARY OF THE INVENTION

In its principle embodiment, the present invention provides a compound represented by Formula I

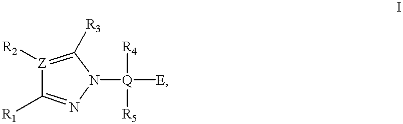

or a pharmaceutically acceptable salt or prodrug thereof, where

R₁ and R₃ are independently selected from

(1) hydrogen,

(2) aryl,

(3) perfluoroalkyl of one to fifteen carbons,

(4) halo,

(5) —CN,

(6) —NO₂,

(7) —OH,

(8) —OG where G is a hydroxyl protecting group,

(9) —CO₂R₆ where R₆ is selected from

(a) hydrogen,

(b) cycloalkyl of three to twelve carbons,

(c) aryl,

(d) aryl substituted with 1, 2, 3, 4, or 5 substituents independently selected from

(i) alkyl of one to fifteen carbons,

(ii) alkoxy of one to fifteen carbons,

(iii) thioalkoxy of one to fifteen carbons,

(iv) halo,

(v) —NO₂, and

(vi) —N₃,

(e) a carboxy protecting group,

(f) alkyl of one to fifteen carbons,

(g) alkyl of one to fifteen carbons substituted with 1, 2, or 3, or 4 substituents independently selected from

(i) alkoxy of one to fifteen carbons,

(ii) thioalkoxy of one to fifteen carbons,

(iii) aryl,

(iv) aryl substituted with 1, 2, 3, 4, or 5 substituents independently selected from alkyl of one to fifteen carbons, alkoxy of one to fifteen carbons, thioalkoxy of one to fifteen carbons, halo, —NO₂, and —N₃,

(v) cycloalkyl of three to twelve carbons, and

(vi) halo,

(h) alkenyl of three to fifteen carbons, provided that a carbon of a carbon-carbon double bond is not attached directly to oxygen,

(i) alkynyl of three to fifteen carbons, provided that a carbon of a carbon-carbon triple bond is not attached directly to oxygen, and

(j) cycloalkyl of three to twelve carbons,

(10) —L₁NR₇R₈ where L₁ is selected from

(a) a covalent bond,

(b) —X° C(X)— where X and X′ are independently O or S,

(c) —C(X)—, and

(d) —NR₆— and

R₇ and R₈ are independently selected from

(a) hydrogen,

(b) alkanoyl where the alkyl part is one to fifteen carbons,

(c) alkoxycarbonyl where the alkyl part is one to fifteen carbons,

(d) alkoxycarbonyl where the alkyl part is one to fifteen carbons and is substituted with 1 or 2 substituents selected from the group consisting of aryl,

(e) cycloalkyl of three to twelve carbons,

(f) aryl,

(g) aryl substituted with 1, 2, 3, 4, or 5 substituents independently selected from

(i) alkyl of one to fifteen carbons,

(ii) alkoxy of one to fifteen carbons,

(iii) thioalkoxy of one to fifteen carbons,

(iv) halo,

(v) —NO₂, and

(vi) —N₃,

(h) —OR₆, provided that only one of R₇ or R₈ is —OR₆,

(i) a nitrogen protecting group,

(j) alkyl of one to fifteen carbons,

(k) alkyl of one to fifteen carbons substituted with 1, 2, or 3, or 4 substituents independently selected from

(i) alkoxy of one to fifteen carbons,

(ii) thioalkoxy of one to fifteen carbons,

(iii) aryl,

(iv) aryl substituted with 1, 2, 3, 4, or 5 substituents independently selected from alkyl of one to fifteen carbons, alkoxy of one to fifteen carbons, thioalkoxy of one to fifteen carbons, halo, —NO₂, and —N₃,

(v) cycloalkyl of three to fifteen carbons,

(vi) halo,

(vii) —CO₂R₆, and

(viii) —OH,

(l) alkenyl of three to fifteen carbons, provided that a carbon of a carbon-carbon double bond is not attached directly to nitrogen,

(m) alkynyl of three to fifteen carbons, provided that a carbon of a carbon-carbon triple bond is not attached directly to nitrogen,

(n) —SO₂-alkyl, and

(o) cycloalkyl of three to twelve carbons, or

R₇ and R₈ together with the nitrogen atom to which they are attached form a ring selected from

(i) aziridine,

(ii) azetidine,

(iii) pyrrolidine,

(iv) piperidine,

(v) piperazine,

(vi) morpholine,

(vii) thiomorpholine, and

(viii) thiomorpholine sulfone

where (i)-(viii) can be optionally substituted with 1, 2, or 3 substituents selected from the group consisting of alkyl of one to fifteen carbons,

(11) —L₂R₉ where L₂ is selected from

(a) —L₁—,

(b) —O—, and

(c) —S(O)ₜ— where t is 0, 1, or 2 and

R₉ is selected from

(a) cycloalkyl of three to twelve carbons,

(b) aryl

(c) aryl substituted with 1, 2, 3, 4, or 5 substituents independently selected from

(i) alkyl of one to fifteen carbons,

(ii) alkoxy of one to fifteen carbons,

(iii) thioalkoxy of one to fifteen carbons,

(iv) halo,

(v) —NO₂, and

(vi) —N₃,

(d) alkyl of one to fifteen carbons,

(e) heterocycle,

(f) alkenyl of two to fifteen carbons, and

(e) alkyl of one to fifteen carbons substituted with 1, 2, or 3, or 4 substituents independently selected from

(i) alkenyl of two to fifteen carbons,

(ii) alkoxy of one to fifteen carbons,

(iii) —CN,

(iv) —CO₂R₆,

(v) —OH, provided that no two —OH groups are attached to the same carbon,

(vi) thioalkoxy of one to fifteen carbons,

(vii) alkynyl of two to fifteen carbons,

(viii) aryl,

(ix) aryl substituted with 1, 2, 3, 4, or 5 substituents independently selected from alkyl of one to fifteen carbons, alkoxy of one to fifteen carbons, thioalkoxy of one to fifteen carbons, halo, —NO₂, and —N₃,

(x) cycloalkyl of three to twelve carbons, and

(xi) halo,

(xii) —NR₇R₈,

(xiii) heterocycle, and

(xiv) heterocycle substituted with 1, 2, or 3, or 4 substituents independently selected from alkyl of one to fifteen carbons, alkoxy of one to fifteen carbons, thioalkoxy of one to fifteen carbons, halo, —NO₂, and —N₃,

(12) alkyl of one to fifteen carbons substituted with 1, 2, 3, 4, or 5 halo substituents,

(13) alkyl of one to fifteen carbons,

(14) alkenyl of two to fifteen carbons,

(15) alkynyl of two to fifteen carbons

where (13)-(15) can be optionally substituted with

(a) (═X),

(b) alkanoyloxy where the alkyl part is one to fifteen carbons,

(c) alkoxy of one to fifteen carbons,

(d) alkoxy of one to fifteen carbons substituted with 1, 2, 3, 4, or 5 substituents selected from the group consisting of halo,

(e) thioalkoxy of one to fifteen carbons,

(f) perfluoroalkoxy of one to fifteen carbons,

(g) —N₃,

(h) —NO₂,

(i) —CN,

(j) —OH,

(k) —OG

(l) cycloalkyl of three to twelve carbons,

(m) halo,

(n) —CO₂R₆,

(o) —L₁NR₇R₈, and

(p) —L₂R₉,

(16) —L₂-heterocycle, and

(17) —L₂-heterocycle where the heterocycle is substituted with 1, 2, 3 or 4 substituents independently selected from

(a) alkyl of one to fifteen carbons,

(b) perfluoroalkyl of one to fifteen carbons,

(c) alkoxy of one to fifteen carbons,

(d) thioalkoxy of one to fifteen carbons,

(e) halo, and

(f) —NO₂,

(18) —NR_{X}C(O)NR_{Y}R_{Z} where R_{X}, R_{Y} and R_{Z} are independently selected from

(a) hydrogen and

(b) alkyl of one to fifteen carbons,

(19) —C(═NR_{X})NR_{Y}R_{Z},

(20) —NR_{X}C(═NR_{X′})NR_{Y}R_{Z} where R_{X}, R_{Y} and R_{Z} are defined previously and R_{X′} is selected from

(a) hydrogen and

(b) alkyl of one to fifteen carbons,

(21) —NR_{X}C(O)OR_{W}, where R_{W} is selected from

(a) alkyl of one to fifteen carbons and

(b) alkenyl of three to fifteen carbons, provided that a carbon of a carbon-carbon double bond is not attached directly to oxygen, and

(22) —OC(O)NR₇R₈;

Z is nitrogen or carbon;

R₂ is absent or is selected from

(1) hydrogen,

(2) —CO₂R₆,

(3) alkyl of one to fifteen carbons,

(4) —C(O)R_{6′} where R₆′ is selected from

(a) alkyl of one to fifteen carbons,

(b) aryl, and

(c) heterocycle,

(5) —C(O)NR_{7′}R_{8′} where R_{7′} and R_{8′} are independently selected from

(a) hydrogen,

(b) alkyl of one to fifteen carbons, or

R_{7′} and R_{8′} together with the nitrogen to which they are attached form a ring selected from

(i) piperidine,

(ii) piperazine,

(iii) morpholine,

(iv) thiomorpholine, and

(v) thiomorpholine sulfone

(6) perfluoroalkyl of one to fifteen carbons,

(7) cycloalkyl of three to ten carbons,

(8) alkyl of one to fifteen carbons substituted with 1, 2, 3, 4, or 5 substituents selected from the group conststing of halo,

(9) alkyl of one to fifteen carbons substituted with

(a) —CN,

(b) —OH, provided that no two —OH groups are attached to the same carbon,

(c) (═X), and

(d) —CO₂R₆, and

(10) halogen;

provided that when X is nitrogen, R₂ is absent;

Q is aryl or heterocycle where, when Q is phenyl, the phenyl is 2-, 3-, or 4- substituted by E relative to the position of attachment of the pyrazole or 1,2,4-triazole ring to the phenyl ring;

R₄ and R₅ are independently selected from

(1) hydrogen,

(2) alkyl of one to fifteen carbons,

(3) alkyl of one to fifteen carbons substituted with 1, 2, 3, 4, or 5 halo substituents,

(4) alkyl of one to fifteen carbons substituted with

(a) —CN,

(b) —CO₂R₆,

(c) —L₁NR₇R₈, and

(d) L₂R₉,

(5) perfluoroalkyl of one to fifteen carbons,

(6) —CN,

(7) —CO₂R₆,

(8) —L₁NR₇R₈,

(9) —L₂R₉,

(10) alkoxy of one to fifteen carbons,

(11) thioalkoxy of one to fifteen carbons,

(12) halo,

(13) —C(═NR₆)NR₇R₈,

(14) —NR₁₂(═NR₆)NR₇R₈ where R₆, R₇, and R₈ are defined previously and R₁₂ is selected from

(a) hydrogen,

(b) cycloalkyl of three to twelve carbons,

(c) aryl,

(d) alkyl of one to fifteen carbons, and

(e) alkyl of one to fifteen carbons substituted with 1, 2, or 3, or 4 substituents independently selected from

(i) alkenyl of two to fifteen carbons,

(ii) alkoxy of one to fifteen carbons,

(iii) thioalkoxy of one to fifteen carbons,

(iv) alkynyl of two to fifteen carbons, and

(v) aryl,

(15) —L₂-heterocycle, and

(16) —L₂-heterocycle where the heterocycle is substituted with 1, 2, 3, or 4 substituents independently selected from

(a) alkyl of one to fifteen carbons,

(b) perfluoroalkyl of one to fifteen carbons,

(c) alkoxy of one to fifteen carbons,

(d) thioalkoxy of one to fifteen carbons,

(e) halo,

(f) —N₃, and

(g) —NO₂;

E is

(1) —L₃-B where L₃ is selected from

(a) a covalent bond,

(b) alkenylene of two to six carbons in the Z or E configuration,

(c) alkynylene of two to six carbons,

(d) —C(X)—,

(e) —N═N—,

(f) —NR₇—,

(g) —N(R₇)C(O)N(R₈)—,

(h) —N(R₇)SO₂N(R₈)—,

(i) —X—,

(j) —(CH₂)ₘO—,

(k) —O(CH₂)ₘ—,

(l) —N(R₇)C(X)—,

(m) —C(X)N(R₇)—,

(n) —S(O)ₜ(CH₂)ₘ—,

(o) —(CH₂)ₘS(O)ₜ—,

(p) —NR₇(CH₂)ₘ—,

(q) —(CH₂)ₘNR₇—,

(r) —NR₇S(O)ₜ—,

(s) —S(O)ₜNR₇—,

(t) —N═C(H)—,

(u) —C(H)═N—,

(v) —ON═CH—,

(w) —CH═NO—

where (g)-(w) are drawn with their left ends attached to Q.

(x) —N(R₇)C(O)N(R₁₀)(R₁₁)— where R₁₀ and R₁₁ together with the nitrogen atom to which they are attached form a ring selected from

(i) morpholine,

(ii) thiomorpholine,

(iii) thiomorpholine sulfone, and

(iv) piperidine

where (i)-(iv) are attached to Q through the nitrogen to which is attached R₇ and to B through a carbon in the ring,

(y) —N(R₇)SO₂N(R₁₀)(R₁₁)—, and

(z) —N(R₇)C(O)N(R₁₀)(R₁₁)— and

B is selected from

(a) alkyl of one to fifteen carbons,

(b) alkenyl of three to fifteen carbons in the E or Z configuration, provided that a carbon of a carbon-carbon double bond is not directly attached to L₃ when L₃ is other than a covalent bond,

(c) alkynyl of three to fifteen carbons,

provided that a carbon of a carbon-carbon triple bond is not directly attached to L₃ when L₃ is other than a covalent bond where (a), (b) and (c), can be optionally substituted with 1, 2, 3, or 4 substituents independently selected from

(i)

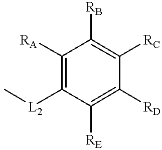

where L₂ is defined previously and R_{A}, R_{B}, R_{C}, R_{D}, and R_{E} are independently selected from

hydrogen,

alkanoyl where the alkyl part is one to fifteen carbons,

alkanoyloxy where the alkyl part is one to fifteen carbons,

alkoxy of one to fifteen carbons,

thioalkoxy of one to fifteen carbons,

alkoxy of one to fifteen carbons substituted with 1, 2, 3, 4, or 5 substituents selected from the group consisting of halo,

perfluoroalkyl of one to fifteen carbons,

perfluoroalkoxy of one to fifteen carbons,

—N₃,

—NO₂,

—CN,

—OH,

—OG,

cycloalkyl of three to fifteen carbons,

halo,

—CO₂R₆

—L₁NR₇R₈

—L₂R₉

alkyl of one to fifteen carbons,

alkyl of one to fifteen carbons substituted with 1, 2, 3, 4, or 5 substituents independently selected from (═X),

alkanoyloxy where the alkyl part is one to fifteen carbons,

alkoxy of one to fifteen carbons,

thioalkoxy of one to fifteen carbons, alkoxy of one to fifteen carbons substituted with 1, 2, 3,4, or 5 halo substituents,

perfluoroalkoxy of one to fifteen carbons,

—N₃,

—NO₂,

—CN, —OH,

provided that no two —OH groups are attached to the same carbon,

—OG,

cycloalkyl of three to fifteen carbons,

halo,

—CO₂R₆,

—L₁NR₇R₈, and

—L₂-heterocycle, and

L₂-heterocycle where the heterocycle is substituted with 1, 2, 3, or 4 substituents independently selected from alkyl of one to fifteen carbons, perfluoroalkyl of one to fifteen carbons, alkoxy of one to fifteen carbons, thioalkoxy of one to fifteen carbons, halo, —NR_{X}C(O)NR_{Y}R_{Z}, —C(═NRX)R_{Y}R_{Z}, —NO₂, and —N₃,

(ii) (═X)

(iii) alkanoyloxy where the alkyl part is one to fifteen carbons,

(iv) alkoxy of one to fifteen carbons,

(v) alkoxy of one to fifteen carbons substituted with 1, 2, 3, 4, or 5 substituents selected from the group consisting of halo,

(vi) thioalkoxy of one to fifteen carbons,

(vii) perfluoroalkoxy of one to fifteen carbons,

(viii) —N₃,

(ix) —NO₂,

(x) —CN,

(xi) —OH, provided that no two -OH groups are attached to the same carbon,

(xii) —OG,

(xiii) cycloalkyl of three to fifteen carbons,

(xiv) halo,

(xv) —CO₂R₆,

(xvi) —L₁NR₇R₈,

(xvii) perfluoroalkyl of one to fifteen carbons,

(xviii) —L₂-heterocycle, and

(xix) —L₂-heterocycle where the heterocycle is substituted with 1, 2, 3, or 4 substituents independently selected from (═X),

alkanoyl where the alkyl part is one to fifteen carbons,

alkanoyloxy where the alkyl part is one to fifteen carbons,

alkoxy of one to fifteen carbons,

alkoxy of one to fifteen carbons substituted with 1, 2, 3, 4, or 5 substituents selected from the group consisting of halo,

thioalkoxy of one to fifteen carbons,

perfluoroalkyl of one to fifteen carbons,

perfluoroalkoxy of one to fifteen carbons,

—N₃,

—NO₂,

—CN,

—OH,

provided that no two —OH groups are attached to the same carbon,

—OG,

cycloalkyl of three to fifteen carbons,

halo,

—CO₂R₆,

—L₁NR₇R₈, and

—L₂R₉,

(d) cycloalkyl of three to twelve carbons,

(e) cycloalkenyl of four to twelve carbons, provided that a carbon of a carbon-carbon-double bond is not attached directly to L₃ when L₃ is other than a covalent bond

where (d) and (e) can be optionally substituted with 1, 2, 3, 4, or 5 substituents independently selected from

(i) alkyl of one to fifteen carbons,

(ii) aryl,

(iii) alkoxy of one to fifteen carbons,

(iv) thioalkoxy of one to fifteen carbons,

(v) halo,

(vi) —OH, provided that no two —OH groups are attached to the same carbon,

(vii) oxo,

(viii) perfluoroalkyl,

(ix) heterocycle, and

(x) heterocycle substituted with 1, 2, 3, 4, or 5 substituents independently selected from alkyl of one to fifteen carbons, perfluoroalkyl of one to fifteen carbons, alkoxy of one to fifteen carbons, thioalkoxy of one to fifteen carbons, halo, —NO₂, and —N₃,

(f)

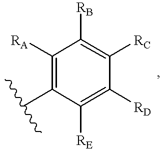

provided that when R₁ and R₃ are both perfluoroalkyl of one carbon, Z is carbon, R₂ is hydrogen, Q is phenyl that is 4-substituted by E relative to the position of attachment of the pyrazole ring to the phenyl group, R4 and R₅ are hydrogen, E is —L₃-B, L₃ is —N(R₇)C(X)—, R₇ is hydrogen, X is oxygen, and RA, RB, RD, and RE are hydrogen, RC is other than chloro, and

(g) heterocycle where the heterocycle can be optionally substituted with 1, 2, 3, or 4 substituents independently selected from

(i) (═X),

(ii) alkanoyl where the alkyl part is one to fifteen carbons,

(iii) alkanoyloxy where the alkyl part is one to fifteen carbons,

(iv) alkoxy of one to fifteen carbons,

(v) alkoxy of one to fifteen carbons substituted with 1, 2, 3, 4, or 5 substituents selected from the group consisting of halo,

(vi) halo,

(vii) thioalkoxy of one to fifteen carbons,

(viii) perfluoroalkyl of one to fifteen carbons,

(ix) perfluoroalkoxy of one to fifteen carbons,

(x) —N₃,

(xi) —NO₂,

(xii) —CN,

(xiii) —OH, provided that no two —OH groups are attached to the same carbon,

(xiv) —OG,

(xv) cycloalkyl of three to fifteen carbons,

(xvi) halo,

(xvii) —CO₂R₆,

(xviii) alkyl optionally substituted with —OH,

(xix) —L₁NR₇R₈, and

(xx) —L₂R₉, and

(2)

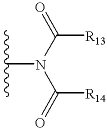

where R₁₃ and R14 are independently selected from

(a) hydrogen,

(b) alkyl of one to fifteen carbons,

(c) alkenyl of three to fifteen carbons in the E or Z configuration, provided that a carbon of a carbon-carbon double bond is not attached directly to the C(═O) group,

(d) alkynyl of three to fifteen carbons, provided that a a carbon-carbon triple bond is not directly attached to the C(═O) group

where (b), (c), and (d) can be optionally substituted with 1, 2, 3, or 4 substituents independently selected from

(i)

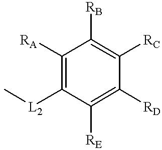

(ii) (═X),

(iii) alkanoyloxy where the alkyl part is one to fifteen carbons,

(iv) alkoxy of one to fifteen carbons,

(v) alkoxy of one to fifteen carbons substituted with 1, 2, 3, 4, or 5 substituents selected from the group consisting of halo,

(vi) thioalkoxy of one to fifteen carbons,

(vii) perfluoroalkoxy of one to fifteen carbons,

(viii) —N₃,

(ix) —NO₂,

(x) —CN,

(xi) —OH, provided that no two —OH groups are attached to the same carbon,

(xii) —OG,

(xiii) cycloalkyl of three to fifteen carbons,

(xiv) halo,

(xv) —CO₂R₆,

(xvi) —L₁NR₇R₈,

(xvii) perfluoroalkyl of one to fifteen carbons,

(xviii) —L₂-heterocycle, and

(xix) —L₂-heterocycle where the heterocycle is substituted with 1, 2, 3,or 4 substituents independently selected from (═X)

alkanoyl where the alkyl part is one to fifteen carbons,

alkanoyloxy where the alkyl part is one to fifteen carbons,

alkoxy of one to fifteen carbons,

alkoxy of one to fifteen carbons substituted with 1, 2, 3, 4, or 5 substituents selected from the group consisting of halo,

thioalkoxy of one to fifteen carbons,

perfluoroalkyl of one to fifteen carbons,

perfluoroalkoxy of one to fifteen carbons,

—N₃,

—NO₂,

—CN,

—OH,

provided that no two —OH groups are attached to the same carbon,

—OG,

cycloalkyl of three to fifteen carbons,

halo,

—CO₂R₆,

—L₁NR₇R₈,

—L₂R₉,

(e) cycloalkyl of three to twelve carbons,

(f) cycloalkenyl of four to twelve carbons, provided that a carbon of a carbon-carbon double bond is not attached directly to the C(═O) group

where (e) and (f) can be optionally substituted with 1, 2, 3, 4, or 5 substituents independently selected from

(i) alkyl of one to fifteen carbons,

(ii) aryl,

(iii) alkoxy of one to fifteen carbons,

(iv) thioalkoxy of one to fifteen carbons,

(v) halo,

(vi) —OH, provided that no two —OH groups are attached to the same carbon,

(vii) heterocycle, and

(viii) heterocycle substituted with 1, 2, 3, 4, or 5 substituents independently selected from alkyl of one to fifteen carbons, perfluoroalkyl of one to fifteen carbons, alkoxy of one to fifteen carbons, thioalkoxy of one to fifteen carbons, halo, —NO₂, and —N₃,

(g) heterocycle, and

(h) heterocycle substituted with 1, 2, 3, or 4 substituents independently selected from

(i) (═X),

(ii) alkanoyl where the alkyl part is one to fifteen carbons,

(iii) alkanoyloxy where the alkyl part is one to fifteen carbons,

(iv) alkoxy of one to fifteen carbons,

(v) alkoxy of one to fifteen carbons substituted with 1, 2, 3, 4, or 5 substituents selected from the group consisting of halo,

(vi) thioalkoxy of one to fifteen carbons,

(vii) perfluoroalkyl of one to fifteen carbons,

(viii) perfluoroalkoxy of one to fifteen carbons,

(ix) —N₃,

(x) —NO₂,

(xi) —CN,

(xii) —OH, provided that no two —OH groups are attached to the same carbon,

(xiii) —OG,

(xiv) cycloalkyl of three to fifteen carbons,

(xv) halo,

(xvi) —CO₂R₆,

(xvii) —L₁NR₇R₈,

(xviii) —L₂R₉,

provided that at least one of R₁₃ and R₁₄ is other than hydrogen, or R₁₃ and R14 together with the nitrogen to which they are attached form a ring selected from

(a) succinimidyl,

(b) maleimidyl,

(c) glutarimidyl,

(d) phthalimidyl,

(e) naphthalimidyl,

(f)

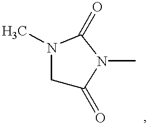

(g)

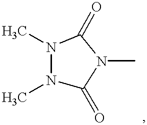

(h)

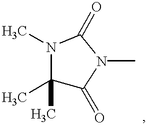

(i)

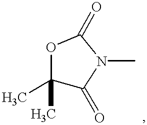

(j)

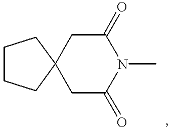

(k)

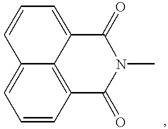

(l)

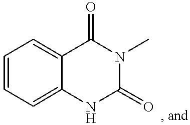

(m)

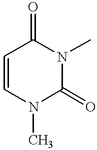

where (a)-(m) can be optionally substituted with 1, 2, 3, 4, or 5 substituents selected from halo and —L₂R₉.

In another embodiment, the present invention also relates to a method of inhibiting Interleukin-2, Interleukin-4, and Interleukin-5 production in a mammal comprising administering a therapeutically effective amount of a compound of Formula I.

In yet another embodiment, the present invention also relates to a method of treating immunologically-mediated diseases in a mammal comprising administering a therapeutically effective amount of a compound of Formula I.

In still yet another embodiment, the present invention relates to pharmaceutical compositions which comprise a therapeutically effective amount of a compound of Formula I in combination with a pharmaceutically acceptable carrier.

Compounds of the invention include but are not limited to

N-[4-[3,5-bis(trifluoromethyl)-1H-pyrazol-1-yl]phenyl]cyclopropanecarboxamide,

N-[4-[3,5-bis(trifluoromethyl)-1H-pyrazol-1-yl]phenyl]-2,2,3,3-tetramethylcyclopropane-carboxamide,

N-[4-[3,5-bis(trifluoromethyl)-1H-pyrazol-1-yl]phenyl]-2,2-dichloro-1-methylcyclopropanecarboxamide,

N-[4-[3,5-bis(trifluoromethyl)-1H-pyrazol-1-yl]phenyl]-2-oxo-6-pentyl-2H-pyran-3-carboxamide,

N-[4-[3,5-bis(trifluoromethyl)-1H-pyrazol-1-yl]phenyl]-3,5-difluorobenzenesulfonamide,

N-[4-[3,5-bis(trifluoromethyl)-1H-pyrazol-1-yl]phenyl]-1-cyclohexene-1-carboxamide,

N-[4-[3,5-bis(trifluoromethyl)-1H-pyrazol-1-yl]phenyl]-2-methylcyclopropanecarboxamide,

N-[4-[3,5-bis(trifluoromethyl)-1H-pyrazol-1-yl]phenyl]-5-(3,5-dichlorophenoxy)-2-furancarboxamide,

N-[4-[3,5-bis(trifluoromethyl)-1H-pyrazol-1-yl]phenyl]-1-methyl-2-cyclohexene-1-carboxamide,

N-[4-[3,5-bis(trifluoromethyl)-1H-pyrazol-1-yl]phenyl]-1-cyclopentene-1-carboxamide,

N-[4-[3,5-bis(trifluoromethyl)-1H-pyrazol-1-yl]phenyl]-3-methoxycyclohexanecarboxamide,

N-[4-[3,5-bis(trifluoromethyl)-1H-pyrazol-1-yl]phenyl]-2-butynamide,

ethyl 3-[[[[4-[3,5-bis(trifluoromethyl)-1H-pyrazol-1-yl]phenyl]aamino]carbonyl]-amino]benzoate,

N-[4-[3,5-bis(trifluoromethyl)-1H-pyrazol-1-yl]phenyl]-3-furancarboxamide,

N-[4-[3,5-bis(trifluoromethyl)-1H-pyrazol-1-yl]phenyl]-2-methyl-3-nitrobenzamide,

N-[4-[3,5-bis(trifluoromethyl)-1H-pyrazol-1-yl]phenyl]-N′-(3-cyanophenyl)urea,

N-[4-[3,5-bis(trifluoromethyl)-1H-pyrazol-1-yl]phenyl]-1-hydroxycyclopropanecarboxamide,

N-[4[3,5-bis(trifluoromethyl)-1H-pyrazol-1-yl]phenyl]cycloheptanecarboxamide,

N-[4-[3,5-bis(trifluoromethyl)-1H-pyrazol-1-yl]phenyl]-2-benzofurancarboxamide,

N-[4-[3,5-bis(trifluoromethyl)-1H-pyrazol-1-yl]phenyl]-5-fluoro-1H-indole-2-carboxamide,

(E)-N-[4-[3,5-bis(trifluoromethyl)-1H-pyrazol-1-yl]phenyl]-3-(2-chlorophenyl)-2-propenamide,

2-benzoyl-N-[4-[3,5-bis(trifluoromethyl)-1H-pyrazol-1-yl]phenyl]benzamide,

3a(S)-(3aα,4β,6aα)-N-[4-[3,5-bis(trifluoromethyl)-1H-pyrazol-1-yl]phenyl]hexahydro-2-oxo-1H-thieno[3,4-d]imidazole-4-pentanamide,

N-[4-[3,5-bis(trifluoromethyl)-1H-pyrazol-1-yl]phenyl]-3-iodobenzamide,

exo-N-[4-[3,5-bis(trifluoromethyl)-1H-pyrazol-1-yl]phenyl]bicyclo[2.2.1]hept-5-ene-2-carboxamide,

N-[4-[3,5-bis(trifluoromethyl)-1H-pyrazol-1-yl]phenyl]-2-methylcyclohexanecarboxamide,

phenylmethyl [1-[[[4-[3,5-bis(trifluoromethyl)-1H-pyrazol-1-yl]phenyl]amino]-carbonyl]propyl]carbamate,

N-[4-[3,5-bis(trifluoromethyl)-1H-pyrazol-1-yl]phenyl]-3-cyclohexene-1-carboxamide,

4-[3,5-bis(trifluoromethyl)-1H-pyrazol-1-yl]-N-(4-fluorophenyl)benzamide,

N-[4-[3,5-bis(trifluoromethyl)-1H-pyrazol-1-yl]phenyl]-N′-(3-nitropenyl)urea,

N-[4-[3,5-bis(trifluoromethyl)-1H-pyrazol-1-yl]phenyl]-N′-(4-fluorophenyl)urea,

N-[4-[3,5-bis(trifluoromethyl)-1H-pyrazol-1-yl]phenyl]-N′-[4-(trifluoromethyl)-phenyl]urea,

N-[4-[3,5-bis(trifluoromethyl)-1H-pyrazol-1-yl]phenyl]-N′-(3,5 -dimethylphenyl)urea,

N-[4-[3,5-bis(trifluoromethyl)-1H-pyrazol-1-yl]phenyl]-1-methylcyclopropanecarboxamide,

N-[4-[3,5-bis(trifluoromethyl)-1H-pyrazol-1-yl]phenyl]-N′-phenylurea,

N-[4-[3,5-bis(trifluoromethyl)-1H-pyrazol-1-yl]phenyl]-N′-(3-chloro-2-methylphenyl)urea,

N-[4-[3,5-bis(trifluoromethyl)-1H-pyrazol-1-yl]phenyl]-N′-[4-(butyloxyphenyl)urea,

N-[4-[3,5-bis(trifluoromethyl)-1H-pyrazol-1-yl]phenyl]-N′-(2-methyl-3-nitrophenyl)urea,

N-[4-[3,5-bis(trifluoromethyl)-1H-pyrazol-1-yl]phenyl]-N′-(2-chloro-4-nitrophenyl)urea,

N-(4-acetylphenyl)-N′-[4-[3,5-bis(trifluoromethyl)-1H-pyrazol-1-yl]phenyl]urea,

N-[4-[3,5-bis(trifluoromethyl)-1H-pyrazol-1-yl]phenyl]-n′-(4-methyl-2-nitrophenyl)urea,

N-[4-[3,5-bis(trifluoromethyl)-1H-pyrazol-1-yl]phenyl]-5-methyl-2-thiophene-carboxamide,

N-[4-[3,5-bis(trifluoromethyl)-1H-pyrazol-1-yl]phenyl]-N′-(4-bromo-2,6-dimethylphenyl)urea,

N-[4-[3,5-bis(trifluoromethyl)-1H-pyrazol-1-yl]phenyl]-4-(1H-pyrrol-1-yl)benzamide,

N-[4-[3,5-bis(trifluoromethyl)-1H-pyrazol-1-yl]phenyl]-N′-heptylurea,

N-[4-[3,5-bis(trifluoromethyl)-1H-pyrazol-1-yl]phenyl]-N′-(4-chloro-2-nitrophenyl)urea,

N-[4-[3,5-bis(trifluoromethyl)-1H-pyrazol-1-yl]phenyl]-7-methoxy-2-benzofurancarboxamide,

N-[4-[3,5-bis(trifluoromethyl)-1H-pyrazol-1-yl]phenyl]-N′-2-methyl-5-nitrophenyl)urea,

N-[4-[3,5-bis(trifluoromethyl)-1H-pyrazol-1-yl]phenyl]-3-(hydroxymethyl)benzamide,

N-[4-[3,5-bis(trifluoromethyl)-1H-pyrazol-1-yl]phenyl]-2-cyanoacetamide,

N-[4-[3,5-bis(trifluoromethyl)-1H-pyrazol-1-yl]phenyl]-2-cyclohexane-1-carboxamide,

N-[4-[3,5-bis(trifluoromethyl)-1H-pyrazol-1-yl]phenyl]-4-methylcyclohexanecarboxamide,

N-[4-[3,5-bis(trifluoromethyl)-1H-pyrazol-1-yl]phenyl]-α-methoxy-α-(trifluoromethyl)benzeneacetamide,

N-[4-[3,5-bis(trifluoromethyl)-1H-pyrazol-1-yl]phenyl]heptanamide,

N-[4-[3,5-bis(trifluoromethyl)-1H-pyrazol-1-yl]phenyl]-2-phenoxybenzamide,

3-amino-N-[4-[3,5-bis(trifluoromethyl)-1H-pyrazol-1-yl]phenyl]benzamide,

4-amino-N-[4-[3,5-bis(trifluoromethyl)-1H-pyrazol-1-yl]phenyl]benzamide,

4-azido-N-[4-[3,5-bis(trifluoromethyl)-1H-pyrazol-1-yl]phenyl]benzamide,

N-[4-[3,5-bis(trifluoromethyl)-1H-pyrazol-1-yl]-2-thiopheneacetamide,

N-[3,5-bis(trifluoromethyl)-1H-pyrazol-1-yl]phenyl]-1-tricyclo[3.3.1.1^{3,7}]decane-carboxmide,

N-[4-[3,5-bis(trifluoromethyl)-1H-pyrazol-1-yl]phenyl]-N²-[(1,1-dimethylethoxy)-carbonyl]-1-asparagine, phenylmethyl ester,

1,1-dimethylethyl [7-[[4-[3,5-bis(trifluoromethyl)-1H-pyrazol-1-yl]phenyl]amino]-7-oxoheptyl]carbamate,

N-[4-[3,5-bis(trifluoromethyl)-1H-pyrazol-1-yl]phenyl]-3-(methylthio)propanamide,

N-[4-[3,5-bis(trifluoromethyl)-1H-pyrazol-1-yl]phenyl]-1-naphthylenecarboxamide,

N-[4-[3,5-bis(trifluoromethyl)-1H-pyrazol-1-yl]phenyl]-4-cyanobenzamide,

N-[4-[3,5-bis(trifluoromethyl)-1H-pyrazol-1-yl]phenyl]-2-phenylcyclopropane-carboxamide,

N-[4-[3,5-bis(trifluoromethyl)-1H-pyrazol-1-yl]phenyl]-4-iodobenzamide,

N-[4-[3,5-bis(trifluoromethyl)-1H-pyrazol-1-yl]phenyl]-3-chloropropanamide,

N-[4-[3,5-bis(trifluoromethyl)-1H-pyrazol-1-yl]phenyl]-4-methoxybenzamide,

N-[4-[3,5-bis(trifluoromethyl)-1H-pyrazol-1-yl]phenyl]-2-ethylhexanamide,

N-[4-[3,5-bis(trifluoromethyl)-1H-pyrazol-1-yl]phenyl]-4-hydroxybenzamide,

N-[4-[3,5-bis(trifluoromethyl)-1H-pyrazol-1-yl]phenyl]-4-(hexyloxy)benzamide,

N-[4-[3,5-bis(trifluoromethyl)-1H-pyrazol-1-yl]phenyl]-3-methylbenzamide,

2-(acetyloxy)-N-[4-[3,5-bis(trifluoromethyl)-1H-pyrazol-1-yl]phenyl]benzamide,

N-[4-[3,5-bis(trifluoromethyl)-1H-pyrazol-1-yl]phenyl]-N′-(4-bromo-2-methylphenyl)urea,

N-[4-[3,5-bis(trifluoromethyl)-1H-pyrazol-1-yl]phenyl]-2,4,6-trimethylbenzamide,

N-[4-[3,5-bis(trifluoromethyl)-1H-pyrazol-1-yl]phenyl]-N′-(4-chloro-3-nitrophenyl)urea,

N-[4-[3,5-bis(trifluoromethyl)-1H-pyrazol-1-yl]phenyl]-2-chloro-N-methylbenzamide,

N-[4-[3,5-bis(trifluoromethyl)-1H-pyrazol-1-yl]phenyl]-2-chloro-4-nitro-N-methylbenzamide,

N-[4-[3,5-bis(trifluoromethyl)-1H-pyrazol-1-yl]phenyl]-2-chlorobenzenemethanamine,

N-[4-[3,5-bis(trifluoromethyl)-1H-pyrazol-1-yl]phenyl]-1-methyl-5-nitro-1H-pyrazole-4-carboxamide,

N-[4-[3,5-bis(trifluoromethyl)-1H-pyrazol-1-yl]phenyl]-4-fluorobenzenemethanamine,

N-[4-[3,5-bis(trifluoromethyl)-1H-pyrazol-1-yl]phenyl]-4-bromobenzamide,

N-[4-[3,5-bis(trifluoromethyl)-1H-pyrazol-1-yl]phenyl]-2-(dimethylamino)benzamide,

N-[4-[3,5-bis(trifluoromethyl)-1H-pyrazol-1-yl]phenyl]-3-(dimethylamino)benzamide,

N-[4-[3,5-bis(trifluoromethyl)-1H-pyrazol-1-yl]phenyl]-4-(trifluoromethyl)benzamide,

N-[4-[3,5-bis(trifluoromethyl)-1H-pyrazol-1-yl]phenyl]-4-fluorobenzamide,

N-[4-[3,5-bis(trifluoromethyl)-1H-pyrazol-1-yl]phenyl]-2-chlorobenzamide,

N-[4-[3,5-bis(trifluoromethyl)-1H-pyrazol-1-yl]phenyl]benzamide,

N-[4-[3,5-bis(trifluoromethyl)-1H-pyrazol-1-yl]phenyl]-4-nitrobenzamide, 4-[3,5-bis(trifluoromethyl)-1H-pyrazol-1-yl]-N-(4-fluorophenyl)benzenemethanamine,

3-[4-[[[3,5-bis(trifluoromethyl)-1H-pyrazol-1-yl]phenyl]methyl]amino]benzonitrile,

N-[4-[3,5-bis(trifluoromethyl)-1H-pyrazol-1-yl]phenyl]-2-methylbenzamide,

(E)-N-[4-[3,5-bis(trifluoromethyl)-1H-pyrazol-1-yl]phenylmethylene]-2,4-difluoro benzenamine,

N-[4-[3,5-bis(trifluoromethyl)-1H-pyrazol-1-yl]phenyl]-3-4-dimethoxybenzamide,

N-[4-[3,5-bis(trifluoromethyl)-1H-pyrazol-1-yl]phenyl]cyclopentanepropanamide,

N-[4-[3,5-bis(trifluoromethyl)-1H-pyrazol-1-yl]phenyl]-4-methylbenzamide,

N-[4-[3,5-bis(trifluoromethyl)-1H-pyrazol-1-yl]phenyl]-3-(trifluoromethyl)benzamide,

N-[4-[3,5-bis(trifluoromethyl)-1H-pyrazol-1-yl]phenyl]-3-methyl-2-butenamide,

N-[4-[3,5-bis(trifluoromethyl)-1H-pyrazol-1-yl]phenyl]-2-hydroxybenzamide,

N-[4-[3,5-bis(trifluoromethyl)-1H-pyrazol-1-yl]phenyl]-3-hydroxybenzamide,

N-[4-[3,5-bis(trifluoromethyl)-1H-pyrazol-1-yl]phenyl]-2,4-dimethyl-5-thiazolecarboxamide,

N-[4-[3,5-bis(trifluoromethyl)-1H-pyrazol-1-yl]phenyl]-3-pyridinecarboxamide,

N-[4-[3,5-bis(trifluoromethyl)-1H-pyrazol-1-yl]phenyl]-4-(hydroxymethyl)benzamide,

4-[3,5-bis(trifluoromethyl)-1H-pyrazol-1-yl]-N-(2,4-difluorophenyl)benzenemethanamine,

N-[4-[3,5-bis(trifluoromethyl)-1H-pyrazol-1-yl]phenyl]-4-(methylsulfonyl)benzamide,

N-[4-[3,5-bis(trifluoromethyl)-1H-pyrazol-1-yl]phenyl]-2-iodobenzamide,

N-[4-[3,5-bis(trifluoromethyl)-1H-pyrazol-1-yl]phenyl]-4-heptybenzamide,

N-[4-[3,5-bis(trifluoromethyl)-1H-pyrazol-1-yl]phenyl]-2-furancarboxamide,

N-[4-[3,5-bis(trifluoromethyl)-1H-pyrazol-1-yl]phenyl]-2-fluorobenzamide,

N-[4-[3,5-bis(trifluoromethyl)-1H-pyrazol-1-yl]phenyl]-N′-methyl-1,2-benzenedicarboxamide,

N-[4-[3,5-bis(trifluoromethyl)-1H-pyrazol-1-yl]phenyl]-4-pyridinecarboxamide,

N-[4-[3,5-bis(trifluoromethyl)-1H-pyrazol-1-yl]phenyl]-4-chloro-2-nitrobenzamide,

N-[4-[3,5-bis(trifluoromethyl)-1H-pyrazol-1-yl]phenyl]-4-cinnolinecarboxamide,

4-acetyl-N-[4-[3,5-bis(trifluoromethyl)-1H-pyrazol-1-yl]phenyl]benzamide,

1,1 -dimethylethyl 4-[[[4-[3,5-bis(trifluoromethyl)-1H-pyrazol-1-yl]phenyl]-amino]carbonyl]-1-piperidinecarboxylate,

N-[4-[3,5-bis(trifluoromethyl)-1H-pyrazol-1-yl]phenyl]-2-pyridinecarboxamide,

N-[4-[3,5-bis(trifluoromethyl)-1H-pyrazol-1-yl]phenyl]-4-(diethylamino)benzamide,

N-[4-[3,5-bis(trifluoromethyl)-1H-pyrazol-1-yl]phenyl]cyclopentanecarboxmide,

N-[4-[3,5-bis(trifluoromethyl)-1H-pyrazol-1-yl]phenyl]cyclohexanecarboxmide,

N-[4-[3,5-bis(trifluoromethyl)-1H-pyrazol-1-yl]phenyl]-4-piperidinecarboxamide,

N-[4-[3,5-bis(trifluoromethyl)-1H-pyrazol-1-yl]phenyl]-3-(methylsulfonyl)benzamide,

N-[4-[3,5-bis(trifluoromethyl)-1H-pyrazol-1-yl]phenyl]-2-(trifluoromethyl)benzamide,

3-[[[4-[3,5-bis(trifluoromethyl)-1H-pyrazol-1-yl]phenyl]methyl]amino]benzonitrile,

methyl 3-[[[4-[3,5-bis(trifluoromethyl)-1H-pyrazol-1-yl]phenyl]amino]carbonyl]benzoate,

N-[4-[3,5-bis(trifluoromethyl)-1H-pyrazol-1-yl]phenyl]-3-chlorobenzamide,

N-[4-[3,5-bis(trifluoromethyl)-1H-pyrazol-1-yl]phenyl]-2-thiophenecarboxamide,

(E)-3-[2-[4-[3,5-bis(trifluoromethyl)-1H-pyrazol-1-yl]phenyl]ethenyl]benzonitrile,

N-[4-[3,5-bis(trifluoromethyl)-1H-pyrazol-1-yl]phenyl]-1,4-benzenedicarboxamide,

N-[4-[3,5-bis(trifluoromethyl)-1H-pyrazol-1-yl]phenyl]-3,5-dinitrobenzamide,

N-[4-[3,5-bis(trifluoromethyl)-1H-pyrazol-1-yl]phenyl]-2,4-difluorobenzamide,

N-[4-[3,5-bis(trifluoromethyl)-1H-pyrazol-1-yl]phenyl]-2-nitrobenzamide,

N-[4-[3,5-bis(trifluoromethyl)-1H-pyrazol-1-yl]phenyl]-3-cyanobenzamide,

N-[4-[3,5-bis(trifluoromethyl)-1H-pyrazol-1-yl]phenyl]-1,3-benzenedicarboxamide,

(Z)-3-[2-[4-[3,5-bis(trifluoromethyl)-1H-pyrazol-1-yl]phenyl]ethenyl]benzonitrile,

N-[4-[3,5-bis(trifluoromethyl)-1H-pyrazol-1-yl]phenyl]-3-nitrobenzamide,

3-(aminosulfonyl)-N-[4-[3,5-bis(trifluoromethyl)-1H-pyrazol-1-yl]phenyl]benzamide,

methyl 4-[[[4-[3,5-bis(trifluoromethyl)-1H-pyrazol-1-yl]phenyl]amino]carbonyl]benzoate,

N-[4-[3,5-bis(trifluoromethyl)-1H-pyrazol-1-yl]phenyl]-2-methoxybenzamide,

N-[4-[3,5-bis(trifluoromethyl)-1H-pyrazol-1-yl]phenyl]-3-bromobenzamide,

N-[4-[3,5-bis(trifluoromethyl)-1H-pyrazol-1-yl]phenyl]-3-methoxybenzamide,

N-[4-[3,5-bis(trifluoromethyl)-1H-pyrazol-1-yl]phenyl]-3-fluorobenzamide,

N-[4-[3,5-bis(trifluoromethyl)-1H-pyrazol-1-yl]phenyl]-2-bromobenzamide,

N-[4-[3,5-bis(trifluoromethyl)-1H-pyrazol-1-yl]phenyl]-1,3-benzodioxole-5-carboxamide,

N-[4-[3,5-bis(trifluoromethyl)-1H-pyrazol-1-yl]phenyl]-2,6-dichloro-3-pyridinecarboxamide,

N-[4-[3,5-bis(trifluoromethyl)-1H-pyrazol-1-yl]phenyl]-2-chloro-3-pyridinecarboxamide,

N-[4-[3,5-bis(trifluoromethyl)-1H-pyrazol-1-yl]phenyl]-2-chloro-6-methyl-3-pyridinecarboxamide,

N-[4-[3,5-bis(trifluoromethyl)-1H-pyrazol-1-yl]phenyl]-4-fluoro-γ-oxobenzenebutanamide,

N-[4-[3,5-bis(trifluoromethyl)-1H-pyrazol-1-yl]phenyl]-1,2,3,4-tetrahydro-2-naphthalenecarboxamide,

(E)-1-[4-[2-(2-chlorophenyl)ethenyl]phenyl]-3,5-bis(trifluoromethyl)-1H-pyrazole,

N-[4-[3,5-bis(trifluoromethyl)-1H-pyrazol-1-yl]phenyl]-2-(4-chlorophenoxy)-2-methylpropanamide,

N-[4-[3,5-bis(trifluoromethyl)-1H-pyrazol-1-yl]phenyl]acetamide,

4-[[[4-[3,5-bis(trifluoromethyl)-1H-pyrazol-1-yl]phenyl]amino]carbonyl]benzoic acid,

phenylmethyl N-[4-[3,5-bis(trifluoromethyl)-1H-pyrazol-1-yl]phenyl]amino]-4-oxobutyl]carbamate,

3-[[[4-[3,5-bis(trifluoromethyl)-1H-pyrazol-1-yl]phenyl]amino]carbonyl]benzoic acid,

N-[4-[3,5-bis(trifluoromethyl)-1H-pyrazol-1-yl]phenyl]-2,6-difluorobenzamide,

N-[4-[3,5-bis(trifluoromethyl)-1H-pyrazol-1-yl]phenyl]-3-bromo-2-thiophenecarboxamide,

N-[4-[3,5-bis(trifluoromethyl)-1H-pyrazol-1-yl]phenyl]-3-methyl-2-thiophenecarboxamide,

2-amino-N-[4-[3,5-bis(trifluoromethyl)-1H-pyrazol-1-yl]phenyl]benzamide,

N-[4-[3,5-bis(trifluoromethyl)-1H-pyrazol-1-yl]phenyl]-2-fluoro-3-pyridinecarboxamide,

N-[4-[3,5-bis(trifluoromethyl)-1H-pyrazol-1-yl]phenyl]-3-chloro-4-(methylsulfonyl)-2-thiophenecarboxamide,

N-[4-[3,5-bis(trifluoromethyl)-1H-pyrazol-1-yl]phenyl]-1H-pyrrole-2-carboxamide,

N-[4-[3,5-bis(trifluoromethyl)-1H-pyrazol-1-yl]phenyl]-3,6-dichloro-2-pyridinecarboxamide

N-[4-[3,5-bis(trifluoromethyl)-1H-pyrazol-1-yl]phenyl]-2-(2-nitrophenoxy)acetamide,

N-[4-[3,5-bis(trifluoromethyl)-1H-pyrazol-1-yl]phenyl]-4-chlorobenzeneacetamide,

N-[4-[3,5-bis(trifluoromethyl)-1H-pyrazol-1-yl]phenyl]-1H-indole-2-acetamide,

(E)-N-[4-[3,5-bis(trifluoromethyl)-1H-pyrazol-1-yl]phenyl]-3-(2-thienyl)-2-propenamide,

N-[4-[3,5-bis(trifluoromethyl)-1H-pyrazol-1-yl]phenyl]pyazinecarboxamide,

1,1-dimethylethyl [[4-[4-[3,5-bis(trifluoromethyl)-1H-pyrazol-1-yl]phenyl]amino]-4-oxobutyl]carbamate,

1-acetyl-N-[4-[3,5-bis(trifluoromethyl)-1H-pyrazol-1-yl]phenyl]-4-piperidinecarboxamide,

N-[4-[3,5-bis(trifluoromethyl)-1H-pyrazol-1-yl]phenyl]butanamide,

N-[4-[3,5-bis(trifluoromethyl)-1H-pyrazol-1-yl]phenyl]-4-chloro-2-methoxybenzarnide,

N-[4-[3,5-bis(trifluoromethyl)-1H-pyrazol-1-yl]phenyl]-α-methyl-4-(2-thienyl-carbonyl)benzeneacetamide,

N-[4-[3,5-bis(trifluoromethyl)-1H-pyrazol-1-yl]phenyl]-α-methyl-4-(2-thienyl carbonyl)benzeneacetamide,

N-[4-[3,5-bis(trifluoromethyl)-1H-pyrazol-1-yl]phenyl]-2-methoxy-4-(methythio)benzamide,

N-[4-[3,5-bis(trifluoromethyl)-1H-pyrazol-1-yl]phenyl]-4-hydroxy-3-nitrobenzamide,

N-[4-[3,5-bis(trifluoromethyl)-1H-pyrazol-1-yl]phenyl]-3,4-dihydroxybenzamide,

N-[4-[3,5-bis(trifluoromethyl)-1H-pyrazol-1-yl]phenyl]-2-hydroxy-6-methoxybenzamide,

N-[4-[3,5-bis(trifluoromethyl)-1H-pyrazol-1-yl]phenyl]-2,4-bis(trifluoromethyl)benzamide,

N-[4-[3,5-bis(trifluoromethyl)-1H-pyrazol-1-yl]phenyl]-5-methyl-4-isoxazolecarboxamide,

4-[3,5-bis(trifluoromethyl)-1H-pyrazol-1-yl]-N-(2-chlorophenyl)benzamide,

4-[3,5-bis(trifluoromethyl)-1H-pyrazol-1-yl]-N-(3-cyanophenyl)benzamide,

4-[3,5-bis(trifluoromethyl)-1H-pyrazol-1-yl]-N-(2,4-difluorophenyl)benzamide,

4-[3,5-bis(trifluoromethyl)-1H-pyrazol-1-yl]-N-(2-cyanophenyl)benzamide,

N-[4-[5-[3,5-dimethyl-1H-1,2,4-triazol-1-yl)-3-(trifluoromethyl)-1H-pyrazol-1-yl]phenyl]-4-isoxazolecarboxamide,

4-[3,5-bis(trifluoromethyl)-1H-pyrazol-1-yl]-N-(2-nitrophenyl)benzamide,

4-[3,5-bis(trifluoromethyl)-1H-pyrazol-1-yl]-N-(2,6-difluorophenyl)benzamide,

4-[3,5-bis(trifluoromethyl)-1H-pyrazol-1-yl]-N-(2-bromophenyl)benzamide,

4-[3,5-bis(trifluoromethyl)-1H-pyrazol-1-yl]-N-(4-cyanophenyl)benzamide,

4-[3,5-bis(trifluoromethyl)-1H-pyrazol-1-yl]-N-(4-pyridinyl)benzamide,

N-[4-[3,5-bis(trifluoromethyl)-1H-pyrazol-1-yl]phenyl]-4-fluoro-3-(trifluoromethyl)benzamide,

N-[2-(aminocarbonyl)phenyl]-4-[3,5-bis(trifluoromethyl)-1H-pyrazol-1-yl]benzamide,

N-[3-[3,5-bis(trifluoromethyl)-1H-pyrazol-1-yl]phenyl]-4-chlorobenzeneacetamide,

N-[3-[3,5-bis(trifluoromethyl)-1H-pyrazol-1-yl]phenyl]-2,3-dichlorobenzamide,

N-[3-[3,5-bis(trifluoromethyl)-1H-pyrazol-1-yl]phenyl]-2-chloro-5-nitrobenzamide,

N-[4-[3,5-bis(trifluoromethyl)-1H-pyrazol-1-yl]phenyl]-4-fluoro-3-nitrobenzamide,

N-[4-[3,5-bis(trifluoromethyl)-1H-pyrazol-1-yl]phenyl]-4-fluoro-3-nitrobenzamide,

N-[4-[3,5-bis(trifluoromethyl)-1H-pyrazol-1-yl]phenyl]-4-fluoro-2-(trifluoromethyl)-benzamide,

N-[3-[3,5-bis(trifluoromethyl)-1H-pyrazol-1-yl]phenyl]-4-fluorobenzamide,

N-[3-[3,5-bis(trifluoromethyl)-1H-pyrazol-1-yl]phenyl]-2,4-difluorobenzamide,

N-[3-[3,5-bis(trifluoromethyl)-1H-pyrazol-1-yl]phenyl]-3-cyanobenzamide,

N-[4-[3,5-bis(trifluoromethyl)-1H-pyrazol-1-yl]phenyl]-2-bromo-3-nitrobenzamide,

N-[4-[3,5-bis(trifluoromethyl)-1H-pyrazol-1-yl]phenyl]-2-chloro-4-fluorobenzamide,

N-[4-[3,5-bis(trifluoromethyl)-1H-pyrazol-1-yl]phenyl]-2-chloro-4-(methylsulfonyl)-benzamide,

N-[4-[3,5-bis(trifluoromethyl)-1H-pyrazol-1-yl]phenyl]-2,5-dichlorobenzamide,

N-[4-[3,5-bis(trifluoromethyl)-1H-pyrazol-1-yl]phenyl]-2,3-difluorobenzamide,

N-[4-[3,5-bis(trifluoromethyl)-1H-pyrazol-1-yl]phenyl]-3-chloro-4-fluorobenzamide,

N-[4-[3,5-bis(trifluoromethyl)-1H-pyrazol-1-yl]phenyl]-2,5-difluorobenzamide,

N-[4-[3,5-bis(trifluoromethyl)-1H-pyrazol-1-yl]phenyl]-2-chloro-6-fluorobenzamide,

N-[4-[3,5-bis(trifluoromethyl)-1H-pyrazol-1-yl]phenyl]-2-fluoro-6-(trifluoromethyl)-benzamide,

N-[4-[3,5-bis(trifluoromethyl)-1H-pyrazol-1-yl]phenyl]-3-chloro-2-fluorobenzamide,

N-[4-[3,5-bis(trifluoromethyl)-1H-pyrazol-1-yl]phenyl]-2-chloro-4-methoxybenzamide,

N-[4-[3,5-bis(trifluoromethyl)-1H-1-pyrazol-1-yl]phenyl]-2,6-dichloro-3-nitrobenzamide,

N-[4-[3,5-bis(trifluoromethyl)-1H-pyrazol-1-yl]phenyl]-4-bromo-2-chlorobenzamide,

N-[4-[3,5-bis(trifluoromethyl)-1H-pyrazol-1-yl]phenyl]-3-4-difluorobenzamide,

N-[4-[3,5-bis(trifluoromethyl)-1H-pyrazol-1-yl]phenyl]-2-bromo-5-methoxybenzamide,

N-[4-[3,5-bis(trifluoromethyl)-1H-pyrazol-1-yl]phenyl]-4-chloro-2-hydroxybenzamide,

N-[4-[3,5-bis(trifluoromethyl)-1H-pyrazol-1-yl]phenyl]-3-bromo-4-methoxybenzamide,

N-[4-[3,5-bis(trifluoromethyl)-1H-pyrazol-1-yl]phenyl]-3-bromo-4-hydroxybenzamide,

N-[4-[3,5-bis(trifluoromethyl)-1H-pyrazol-1-yl]phenyl]-2-chloro-4,5-difluorobenzamide,

N-[3-[3,5-bis(trifluoromethyl)-1H-pyrazol-1-yl]phenyl]-2,6-difluorobenzamide,

N-[4-[3,5-bis(trifluoromethyl)-1H-pyrazol-1-yl]phenyl]-4-chloro-2,5-difluorobenzamide,

N-[4-[3,5-bis(trifluoromethyl)-1H-pyrazol-1-yl]phenyl]-2,3,4-trifluorobenzamide,

N-[4-[3,5-bis(trifluoromethyl)-1H-pyrazol-1-yl]phenyl]-3,4,5-trifluorobenzamide,

N-[4-[3,5-bis(trifluoromethyl)-1H-pyrazol-1-yl]phenyl]-2,4,5-trifluorobenzamide,

N-[4-[3,5-bis(trifluoromethyl)-1H-pyrazol-1-yl]phenyl]-2,4,6-trifluorobenzamide,

N-[4-[3,5-bis(trifluoromethyl)-1H-pyrazol-1-yl]phenyl]-2,6-difluoro-3-nitrobenzamide,

N-[4-[3,5-bis(trifluoromethyl)-1H-pyrazol-1-yl]phenyl]-2,3,5-trifluorobenzamide,

N-[4-[3,5-bis(trifluoromethyl)-1H-pyrazol-1-yl]phenyl]-2,4-dichloro-6-fluorobenzamide,

N-4-[3,5-bis(trifluoromethyl)-1H-pyrazol-1-yl]phenyl-2,4-dichloro-3,5-dinitrobenzamide,

N-[4-[3,5-bis(trifluoromethyl)-1H-pyrazol-1-yl]phenyl]-2,3,5,6-tetrafluorobenzamide,

N-[4-[3,5-bis(trifluoromethyl)-1H-pyrazol-1-yl]phenyl]-2,3,4,5-tetrafluorobenzamide,

N-[4-[3,5-bis(trifluoromethyl)-1H-pyrazol-1-yl]phenyl]-4-bromo-2,3,5,6-tetrafluorobenzamide,

N-[4-[3,5-bis(trifluoromethyl)-1H-pyrazol-1-yl]phenyl]-5-methyl-2-nitrobenzamide,

N-[3-[3,5-bis(trifluoromethyl)-1H-pyrazol-1-yl]phenyl]-4-cyanobenzamide,

N-[4-[3,5-bis(trifluoromethyl)-1H-pyrazol-1-yl]phenyl]-3-thiophenecarboxamide,

N-[4-[3,5-bis(trifluoromethyl)-1H-pyrazol-1-yl]phenyl]-5-isoxazolecarboxamide,

N-[4-[3,5-bis(trifluoromethyl)-1H-pyrazol-1-yl]phenyl]tetrahydro-2-furancarboxamide,

N-[4-[3,5-bis(trifluoromethyl)-1H-pyrazol-1-yl]phenyl]-2-pyrrolidinecarboxamide,

N-[4-[3,5-bis(trifluoromethyl)-1H-pyrazol-1-yl]phenyl]tetrahydro-3-furancarboxamide,

N-[4-[3,5-bis(trifluoromethyl)-1H-pyrazol-1-yl]phenyl]-1,2,3-thiadiazole-5-carboxamide,

N-[4-[3,5-bis(trifluoromethyl)-1H-pyrazol-1-yl]phenyl]-2-chloro-4-pyridinecarboxamide,

1,1-dimethylethyl 2-[[[4-[3,5-bis(trifluoromethyl)-1H-pyrazol-1-yl]phenyl]amino]carbonyl]-1-pyrrolidinecarboxylate,

N-[4-[3,5-bis(trifluoromethyl)-1H-pyrazol-1-yl]phenyl]-5-nitro-2-furancarboxamide,

N-[4-[3,5-bis(trifluoromethyl)-1H-pyrazol-1-yl]phenyl]-1-methyl-1H-pyrrole-2-carboxamide,

N-[4-[3,5-bis(trifluoromethyl)-1H-pyrazol-1-yl]phenyl]-6-chloro-3-pyridinecarboxamide,

N-[4-[3,5-bis(trifluoromethyl)-1H-pyrazol-1-yl]phenyl]-5-bromo-2-furancarboxamide,

N-[4-[3,5-bis(trifluoromethyl)-1H-pyrazol-1-yl]phenyl]-3-methyl-2-furancarboxamide,

N-[4-[3,5-bis(trifluoromethyl)-1H-pyrazol-1-yl]phenyl]-5-chloro-2-thiophenecarboxamide,

N-[4-[3,5-bis(trifluoromethyl)-1H-pyrazol-1-yl]phenyl]tetrahydro-5-oxo-2-furancarboxamide,

N-[4-[3,5-bis(trifluoromethyl)-1H-pyrazol-1-yl]phenyl]-5-oxo-2-pyrrolidinecarboxamide,

N-[4-[3,5-bis(trifluoromethyl)-1H-pyrazol-1-yl]phenyl]-5-bromo-3-pyridinecarboxamide,

N-[4-[3,5-bis(trifluoromethyl)-1H-pyrazol-1-yl]phenyl]-5-nitro-3-thiophenecarboxamide,

1,1-dimethylethyl-4-[[[4-[3,5-bis(trifluoromethyl)-1H-pyrazol-1-yl]phenyl]amino]-carbonyl]-3-thiazolidinecarboxylate,

N-[4-[3,5-bis(trifluoromethyl)-1H-pyrazol-1-yl]phenyl]-5-methoxy-3-thiophenecarboxamide,

N-[2-[3,5-bis(trifluoromethyl)-1H-pyrazol-1-yl]-5-pyridinyl]-2-chlorobenzamide,

N-[2-[3,5-bis(trifluoromethyl)-1H-pyrazol-1-yl]-5-pyridinyl]-3-cyanobenzamide,

N-[2-[3,5-bis(trifluoromethyl)-1H-pyrazol-1-yl]-5-pyridinyl]-2-chloro-4,5-difluorobenzamide,

N-[4-[3,5-bis(trifluoromethyl)-1H-pyrazol-1-yl]phenyl]-2,3-dibromo-5-thiophenecarboxamide,

N-[4-[3,5-bis(trifluoromethyl)-1H-pyrazol-1-yl]phenyl]-3-fluoro-4-pyridinecarboxamide,

N-[4-[3,5-bis(trifluoromethyl)-1H-pyrazol-1-yl]phenyl]-1-methyl-1H-pyrazole-4-carboxamide,

N-[4-[3,5-bis(trifluoromethyl)-1H-pyrazol-1-yl]phenyl]-5-chloro-4-methoxy-3-thiophenecarboxamide,

N-[4-[3,5-bis(trifluoromethyl)-1H-pyrazol-1-yl]phenyl]-5,6-dichloro-3-pyridinecarboxamide

N-[4-[3,5-bis(trifluoromethyl)-1H-pyrazol-1-yl]phenyl]-2,6-dichloro-4-pyridinecarboxamide,

N-[4-[3,5-bis(trifluoromethyl)-1H-pyrazol-1-yl]phenyl]-2,5-dichloro-3-pyridinecarboxamide,

N-[4-[3,5-bis(trifluoromethyl)-1H-pyrazol-1-yl]-3-(trifluoromethyl)phenyl]-4-chlorobenzamide,

N-[4-[3,5-bis(trifluoromethyl)-1H-pyrazol-1-yl]-2-fluorophenyl]-2,4-difluorobenzamide,

N-[2,4-bis[3,5-bis(trifluoromethyl)-1H-pyrazol-1-yl]phenyl]-2,4-difluorobenzamide,

methyl 2-[3,5-bis(trifluoromethyl)-1H-pyrazol-1-yl]-5-[(5-bromo-2-chlorobenzoyl)amino]benzoate,

N-[4-[3,5-bis(trifluoromethyl)-1H-pyrazol-1-yl]-3-(trifluoromethyl)phenyl]-3,5-dimethyl-4-isoxazolecarboxamide,

N-[4-[3,5-bis(trifluoromethyl)-1H-pyrazol-1-yl]-3-(trifluoromethyl)phenyl]-4-methyl-1,2,3-thiadiazole-5-carboxamide,

N-[4-[3,5-bis(trifluoromethyl)-1H-pyrazol-1-yl]-3-chlorophenyl]-3,5-dimethyl-4-isoxazolecarboxamide,

N-[4-[3,5-bis(trifluoromethyl)-1H-pyrazol-1-yl]-2-(trifluoromethyl)phenyl]-3,5-dimethyl-4-isoxazolecarboxamide,

N-[4-[3,5-bis(trifluoromethyl)-1H-pyrazol-1-yl]-2-methylphenyl]-4-methyl-1,2,3-thiadiazole-5-carboxamide,

N-[4-[3,5-bis(trifluoromethyl)-1H-pyrazol-1-yl]-2-methoxyphenyl]-4-methyl-1,2,3-thiadiazole-5-carboxamide,

4-chloro-N-[4-[5-methyl-3-(trifluoromethyl)-1H-pyrazol-1-yl]phenyl]benzamide,

4-methyl-N-[4-[5-methyl-3-(trifluoromethyl)-1H-pyrazol-1-yl]phenyl]-1,2,3-thiadiazole-5-carboxamide,

3,5-dimethyl-N-[4-[5-methyl-3-(trifluoromethyl)-1H-pyrazol-1-yl]phenyl]-4-isoxazolecarboxamide,

4-chloro-N-[4-(5-methyl-1H-pyrazol-1-yl)phenyl]benzamide,

4-methyl-N-[4-(5-methyl-1H-pyrazol-1-yl)phenyl]-1,2,3-thiadiazole-5-carboxamide,

3,5-dimethyl-N-[4-(5-methyl-1H-pyrazol-1-yl)phenyl]-4-isoxazolecarboxamide,

3,5-dimethyl-N-[4-[3-(trifluoromethyl)-1H-pyrazol-1-yl)phenyl]-4-isoxazolecarboxamide,

N-[4-[5-hydroxy-3-(trifluoromethyl)-1H-pyrazol-1-yl]phenyl]-4-methyl-1,2,3-thiadiazole-5-carboxamide,

N-[4-[5-(3,5-dimethyl-1H-1,2,4-triazol-1-yl)-3-(trifluoromethyl)-1H-pyrazol-1-yl]phenyl]-1,2,3-thiadiazole-5-carboxamide,

3-amino-N-(4-(3,5-bis(trifluoromethyl)-1H-pyrazol-1-yl)phenyl)isonicotinamide,

N-(4-(3,5-bis(trifluoromethyl)-1H-pyrazol-1-yl)phenyl)-3-chloro-5-methoxyisonicotinamide,

N-(6-(3,5-bis(trifluoromethyl)-1H-pyrazol-1-yl)-3-pyridinyl)-2-fluorobenzamide,

methyl 2-(3,5-bis(trifluoromethyl)-1H-pyrazol-1-yl)-5-((2-fluorobenzoyl)amino)benzoate,

4-(aminomethyl)-N-(4-(3,5-bis(trifluoromethyl)-1H-pyrazol-1-yl)phenyl)-2-chlorobenzamide,

N-(4-(3,5-bis(trifluoromethyl)-1H-pyrazol-1-yl)phenyl)-2-methylacrylamide,

N-(4-(3,5-bis(trifluoromethyl)-1H-pyrazol-1-yl)phenyl)-4-chloro-2-fluorobenzamide,

N-(5-(3,5-bis(trifluoromethyl)-1H-pyrazol-1-yl)-2-pyridinyl)-2-fluorobenzamide,

N-(3-amino-4-(3,5-bis(trifluoromethyl)-1H-pyrazol-1-yl)phenyl)-2-fluorobenzamide,

N-(4-(3,5-bis(trifluoromethyl)-1H-pyrazol-1-yl)-3-cyanophenyl)-2-fluorobenzamide,

N-(4-(5-cyano-3-(trifluoromethyl)-1H-pyrazol-1-yl)phenyl)-2-fluorobenzamide,

2-fluoro-N-(4-(5-(2-furyl)-3-(trifluoromethyl)-1H-pyrazol-1-yl)phenyl)benzamide,

N-(4-(5-cyano-3-(trifluoromethyl)-1H-pyrazol-1-yl)phenyl)-4-methyl-1,2,3-thiadiazole-5-carboxamide,

N-(4-(5-cyano-3-(trifluoromethyl)-1H-pyrazol-1-yl)phenyl)isonicotinamide,

N-(4-(5-cyano-3-(trifluoromethyl)-1H-pyrazol-1-yl)phenyl)-3-fluoroisonicotinamide,

N-(4-(5-acetyl-3-(trifluoromethyl)-1H-pyrazol-1-yl)phenyl)-2-fluorobenzamide,

N-(4-(5-cyano-3-(trifluoromethyl)-1H-pyrazol-1-yl)phenyl)-2-fluoronicotinamide,

N-(4-(5-cyano-3-(trifluoromethyl)-1H-pyrazol-1-yl)phenyl)-2,3,5-trifluorobenzamide,

2-fluoro-N-(4-(5-(2-thienyl)-3-(trifluoromethyl)-1H-pyrazol-1-yl)phenyl)benzamide,

2-fluoro-N-(4-(5-(methylsulfanyl)-3-(trifluoromethyl)-1H-pyrazol-1-yl)phenyl)benzamide,

2-fluoro-N-(4-(5-(3-pyridinyl)-3-(trifluoromethyl)-1H-pyrazol-1-yl)phenyl)benzamide,

3-fluoro-N-(4-(5-(2-thienyl)-3-(trifluoromethyl)-1H-pyrazol-1-yl)phenyl)isonicotinamide,

N-(4-(5-methoxy-3-(trifluoromethyl)-1H-pyrazol-1-yl)phenyl)isonicotinamide,

2-fluoro-N-(4-(5-methoxy-3-(trifluoromethyl)-1H-pyrazol-1-yl)phenyl)benzamide,

N-(4-(5-methoxy-3-(trifluoromethyl)-1H-pyrazol-1-yl)phenyl)-4-methyl-1,2,3-thiadiazole-5-carboxamide,

N-(4-(5-acetyl-3-(trifluoromethyl)-1H-pyrazol-1-yl)phenyl)-2-fluoronicotinamide,

2-fluoro-N-(4-(5-(methylsulfanyl)-3-(trifluoromethyl)-1H-pyrazol-1-yl)phenyl)nicotinamide,

2-fluoro-N-(4-(5-methoxy-3-(trifluoromethyl)-1H-pyrazol-1-yl)phenyl)nicotinamide,

N-(4-(5-ethoxy-3-(trifluoromethyl)-1H-pyrazol-1-yl)phenyl)-2-fluoronicotinamide,

3-fluoro-N-(4-(5-(methylsulfanyl)-3-(trifluoromethyl)-1H-pyrazol-1-yl)phenyl)isonicotinamide,

3-fluoro-N-(4-(5-methoxy-3-(trifluoromethyl)-1H-pyrazol-1-yl)phenyl)isonicotinamide,

N-(4-(5-(difluoromethoxy)-3-(trifluoromethyl)-1H-pyrazol-1-yl)phenyl)isonicotinamide,

N-(4-(5-(difluoromethoxy)-3-(trifluoromethyl)-1H-pyrazol-1-yl)phenyl)-4-methyl-1,2,3-thiadiazole-5-carboxamide,

N-(4-(5-(difluoromethoxy)-3-(trifluoromethyl)-1H-pyrazol-1-yl)phenyl)-2-fluoronicotinamide,

N-(4-(5-chloro-3-(trifluoromethyl)-1H-pyrazol-1-yl)phenyl)-2-fluoronicotinamide,

2-fluoro-N-(4-(5-nitro-3-(trifluoromethyl)-1H-pyrazol-1-yl)phenyl)benzamide,

N-(4-(5-(difluoromethoxy)-3-(trifluoromethyl)-1H-pyrazol-1-yl)phenyl)-3-fluoroisonicotinamiide,

N-(4-(5-chloro-3-(trifluoromethyl)-1H-pyrazol-1-yl)phenyl)-3-fluoroisonicotinamide,

N-(4-(5-bromo-3-(trifluoromethyl)-1H-pyrazol-1-yl)phenyl)-3-fluoroisonicotinamide,

3-fluoro-N-(4-(5-nitro-3-(trifluoromethyl)-1H-pyrazol-1-yl)phenyl)isonicotinamide,

N-(4-(5-bromo-3-(trifluoromethyl)-1H-pyrazol-1-yl)phenyl)-4-methyl-1,2,3-thiadiazole-5-carboxamide,

N-(4-(5-chloro-3-(trifluoromethyl)-1H-1,2,4-triazol-1-yl)phenyl)-3-fluoroisonicotinamide,

3-fluoro-N-(4-(5-(1-methyl-1H-pyrrol-3-yl)-3-(trifluoromethyl)-1H-pyrazol-1-yl)phenyl)isonicotinamide,

3-chloro-N-(4-(5-chloro-3-(trifluoromethyl)-1H-pyrazol-1-yl)phenyl)isonicotinamide,

N-(4-(5-bromo-3-(trifluoromethyl)-1H-pyrazol-1-yl)phenyl)-2,3-difluorobenzamide,

N-(4-(5-bromo-3-(trifluoromethyl)-1H-pyrazol-1-yl)phenyl)-3-chloroisonicotinamide,

2-chloro-N-(4-(5-cyano-3-(trifluoromethyl)-1H-pyrazol-1-yl)phenyl)benzamide,

3-chloro-N-(4-(5-cyano-3-(trifluoromethyl)-1H-pyrazol-1-yl)phenyl)isonicotinamide,

N-(4-(5-(difluoromethoxy)-3-(trifluoromethyl)-1H-pyrazol-1-yl)phenyl)-2-fluorobenzamide,

2-chloro-N-(4-(5-(difluoromethoxy)-3-(trifluoromethyl)-1H-pyrazol-1-yl)phenyl)benzamide,

N-(4-(5-(difluoromethoxy)-3-(trifluoromethyl)-1H-pyrazol-1-yl)phenyl)-2,3-difluorobenzamide,

3-fluoro-N-(4-(3-(4-pyridinyl)-5-(trifluoromethyl)-1H-pyrazol-1-yl)phenyl)isonicotinamide, N-(4-(5-(difluoromethyl)-3-(3 -pyridinyl)-1H-pyrazol-1-yl)phenyl)-3-fluoroisonicotinamide,

N-(4-(5-cyano-3-(2-pyridinyl)-1H-pyrazol-1-yl)phenyl)-3-fluoroisonicotinamide,

N-(4-(5-cyano-3-(3-pyridinyl)-1H-pyrazol-1-yl)phenyl)-4-methyl-1,2,3-thiadiazole-5-carboxamide,

3-fluoro-N-(4-(5-nitro-3-(3-pyridinyl)-1H-pyrazol-1-yl)phenyl)isonicotinamide,

4-methyl-N-(4-(5-nitro-3-(3-pyridinyl)-1H-pyrazol-1-yl)phenyl)-1,2,3-thiadiazole-5-carboxamide,

N-(4-(5-cyano-3-(1,3-thiazol-2-yl)-1H-pyrazol-1-yl)phenyl)-3-fluoroisonicotinamide,

N-(4-(3-(5-bromo-3-pyridinyl)-5-(difluoromethoxy)-1H-pyrazol-1-yl)phenyl)-3-fluoroisonicotinamide,

N-(4-(5-cyano-3-(1,3-thiazol-2-yl)-1H-pyrazol-1-yl)phenyl)-4-methyl-1,2,3-thiadiazole-5-carboxamide,

N-(4-(5-cyano-3-(1,3-thiazol-2-yl)-1H-pyrazol-1-yl)phenyl)-3-fluoroisonicotinamide,

4-methyl-N-(4-(3-(1,3-thiazol-2-yl)-5-(trifluoromethyl)-1H-pyrazol-1-yl)phenyl)-1,2,3-thiadiazole-5-carboxamide,

N-(4-(3-(2,4-dimethyl-1,3-thiazol-5-yl)-5-(trifluoromethyl)-1H-pyrazol-1-yl)phenyl)-3-fluoroisonicotinamide,

3-fluoro-N-(4-(3-tetrahydro-2-furanyl-5-(trifluoromethyl)-1H-pyrazol-1-yl)phenyl)isonicotinamide,

N-(4-(5-chloro-3-(1,3-thiazol-2-yl)-1H-pyrazol-1-yl)phenyl)-3-fluoroisonicotinamide,

N-(4-(5-chloro-3-(3-furyl)-1H-pyrazol-1-yl)phenyl)-4-methyl-1,2,3-thiadiazole-5-carboxamide,

N-(4-(5-chloro-3-(3-furyl)-1H-pyrazol-1-yl)phenyl)-3-fluoroisonicotinamide,

N-(4-(5-cyano-3-tetrahydro-2-furanyl-1H-pyrazol-1-yl)phenyl)-3-fluoroisonicotinamide,

N-(4-(5-(difluoromethoxy)-3-(1-methyl-1H-pyrrol-3-yl)-1H-pyrazol-1-yl)phenyl)-3-fluoroisonicotinamide,

N-(4-(5-(difluoromethoxy)-3-(3-furyl)-1H-pyrazol-1-yl)phenyl)-3-fluoroisonicotinamide, and

N-(4-(5-(difluoromethoxy)-3-(1-methyl-1H-pyrrol-2-yl)-1H-pyrazol-1-yl)phenyl)-3-fluoroisonicotinamide.

### DETAILED DESCRIPTION OF THE INVENTION

### Definition of Terms

The term “alkanoyl” refers to an alkyl group attached to the parent molecular group through a carbonyl group.

The term “alkanoyloxy” refers to an alkanoyl group attached to the parent molecular group through an oxygen atom.

The term “alkenyl” refers to a monovalent straight or branched chain group derived from a hydrocarbon of two to fifteen carbons having at least one carbon-carbon double bond. The alkenyl groups of this invention can be optionally substituted.

The term “alkenylene” refers to a divalent straight or branched chain group derived from a hydrocarbon of two to fifteen carbons having at least one carbon-carbon double bond.

The term “alkoxy” refers to an alkyl group attached to the parent molecular group through an oxygen atom.

The term “alkyl” refers to a monovalent straight or branched chain group derived from an saturated hydrocarbon of one to fifteen carbons. The alkyl groups of this invention can be optionally substituted.

The term “alkylene” refers to a divalent group derived from a straight or branched chain saturated hydrocarbon of one to fifteen carbons.

The term “alkynyl” refers to a monovalent straight or branched chain group derived from a hydrocarbon of one to fifteen carbons having at least one carbon-carbon triple bond. The alkynyl groups of this invention can be optionally substituted.

The term “alkynylene” refers to a divalent group derived from a straight or branched chain hydrocarbon of one to fifteen carbons having at least one carbon-carbon triple bond.

The term “amino” refers to —NH₂.

The term “amino protecting group” refers to groups intended to protect an amino group against undersirable reactions during synthetic procedures. Commonly used amino protecting groups are disclosed in Greene, “Protective Groups In Organic Synthesis,” (John Wiley & Sons, New York (1981)), which is hereby incorporated by reference. Preferred N-protecting groups are formyl, acetyl, benzoyl, pivaloyl, t-butylacetyl, phenylsulfonyl, benzyl, t-butyloxycarbonyl (Boc), benzyloxycarbonyl (Cbz), and allylcarbonyloxy (Alloc).

The term “aryl” refers to a mono- or bicyclic carbocyclic ring system having at least one aromatic ring that can be optionally substituted. The aryl group can be fused to a cyclohexane, cyclohexene, cyclopentane or cyclopentene ring in which case the aryl group can be attached through the ring to which it is attached or through the aromatic ring itself.

The term “carboxy protecting group” refers to a carboxylic acid protecting ester or amide group typically employed to block or protect the carboxylic acid functionality while the reactions involving other functional sites of the compound are performed. Carboxy protecting groups are disclosed in Greene, “Protective Groups in Organic Synthesis”. Additionally, a carboxy protecting group can be used as a prodrug whereby the carboxy protecting group can be readily cleaved in vivo, for example by enzymatic hydrolysis, to release the biologically active parent. Such carboxy protecting groups are well-known to those skilled in the art, having been extensively used in the protection of carboxyl groups in the penicillin and cephalosporin fields as described in U.S. Pat. Nos. 3,840,556 and 3,719,667 which are hereby incorporated by reference.

The term “cycloalkenyl” refers to a monovalent cyclic or bicyclic hydrocarbon of three to fifteen carbons having at least one carbon-carbon double bond. The cycloalkenyl groups of this invention can be optionally substituted.

The term “cycloalkyl” refers to a monovalent saturated cyclic or bicyclic hydrocarbon of three to fifteen carbons. The cycloalkyl groups of this invention can be optionally substituted.

The term “halo” refers to F, Cl, Br, or I.

The terms “heterocycle,” or “heterocyclic” refer to a 4-, 5-, 6- or 7-membered ring containing one, two or three heteroatoms independently selected from the group consisting of nitrogen, oxygen and sulfur. The 4- and 5-membered rings have 0, 1, or 2 double bonds and the 6- and 7-membered rings have 0, 1, 2, or 3 double bonds. The nitrogen and sulfur atoms can be optionally oxidized, and the nitrogen atom can be optionally quaternized. The term “heterocycle”60 also includes bicyclic, tricyclic, and tetracyclic groups in which a heterocyclic ring is fused to one or two rings selected from an aryl ring, a cyclohexane ring, a cyclohexene ring, a cyclopentane ring, a cyclopentene ring or another monocyclic heterocyclic ring. Heterocycles of this type can be attached through the ring to which they are fused or through the heterocyclic ring itself. Heterocycles include, but are not limited to, acridinyl, benzirnidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolinyl, pyrrolyl, quinolinyl, quinoxaloyl, tetrahydrofuryl, tetrahydroisoquinolyl, tetrahydroquinolyl, tetrazolyl, thiadiazolyl, thiazolidinyl, thiazolyl, thienyl, thiomorpholinyl, triazolyl, and the like.

Heterocyclics also include bridged bicyclic groups where a monocyclic heterocyclic group is bridged by an alkylene group such as

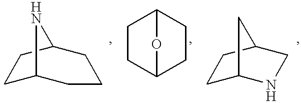

and the like.

Heterocyclics also include compounds of the formula

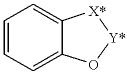

where X* is selected from —CH₂—, —CH₂O— and —O—, and Y* is selected from —C(O)— and —(C(R″)₂)ᵥ—, where R″ is hydrogen or alkyl of one to four carbons and v is 1, 2, or 3. The heterocycles of this invention can be optionally substituted.

The term “hydroxyl” refers to —OH.

The term “hydroxyl protecting group” refers to a protecting ester or ether group typically employed to block or protect the hydroxyl group while reactions involving other functional sites of the compound are performed. Hydroxyl protecting groups are disclosed in Greene, “Protective Groups in Organic Synthesis,” (John Wiley & Sons, New York (1981)).

Ther term “perfluoroalkyl” refers to an alkyl group wherein all of the hydrogens have been substituted with fluorides.

The term “pharmaceutically acceptable prodrugs” refers to those prodrugs of the compounds of the present invention which are, within the scope of sound medical judgement, suitable for use in contact with the tissues of humans and lower mammals without undue toxicity, irritation, and allergic response, are commensurate with a reasonable benefit/risk ratio, and are effective for their intended use, as well as the zwitterionic forms, where possible, of the compounds of the invention.

The term “prodrug” refers to compounds which are rapidly transformed in vivo to the parent compound of the above formula, for example, by hydrolysis in blood. A thorough discussion is provided in T. Higuchi and V. Stella, “Pro-drugs as Novel Delivery Systems,” Vol. 14 of the A.C.S. Symposium Series, and in Edward B. Roche, ed., “Bioreversible Carriers in Drug Design,” American Pharmaceutical Association and Pergamon Press, 1987, both of which are hereby incorporated by reference.

The term “thioalkoxy” refers to an alkyl group attached to the parent molecular group through a sulfur atom.

Compounds of the present invention may exist as stereoisomers where asymmetric or chiral centers are present. The present invention contemplates various stereoisomers and mixtures thereof. Stereoisomers include enantiomers and diastereomers. Individual stereoisomers of compounds of the present invention can be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic columns.

Geometric isomers may also exist in the compounds of the present invention. The present invention contemplates the various geometric isomers and mixtures thereof resulting from the arrangement of substituents around a carbon-carbon double bond or disposition of substituents around a ring. Substituents around a carbon-carbon double bond are designated as being in the Z or E configuration where the term “Z” refers to substituents on the same side of the carbon-carbon double bond and the term “E” refers to substituents on opposite sides of the carbon-carbon double bond.

Compounds of the present invention can exist as rotamers. Rotamers are formed from hinderance around an amide bond to provide 2 or more distinct compounds which can be separated by means well-known to those skilled in the art.

### Determination of Biological Activity

### Cell and Culture Conditions

Human peripheral blood mononuclear cells were cultured in RPMI 1640 medium supplemented with 10 μg/ml gentamicin, 50 μM 2-mercaptoethanol, 1X MEM non-essential amino acids (Sigma Chemical Co., St. Louis, Mo.), 100 U/ml sodium penicillin G, 100 μg/ml streptomycin sulfate, 2 mM L-glutamine, 1 mM sodium pyruvate (Life Technologies, Grand Island, N.Y.) and 10% fetal bovine serum (Hyclone, Logan, Utah) at 37° C. with 5% CO₂.

### Preparation of Human Peripheral Blood Mononuclear Cells

The procedure in Current Protocols in Immunology, Volume 1, Published by the Greene Publishing Associates and John Wiley & Sons, Inc, Edited by Richard Coico, 1994, hereby incorporated by reference, was followed. Briefly, 50 ml of blood from human volunteers was collected in heparinized syringes and mixed. Blood was diluted 1:1 in Dulbecco's phosphate buffered saline (D-PBS) (Life Technologies, Grand Island, N.Y.) and mixed. PBS-blood mixture was overlaid into 50 ml centrifuge tubes containing 15 ml Histopaque 1077 (Sigma Chemical Co., St. Louis, Mo.) and centrifuged at 500 X G for 30 minutes at room temperature. Cells at the interface from each Histopaque tube were removed and mixed with 5 ml of D-PBS. Each cell suspension was diluted to 50 ml with D-PBS, mixed and centrifuged at 400 X G for 15 minutes at room temperature. After most of the supernatant was removed, cells were resuspended to 40 ml with D-PBS per tube (2 tubes per donor). Cells were centrifuged at 400 X G for 10 minutes at room temperature. Pellets were resuspended in 10 ml of supplemented RPMI 1640 and cell number determined with a Coulter counter. Cells were diluted to a concentration of 0.5×10⁶ cells per mL.

### Human Concanavalin-A Proliferation Assay (Con HU Assay)

The procedure in Current Protocols in Immunology, Volume 1, Published by the Greene Publishing Associates and John Wiley & Sons, Inc, Edited by Richard Coico, 1994, hereby incorporated by reference, was followed. Briefly, test compounds were added to appropriate wells on 96-well tissue culture plates (Corning Glass Works, Corning, N.Y.) in 20 pl of supplemented RPMI 1640. Human peripheral blood mononuclear cells were added to each well in 100 μl volumes (final cell concentration equal to 50,000 cells per well). After 15 minutes, 100 μl of 5 μg/ml concanavalin-A (Sigma Chemical Co., St. Louis, Mo.) in supplemented RPMI 1640 was added to a final concentration of 2.5 μg/ml. Plates were incubated for 3 days at 37° C. with 5% CO₂. On day 3, plates were pulsed with 0.5 μCi/well tritiated thymidine (New England Nuclear, Boston, Mass.). After 6 hours, plates were harvested on a Tomatec 96-well havester (Orange, Conn.). Glass filter mats were counted on a Matrix 9600 direct beta counter (Packard, Meriden, Conn.).

TABLE 1
Inhibitory Potency of Representative Compounds in the Human
Concanavalin-A Proliferation Assay (Con HU)

| | % Inhibition of | |
|---|---|---|
| | proliferation (at | |
| | concentrations of | |
| Example Number | 1,10, or 100 μm) | Con HU IC₅₀ (nM) |
| 1 | 68(1) | — |
| 2 | 1(1) | — |
| 3 | 100(10) | 328 |
| 4 | 82(100) | 28806 |
| 5 | 100(100) | 33666 |
| 6 | 100(10) | 98 |
| 7 | 97(10) | 403 |
| 9 | 35(1) | — |
| 10 | 100(10) | 114 |
| 11 | 100(10) | 376 |
| 12 | 98(10) | 427 |
| 13 | 99(100) | 2843 |
| 14 | 97(100) | 538 |
| 15 | 99(100) | 274 |
| 16 | 100(100) | 4186 |
| 17 | 18(1) | — |
| 18 | 100(100) | 374 |
| 19 | 9(1) | — |
| 20 | 10(1) | — |
| 21 | 2(1) | — |
| 22 | 14(1) | — |
| 23 | 4(1) | — |
| 24 | 98(100) | 301 |
| 25 | 100(10) | 396 |
| 26 | 24(1) | — |
| 27 | 100(10) | 299 |
| 28 | 12(1) | — |
| 29 | 100(10) | 51 |
| 30 | 6(1) | — |
| 31 | 100(100) | 4038 |
| 32 | 100(100) | 8436 |
| 35 | 65(1) | — |
| 40 | 36(1) | — |
| 41 | 100(10) | 343 |
| 42 | 9(1) | — |
| 43 | 98(10) | 354 |
| 44 | 29(1) | — |
| 45 | 18(1) | — |
| 46 | 10(1) | — |
| 48 | 85(1) | 468 |
| 49 | 9(1) | — |
| 50 | 2(1) | — |
| 51 | 100(10) | 778 |
| 52 | 26(1) | — |
| 53 | 99(100) | 202 |
| 54 | 12(1) | — |
| 55 | 13(1) | — |
| 56 | 32(1) | — |
| 57 | 5(1) | — |
| 58 | 97(10) | 484 |
| 59 | 54(1) | — |
| 60 | 100(100) | 296 |
| 65 | 98(100) | 1823 |
| 66 | 97(100) | 1044 |
| 67 | 100(100) | 254 |
| 68 | 99(100) | 2437 |
| 69 | 98(100) | 506 |
| 70 | 100(100) | 913 |
| 71 | 87(10) | 544 |
| 73 | 93(10) | 388 |
| 74 | 83(100) | 22826 |
| 75 | 100(100) | 368 |
| 76 | 100(100) | 3173 |
| 77 | 93(10) | 655 |
| 78 | 95(100) | 607 |
| 79 | 9(1) | — |
| 80 | 43(1) | — |
| 81 | 30(1) | — |
| 82 | 100(100) | 468 |
| 83 | 99(10) | 71 |
| 84 | 76(1) | 712 |
| 85 | 93(100) | 275 |
| 86 | 33(1) | — |
| 87 | (−)52(1) | — |
| 88 | 29(1) | — |
| 89 | 99(100) | 1232 |
| 90 | 98(100) | 144 |
| 91 | 99(100) | 138 |
| 92 | 92(100) | 673 |
| 93 | 100(100) | 365 |
| 94 | 3(10) | — |
| 95 | 47(10) | 7280 |
| 96 | 99(100) | 338 |
| 97 | 20(10) | — |
| 98 | 94(100) | 4923 |
| 100 | 75(100) | 2154 |
| 101 | 100(100) | 2227 |
| 102 | 100(100) | 503 |
| 103 | 14(1) | — |
| 104 | 99(100) | 394 |
| 105 | 100(10) | 387 |
| 106 | 100(10) | 237 |
| 107 | 99(10) | 304 |
| 108 | 18(1) | — |
| 109 | 45(1) | — |
| 110 | 99(10) | 314 |
| 111 | 76(100) | 41000 |
| 112 | (−)2(1) | — |
| 114 | 98(100) | 84 |
| 115 | 71(100) | 51313 |
| 116 | 100(10) | 154 |
| 117 | 100(100) | 158 |
| 119 | 100(10) | 572 |
| 120 | 100(100) | 488 |
| 121 | 53(100) | 10565 |
| 122 | 15(1) | — |
| 123 | 99(100) | 256 |
| 124 | 99(100) | 285 |
| 125 | 6(1) | — |
| 126 | 79(10) | 4906 |
| 127 | 100(100) | 487 |
| 128 | 25(1) | — |
| 129 | 100(100) | 380 |
| 130 | 100(100) | 336 |
| 132 | 56(10) | 6215 |
| 133 | 97(10) | 315 |
| 134 | 100(100) | 2770 |
| 135 | 100(100) | 207 |
| 136 | 99(100) | 222 |
| 137 | 98(100) | 120 |
| 138 | 99(10) | 364 |
| 139 | 5(10) | — |
| 140 | 100(100) | 298 |
| 141 | 4(1) | — |
| 142 | 99(10) | 489 |
| 143 | 100(100) | 1675 |
| 144 | 100(100) | 240 |
| 145 | 94(100) | 1593 |
| 146 | 98(100) | 269 |
| 147 | 99(100) | 71 |
| 148 | 94(100) | 529 |
| 149 | 100(100) | 336 |
| 150 | 100(100) | 244 |
| 151 | 99(100) | 295 |
| 154 | 52(10) | 3817 |
| 156 | 16(1) | — |
| 157 | 42(10) | 6761 |
| 158 | 30(1) | — |
| 159 | 93(10) | 2928 |
| 160 | 1(1) | — |
| 161 | 99(100) | 231 |
| 162 | 100(10) | 44 |
| 163 | 98(100) | 235 |
| 164 | 99(10) | 39 |
| 165 | 57(10) | 6703 |
| 166 | 11(1) | — |
| 167 | 100(100) | 279 |
| 168 | 20(1) | — |
| 169 | 2(1) | — |
| 170 | 25(1) | — |
| 171 | 12(1) | — |
| 172 | 48(1) | — |
| 173 | 28(1) | — |
| 174 | 99(10) | 730 |
| 175 | 100(100) | 562 |
| 176 | 12(1) | — |
| 177 | 4(1) | — |
| 178 | 14(1) | — |
| 179 | 47(10) | 8871 |
| 180 | 95(10) | 2872 |
| 181 | 100(10) | 2240 |
| 182 | 83(10) | 4668 |
| 183 | 94(10) | 542 |
| 184 | 90(10) | 420 |
| 185 | 26(1) | — |
| 186 | 14(1) | — |
| 187 | 86(1) | 362 |
| 188 | 87(10) | 485 |
| 189 | 24(1) | — |
| 190 | 3(1) | — |
| 191 | 99(1) | 116 |
| 192 | 10(1) | — |
| 193 | 8(1) | — |
| 194 | 23(1) | — |
| 195 | 22(1) | — |
| 196 | 11(1) | — |
| 197 | 1(1) | — |
| 198 | 4(1) | — |
| 199 | 30(1) | — |
| 200 | 26(1) | — |
| 201 | 100(10) | 364 |
| 202 | 6(1) | — |
| 203 | 1(1) | — |
| 204 | 12(1) | — |
| 205 | 100(10) | 196 |
| 206 | 100(10) | 61 |
| 207 | 100(10) | 372 |
| 208 | 99(10) | 149 |
| 209 | 99(10) | 33 |
| 210 | 100(10) | 239 |
| 211 | 98(10) | 39 |
| 212 | 99(10) | 70 |
| 213 | 100(10) | 434 |
| 214 | 100(10) | 68 |
| 215 | 100(10) | 126 |
| 216 | 100(10) | 267 |
| 217 | 100(10) | 218 |
| 218 | 100(10) | 136 |
| 219 | 100(10) | 214 |
| 220 | 100(10) | 4232 |
| 221 | 98(10) | 411 |
| 222 | 100(10) | 760 |
| 223 | 100(10) | 93 |
| 224 | 2(1) | — |
| 225 | 98(10) | 154 |
| 226 | 100(10) | 43 |
| 227 | 100(10) | 257 |
| 228 | 99(10) | 147 |
| 229 | 99(10) | 40 |
| 230 | 64(1) | — |
| 231 | 100(10) | 25 |
| 232 | 99(10) | 172 |
| 233 | 100(10) | 340 |
| 234 | 99(10) | 61 |
| 235 | 100(10) | 107 |
| 236 | 100(10) | 180 |
| 237 | 100(100) | 368 |
| 238 | 3(1) | — |
| 239 | 55(10) | — |
| 240 | 84(10) | 507 |
| 241 | 10(1) | — |
| 242 | 3(1) | — |
| 243 | 99(10) | 390 |
| 244 | 94(10) | 523 |
| 245 | 100(100) | 279 |
| 246 | 47(1) | — |
| 247 | 97(10) | 375 |
| 248 | 100(10) | 143 |
| 249 | 100(10) | 182 |
| 250 | 100(100) | 173 |
| 251 | 50(1) | — |
| 252 | 36(1) | — |
| 253 | 94(10) | 447 |
| 254 | 9(1) | — |
| 255 | 12(1) | — |
| 256 | 100(10) | 250 |
| 257 | 100(10) | 387 |
| 258 | 16(1) | — |
| 259 | 5(1) | — |
| 260 | 96(10) | 369 |
| 261 | 37(1) | — |
| 262 | 100(10) | 419 |
| 263 | 100(10) | 2932 |
| 264 | 100(10) | 42 |
| 265 | 90(10) | 3808 |
| 266 | 100(100) | 322 |
| 267 | 38(1) | — |
| 268 | 100(10) | 347 |
| 269 | 99(10) | 392 |
| 270 | 100(10) | 228 |
| 271 | 14(1) | — |
| 272 | 10(1) | — |
| 273 | 12(1) | — |
| 274 | 100(10) | 2181 |
| 275 | 8(1) | — |
| 276 | 16(1) | — |
| 277 | 99(100) | 1063 |
| 278 | 4(1) | — |
| 279 | 6(1) | — |
| 280 | 16(10) | — |
| 281 | 94(100) | 1063 |
| 282 | 100(100) | 248 |
| 283 | 99(100) | 1045 |
| 287 | 24(1) | |
| 288 | 16(1) | |
| 289 | 22(1) | |
| 290 | | 373 |
| 291 | | 411 |
| 292 | | 258 |
| 293 | | 409 |
| 294 | | 299 |
| 295 | | 232 |
| 296 | | 44 |
| 297 | | 251 |
| 298 | | 332 |
| 299 | | 269 |
| 300 | | 79 |
| 301 | | 232 |
| 302 | | 358 |
| 303 | | 487 |
| 304 | | 266 |
| 305 | | 170 |
| 306 | | 265 |
| 307 | | 79 |
| 308 | | 309 |
| 309 | | 32 |
| 310 | | 335 |
| 311 | | 323 |
| 312 | | 298 |
| 313 | | 66 |
| 314 | | 246 |
| 315 | | 320 |
| 316 | | 41 |
| 317 | | 186 |
| 318 | | 258 |
| 319 | | 219 |
| 320 | | 43 |
| 321 | | 185 |
| 322 | | 238 |
| 323 | | 238 |
| 324 | | 89 |
| 325 | | 172 |
| 326 | | 166 |
| 327 | | 82 |
| 328 | | 75 |
| 329 | | 303 |
| 330 | | 169 |
| 331 | | 220 |
| 332 | | 44 |
| 333 | | 410 |
| 334 | | 297 |
| 335 | | 103 |
| 336 | | 1826 |
| 337 | | 221 |
| 338 | | 164 |
| 339 | | 369 |
| 340 | | 251 |
| 341 | | 191 |
| 342 | | 238 |
| 343 | | 250 |
| 344 | | 251 |
| 345 | | 273 |
| 346 | | 117 |
| 347 | | 288 |
| 348 | | 114 |
| 349 | | 224 |
| 350 | | 240 |
| 351 | | 309 |
| 352 | | 141 |
| 353 | | 174 |
| 354 | | 75 |
| 355 | | 282 |
| 356 | | 247 |
| 357 | | 1855 |
| 358 | | 203 |
| 359 | | 261 |
| 360 | | 329 |

### CD3 and CD28 Activation of Peripheral Blood T Cells and Determination of Secreted IL-2 Levels (C28 HU Assay)

Human peripheral blood mononuclear cells (PBMC) were isolated by Ficoll-Hypaque seperation. PBMCs were stimulated with a combination of immobilized anti-CD3 and soluble anti CD28 mAbs as described in Faltynek, et al. J. Enzyme Inhibition 1995, 9, 111-122, hereby incorporated by reference. Following a 24 hour incubation, cell supernatants were harvested and IL-2 levels were determined. 100 μl of 5 μg/ml monoclonal murine anti-human IL-2 antibody (Biosource International) in D-PBS was added to 96 well Maxisorb plates (Nunc) and incubated at 4° C. overnight. Plates were washed 4 times with D-PBS containing 0.05% Tween 20 (wash buffer) and blocked with D-PBS containing 1% BSA and 10 mM NaN₃ (Diluent/Blocking buffer) for 1-3 hours at room temperature or overnight at 4° C. Plates were washed and recombinant human IL-2 diluted (at 10,000, 5,000, 2,500, 1,250, 625, 312.5, 156.25, 78, 39, 20 pg/ml) in diluent/blocking buffer containing a matched percentage of complete RPMI 1640 medium as the unknown samples. Tissue culture supernatant at various dilutions were added in triplicate at 100 μl/well. Plates were incubated for 2 hours at room temperature and washed 4 times with wash buffer. 100 pl of rabbit anti-human IL-2 (10 μg/ml, Genzyme) was added and incubated for 1 hour at room temperature. The incubation was followed by 4 washes and subsequent addition of 100 pl of 1:2000 dilution of alkaline phosphatase-conjugated goat anti-rabbit F(ab′)₂ (Biosource International). After 1 hour the plates were washed 4 times and 100 μl of pNPP (Southern Biotech or Sigma) at 1 mg/ml in buffer was added. Color development was allowed to proceed at room temperature for 20 minutes before addition of 50 μl of 2 N NaOH. Absorbance at 405 nm was determined using a plate reader (Molecular Devices). IL-2 concentrations were calcualted using SoftMax (Molecular Devices) based on the IL-2 standard solutions.

TABLE 2
Inhibition of IL-2 Secretion by Representative
Compounds in the C28 Assay

| | % Inhibition of IL-2 | | |
|---|---|---|---|
| | secretion | | |
| | (At concentrations of | | C28 Assay |
| Example Number | 1, 10, or 100 μM) | | IC₅₀ (nM) |
| 8 | 20 | (1) | — |
| 24 | 83 | (1) | 380 |
| 33 | 6 | (1) | — |
| 34 | 13 | (100) | — |
| 36 | 10 | (1) | — |
| 38 | 16 | (1) | — |
| 39 | 14 | (1) | — |
| 47 | 2 | (1) | — |
| 61 | 32 | (1) | — |
| 62 | 96 | (100) | 5035 |
| 63 | 19 | (1) | — |
| 64 | 86 | (100) | 22274 |
| 72 | 11 | (1) | — |
| 99 | 9 | (1) | — |
| 113 | 19 | (1) | — |
| 118 | 27 | (1) | — |
| 131 | 32 | (1) | — |
| 152 | 3 | (1) | — |
| 153 | 94 | (100) | 457 |
| 155 | 8 | (1) | — |
| 250 | 97 | (100) | 102 |
| 266 | 88 | (100) | 314 |
| 284 | 17 | (1) | — |
| 285 | 26 | (1) | — |
| 286 | 2 | (1) | — |

Measurement of IL-5 and IL-4 Levels in Human T Cells (IL-4 and IL-5 Assays) Human T cells (HUT 78) were cultured to 1×10⁶/mL in RPMI 1640 medium containing 10% fetal calf serum, 100 U/mL penicillin and 100 μg/mL streptomycin. Cultures were then centrifuged, to pellet the cells, and cells resuspended in fresh medium to the same density. 0.2 mL samples of cells were incubated in 96-well plates with 8 μL of various concentrations of compound freshly diluted with the above medium from 100 mM solvent stocks (ethanol or DMSO). Immediately after addition of compound, cells were stimulated by addition of 2 ng/mL phorbol 12-myristate 13-acetate (1 μL of freshly prepared solution of stock (in DMSO) diluted with the above medium added to cells) and 750 μg/mL anti-CD3 (pre-coated at 4° C. overnight). Cell cultures were incubated at 37 OC for 32 hours, then cells pelleted by centrifugation and the supernatants harvested for ELISA. IL-4 and IL-5 ELISA's were performed according to standard procedures. Inhibition was calculated relative to cytokine levels produced from control stimulated cells not treated with compound.

TABLE 3
Inhibition of IL-4 and IL-5 Secretion in Human T Cells by
Representative Compounds and Comparison with FK-506

| | IL-4 Inhibition | IL-5 Inhibition |
|---|---|---|
| Example Number | IC₅₀ (nM) | IC₅₀ (nM) |
| FK-506 | 0.7 | 0.5 |
| 24 | 150 | 150 |
| 250 | 50 | 80 |
| 266 | 110 | 150 |
| 209 | 5 | 38 |
| 6 | 8 | 50 |
| 264 | 4.8 | 22 |

As shown in Tables 1, 2 and 3, the compounds are useful for inhibiting cytokine (IL-2, IL-4 and IL-5) production and cellular proliferation in stimulated human T cell lines or human peripheral blood mononuclear cells and therefore have utility in the treatment of diseases that are prevented by or ameliorated with cytokine inhibitors.

The compounds of the invention, including but not limited to those specified in the examples, possess immunomodulatory activity in mammals, especially humans. As immunosuppressants, the compounds of the present invention are useful for the treatment and prevention of immune-mediated diseases such as the resistance to transplantation of organs or tissue such as heart, kidney, liver, medulla ossium, skin, cornea, lung, pancreas, intestinum tenue, limb, muscle, nerves, duodenum, small-bowel, pancreatic-islet-cell, and the like; graft-versus-host diseases brought about by medulla ossium transplantation; autoimmune diseases such as rheumatoid arthritis, systemic lupus erythematosus, Hashimoto's thyroiditis, multiple sclerosis, myasthenia gravis, type I diabetes, uveitis, allergic encephalomyelitis, glomerulonephritis, and the like. Further uses include the treatment and prophylaxis of inflammatory and hyperproliferative skin diseases and cutaneous manifestations of immunologically-mediated illnesses, such as psoriasis, atopic dermatitis, contact dermatitis and further eczematous dermatitises, seborrhoeis dermatitis, lichen planus, pemphigus, bullous pemphigoid, epidermolysis bullosa, urticaria, angioedemas, vasculitides, erythemas, cutaneous eosinophilias, lupus erythematosus, acne and alopecia areata; various eye diseases (autoimmune and otherwise) such as keratoconjunctivitis, vernal conjunctivitis, uveitis associated with Behcet's disease, keratitis, herpetic keratitis, conical cornea, dystrophia epithelialis corneae, corneal leukoma, and ocular pemphigus. In addition reversible obstructive airway disease, which includes conditions such as asthma (for example, bronchial asthma, allergic asthma, intrinsic asthma, extrinsic asthma and dust asthma), particularly chronic or inveterate asthma (for example, late asthma and airway hyper-responsiveness), bronchitis, allergic rhinitis, and the like are targeted by compounds of this invention. Inflammation of mucosa and blood vessels such as gastric ulcers, vascular damage caused by ischemic diseases and thrombosis. Moreover, hyperproliferative vascular diseases such as intimal smooth muscle cell hyperplasia, restenosis and vascular occlusion, particularly following biologically- or mechanically- mediated vascular injury, could be treated or prevented by the compounds of the invention. Other treatable conditions include but are not limited to ischemic bowel diseases, inflammatory bowel diseases, necrotizing enterocolitis, intestinal inflammations/allergies such as Coeliac diseases, proctitis, eosinophilic gastroenteritis, mastocytosis, Crohn's disease and ulcerative colitis; nervous diseases such as multiple myositis, Guillain-Barre syndrome, Meniere's disease, polyneuritis, multiple neuritis, mononeuritis and radiculopathy; endocrine diseases such as hyperthyroidism and Basedow's disease; hematic diseases such as pure red cell aplasia, aplastic anemia, hypoplastic anemia, idiopathic thrombocytopenic purpura, autoimmune hemolytic anemia, agranulocytosis, pernicious anemia, megaloblastic anemia and anerythroplasia; bone diseases such as osteoporosis; respiratory diseases such as sarcoidosis, fibroid lung and idiopathic interstitial pneumonia; skin disease such as dermatomyositis, leukoderma vulgaris, ichthyosis vulgaris, photoallergic sensitivity and cutaneous T cell lymphoma; circulatory diseases such as arteriosclerosis, atherosclerosis, aortitis syndrome, polyarteritis nodosa and myocardosis; collagen diseases such as scleroderma, Wegener's granuloma and Sjogren's syndrome; adiposis; eosinophilic fasciitis; periodontal disease such as lesions of gingiva, periodontium, alveolar bone and substantia ossea dentis; nephrotic syndrome such as glomerulonephritis; male pattern aleopecia or alopecia senilis by preventing epilation or providing hair germination and/or promoting hair generation and hair growth; muscular dystrophy; Pyoderma and Sezary's syndrome; Addison's disease; active oxygen-mediated diseases, as for example organ injury such as ischemia-reperfusion injury of organs (such as heart, liver, kidney and digestive tract) which occurs upon preservation, transplantation or ischemic disease (for example, thrombosis and cardiac infarction); intestinal diseases such as endotoxin-shock, pseudomembranous colitis and colitis caused by drug or radiation; renal diseases such as ischemic acute renal insufficiency and chronic renal insufficiency; pulmonary diseases such as toxinosis caused by lung-oxygen or drug (for example, paracort and bleomycins), lung cancer and pulmonary emphysema; ocular diseases such as cataracta, siderosis, retinitis, pigmentosa, senile macular degeneration, vitreal scarring and corneal alkali burn; dermatitis such as erythema multiforme and others such as sinusitis, gingivitis, periodontitis, sepsis, pancreatitis, diseases caused by environmental pollution (for example, air pollution), aging, carcinogenesis, metastasis of carcinoma and hypobaropathy; diseases caused by histamine or leukotriene-C₄ release; Behcet's disease such as intestinal-, vasculo- or neuro-Behcet's disease, and also Behcet's which affects the oral cavity, skin, eye, vulva, articulation, epididymis, lung, kidney and so on. Furthermore, the compounds of the invention are useful for the treatment and prevention of hepatic disease such as immunogenic diseases (for example, chronic autoimmune liver diseases such as autoimmune hepatitis, primary biliary cirrhosis and sclerosing cholangitis), partial liver resection, acute liver necrosis (e.g. necrosis caused by toxin, viral hepatitis, shock or anoxia), B-virus hepatitis, non-A/non-B hepatitis, cirrhosis (such as alcoholic cirrhosis) and hepatic failure such as fulminant hepatic failure, late-onset hepatic failure and “acute-on-chronic” liver failure (acute liver failure on chronic liver diseases), and moreover are useful for various diseases because of their useful activity such as augmention of chemotherapeutic effect, cytomegalovirus infection, particularly HCMV infection, anti-inflammatory activity, sclerosing and fibrotic diseases such as nephrosis, scleroderma, pulmonary fibrosis, arteriosclerosis, congestive heart failure, ventricular hypertrophy, post-surgical adhesions and scarring, stroke, myocardial infarction and injury associated with ischemia and reperfusion, and the like.

The present invention also provides pharmaceutical compositions that comprise compounds of the present invention formulated together with one or more non-toxic pharmaceutically acceptable carriers. The pharmaceutical compositions can be specially formulated for oral administration in solid or liquid form, for parenteral injection or for rectal administration.

The pharmaceutical compositions of this invention can be administered to humans and other animals orally, rectally, parenterally, intracisternally, intravaginally, intraperitoneally or topically (such as powders, ointments or drops), bucally or as an oral or nasal spray. The term “parenteral” administration refers to modes of administration that include intravenous, intramuscular, intraperitoneal, intrasternal, subcutaneous and intraarticular injection and infusion.

Pharmaceutical compositions of this invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants.

These compositions may also contain adjuvants such as preservative, wetting agents, emulsifying agents and dispersing agents. Prevention of the action of microorganisms can be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form can be brought about by the inclusion of agents (such as aluminum monostearate and gelatin) that delay absorption.

In some cases, in order to prolong the effect of the drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution that, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions that can be dissolved or dispersed in sterile water or other sterile injectable media just prior to use.

Solid dosage forms for oral administration include capsules, tablets, pills, powders and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols, and the like.

The solid dosage forms of tablets, dragees, capsules, pills, and granules can be prepared with coatings and shells such as enteric coatings and other coatings well known in the pharmaceutical formulating art. They may optionally contain opacifying agents and may also be of a composition such that they release the active ingredient(s) only, or preferentially, embedding compositions that can be used include polymeric substances and waxes.

The active compounds may also be in micro-encapsulated form, if appropriate, with one or more of the above-mentioned excipients.

The compounds of the present invention may be used in the form of pharmaceutically acceptable salts derived from inorganic or organic acids. By “pharmaceutically acceptable salt” is meant those salts which are, within the scope of sound medical judgement, suitable for use in contact with the tissues of humans and lower animals without undue toxicity, irritation, allergic response and the like and are commensurate with a reasonable benefit/risk ratio.

Pharmaceutically acceptable salts are well-known in the art. For example, S. M. Berge, et al. describe pharmaceutically acceptable salts in detail in J. Pharmaceutical Sciences, 1977, 66: 1 et seq. The salts may be prepared in situ during the final isolation and purification of the compounds of the invention or separately by reacting a free base function with a suitable acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate, lactate, maleate, methanesulfonate, nicotinate, 2-naphthalenesulfonate, oxalate, palmoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like.

Liquid dosage forms for oral administration include pharmaceutically acceptable emulsions, solutions, suspensions, syrups and elixirs. In addition to the active compounds, the liquid dosage forms may contain inert diluents commonly used in the art such as water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan and mixtures thereof.

Besides inert diluents, the oral compositions may also include adjuvants such as wetting agents, emulsifying and suspending agents, sweetening, flavoring and perfuming agents.

Suspensions, in addition to the active compounds, may contain suspending agents such as ethoxylated isostearyl alcohols, polyoxyethylene sorbitol and sorbitan esters, microcrystalline cellulose, aluminum metahydroxide, bentonite, agar-agar, and tragacanth and mixtures thereof.

Compositions for rectal or vaginal administration are preferably suppositories that can be prepared by mixing the compounds of this invention with suitable non-irritating excipients or carriers such as cocoa butter, polyethylene glycol or a suppository wax that are solid at room temperature but liquid at body temperature and therefore melt in the rectum or vaginal cavity and release the active compound.

Compounds of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and the phosphatidyl cholines (lecithins), both natural and synthetic.

Methods to form liposomes are known in the art. See, for example, Prescott, Ed., “Methods in Cell Biology,” Volume XIV, Academic Press, New York, N.Y. (1976), p. 33 et seq.

Compounds of the present invention may also be coadministered with one or more immunosuppressant agents. The immunosuppressant agents within the scope of this invention include, but are not limited to, IMURAN® (azathioprine sodium), brequinar sodium, SPANIDIN® (gusperimus trihydrochloride, also known as deoxyspergualin), mizoribine (also known as bredinin), CELLCEPT® (mycophenolate mofetil), Cyclosporin A in its various formulations (NEORAL®, SANDIMMUNE®, and generic formulations), PROGRAF® (tacrolimus, also known as FK-506), RAPAMUNE® (sirolimus also known as rapamycin),and leflunomide (also known as HWA-486), glucocorticoids, such as prednisolone and its derivatives, antibody therapies such as orthoclone (OKT3) and Zenapax®, and antithymyocyte globulins, such as thymoglobulins.

Dosage forms for topical administration of a compound of this invention include powders, sprays, ointments and inhalants. The active compound is mixed under sterile conditions with a pharmaceutically acceptable carrier and any needed preservatives, buffers or propellants that can be required. Opthalmic formulations, eye ointments, powders and solutions are also contemplated as being within the scope of this invention. Actual dosage levels of active ingredients in the pharmaceutical compositions of this invention can be varied so as to obtain an amount of the active compound(s) that is effective to achieve the desired therapeutic response for a particular patient, compositions, and mode of administration. The selected dosage level will depend upon the activity of the particular compound, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved. Generally dosage levels of about 1 to about 50, more preferably of about 5 to about 20 mg, of active compound per kilogram of body weight per day when administered orally to a mammalian patient. If desired, the effective daily dose can be divided into multiple doses for purposes of administration, e.g. two to four separate doses per day.

### Preparation of Compounds of This Invention

The compounds of this invention can be prepared by a variety of synthetic routes. Representative procedures are shown in Schemes 1-12 wherein R₁, R₂, R₃, R₄, R₅, L₃, Q, B and E are defined above unless otherwise indicated.

### Abbreviations

Abbreviations that have been used in the descriptions of the schemes and the examples that follow are: THF for tetrahydrofuran; DMF for N,N-dimethylformamide; DMSO for dimethylsulfoxide; Boc for tert-butylcarbonyloxy; DCC for dicyclohexylcarbodiimide; EDC for 1-(3-dimethylaminopropyl)-3-ethylcarbodiimide hydrochloride; HBTU for O-benzotriazol-yl-N,N,N′N′-tetramethyluronium hexafluorophosphate; and DMAP for 4-dimethylaminopyridine. Starting materials, reagents and solvents were purchased from Aldrich Chemical Company (Milwaukee, Wis.), Maybridge Chemical Company (Tintagel, Cornwall, U.K.), Lancaster (Windham, N.H.), Sigma (St. Louis, Mo.), ACROS, and Chess (Mannheim, Germany).

### Description of Intermediates in the Schemes

Compounds of Formula I are designated by the small-case numbers (i), (ii), (iii), (iv), etc. The small-case letters (“-a,” “-b,” and “-c”) that follow the small-case numbers indicate the disposition of the substituent E on ring Q relative to the position of the pyrazole or triazole ring as defined in the schemes 1-12. Intermediates in the syntheses of compounds of Formula I are further designated by a capital letter (A, B, C, etc).

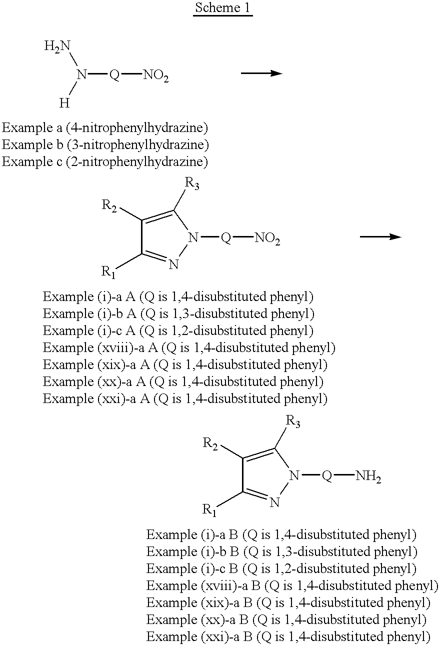

As shown in Scheme 1, the two-step construction of the 1,4- (“-a”), 1,3- (“-b”), and 1,2-disubstituted (“-c”) anilines that served as precursors to compounds of Formula I began with condensation of 1,4-, 1,3- or 1,2-nitrophenylhydrazine (“a,” “b,” and “c” respectively) with appropriately substituted 2,4-pentanediones in the presence of an acid catalyst such as p-toluenesulfonic acid, HCl, or H₂SO₄ to provide nitro intermediates (i)-a A, (i)-b A, (i)-c A, (xviii)-a A, (xix)-a A, (xx)-a A, and (xxi)-a A. Conversion of the nitro intermediates to the corresponding aniline precursors (i)-a B, (i)-b B, (i)-c B, (xviii)-a B, (xix)-a B, (xx)-a B, and (xxi)-a B was accomplished with hydrogen gas in the presence of a catalyst, preferably palladium on carbon. An alternative method was reduction with tin(II) chloride in the presence of acid, preferably hydrochloric acid, at elevated temperature. A more preferred method of reduction was with iron powder with ammonium chloride in ethanol/water.

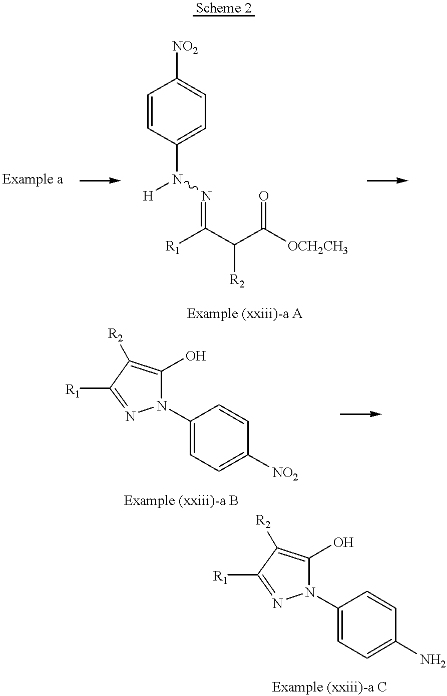

As shown in Scheme 2, replacement of the pentanedione in Scheme 1 with the appropriately substituted acetoacetate followed by ring closure with a non-nucleophilic base such as K₂CO₃ provided nitro intermediate (xxxiii)-a B which was converted to aniline precursor (xxxiii)-a C with reducing agents such as those described in Scheme 1.

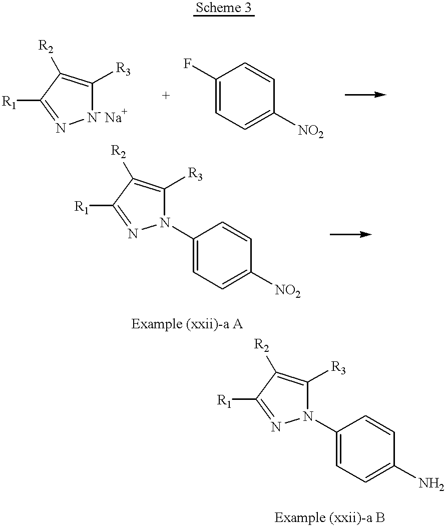

As shown in Scheme 3, an alternative route to aniline precursors was direct displacement of a leaving group, preferably fluoride, from 4-fluoronitrobenzene by the sodium salt of a preformed, substituted pyrazole ring followed by conversion of the nitro intermediate (xxii)-a A to aniline precursor (xxii)-a B with reducing agents such as those described in Scheme 1.

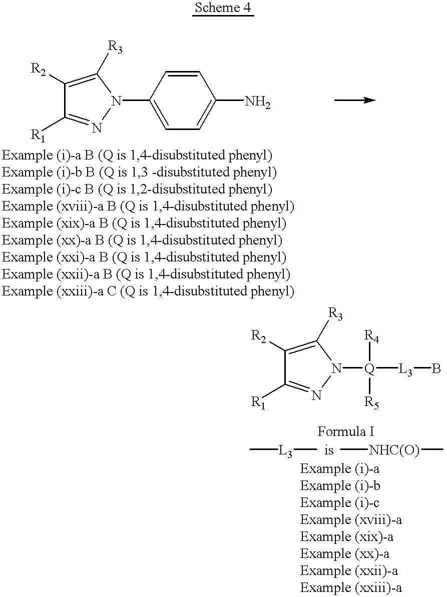

As shown in Scheme 4, conversion of the aniline precursors to compounds of Formula I was achieved by treatment of the anilines exemplified by examples (i)-a B, (i)-b B, (i)-c B, (xviii)-a B, (xix)-a B, (xx)-a B, (xxi)-a B, (xxii)-a B, and (xxiii)-a C with acid chlorides in the presence of base such as triethylamine, diisopropylethylamine or pyridine in dichloromethane. The same aniline intermediates may be reacted with carboxylic acids in dichloromethane in the presence of coupling agents such as DCC, HBTU or EDC with DMAP, preferably EDC with DMAP.

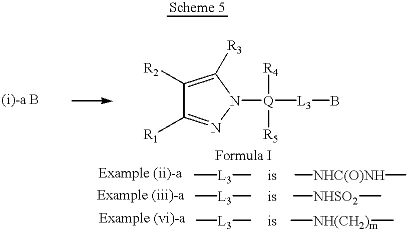

As shown in Scheme 5, conversion of Example (i)-a B to compounds of Formula I, as exemplified by examples (ii)-a, (iii)-a, and (vi)-a, was achieved by treatment of Example (i)-a B with isocyanates, sulfonyl chlorides, or aldehydes in the presence of appropriate reducing agents, respectively.

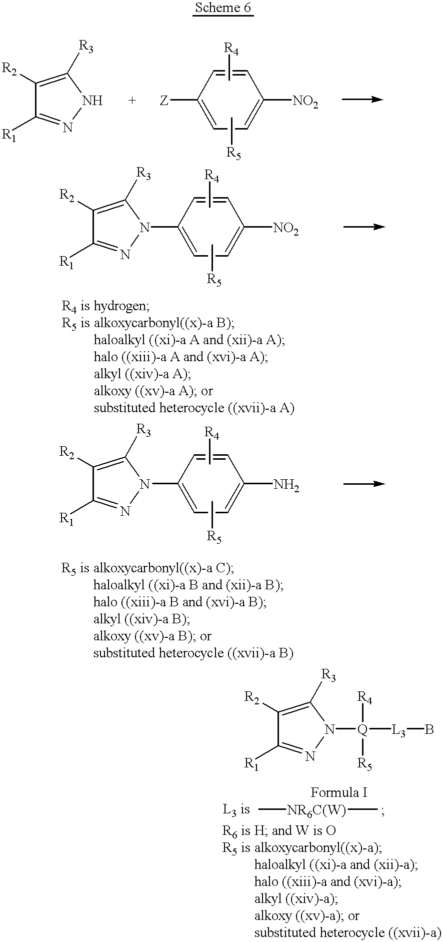

As shown in Scheme 6, the displacement and reduction chemistry described in Scheme 3 for the synthesis of the aniline precursors (where R₄ and R₅ are hydrogen) was also employed for the synthesis of aniline precursors where at least one of R₄ and R₅ is other than hydrogen. Anilines (x)-a C, (xi)-a B, (xii)-a B, (xiii)-a B, (xiv)-a B, (xv)-a B, (xvi)-a B, and (xvii)-a B were then converted to compounds of Formula I (exemplified by (x)-a, (xi)-a, (xii)-a, (xiii)-a, (xiv)-a, (xv)-a, (xvi)-a, and (xvii)-a by the coupling conditions described in Scheme 4.

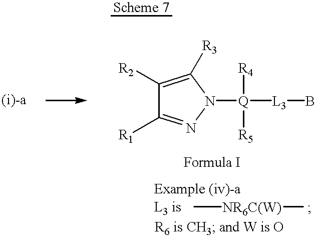

Compounds with modified, preformed linker groups are exemplified in Scheme 7. Compounds of Formula I (exemplified by Example (i)-a ) were alkylated at the amide bond nitrogen with methyl iodide in the presence of base, preferably potassium hydroxide to provide compounds of Formula I exemplified by Example (iv)-a.

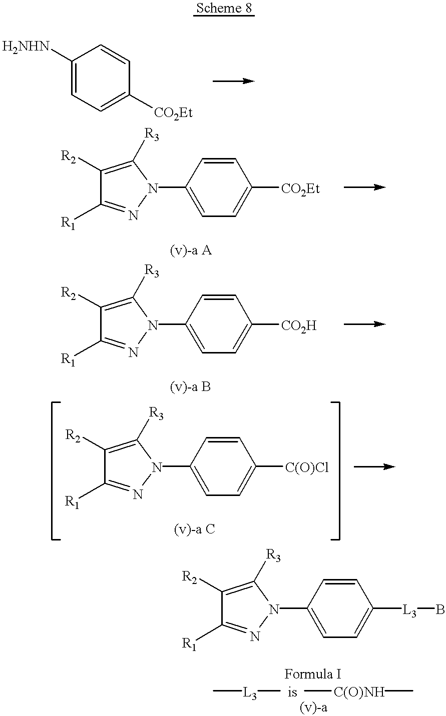

As shown in Scheme 8, compounds of Formula I derived from intermediates other than anilines were prepared from intermediate ester Example (v)-a A. Construction of the pyrazole ring from ethyl 4-hydrazinobenzoate according to Example (i)-a (Method 1) provided Example (v)-a A which was then hydrolyzed to carboxylic acid (v)-a B with base, preferably sodium hydroxide. Example (v)-a B was then elaborated to compounds of Formula I by conversion to the acid chloride (v)-a C with reagents such as thionyl chloride followed by treatment with amines in the presence of a base such as pyridine or triethylamine.

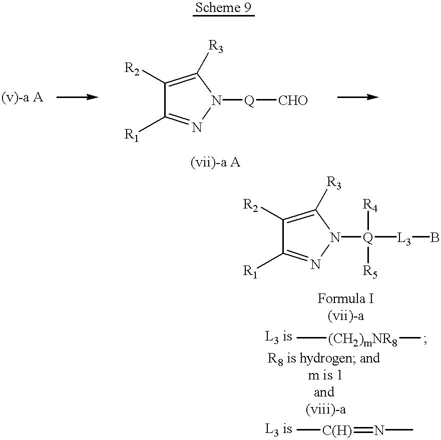

As shown in Scheme 9, Example (v)-a A was converted to aldehyde (vii)-a A by treatment with a reducing agent, preferably DIBAl-H at reduced temperature. Example (vii)-a A was then elaborated to compounds of Formula I by reductive amination or condensation (Example (vii)-a and Example (viii)-a, respectively by Method 13).

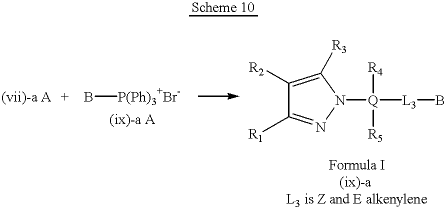

As shown in Scheme 10, treatment of Example (vii)-a A with ylides such as Example (ix)-a A also provided compounds of Formula I (exemplified by (ix)-a).

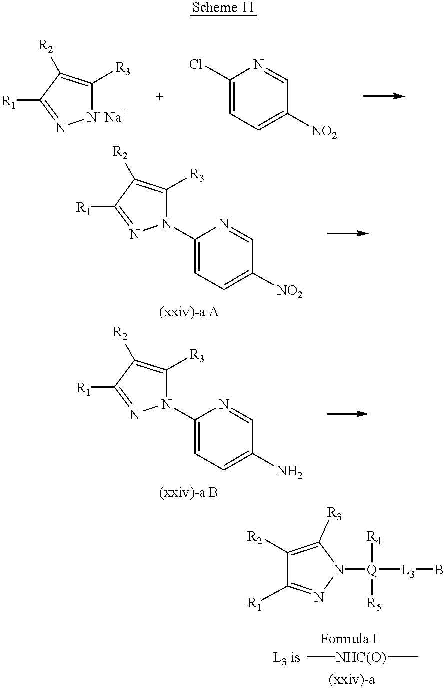

As shown in Scheme 11, the displacement and reduction chemistry described in Scheme 3 for the synthesis of the aniline precursors was also employed for the synthesis of precursors of compounds of Formula I where Q is a heterocycle, such as pyridine. 2-Chloro-5-nitropyridine was converted to nitro precursor (xxiv)-a A by treatment with the sodium salt of a preformed, substituted pyrazole ring. Example (xxiv)-a A was converted to aniline intermediate (xxiv)-a B by the reduction chemistry described in Scheme 1 then to compounds of Formula I by the coupling chemistry described in Scheme 4.

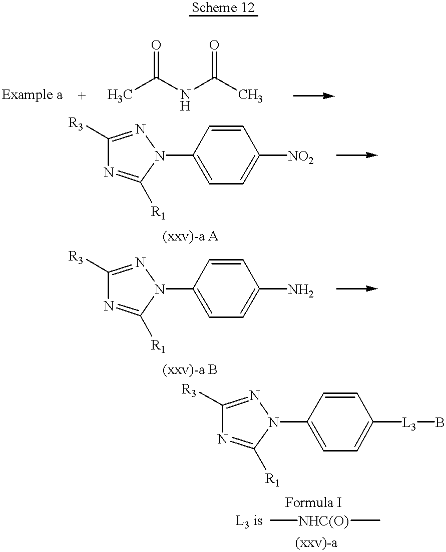

As shown in Scheme 12, construction of the substituted triazole rings of the compounds of Formula I was achieved by treatment of Example a (4-nitrophenylhydrazine) with diacetamide in the presence of acid, preferably sulfuric acid, to provide nitro intermediate

(xxv)-a A. Example (xxv)-a A was converted to aniline intermediate (xxv)-a B with reducing agents such as those described in Scheme 1. Example (xxv)-a B was then converted to compounds of Formula I by the coupling chemistry described in scheme 4.

### Example (i)-a, (i)-b, and (i)-c

### Compounds of Formula I where R₁ and R₃ are CF₃; R₂ is H; Z is carbon; Q is 1,4-, 1,3-, and 1,2-disubstituted phenyl; R₄ and R₅ are hydrogen: L₃ is —N(R₆)C(W)— where W is O and R₆ is H

### Example (i)-a A, (i)-b A, and (i)-c A (Method 1)

A solution of a, b, or c (1 equivalent), 1,1,1,5,5,5-hexafluoro-2,4-pentanedione (1.2 equivalents), and p-toluenesulfonic acid (1 mmol) in toluene was refluxed for 18 hours in a Dean-Stark apparatus, diluted with ethyl acetate, washed sequentially with 1M HCl and saturated aqueous NaHCO₃, dried (MgSO₄), and concentrated. The residue was purified by flash chromatography on silica gel with ethyl acetate/hexane to provide the desired compounds.

### Example (i)-a A, (i)-b A, and (i)-c A (Method 2)

A solution of a, b, or c (1 equivalent) and 1,1,1,5,5,5-hexafluoro-2,4-pentanedione (1.2 equivalents) in 4M hydrochloric acid (10-12 equivalents) and ethanol was refluxed overnight and concentrated. The residue was dissolved into ethyl acetate, washed sequentially with 1M HCl and brine, dried (Na₂SO₄), and concentrated to provide the desired compounds.

(Example (i)-a A) ¹H NMR (DMSO-d₆, 300 MHz) δ8.55-8.38 (dt, 2H), 7.78-7.74 (dt, 2H), 7.17 (s, 1H);

MS (DCI/NH₃) m/e 313 (reduced to aniline in MS, M+NH₄)⁺.

(Example (i)-b A) ¹H NMR (DMSO-d₆, 300 MHz) δ8.6 (t, 1H), 8.5 (m, 1H), 8.2 (dd, 1H), 8.0 (t, 1H), 7.9 (s, 1H);

(Example (i)-c A) ¹H NMR (DMSO-d₆, 300 MHz) δ8.38 (dd, 1H), 8.08-7.99 (m, 4H).

### Example (i)-a B, (i)-b B, and (i)-c B (Method 3)

A solution of (i)-(a, b, or c) A in ethyl acetate was treated with SnCl₂ (4 equivalents) at reflux (in some cases, the addition of concentrated hydrochloric acid (catalytic to 1 equivalent) led to a cleaner reduction), cooled, washed with saturated NaHCO₃, dried (Na₂SO₄), and concentrated. The residue was purified by column chromatography on silica gel with ethyl acetate/hexane or acetone/hexane to provide the desired compounds.

### Example (i)-a B, (i)-b B, and (i)-c B (Method 4)

A solution of (i)-(a, b, or c) A and 5-10% palladium on carbon in ethyl acetate was hydrogenated at 1-4 atm, filtered through a short silica gel plug, and concentrated to provide the desired compounds.

(Example (i)-a B) ¹H NMR (DMSO-d₆, 300 MHz) δ7.69 (s, 1H), 7.16 (d, 2H), 6.64 (d, 2H), 5.68 (s, 2H).

(Example (i)-b B) ¹H NMR (DMSO-d₆, 300 MHz) δ7.8 (s, 1H), 7.2 (t, 1H), 6.8 (d, 1H), 6.7 (s, 1H), 6.6 (d, 1H), 5.6 (s, 2H);

(Example (i)-c B) ¹H NMR (DMSO-d₆, 300 MHz) δ7.73 (s, 1H), 7.25 (t, 1H), 7.11 (d, 1H), 6.84 (d, 1H), 6.6 (t, 1H), 5.27 (s, 2H).

### Example (i)-a

### Compounds of Formula I (Method 5)

A solution of (i)-a B (1 equivalent), B-C(O)Cl (2 equivalents), and polyvinylpyridine in dichloromethane in a capped test tube was shaken overnight, treated with a primary benzyl amine resin, preferably Aminomethyl Resin-HCl (Midwest Bio-Tech, Fishers, Ind.) shaken for an additional 2 hours, eluted through a silica gel plug with acetone, and concentrated. The residue was purified by flash chromatography on silica gel to provide the desired compounds.

### Example (i)-a

### Compounds of Formula I (Method 6)

A solution of (i)-a B (1 equivalent), B-C(O)Cl (1-1.5 equivalents), and base (preferably pyridine or triethylamine, 1-10 equivalents) in an appropriate solvent, preferably dichloromethane or THF, was shaken overnight in a capped test tube, diluted with ethyl acetate, washed with saturated NaHCO₃ and 1M HCl, and concentrated. The residue was purified by flash chromatography on silica gel to provide the desired compounds.

### Example (i)-a

### Compounds of Formula I (Method 7)

Example (i)-a B (1 equivalent), the appropriate carboxylic acid (B-CO₂H, 1-2 equivalents), and EDC (1-1.5 equivalents), and DMAP (catalytic to 1 equivalent) in dichloromethane was shaken in a capped test tube for 18 hours at a temperatures between 25 and 60° C., extracted with 1N hydrochloric acid and water, dried (Na₂SO₄), and concentrated. The residue was purified by flash chromatography on silica gel to provide the desired compounds.

### Example (i)-b

### Compounds of Formula I where

### R₁ and R₃ are CF₃; R₂ is H; Z is carbon; Q is 1,3-disubstituted phenyl: R₄ and R₅ are hydrogen; L₃ is —N(R₆)C(W)—; W is O and R₆ is H

Example (i)-b B was processed as in Example (i)-a B (Method 5, 6, or 7) to provide the desired compounds.

### Example (i)-c

### Compounds of Formula I where

### R₁ and R₃ are CF₃LR₂ is H; Z is carbon; Q is 1,2-disubstituted phenyl; R₄ and R₅ are

### hydrogen: L₃ is —N(R₆)C(W)— where W is O and R₆ is H

Example (i)-c B was processed as in Example (i)-a B (Method 5, 6, or 7) to provide the desired compounds.

### Example (ii)-a (Method 8)

### Compounds of Formula I where

### R₁ and R₃ are CF₃; R₂ is H; Z is carbon; Q is 1,4-disubstituted phenyl: R₄ and R₅ are

### hydrogen, L₃ is —N(R₆)C(O)N(R₇)— where R₆ and R₇ are H

A mixture of (i)-a B (1 equivalent) and an isocyanate (B-N═C═O, 1 equivalent) in toluene was stirred at room temperature for 18 hours. The precipitate was collected by filtration, rinsed with a nonpolar solvent, preferably toluene or hexane, and dried to provide the desired compounds.

### Example (iii)-a (Method 9)

### Compounds of Formula I where

### R₁ and R₃ are CF₃;R₂ is H; Z is carbon; Q is 1,4-disubstituted phenyl; R₄ and R₅ are hydrogen; L₃ is —NR₆S(O)ₚ—, p is 2 and R₆ is H

A mixture of (i)-a B (1 equivalent), a sulfonyl chloride (B-SO₂Cl, 1-1.2 equivalents) and pyridine (3-4 equivalents) in dichloromethane at room temperature was shaken or stirred for 18 hours and purified by extractive workup or flash column chromatography on silica gel to provide the desired compounds.

### Example (iv)-a (Method 10)

### Compounds of Formula I where R₁ and R₃ are CF₃; R₂ is H; Z is carbon: Q is 1,4-disubstituted phenyl; R₄ and R₅ are hydrogen; L₃ is —N(R₆)C(W)—; W is O; and R₆ is methyl

A solution of Example (i)-a (1 equivalent) and iodomethane (4 equivalents) in THF was treated with KOH powder (5 equivalents), heated to reflux for 6 hours, cooled to room temperature (or stirred at room temperature for 20 hours), filtered and concentrated. The residue was purified by flash chromatography on silica gel to provide the desired compounds.

### Example (v)-a

### Compounds of Formula I where R₁ and R₃ are CF₃;R₂ is H; Z is carbon, Q is 1,4-disubstituted phenyl; R₄ and R₅ are hydrogen, L₃ is —C(W)N(R₆)—; W is O; and R₆ is H

### Example (v)-a A

A solution of ethyl 4-hydrazinobenzoate (1 equivalent) and 1,1,1,5,5,5-hexafluoro-2,4-pentanedione (1.1 equivalents) in 4M HCl/ethanol were heated to reflux for 18 hours and concentrated. The residue was dissolved in dichloromethane and eluted through a silica gel plug with dichloromethane to provide the desired compound.

¹H NMR (300 MHz, DMSO-d₆) δ8.17 (d, 2H), 7.91 (s, 1H), 7.8 (d, 2H), 4.38 (q, 2H), 1.35 (t, 3H).

### Example (v)-a B

A solution of Example (v)-a A (1 equivalent) and NaOH (5 equivalents) in ethanol was heated to reflux for 2 hours, concentrated, redissolved in water, acidified with 1N HCl to pH-4, and extracted with diethyl ether. The extract was washed with brine, dried (Na₂SO₄), and concentrated to provide the desired compound.

¹H NMR (DMSO-d₆, 300 MHz) δ13.4 (bs, 1H), 8.15 (d, 2H), 7.88 (s, 1H), 7.77 (d, 2H).

### Example (v)-a C

### Compounds of Formula I

A solution of Example (v)-a B (1 equivalent) in thionyl chloride (22 equivalents) was heated to reflux for 3 hours and concentrated.

### Example (v)-a (Method 11)

### Compounds of Formula I

Example (v)-a C in dichloromethane was treated with amine (H₂N-B, 1 equivalent) in the presence of pyridine (4 equivalents), and purified by flash chromatography on silica gel to provide the desired compounds.

### Example (vi)-a (Method 12)

### Compounds of Formula I where R₁ nd R₃ CF₃; R₂ is H; Z is carbon; Q is 1,4-disubstituted phenyl; R₄ and R₅ are hydrogen; L₃ is —NR₆(alkylene)ₘ—, R₆ is hydrogen, and m is 1

A slurry of Example (i)-a B (1 equivalent) and the appropriate aldehyde (B-CHO, 1.2 equivalents) in dichloromethane (20 mL) was treated with a catalytic amount of p-toluenesulfonic acid monohydrate (0.01 equivalents), stirred at room temperature for 30 minutes, treated with sodium triacetoxyborohydride (1.5 equivalents), stirred for 12 hours, diluted with dichloromethane, washed with brine, dried (Na₂SO₄), and concentrated. The residue was purified by HPLC with 10% acetone/90% hexanes to provide the desired compounds.

### Example (vii)-a

### Compounds of Formula I where R₁ and R₃ are CF₃; R₂ is H; Z is carbon; Q is 1,4-disubstituted phenyl; R₄ and R₅ are hydrogen; L₃ is —(alkylene)ₘNR₆—, R₆ is hydrogen, and m is 1

### and

### Example (viii)-a Compounds of Formula I where R₁ and R₃ are CF₃; R₂ is H; Z is carbon; Q is 1,4-disubstituted phenyl, R₄and R₅ are hydrogen; L₃ is —C(H)═N—

### Example (vii)-a A

Example (v)-a A (I equivalent) in toluene at −78° C. was treated with DIBAl-H (1.5 M solution in toluene, 1.1 equivalent), stirred for 30 minutes, treated with water, warmed to room temperature, treated with 2 M sodium hydroxide, stirred for 30 minutes, and extracted with diethyl ether. The extract was washed with brine, dried (MgSO₄) and concentrated. The residue was passed through a silica gel plug (70-230 mesh, 100 mL) with 20% acetone/hexanes then purified by normal phase HPLC with 20% acetone/hexanes to provide the desired compound.

¹H NMR (300 MHz, DMSO-d₆) δ10.10 (s, 1H), 8.20-8.10 (m, 2H), 7.90 (d, 2H), 7.85 (s, 1H);

MS (DCI/NH₃) 308 (M+NH₄-H₂O)⁺.

### Example (vii)-a and Example (viii)-a (Method 13)

### Compounds of Formula I

A mixture of Example (vii)-a A (1 equivalent) and the appropriate amine (B-NH₂, 1.1 equivalent) in dichloroethane (3 mL) at room temperature was treated sequentially with acetic acid (1.0 equivalent) and sodium triacetoxyborohydride (1.5 equivalents), shaken for 4 hours at room temperature, washed with brine, eluted through a MgSO₄/silica gel plug with 10% acetone/hexanes, concentrated, and purified on silica gel with 10% acetone/hexanes to provide a mixture of the desired compounds.

### Example (ix)-a

### Compounds of Formula I where R₁ and R₃ are CF₃; R₂ is H; Z is carbon; Q is 1,4-disubstituted phenyl; R₄ and R₅ are hydrogen; L₃ is alkenylene

### Example (ix)-a A

A solution of halide (B-Br where B is C₁-C₆ alkyl substituted with substituted aryl, 1 equivalent) and triphenylphosphine (1.2 equivalents) in toluene was heated to reflux for 2 hours, filtered, washed with toluene and dried under vacuum to provide the desired compounds.

Example (ix)-a Compounds of Formula I (Method 14)

A solution of sodium methoxide (prepared by the addition of sodium metal (1.06 equivalents) in methanol) was treated with Example (ix)-a A (1.0 equivalents) stirred at room temperature for 30 minutes, treated with Example (vii)-a A (1 equivalent), heated to reflux for 2 hours, cooled, treated with brine and extracted with diethyl ether. The extract was dried (Na₂SO₄), and concentrated. The residue was purified by HPLC eluting with acetone/hexanes to provide the desired compounds as a mixtures of Z (major) and E (minor) isomers.

### Example (x)-a

### Compounds of Formula I where R₁ and R₃ are CF₃; R₂ is H; Z is carbon, Q is 1,4-disubstituted phenyl, R₄ is hydrogen: R₅ is alkoxycarbonyl; L₃ is —N(R₆)C(W)—; W is O; R₆ is hydrogen

### Example (x)-a A

2-Fluoro-5-nitrobenzoic acid in 3:1 methanol/THF at 0° C. was treated dropwise with (trimethylsilyl)diazomethane to a yellow endpoint, stirred for 36 hours at room temperature, treated with acetic acid, and concentrated. The residue was dissolved in ethyl acetate, washed with 2M sodium hydroxide, dried (Na₂SO₄), and concentrated to provide the desired compound.

### Example (x)-a B

A slurry of sodium hydride (1 equivalent) in DMF was treated sequentially with N-3,5-bis(trifluoromethyl)pyrazole (1 equivalent) in DMF and Example (x)-a A (1 equivalent) in DMF, heated to 45° C. for 10 hours, cooled to room temperature, treated with water, and extracted with ethyl acetate. The extract was washed with 1M HCl, dried (Na₂SO₄), and concentrated. The residue was purified on silica gel with 20-70% ethyl acetate/hexanes to provide the desired compound.

¹H NMR (300 MHz, DMSO-d₆) δ8.76-8.64 (m, 2H) 8.17 (d, 1H), 7.94 (s, 1H), 3.70 (s, 3H).

### Example (x)-a C

Example (x)-a B was processed by Method 3 to provide the desired compound. mp 45-47 ° C.;

¹H NMR (300 MHz, DMSO-d₆) δ7.6 (s, 1H), 7.20 (d, 1H), 7.13 (d, 1H), 6.76 (dd, 1H), 5.92 (s, 2H), 3.46 (s, 3H).

### Example (x)-a Compounds of Formula I

Example (x)-a C was processed by Method 5, 6, or 7 to provide the desired compounds of Formula I.

### Example (xi)-a

### Compounds of Formula I where R₁ and R₃ are CF₃; R₂ is H; Z is carbon; Q is 1,4-disubstituted phenyl; R₄ is hydrogen; R₅ is CF₃; L₃ is —N(R₆)C(W)—; W is O; R₆ is hydrogen

### Example (xi)-a A

4-Bromo-3-trifluoromethylnitrobenzene was processed as in Example (x)-a B to provide the desired compound.

### Example (xi)-a B

Example (x)-a A was processed by Method 3 to provide the desired compound.

¹H NMR (DMSO-d₆, 300 MHz) δ7.78 (s, 1H), 7.4 (d, 1H), 7.03 (d, 1H), 6.84 (dd, 1H), 6.25 (s, 2H).

### Example (xi)-aCompounds of Formula I

Example (xi)-a B was processed by Method 5, 6, or 7 to provide the desired compounds of Formula I.

### Example (xii)-a

### Compounds of Formula I where R₁ and R₃ are CF₃; R₂ is H; Z is carbon; Q is 1,4-disubstituted phenyl; R₄ is hydrogen; R₅ is CF₃; L₃ is —N(R₆)C(W)—; W is O; R₆ is hydrogen

### Example (xii)-a A 4-Fluoro-2-trifluoromethylnitrobenzene was processed as in Example (x)-a B to provide the desired compound.

Example (xii)-a B

Example (xii)-a A was processed by Method 3 to provide the desired compound.

¹H NMR (DMSO-d₆, 300 MHz) δ7.75 (s, 1H), 7.6 (d, 1H), 7.46 (dd, 1H), 6.95 (d, 1H), 6.22 (s, 2H).

### Example (xii)-aCompounds of Formula I

Example (xii)-a B was processed by Method 5, 6, or 7 to provide the desired compounds of Formula I.

### Example (xiii)-a

### Compounds of Formula I where R₁ and R₃ are CF₃; R₂ is H; Z is carbon; Q is 1,4-disubstituted phenyl; R₄ is hydrogen; R₅ is halo; L₃ is —N(R₆)C(W)—; W is O; R₆ is hydrogen

### Example (xiii)-a A

4-Bromo-3-chloronitrobenzene was processed as in Example (x)-a B to provide the desired compound.

### Example (xiii)-a B

Example (xiii)-a A was processed by Method 3 to provide the desired compound.

¹H NMR (DMSO-d₆, 300 MHz) δ7.76 (s, 1H), 7.32 (d, 1H), 6.8 (d, 1H), 6.1 (dd, 1H), 6.04 (s, 2H).

### Example (xiii)-aCompounds of Formula I

Example (xiii)-a B was processed by Method 5, 6, or 7 to provide the desired compounds of Formula I.

### Example (xiv)-a

### Compounds of Formula I where R₁ and R₃ are CF₃; R₂ is H; Z is carbon; Q is 1,4-disubstituted phenyl; R₄ is hydrogen; R₅ is methyl; L₃ is —N(R₆)C(W)—; W is O; R₆ is hydrogen

### Example (xiv)-a A

4-Fluoro-2-methylnitrobenzene was processed as in Example (x)-a B to provide the desired compound.

### Example (xiv)-a B

Example (xiv)-a A was processed by Method 3 to provide the desired compound.

¹H NMR (DMSO-d₆, 300 MHz) δ7.68 (s, 1H), 7.1 (d, 1H), 7.06 (dd, 1H), 6.68 (d, 1H), 5.4 (s, 2H), 2.08 (s, 3H).

### Example (xiv)-aCompounds of Formula I

Example (xiv)-a B was processed by Method 5, 6, or 7 to provide the desired compounds of Formula I.

### Example (xv)-a

### Compounds of Formula I where R₁ and R₃ are CF₃; R is H; Z is carbon; Q is 1,4-disubstituted phenyl; R₄ is hydrogen; R₅ is alkoxy; L₃ is —N(R₆)C(W)—; W is O; R₆ is hydrogen

### Example (xv)-a A

4-Fluoro-2-methoxynitrobenzene was processed as in Example (x)-a B and purified by flash chromatography on silica gel with 1:70:30 ethyl acetate/pentane/dichloromethane to provide the desired compound.

Example (xv)-a B

Example (xv)-a A was processed by Method 3 to provide the desired compound.

1H NMR (DMSO-d₆, 300 MHz) δ7.73 (s, 1H), 6.99 (d, 1H), 6.85 (dd, 1H), 6.7 (d, 1H, 3.88 (s, 3H).

### Example (xv)-aCompounds of Formula I

Example (xv)-a B was processed by Method 5, 6, or 7 to provide the desired compounds of Formula I.

### Example (xvi)-a

### Compounds of Formula I where R₁ and R₃ are CF₃; R₂ is H; Z is carbon; Q is 1,4-disubstituted phenyl; R₄ is hydrogen; R₅ is halo; L₃ is —N(R₆)C(W)—; W is O; R₆ is hydrogen

### and

### Example (xvii)-aR₁ and R₃ are CF₃; R₂ is H; Z is carbon; Q is 1,4-disubstituted phenyl; R₄ is hydrogen; R₅ is substituted heterocycle; L₃ is —N(R₆)C(W)—; W is O; R₆ is hydrogen

### Example (xvi)-a A and Example (xvii)-a A

2,4-Difluoronitrobenzene was processed as in Example (x)-a B to provide a mixture of the desired compounds.

### Example (xvi)-a B and Example (xvii)-a B

Examples (xvi)-a A and Example (xvii)-a A were processed by Method 3 to provide a mixture the desired compounds.

¹H NMR (DMSO-d₆, 300 MHz) (mixture of (xvi)-a B and (xvii)-a B ) (xvi)-a B: δ7.74 (s, 1H), 7.19 (m, 2H), 6.84 (dd, 1H), 5.18 (s, 2H) and (xvii)-a B: 8 7.74 (s, 1H), 7.72 (s, 1H), 7.52 (d, 1H), 7.48 (dd, 1H), 6.94 (d, 1H), 5.95 (s, 2H).

### Example (xvi)-a and Example (xvii)-a

### Compounds of Formula I

Example (xvi)-a B and Example (xvii)-a B were processed by Method 5, 6, or 7 to provide a mixture the desired compounds of Formula I which were separated by column chromatography.

### (xviii)-a

### Compounds of Formula I where R₁ is CH₃; R₂ is H; R₃ is CF₃; Z is carbon; Q is 1,4-disubstituted phenyl; R₄ and R₅ are hydrogen; L₃ is —N(R₆)C(W)—; W is O; and R₆ is H

### and

### (xix)-a

### R₁ is CF₃; R₂ is H; R₃ is CH₃; Z is carbon; Q is 1,4-disubstituted phenyl; R₄and R₅ are hydrogen; L₃ is —N(R₆)C(W)—; W is O; and R₆ is H

### Example (xviii)-a A and Example (xix)-a A

A solution of 4-nitrophenylhydrazine (5 g, 32.5 mmol) and 1,1,1-trifluoro-2,4-pentanedione (4.97 g, 32.5 mmol) in ethanol (200 mL) was treated with concentrated sulfuric acid (1 mL), refluxed for 1 hour, and concentrated. The residue was dissolved in ethyl acetate, washed with water and brine, dried (MgSO₄), and concentrated. The residue was purified on silica gel eluting with 3.5% ethyl acetate/pentane to provide the desired compounds.

(xviii)-a A: 1H NMR (DMSO-d₆, 300 MHz) δ8.42 (d, 2H), 7.9 (d, 2H), 7.1 (s, 1H), 2.33 (s, 3H) and (xix)-a A: ¹H NMR (DMSO-d₆, 300 MHz δ8.42 (d, 2H), 7.94 (d, 2H), 6.88 (s, 1H), 2.46 (s, 3H).

### Example (xviii)-a B

A solution of Example (xviii)-a A was processed by Method 3 to provide the desired compound.

¹H NMR (DMSO-d₆, 300 MHz) δ7.05 (d, 2H), 6.77 (s, 1H), 6.62 (d, 2H), 2.24 (s, 3H).

### Example (xix)-a B

Example (xix)-a A was processed by Method 3 to provide the desired compound.

¹H NMR (DMSO-d₆, 300 MHz) δ7.13 (d, 2H), 6.65 (s, 1H), 6.64 (d, 2H), 5.48 (s, 1H), 2.24 (s, 3H).

### (xviii)-a

### Compounds of Formula I

### and

### (xix)-a

### Compounds of Formula I

Example (xviii)-a B and Example (xix)-a B were each processed by Method 5, 6, or 7 to provide the desired compounds of Formula I.

### (xx)-a

### Compounds of Formula I where R₁ is CH₃; R₂ and R₃ are H; Z is carbon; Q is 1,4-disubstituted phenyl; R₄ and R₅ are hydrogen; L₃ is —N(R₆)C(W)—; W is O; and R₆ is H

### and

### (xxi)-a

### R₁ and R₂ are H; R₃ is CH₃; Z is carbon; Q is 1,4-disubstituted phenyl; R₄and R₅ are hydrogen; L₃ is —N(R₆)C(W)—; W is O; and R₆ is H

### Example (xx)-a A and (xxil-a A

4-Nitrophenylhydrazine and acetylacetaldehyde dimethylacetal were processed as in Example (xviii)-a A/Example (xix)-a A and purified by flash chromatography on silica gel with 0.5:5:5 ethyl acetate/dichloromethane/pentane to provide the desired compounds.

(xx)-a A: ¹H NMR (CDCl₃, 500 MHz) δ8.32 (d, 2H), 7.93 (d, 1H), 7.84 (d, 2H), 6.36 (d, 1H), 2.4 (s, 3H) and (xxi)-a A: ¹H NMR (CDCl₃, 500 MHz) δ8.36 (d, 2H), 7.73 (d, 2H), 7.64 (d, 1H), 6.28 (d, 1H), 2.48 (s, 3H).

### Example (xx)-a B

Example (xx)-a A was processed by Method 3 to provide the desired compound.

¹H NMR (CDCl₃, 300 MHz) δ7.68 (d, 1H), 7.05 (d, 2H), 6.75 (d, 2H), 6.20 (d, 1H), 2.38 (s, 3H).

### Example (xxi)-a B

Example (xxi)-a A was processed by Method 3 to provide the desired compound.

¹H NMR (DMSO-d₆, 300 MHz) δ7.42 (s, 1H), 7.08 (dd, 2H), 6.62 (dd, 2H), 6.17 (s, 1H), 5.3 (br s , 2H), 2.22 (s, 3H).

### (xx)-a

### Compounds of Formula I

### and

### (xxi)-a

### Compounds of Formula I

Example (xx)-a B and Example (xxi)-a B were each processed by Method 5, 6, or 7 to provide the desired compounds of Formula I.

### Example (xxii)-a

### Compounds of Formula I where

### R₁ is CF₃; R₂ and R₃ are H; Z is carbon; O is 1,4-disubstituted phenyl; R₄ and R₅ are hydrogen; L₃ is —N(R₆)C(W)—; W is O; and R₆ is H

### Example (xxii)-a A

A solution of 3-trifluoromethylpyrazole (1 g, 7.4 mmol) in DMF (10 mL) at 0° C. was treated with NaH (60% in oil, 382 mg, 9.6 mmol), stirred at room temperature for 30 minutes, treated with 4-fluoronitrobenzene (1.04 g, 7.4 mmol), stirred for 18 hours, treated with water, and extracted with ethyl acetate. The extract was washed with brine, dried (MgSO₄) and concentrated. The residue was purified on silica gel with 9% ethyl acetate/pentane to provide the desired compound.

1H NMR (DMSO-d₆, 300 MHz) δ8.9 (m, 1H), 8.43 (d, 2H), 8.2 (d, 2H), 7.2 (d, 1H).

### Example (xxii)-a B

Example (xxii)-a A was processed by Method 3 to provide the desired compound.

¹H NMR (DMSO-d₆, 300 MHz) δ8.42 (m, 1H), 7.45 (d, 2H), 6.92 (d, 1H), 6.65 (d, 2H), 5.4 (m, 2H).

### Example (xxii)-a

### Compounds of Formula I

Example (xxii)-a B was processed by Method 5, 6, or 7 to provide the desired compounds of Formula I.

### Example (xxiii)-a

### Compounds of Formula I where R₁ is CF₃; R₂ is H; R₃ is hydroxyl; Z is carbon: Q is 1,4-disubstituted phenyl; R₄ and R₅ are hydrogen: L₃ is —N(R₆)C(W)—; W is O; and R₆ is H

### Example (xxiii)-a A

A solution of ethyl 4,4,4-trifluoroacetoacetate (10 g, 54 mnmol) and 4-nitrophenylhydrazine (8.3 g, 54 mmol) in ethanol (200 mL) was treated with concentrated sulfuric acid (0.5 ml), refluxed for 25 minutes, and concentrated. The residue was dissolved in ethyl acetate, washed with brine, dried (MgSO₄), and concentrated to provide the desired compound.

¹HNMR (CDCl₃, 300 MHz) δ9.8 (s, 1H), 8.21 (d, 2H), 7.23 (d, 2H), 4.27 (q, 2H), 3.56 (s, 2H), 1.24 (t, 3H).

### Example (xxiii)-a B

A solution of Example (xxiii)-a A (7.7 g, 24.2 mmol) in 2:1 ethanol:dichloromethane (300 mL) was treated with anhydrous K₂CO₃ (6.7 g, 48.4 mmol), stirred at room temperature for 18 hours, and concentrated. The residue was neutralized with dilute HCl, extracted with ethyl acetate, washed with brine, dried (MgSO₄), and concentrated. The residue was flash chromatographed on silica gel with 5% methanol/dichloromethane to provide the desired compound.

1H NMR (DMSO-d₆, 300 MHz) δ8.38 (d, 2H), 8.15 (d, 2H), 5.9 (s, 1H).

### Example (xxiii)-a C

Example (xxiii)-a B was processed by Method 3 to provide the desired compound.

1H NMR (DMSO-d₆, 300 MHz) δ7.24 (d, 2H), 6.62 (d, 2H), 5.85 (s, 1H ), 5.4 (m, 2H).

### Example (xxiii)-a

### Compounds of Formula I

Example (xxiii)-a C was processed by Method 5, 6, or 7 to provide the desired compounds of Formula I.

### Example (xxiv)-a

### Compounds of Formula I where R₁ and R₃ are CF₃; R₂ is H; Z is carbon; Q is 1,4-disubstituted pyridine; R₄ and R₅ are hydrogen; L₃ is —N(R₆)C(W)— where W is O and R₆ is H

### Example (xxiv)-a A

N-3,5-bis(trifluoromethyl)pyrazole was processed as in Example (x)-a B but substituting 2-chloro-5-nitropyridine for Example (x)-a A to provide the desired compound.

1H NMR (DMSO-d₆, 300 MHz) δ9.39 (d, 1H), 8.88 (dd, 1H), 8.21 (d, 2H), 8.02 (s, 1H).

### Example (xxiv)-a B

Example (xxiv)-a A was processed by Method 3 to provide the desired compound.

¹H NMR (DMSO-d₆, 300 MHz) δ7.82(d, 1H), 7.72 (s, 1H), 7.43 (d, 1H), 7.14 (dd, 1H), 5.90 (s, 2H).

### Example (xxiv)-a

### Compounds of Formula I

Example (xxiv)-a B was processed by Method 5, 6, or 7 to provide the desired compounds of Formula I.

### Example (xxv)-a

### Compounds of Formula I where R₁ and R₃ are CH₃; Z is nitrogen; Q is 1,4-disubstituted phenyl; R₄ and R₅ are hydrogen; L₃ is —N(R₆)C(W)— where W is O and R₆ is H

### Example (xxv)-a A

A solution of 4-nitrophenylhydrazine (2 g, 13.1 mmol) and diacetamide (1.32 g, 13.1 mmol) in ethanol (80 mL) was treated with concentrated sulfuric acid (0.5 ni), refluxed for 1 hour, and concentrated. The residue was dissolved in ethyl acetate, washed with water and brine, dried (MgSO₄) and concentrated. Purification of the residue by flash chromatography on silica gel with 3:2:5 ethyl acetate/pentane/dichloromethane provided the desired compound. ¹H NMR (DMSO-d₆, 30 MHz) δ8.4(d, 2H) 7.9(d, 2H), 2.54 (s, 3H), 2.33 (s, 3H).

### Example (xxv)-a B

Example (xxv)-a A was processed by Method 3 to provide the desired compound. ¹H NMR (DMSO-d₆, 300 MHz) δ7.18 (d, 2H), 6.7 (d, 1H), 5.45 (s, 2H), 2.35 (s, 3H), 2.27 (s, 3H).

### Example (xxv)-a

### Compounds of Formula I

Example (xxv)-a B was processed by Method 5, 6, or 7 to provide the desired compounds of Formula I.

### Example 1

### N-[4-[3,5-bis(trifluoromethyl)-1H-pyrazol-1-yl]phenyl]cyclopropanecarboxamide

Example (i)-a B was processed as in Example (i)-a (Method 5, 6, or 7) to provide the title compound.

mp 204-205 ° C.;

MS (DCI/NH₃) m/e 381 (M+NH₄)⁺;

¹H NMR (DMSO-d₆, 300 MHz) δ10.52 (s, 1H), 7.79 (d, 2H), 7.78 (s, 1H), 7.53 (d, 2H), 1.81 (m, 1H), 0.85 (d, 4H).

### Example 2

### N-[4-[3,5-bis(trifluoromethyl)-1H-pyrazol-1-yl]phenyl]-2.2.3.3-tetramethylcyclopropanecarboxamide

Example (i)-a B was processed as in Example (i)-a (Method 5, 6, or 7) to provide the title compound.

mp 171-172 ° C.;

MS (DCI/NH₃) m/e 437 (M+NH₄)⁺;

¹H NMR (DMSO-d₆, 300 MHz) δ7.78 (s, 1H), 7.77 (d, 2H), 7.5 (d, 2H), 1.33 (s, 1H), 1.26 (s, 6H), 1.2 (s, 6H).

### Example 3

### N-[4-[3,5-bis(trifluoromethyl)-1H-pyrazol-1-yl]phenyl]-2,2-dichloro-1-methylcyclopropanecarboxamide

Example (i)-a B was processed as in Example (i)-a (Method 5, 6, or 7) to provide the title compound.

mp 143-145 ° C.;

MS (ESI−) m/e 445 (M−H)⁻;

1H NMR (CDCl₃, 300 MHz) δ7.65 (d, 2H), 7.50 (d, 3H), 7.15 (s, 1H), 2.35 (d, 1H), 1.65 (s, 3H), 1.45 (d, 1H).

### Example 4

### N-[4-[3,5-bis(trifluoromethyl)-1H-pyrazol-1-yl]phenyl]-2-oxo-6-pentyl-2H-pyran-3-carboxamide

Example (i)-a B was processed as in Example (i)-a (Method 5, 6, or 7) to provide the title compound.

mp 140-141 ° C.;

MS (DCI/NH₃) m/e 488 (M+H)⁺;

1H NMR (DMSO-d₆, 300 MHz) δ8.4 (d, 1H), 7.92 (d, 2H), 7.83 (s, 1H), 7.62 (d, 2H), 6.62 (d, 1H), 2.65 (t, 2H), 1.65 (m, 2H), 1.33 (m, 4H), 0.89 (t, 3H).

### Example 5

### N-[4-[3,5-bis(trifluoromethyl)-1H-pyrazol-1-yl]phenyl]-3,5-difluorobenzenesulfonamide

Example (i)-a B was processed as in Example (iii)-a (Method 9) to provide the title compound.

MS (DCI/NH₃) m/e 489 (M+NH₄)⁺;

¹H NMR (DMSO-d₆, 300 MHz) δ11.19 (s, 1H), 8.03-7.95 (m, 1H), 7.79 (s, 1H), 7.60-7.55 (m, 1H), 7.52 (d, 2H), 7.33-7.29 (m, 1H), 7.28 (d, 2H).

### Example 6

### N-[4-[3,5-bis(trifluoromethyl)-1H-pyrazol-1-yl]phenyl]-1-cyclohexene-1-carboxamide

Example (i)-a B was processed as in Example (i)-a (Method 5, 6, or 7) to provide the title compound.

mp 146-148 ° C.;

MS (ESI−) 402 (M−H)⁻;

¹H NMR (CDCl₃, 300 MHz) δ7.65 (d, 2H), 7.55 (br s, 1H ), 7.45 (d, 2H), 7.05 (s, 1H), 4.78 (m, 1H), 2.40-2.20 (m, 4H), 1.80-1.60 (m, 4H).

### Example 7

### N-[4-[3,5-bis(trifluoromethyl)-1H-pyrazol-1-yl]phenyl]-2-methylcyclopropanecarboxamide

Example (i)-a B was processed as in Example (i)-a (Method 5, 6, or 7) to provide the title compound.

mp 172-173 ° C.;

MS (DCI/NH₃) m/e 395 (M+NH₄)^{+6l ;}

¹H NMR (DMSO-d₆, 300 MHz) δ7.8 (s, 1H), 7.78 (d, 2H), 7.54 (d, 2H), 1.57 (m, 1H), 1.27 (m, 1H), 1.12 (d, 3H), 1.05 (m, 1H), 0.7 (m, 1H);

### Example 8

### N-[4-[3,5-bis(trifluoromethyl)-1H-pyrazol-1-yl]phenyl]-5-(3,5-dichlorophenoxy)-2-furancarboxamide

Example (i)-a B was processed as in Example (i)-a (Method 5, 6, or 7) to provide the title compound.

mp 152-153 ° C.;

MS (DCI/NH₃) m/e 551 (M+H)⁺;

¹H NMR (DMSO-d₆, 300 MHz) δ10.45 (s, 1H), 7.95 (d, 2H), 7.82 (s, 1H), 7.6 (d, 2H), 7.55 (t, 1H), 7.47 (d, 1H), 7.4 (d, 2H), 6.15 (d, 1H).

### Example 9

### N-[4-[3,5-bis(trifluoromethyl)-1H-pyrazol-1-yl]phenyl]-1-methyl-2-cyclohexene-1-carboxamide

Example (i)-a B was processed as in Example (i)-a (Method 5, 6, or 7) to provide the title compound.

mp 108-110 ° C.;

MS (ESI−) m/e 416 (M−H)⁻;

¹H NMR (CDCl₃, 300 MHz) δ7.70 (d, 3H), 7.43 (d, 2H), 7.05 (s, 1H), 5.90-5.65 (m, 2H), 2.63-2.45 (m, 1H), 2.20-1.85 (m, 4H), 1.60-1.60 (m, 1H), 1.25 (s, 3H).

### Example 10

### N-[4-[3,5-bis(trifluoromethyl)-1H-pyrazol-1-yl]phenyl]-1-cyclopentene-1-carboxamide

Example (i)-a B was processed as in Example (i)-a (Method 5, 6, or 7) to provide the title compound.

mp 174-175 ° C.;

MS (DCI/NH₃) m/e 407 (M+NH₄)⁺;

¹H NMR (DMSO-d₆, 300 MHz) δ9.95 (s, 1H), 7.90 (d, 2H), 7.79 (s, 1H), 7.55 (d, 2H), 6.78 (m, 1H), 2.64-2.46 (m, 4H), 1.93 (m, 2H).

### Example 11

### N-[4-[3,5-bis(trifluoromethyl)-1H-pyrazol-1-yl]phenyl]-3-methoxycyclohexanecarboxamide

Example (i)-a B was processed as in Example (i)-a (Method 5, 6, or 7) to provide the title compound.

mp 145-147 ° C.;

MS (DCI/NH₃) m/e 436 (M+H)⁺;

¹H NMR (CDCl₃, 300 MHz) (diasteromers) δ7.85 (br s, 1H), 7.70 (m, 4H), 7.45 (m, 4H), 7.35 (br s, 1H ), 7.05 (s, 2H ), 3.65 (m, 1H), 3.40 (s, 3H), 3.35 (s, 3H), 3.26 (m, 1H), 2.65 (m, 1H), 2.45 -2.25 (m, 1H), 2.15-1.85 (m, 8H), 1.8-1.3 (m, 8H).

### Example 12

### N-[4-[3,5-bis(trifluoromethyl)-1H-pyrazol-1-yl]phenyl]-2-butynamide

Example (i)-a B was processed as in Example (i)-a (Method 5, 6, or 7) to provide the title compound.

mp 220-221 ° C.;

MS (DCI/NH₃) m/e 379 (M+NH₄)⁺;

¹H NMR (DMSO-d₆, 300 MHz) δ7.82 (s, 1H), 7.79 (d, 2H), 7.57 (d, 2H), 2.08 (s, 3H).

### Example 13

### ethyl 3-[[[[4-[3,5-bis(trifluoromethyl)-1H-pyrazol-1-yl]phenyl]amino]carbonyl]amino]-benzoate

Example (i)-a B was processed as in Example (ii)-a (Method 8) to provide the title compound.

mp 211-212 ° C.;

MS (DCI/NH₃) m/e 504 (M+NH₄)⁺;

¹H NMR (DMSO-d₆, 300 MHz) δ9.09 (s, 1H), 8.46 (t, 1H), 8.10 (s, 1H), 7.98 (d, 4H), 7.95 (dt, 1H), 7.84 (d, 2H), 4.68 (q, 2H), 1.67 (t, 3H).

### Example 14

### N-[4-[3,5-bis(trifluoromethyl)-1H-pyrazol-1-yl]phenyl]-3-furancarboxamide

Example (i)-a B was processed as in Example (i)-a (Method 5, 6, or 7) to provide the title compound.

mp 206-207 ° C.;

MS (DCI/NH₃) m/e 390 (M+H)⁺;

¹H NMR (DMSO-d₆, 300 MHz) δ10.25 (s, 1H), 8.43 (s, 1H), 7.95 (d, 2H), 7.83 (s, 2H), 7.6 (d, 2H), 7.03 (s, 1H).

### Example 15

### N-[4-[3,5-bis(trifluoromethyl)-1H-pyrazol-1-yl]phenyl]-2-methyl-3-nitrobenzamide

Example (i)-a B was processed as in Example (i)-a (Method 5, 6, or 7) to provide the title compound.

mp 194-195 ° C.;

MS (DCI/NH₃) m/e 476 (M+NH₄)⁺;

¹H NMR (DMSO-d₆, 300 MHz) δ10.95 (s, 1H), 8.04 (d, 1H), 7.95 (d, 2H), 7.85 (d, 1H), 7.82 (s, 1H), 7.64 (d, 2H,), 7.6 (t, 1H), 2.48 (s, 3H).

### Example 16

### N-[4-[3,5-bis(trifluoromethyl)-1H-pyrazol-1-yl]phenyl]-N′-(3-cyanophenyl)urea

Example (i)-a B was processed as in Example (ii)-a (Method 8) to provide the title compound.

mp 204-205 ° C.;

MS (DCI/NH₃) m/e 457 (M+NH₄)⁺;

¹H NMR (DMSO-d₆, 300 MHz) δ9.24 (s, 1H), 9.16 (s, 1H), 7.98 (t, 1H), 7.79 (s, 1H), 7.73-7.65 (m, 3H), 7.54 (d, 2H), 7.43 (dd, 2H).

### Example 17

### N-[4-[3,5-bis(trifluoromethyl)-1H-pyrazol-1-yl]phenyl]-1-hydroxycyclopropanecarboxamide

Example (i)-a B was processed as in Example (i)-a (Method 5, 6, or 7) to provide the title compound.

mp 198-200 ° C.;

MS (DCI/NH₃) m/e 397 (M+NH₄)⁺;

¹H NMR (DMSO-d₆, 300 MHz) δ10.22 (s, 1H), 7.98 (d, 2H), 7.81 (s, 1H), 7.54 (d, 2H), 6.64 (s, 1H), 1.19 (m, 1H), 1.1 (t, 2H), 1.0 (m, 1H).

### Example 18

### N-[4[3,5-bis(trifluoromethyl)-1H-pyrazol-1-yl]phenyl]cycloheptanecarboxamide

Example (i)-a B was processed as in Example (i)-a (Method 5, 6, or 7) to provide the title compound.

mp 174-175 ° C.;

MS (DCI) m/e 420 (M+H)⁺;

¹H NMR (DMSO-d₆, 300 MHz) δ10.15 (s, 1H), 7.8 (s, 1H), 7.8 (d, 2H), 7.52 (d, 2H), 1.4-1.9 (m, 13H).

### Example 19

### N-[4-[3,5-bis(trifluoromethyl)-1H-pyrazol-1-yl]phenyl]-2-benzofurancarboxamide

Example (i)-a B was processed as in Example (i)-a (Method 5, 6, or 7) to provide the title compound.

mp 213-215 ° C.;

MS (DCI/NH₃) m/e 440 (M+H)⁺;

¹H NMR (DMSO-d₆, 300 MHz) δ10.87 (s, 1H), 8.05 (d, 2H), 7.86 (s, 1H), 7.85 (s, 1H), 7.86 (d, 1H), 7.76 (d, 1H), 7.64 (d, 2H), 7.55 (t, 1H), 7.4 (t, 1H).

### Example 20

### N-[4-[3,5-bis(trifluoromethyl)-1H-pyrazol-1-yl]phenyl]-5-fluoro-1H-indole-2-carboxamide

Example (i)-a B was processed as in Example (i)-a (Method 5, 6, or 7) to provide the title compound.

mp 253-255 ° C.;

MS (DCI/NH₃) m/e 457 (M+H)⁺;

¹H NMR (DMSO-d₆, 300 MHz) δ11.95 (s, 1H), 10.58 (s, 1H), 8.03 (d, 2H), 7.64 (d, 2H), 7.84 (s, 1H), 7.45-7.54 (m, 3H), 7.12 (dt, 1H).

### Example 21

### (E)-N-[4-[3,5-bis(trifluoromethyl)-1H-pyrazol-1-yl]phenyl]-3-(2-chlorophenyl)-2-propenamide

Example (i)-a B was processed as in Example (i)-a (Method 5, 6, or 7) to provide the title compound.

mp 173-175 ° C.;

MS (DCI/NH₃) m/e 460 (M+H)⁺;

¹H NMR (DMSO-d₆, 300 MHz) δ10.7 (s, 1H), 7.94 (d, 1H), 7.92 (d, 2H), 7.83 (s, 1H), 7.81 (m, 1H), 7.61 (d, 2H), 7.59 (m, 1H), 7.47 (m, 2H), 6.93 (d, 1H).

### Example 22

### 2-Benzoyl-N-[4-[3,5-bis(trifluoromethyl)-1H-pyrazol-1-yl]phenyl]benzamide

Example (i)-a B was processed as in Example (i)-a (Method 5, 6, or 7) to provide the title compound.

mp 204-205 ° C.;

MS (DCI/NH₃) m/e 521 (M+NH₄)⁺;

1H NMR (DMSO-d₆, 300 MHz) δ7.9 (d, 3H), 7.8 (s, 1H), 7.69-7.57 (m, 2H), 7.53 (d, 2H), 7.42 (d, 2H), 7.35-7.22 (m, 4H).

### Example 23

### 3a(S)-(3aα,4β,6aα)-N-[4-[3,5-bis(trifluoromethyl)-1H-pyrazol-1-yl]phenyl]hexahydro-2-oxo-1H-thieno[3,4-d]limidazole-4-pentanamide

Example (i)-a B was processed as in Example (i)-a (Method 5, 6, or 7) to provide the title compound.

mp 185-186 ° C.;

MS (ESI) m/e 522 (M+H)⁺;

¹H NMR (DMSO-d₆, 300 MHz) δ10.22 (s, 1H), 7.8 (d, 2H), 7.79 (s, 1H), 7.53 (d, 2H), 6.41 (s, 1H), 6.33 (s, 1H), 4.31 (m, 1H), 4.15 (m, 1H), 3.14 (m, 1H), 2.83 (dd, 1H), 2.59 (d, 1H), 2.37 (t, 2H), 1.35-1.7 (m, 6H).

### 158406 Example 24

### N-4-[3,5-bis(trifluoromethyl)-1H-pyrazol-1-yl]-N-(4-chlorophenyl)benzamide

Example (i)-a B was processed as in Example (i)-a (Method 5, 6, or 7) to provide the title compound.

mp 183-185 ° C.;

¹H NMR (DMSO-d₆, 300 MHz) δ10.69 (s, 1H), 8.23-8.10 (m, 1H), 8.07-8.03 (m, 4H), 7.87 (s, 1H), 7.86-7.72 (m, 4H);

MS (DCI/NH₃) m/e 451 (M+NH₄)⁺.

### Example 25

### N-[4-[3,5-bis(trifluoromethyl)-1H-pyrazol-1-yl]phenyl]-3-iodobenzamide

Example (i)-a B was processed as in Example (i)-a (Method 5, 6, or 7) to provide the title compound.

mp 192-193 ° C.;

MS (DCI/NH₃) m/e 543 (M+NH₄)⁺;

¹H NMR (DMSO-d₆, 300 MHz) δ8.32 (dd, 1H), 7.99 (d, 4H), 7.83 (s, 1H), 7.62 (d, 2H), 7.37 (t, 1H).

### Example 26

### N-[4-[3,5-bis(trifluoromethyl)-1H-pyrazol-1-yl]phenyl]bicyclo[2.2.1]hept-5-ene-2-carboxamide

Example (i)-a B was processed as in Example (i)-a (Method 5, 6, or 7) to provide the title compound.

MS (DCI/NH₃) m/e 433 (M+NH₄)⁺;

¹H NMR (DMSO-d₆, 300 MHz) δ10.12 (s, 1H), 7.80 (s, 1H), 7.78 (d, 2H), 7.51 (d, 2H), 6.19 (dd, 1H) 5.88 (dd, 1H), 3.11-3.05 (m, 1H), 2.89 (s, 1H), 1.88-1.80 (m, 1H), 1.43 (dd, 1H), 1.34 (s, 2H).

### Example 27

### N-[4-[3,5-bis(trifluoromethyl)-1H-pyrazol-1-yl]phenyl]-2-methylcyclohexanecarboxamide

Example (i)-a B was processed as in Example (i)-a (Method 5, 6, or 7) to provide the title compound.

MS (DCI/NH3) m/e 437 (M+NH₄)⁺;

¹H NMR (DMSO-d₆, 300 MHz) δ10.5 (s, 1H), 7.8 (d, 2H), 7.8 (s, 1H), 7.5 (d, 2H), 1.91 (m, 4H), 1.6-1.5 (m, 2H), 1.4-1.2 (m, 4H), 0.9 (d, 3H).

### Example 28

### phenylmethyl [1-[[[4-[3,5-bis(trifluoromethyl)-1H-pyrazol-1-yl]phenyl]amino]carbonyl]propyl]carbamate

Example (i)-a B was processed as in Example (i)-a (Method 5, 6, or 7) to provide the title compound.

mp 158-160° C.;

MS (DCI/NH₃) m/e 515 (M+H)⁺;

¹H NMR (DMSO-d₆, 300 MHz) δ10.38 (s, 1H), 7.82 (d, 2H), 7.8 (s, 1H), 7.62 (d, 1H), 7.55 (d, 2H), 7.3-7.4 (m, 5H), 5.05 (s, 2H), 4.0-4.14 (m, 1H), 1.6-1.8 (m, 2H), 0.93 (t, 3H).

### Example 29

### N-[4-[3,5-bis(trifluoromethyl)-1H-pyrazol-1-yl]phenyl]-3-cyclohexene-1-carboxamide

Example (i)-a B was processed as in Example (i)-a (Method 5, 6, or 7) to provide the title compound.

mp 177-178° C.;

MS (DCI/NH₃) m/e 421 (M+NH₄)⁺;

¹H NMR (DMSO-d₆, 300 MHz) δ7.91 (d, 2H), 7.88 (s, 1H), 7.63 (d, 2H), 5.80 (s, 2H), 2.75 (m, 1H), 2.35-2.20 (m, 2H), 2.22-1.97 (m, 2H), 2.05-1.99 (m, 1H), 1.77-1.62 (m, 1H).

### Example 30

### 4-[3,5-bis(trifluoromethyl)-1H-pyrazol-1-yl]-N-(4-fluorophenyl)benzamide

Example (v)-a C was processed as in Example (v)-a (Method 11) to provide the title compound.

mp 194-195° C.;

MS (DCI/NH₃) m/e 417 (M+H)⁺;

¹H NMR (DMSO-d₆, 300 MHz) δ10.58 (s, 1H), 8.15 (d, 2H), 7.9 (s, 1H), 7.81 (d, 2H), 7.8 (d, 2H), 7.23 (t, 2H).

### Example 31

### N-[4-[3,5-bis(trifluoromethyl)-1H-pyrazol-1-yl]phenyl]-N′-(3-nitrophenyl)urea

Example (i)-a B was processed as in Example (ii)-a (Method 8) to provide the title compound.

mp 203-204° C.;

MS (DCI/NH₃) m/e 477 (M+NH₄)⁺;

¹H NMR (DMSO-d₆, 300 MHz) δ9.42 (s, 1H), 9.38 (s, 1H), 8.58 (t, 1H), 7.84 (d, 1H), 7.80 (s, 1H), 7.77 (d, 1H), 7.70 (d, 2H), 7.60 (d, 1H), 7.53 (d, 2H).

### Example 32

### N-[4-[3,5-bis(trifluoromethyl)-1H-pyrazol-1-yl]phenyl]-N′-(4-fluorophenyl)urea

Example (i)-a B was processed as in Example (ii)-a (Method 8) to provide the title compound.

mp 201-202° C.;

MS (DCI/NH₃) m/e 450 (M+NH₄)⁺;

¹H NMR (DMSO-d₆, 300 MHz) δ9.50 (s, 1H), 9.21 (s, 1H), 8.19 (s, 1H), 8.07 (d, 2H), 7.96-7.83 (m, 4H), 7.55 (t, 2H).

### Example 33

### N-[4-[3,5-bis(trifluoromethyl)-1H-pyrazol-1-yl]phenyl]-N′-[4-(trifluoromethyl)phenyl]urea

Example (i)-a B was processed as in Example (ii)-a (Method 8) to provide the title compound.

mp 210-212° C.;

MS (DCI/NH₃) m/e 516 (M+NH₄)⁺;

¹H NMR (DMSO-d₆, 300 MHz) δ9.11 (s, 1H), 9.01 (s, 1H), 7.78 (s, 1H), 7.67 (d, 2H), 7.59 (d, 2H), 7.53 (d, 2H), 7.31 (d, 2H).

### Example 34

### N-[4-[3,5-bis(trifluoromethyl)-1H-1-pyrazol-1-yl]phenyl]-N′-(3,5-dimethylphenyl)urea

Example (i)-a B was processed as in Example (ii)-a (Method 8) to provide the title compound.

mp>230° C.;

MS (DCI/NH₃) m/e 460 (M+NH₄)⁺;

¹H NMR (DMSO-d₆, 300 MHz) δ9.03 (s, 1H), 8.66 (s, 1H), 7.80 (s, 1H), 7.65 (d, 2H), 7.50 (d, 2H), 7.09 (s, 2H), 6.65 (s, 1H), 2.24 (s, 6H).

### Example 35

### N-[4-[3,5-bis(trifluoromethyl)-1H-pyrazol-1-yl]phenyl]-1-methylcyclopropanecarboxamide

Example (i)-a B was processed as in Example (i)-a (Method 5, 6, or 7) to provide the title compound.

mp 109-110° C.;

MS(DCI/NH₃) m/e 395 (M+NH₄)⁺;

¹H NMR (DMSO-d₆, 300 MHz) δ9.47 (s, 1H), 7.86 (d, 2H), 7.79 (s, 1H), 7.53 (d, 2H), 1.43 (s, 3H), 0.92 (m, 2H), 0.68 (m, 2H).

### Example 36

### N-[4-[3,5-bis(trifluoromethyl)-1H-pyrazol-1-yl]phenyl]-N′-phenylurea

Example (i)-a B was processed as in Example (ii)-a (Method 8) to provide the title compound.

mp 185-187° C.;

MS (DCI/NH₃) m/e 432 (M+NH₄)⁺;

¹H NMR (DMSO-d₆, 300 MHz) δ9.07 (s, 1H), 8.81 (s, 1H), 7.80 (s, 1H), 7.66 (d, 2H), 7.52 (d, 2H), 7.49 (d, 2H), 7.30 (t, 2H), 7.00 (t, 1H).

### Example 38

### N-[4-[3,5-bis(trifluoromethyl)-1H-pyrazol-1-yl]phenyl]-N′-(3-chloro-2-methylphenyl)urea

Example (i)-a B was processed as in Example (ii)-a (Method 8) to provide the title compound.

mp 193-196° C.;

MS (DCI/NH₃) m/e 480 (M+NH₄)⁺;

¹H NMR (DMSO-d₆, 300 MHz) δ9.39 (s, 1H), 8.28 (s, 1H), 7.78 (s, 1H), 7.72 (t, 1H), 7.67 (d, 2H), 7.52 (d, 2H), 7.22-7.15 (m, 2H), 2.31 (s, 3H).

### Example 39

### N-[4-[3,5-bis(trifluoromethyl)-1H-pyrazol-1-yl]phenyl]-N′-[4-(butyloxyphenyl)urea

Example (i)-a B was processed as in Example (ii)-a (Method 8) to provide the title compound.

mp 193-196° C.;

MS (DCI/NH₃) m/e 504 (M+NH₄)⁺;

¹H NMR (DMSO-d₆, 300 MHz) δ9.10 (s, 1H), 8.71 (s, 1H), 7.91 (s, 1H), 7.77 (d, 2H), 7.63 (d, 2H), 7.49 (d, 2H), 7.01 (d, 2H), 4.06 (t, 2H), 1.83-1.78 (m, 2H), 1.57-1.53 (m, 2H), 1.06 (t, 3H).

### Example 40

### N-[4-[3,5-bis(trifluoromethyl)-1H-pyrazol-1-yl]phenyl]-N′-(2-methyl-3-nitrophenyl)urea

Example (i)-a B was processed as in Example (ii)-a (Method 8) to provide the title compound.

mp 235-236° C.;

MS (DCI/NH₃) m/e 491 (M+NH₄)⁺;

¹H NMR (DMSO-d₆, 300 MHz) δ9.46 (s, 1H), 8.46 (s, 1H), 8.05 (dd, 1H), 7.79 (s, 1H), 7.68 (d, 2H), 7.60 (dd, 1H), 7.54 (d, 2H), 7.43 (t, 1H), 2.31 (s, 3H).

### Example 41

### N-[4-[3,5-bis(trifluoromethyl)-1H-pyrazol-1-yl]phenyl]-N′-(2-chloro-4-nitrophenyl)urea

Example (i)-a B was processed as in Example (ii)-a (Method 8) to provide the title compound.

mp 229-230° C.;

MS (ESI−) m/e 492 (M−H)⁻;

¹H NMR (DMSO-d₆, 300 MHz) δ8.93 (s, 1H), 8.56 (d, 1H), 8.38 (d, 1H), 8.14 (dd, 1H), 7.80 (s, 1H), 7.70 (d, 2H), 7.57 (d, 2H).

### Example 42

### N-(4-acetylphenyl)-N′-[4-[3,5-bis(trifluoromethyl)-1H-pyrazol-1-yl]phenyl]urea

Example (i)-a B was processed as in Example (ii)-a (Method 8) to provide the title compound.

mp 230-231° C.;

MS (DCI/NH₃) m/e 474 (M+NH₄)⁺;

¹H NMR (DMSO-d₆, 300 MHz) δ9.26 (s, 1H), 9.21 (s, 1H), 7.93 (d, 2H), 7.81 (s, 1H), 7.68 (d, 2H), 7.61 (d, 2H), 7.54 (d, 2H), 2.55 (S, 3H).

### Example 43

### N-[4-[3,5-bis(trifluoromethyl)-1H-pyrazol-1-yl]phenyl]-N′-(4-methyl-2-nitrophenyl)urea

Example (i)-a B was processed as in Example (ii)-a (Method 8) to provide the title compound.

mp 213-214° C.;

MS (ESI−) m/e 472 (M−H)⁻;

¹H NMR (DMSO-d₆, 300 MHz) δ10.13 (s, 1H), 9.56 (s, 1H), 8.14 (d, 1H), 7.93 (d, 1H), 7.81 (s, 1H), 7.68 (d, 2H), 7.57-7.53 (m, 3H), 2.37 (s, 3H).

### Example 44

### N-[4-[3,5-bis(trifluoromethyl)-1H-pyrazol-1-yl]phenyl]-5-methyl-2-thiophenecarboxamide

Example (i)-a B was processed as in Example (i)-a (Method 5, 6, or 7) to provide the title compound.

mp 200-202° C.;

MS (DCI/NH₃) m/e 437 (M+NH₄)⁺;

¹H NMR (DMSO-d₆, 300 MHz) δ8.0 (d, 2H), 7.9 (d, 1H), 7.8 (s, 1H), 7.6 (d, 2H), 7.0 (d, 1H), 3.3 (s, 3H).

### Example 45

### N-[4-[3,5-bis(trifluoromethyl)-1H-pyrazol-1-yl]phenyl]-N′-(4-bromo-2,6-dimethylphenyl)urea

Example (i)-a B was processed as in Example (ii)-a (Method 8) to provide the title compound.

mp>230° C.;

MS m/e (ESI−) m/e 519 (M−H)⁻;

¹H NMR (DMSO-d₆, 300 MHz) δ9.20 (s, 1H), 7.93 (s, 1H), 7.79 (s, 1H), 7.65 (d, 2H), 7.48 (d, 2H), 7.33 (s, 1H), 2.22 (s, 6H).

### Example 46

### N-[4-[3,5-bis(trifluoromethyl)-1H-pyrazol-1-yl]phenyl]-4-(1H-pyrrol-1-yl)benzamide

Example (i)-a B was processed as in Example (i)-a (Method 5, 6, or 7) to provide the title compound.

mp 249-252° C.;

MS (DCI/NH₃) 482 (M+NH₄)⁺;

¹H NMR (DMSO-d₆, 300 MHz) δ10.58 (br s, 1H), 8.04 (d, 2H), 8.01 (d, 2H), 7.81 (d, 2H), 7.80 (s, 1H), 7.61 (d, 2H), 7.56 (t, 2H), 6.37 (t, 2H).

### Example 47

### N-[4-[3,5-bis(trifluoromethyl)-1H-pyrazol-1-yl]phenyl]-N′-heptylurea

Example (i)-a B was processed as in Example (ii)-a (Method 8) to provide the title compound.

mp 129-130° C.;

MS (DCI/NH₃) m/e 454 (M+NH₄)⁺;

¹H NMR (DMSO-d₆, 300 MHz) δ7.58 (d, 2H), 7.45 (s, 1H), 7.40 (d, 2H), 3.18 (t, 2H), 2.60 (quintet, 4H), 1.40-1.25 (m, 6H), 0.90 (t, 3H).

### Example 48

### N-[4-[3,5-bis(trifluoromethyl)-1H-pyrazol-1-yl]phenyl]-N′-(4-chloro-2-nitrophenyl)urea

Example (i)-a B was processed as in Example (ii)-a (Method 8) to provide the title compound.

mp 225-227° C.;

MS (DCI/NH₃) m/e 511 (M+NH₄)⁺;

¹H NMR (DMSO-d₆, 300 MHz) δ9.69 (s, 1H), 8.30 (d, 1H), 8.17 (d, 1H), 7.82 (s, 1H), 7.85-7.78 (m, 1H), 7.68 (d, 2H), 7.56 (d, 2H).

### Example 49

### N-[4-[3,5-bis(trifluoromethyl)-1H-pyrazol-1-yl]phenyl]-7-methoxy-2-benzofurancarboxamide

Example (i)-a B was processed as in Example (i)-a (Method 5, 6, or 7) to provide the title compound.

mp 109-110° C.;

MS (DCI/NH₃) m/e 487 (M+NH₄)⁺;

¹H NMR (DMSO-d₆, 300 MHz) δ8.0 (d, 2H), 7.8 (s, 2H), 7.6 (d, 2H), 7.4 (dd, 1H), 7.3 (t, 1H), 7.1 (dd, 1H), 4.0 (s, 3H).

### Example 50

### N-[4-[3,5-bis(trifluoromethyl)-1H-pyrazol-1-yl]phenyl]-N′-2-methyl-5-nitrophenyl)urea

Example (i)-a B was processed as in Example (ii)-a (Method 8) to provide the title compound.

mp>235° C.;

MS (DCI/NH₃) m/e 473 (M+H)⁺;

¹H NMR (DMSO-d₆, 300 MHz) δ9.62 (d, 1H), 8.91 (d, 1H), 8.40 (s, 1H), 7.84 (dd, 1H), 7.78 (s, 1H), 7.70 (d, 2H), 7.55 (d, 2H), 7.50 (d, 1H), 2.40 (s, 3H).

### Example 51

### N-[4-[3,5-bis(trifluoromethyl)-1H-pyrazol-1-yl]phenyl]-3-(hydroxymethyl)benzamide

Example 129 was reduced with DIBAL-H as described in Example 107 to provide the title compound.

mp 160-162° C.;

MS (DCI/NH₃) m/e 447 (M+NH₄)⁺;

¹H NMR (DMSO-d₆, 300 MHz) δ10.57 (s, 1H), 8.01 (d, 2H), 7.93 (s, 1H), 7.85 (d, 1H), 7.81 (s, 1H), 7.6 (d, 2H), 7.57 (d, 1H), 7.5 (t, 1H), 5.35 (t, 1H), 4.6 (d, 2H).

### Example 52

### N-[4-[3,5-bis(trifluoromethyl)-1H-pyrazol-1-yl]phenyl]-2-cyanoacetamide

Example (i)-a B was processed as in Example (i)-a (Method 5, 6, or 7) to provide the title compound.

mp 140-143° C.;

MS (DCI/NH₃) m/e 380 (M+NH₄)⁺;

¹H NMR (DMSO-d₆, 300 MHz) δ10.6 (s, 1H), 7.8 (s, 1H), 7.7 (d, 2H), 7.6 (d, 2H), 4.0 (s, 2H).

### Example 53

### N-[4-[3,5-bis(trifluoromethyl)-1H-pyrazol-1-yl]phenyl]-2-cyclohexane-1-carboxamide

Example (i)-a B was processed as in Example (i)-a (Method 5, 6, or 7) to provide the title compound.

mp 126-128° C.;

MS (DCI/NH₃) m/e 421 (M+NH₄)⁺;

¹H NMR (DMSO-d₆, 300 MHz) δ10.3 (br, s, 1H), 7.8 (s, 1H), 7.8 (d, 2H), 7.6 (d, 2H), 5.7 (s, 2H), 2.6 (m, 1H), 2.2 (m, 2H), 2.1 (m, 2H), 1.9 (m, 1H), 1.5 (m, 1H).

### Example 54

### N-[4-[3,5-bis(trifluoromethyl)-1H-pyrazol-1-yl]phenyl]-4-methylcyclohexanecarboxamide

Example (i)-a B was processed as in Example (i)-a (Method 5, 6, or 7) to provide the title compound.

MS (DCI/NH₃) m/e 437 (M+NH₄)⁺;

¹H NMR (DMSO-d₆, 300 MHz) δ12.0 (br, s, 1H), 7.8 (d, 2H), 7.8 (s, 1H), 7.5 (d, 2H), 2.4 (t, 1H), 1.9 (mn, 4H), 1.4 (mn, 4H), 1.3 (m, 1H), 0.9 (d, 3H).

### Example 55

### N-[4-[3,5-bis(trifluoromethyl)-1H-pyrazol-1-yl]phenyl]-α-methoxy-α

### -(trifluoromethyl)benzeneacetamide

Example (i)-a B was processed as in Example (i)-a (Method 5, 6, or 7) to provide the title compound.

mp 127-129° C.;

MS (DCI/NH₃) m/e 529 (M+NH₄)⁺;

1H NMR (DMSO-d₆, 300 MHz) δ8.0 (d, 2H), 7.8 (s, 1H), 7.6 (m, 4H), 7.5 (m, 3H), 3.6 (s, 3H).

### Example 56

### N-[4-[3,5-bis(trifluoromethyl)-1H-pyrazol-1-yl]phenyl]heptanamide

Example (i)-a B was processed as in Example (i)-a (Method 5, 6, or 7) to provide the title compound.

mp 88-90° C.;

MS (DCI/NH₃) m/e 425 (M+NH₄)⁺;

1H NMR (DMSO-d₆, 300 MHz) δ10.2 (s, 1H), 7.8 (s, 1H), 7.8 (d, 2H), 7.6 (d, 2H), 2.4 (t, 2H), 1.6 (t, 2H), 1.3 (m, 6H), 0.9 (t, 3H).

### Example 57

### N-[4-[3,5-bis(trifluoromethyl)-1H-pyrazol-1-yl]phenyl]-2-phenoxybenzamide

Example (i)-a B was processed as in Example (i)-a (Method 5, 6, or 7) to provide the title compound.

mp 108-109° C.;

MS (DCI/NH₃) m/e 509 (M+NH₄)⁺;

¹H NMR (DMSO-d₆, 300 MHz) δ10.7 (s, 1H), 7.9 (d, 2H), 7.8 (s, 1H), 7.7 (dd, 1H), 7.6-7.5 (m, 3H), 7.4-7.3 (m, 3H), 7.2-7.1 (m, 3H), 7.0 (d, 1H).

### Example 58

### 3-Amino-N-[4-[3,5-bis(trifluoromethyl)-1H-pyrazol-1-yl]phenyl]benzamide

Example 58 was prepared from Example 140 using a procedure analogous to that described for Example 59.

mp 203-204° C.;

MS (DCI/NH₃) m/e 432 (M+NH₄)⁺;

¹H NMR (DMSO-d₆, 300 MHz) δ7.98 (d, 2H), 7.80 (s, 1H), 7.58 (d, 2H), 7.18 (t, 1H), 7.13-7.07 (m, 2H), 6.80-6.74 (m, 1H), 5.34 (s, 2H).

### Example 59

### 4-Amino-N-[4-[3,5-bis(trifluoromethyl)-1H-pyrazol-1-yl]phenyl]benzamide

To 15 mL of ethyl acetate and 100 mg of Example 93 was added 8 mg of 10% palladium on carbon catalyst under a nitrogen atmosphere. The mixture was stirred under hydrogen at room temperature for 20 hours, filtered and concentrted to provide a brown oil. The oil was chromatographed on silica gel with ethyl acetate/hexanes (20:80 then 30:70) to provide the title compound as a yellow oil.

mp>240° C.;

MS (DCI/NH₃) m/e 415 (M+H)⁺;

¹H NMR (DMSO-d₆, 300 MHz) δ7.97 (d, 2H), 7.83 (s, 1H), 7.75 (d, 2H), 7.55 (d, 2H), 6.62 (d, 2H), 5.84 (s, 2H).

### Example 60

### 4-Azido-N-[4-[3,5-bis(trifluoromethyl)-1H-pyrazol-1-yl]phenyl]benzamide

Example (i)-a B was processed as in Example (i)-a (Method 5, 6, or 7) to provide the title compound.

mp 182-184° C.;

MS (DCI/NH₃) m/e 458 (M+NH₄)⁺;

¹H NMR (DMSO-d₆, 300 MHz) δ10.6 (s, 1H), 8.1 (d, 2H), 8.0 (d, 2H), 7.8 (s, 1H), 7.6 (d, 2H), 7.3 (d, 2H).

### Example 61

### N-[4-[3,5-bis(trifluoromethyl)-1H-pyrazol-1-yl]-2-thiopheneacetamide

Example (i)-a B was processed as in Example (i)-a (Method 5, 6, or 7) to provide the title compound.

mp 181-183° C.;

MS (DCI/NH₃) m/e 437 (M+NH₄)⁺;

¹H NMR (DMSO-d₆, 300 MHz) δ10.6 (s, 1H), 7.8 (d, 2H), 7.8 (s, 1H), 7.6 (d, 2H), 7.4 (dd, 1H), 7.0 (m, 2H), 3.9 (s, 2H).

### Example 62

### N-[3,5-bis(trifluoromethyl)-1H-pyrazol-1-yl]phenyl]-1-tricyclo[3.3.1.1^{3,7}]-decanecarboxmide

Example (i)-a B was processed as in Example (i)-a (Method 5, 6, or 7) to provide the title compound.

mp 213-214° C.;

MS (DCI/NH₃) m/e 475 (M+NH₄)⁺;

¹H NMR (DMSO-d₆, 300 MHz) δ9.4 (s, 1H), 7.9 (d, 2H), 7.8 (s, 1H), 7.5 (d, 2H), 2.1 (s, 3H), 1.9 (s, 6H), 1.7 (s, 6H).

### Example 63

### N-[4-[3,5-bis(trifluoromethyl)-1H-pyrazol-1-yl]phenyl]-N²-[(1,1-dimethylethoxy)carbonyl]

### -L-asparagine, phenylmethyl ester

Example (i)-a B was processed as in Example (i)-a (Method 5, 6, or 7) to provide the title compound.

mp 195-196° C.;

MS (DCI/NH₃) m/e 601 (M+NH₄)⁺;

¹H NMR (DMSO-d₆, 300 MHz) δ10.4 (s, 1H), 7.8 (s, 1H), 7.8 (d, 2H), 7.4 (d, 2H), 7.3 (m, 5H), 5.1 (s, 2H), 4.5 (m, 1H), 2.9 (m, 1H), 2.7 (m, 1H), 1.4 (s, 9H).

### Example 64

### 1,1 -dimethylethyl [7-[[4-[3,5-bis(trifluoromethyl)-1H-pyrazol-1-yl]phenyl]amino]-7-oxoheptyl]carbamate

Example (i)-a B was processed as in Example (i)-a (Method 5, 6, or 7) to provide the title compound.

mp 86-88° C.;

MS (DCI/NH₃) m/e 540 (M+NH₄)⁺;

¹H NMR (DMSO-d₆, 300 MHz) δ7.9 (s, 1H), 7.9 (d, 2H), 7.5 (d, 2H), 3.3 (m, 12H), 1.2 (s, 9H).

### Example 65

### N-[4-[3,5-bis(trifluoromethyl)-1H-pyrazol-1-yl]phenyl]-3-(methylthio)propanamide

Example (i)-a B was processed as in Example (i)-a (Method 5, 6, or 7) to provide the title compound.

mp 122-123° C.;

MS (DCI/NH₃) m/e 415 (M+NH₄)⁺;

¹H NMR (DMSO-d₆, 300 MHz) δ10.4 (s, 1H), 7.9 (d, 2H), 7.8 (s, 1H), 7.6 (d, 2H), 2.9 (t, 2H), 2.7 (t, 2H), 2.2 (s, 3H).

### Example 66

### N-[4-[3,5-bis(trifluoromethyl)-1H-pyrazol-1-yl]phenyl]-1-naphthylenecarboxamide

Example (i)-a B was processed as in Example (i)-a (Method 5, 6, or 7) to provide the title compound.

mp 170-171° C.;

MS (DCI/NH₃) m/e 467 (M+NH₄)⁺;

¹H NMR (DMSO-d₆, 300 MHz) δ11.0 (s, 1H), 8.2 (m, 1H), 8.1 (d, 1H), 8.0 (m, 3H), 7.8 (d, 1H), 7.8 (s, 1H), 7.6 (m, 5H).

### Example 67

### N-[4-[3,5-bis(trifluoromethyl)-1H-pyrazol-1-yl]phenyl]-4-cyanobenzamide

Example (i)-a B was processed as in Example (i)-a (Method 5, 6, or 7) to provide the title compound.

mp 169-171° C.;

MS (DCI/NH₃) m/e 442 (M+NH₄)⁺;

¹H NMR (DMSO-d₆, 300 MHz) δ10.8 (s, 1H), 8.2 (d, 2H), 8.1 (d, 2H), 8.0 (d, 2H), 7.8 (s, 1H), 7.6 (d, 2H).

### Example 68

### N-[4-[3,5-bis(trifluoromethyl)-1H-pyrazol-1-yl]phenyl]-2-phenylcyclopropanecarboxamide

Example (i)-a B was processed as in Example (i)-a (Method 5, 6, or 7) to provide the title compound.

mp 176-178° C.;

MS (DCI/NH₃) m/e 457 (M+NH₄)⁺;

¹H NMR (DMSO-d₆, 300 MHz) δ10.6 (s, 1H), 7.8 (d, 2H), 7.8 (s, 1H), 7.6 (d, 2H), 7.3 (m, 2H), 7.2 (m, 3H), 2.4 (m, 1H), 2.1 (m, 1H), 1.6 (m, 1H), 1.4 (m, 1H).

### Example 69

### N-[4-[3,5-bis(trifluoromethyl)-1H-pyrazol-1-yl]phenyl]-4-iodobenzamide

Example (i)-a B was processed as in Example (i)-a (Method 5, 6, or 7) to provide the title compound.

mp 196-198° C.;

MS (DCI/NH₃) m/e 543 (M+NH₄)⁺;

¹H NMR (DMSO-d₆, 300 MHz) δ10.6 (s, 1H), 8.0 (d, 2H), 7.9 (d, 2H), 7.8 (s, 1H), 7.7 (d, 2H), 7.6 (d, 2H).

### Example 70

### N-[4-[3,5-bis(trifluoromethyl)-1H-pyrazol-1-yl]phenyl]-3-chloropropanamide

Example (i)-a B was processed as in Example (i)-a (Method 5, 6, or 7) to provide the title compound.

mp 144-145° C.;

MS (DCI/NH₃) m/e 403 (M+NH₄)⁺;

¹H NMR (DMSO-d₆, 300 MHz) δ10.4 (s, 1H), 7.8 (d, 2H), 7.8 (s, 1H), 7.6 (d, 2H), 3.9 (t, 2H), 2.9 (t, 2H).

### Example 71

### N-[4-[3,5-bis(trifluoromethyl)-1H-pyrazol-1-yl]phenyl]-4-methoxybenzamide

Example (i)-a B was processed as in Example (i)-a (Method 5, 6, or 7) to provide the title compound.

mp 205-207° C.;

MS (DCI/NH₃) m/e 447 (M+NH₄)⁺;

¹H NMR (DMSO-d₆, 300 MHz) δ10.5 (s, 1H), 8.1 (d, 1H), 8.0 (d, 2H), 7.9 (s, 1H), 7.6 (d, 2H), 7.2-7.1 (m, 3H), 3.4 (s, 3H).

### Example 72

### N-[4-[3,5-bis(trifluoromethyl)-1H-pyrazol-1-yl]phenyl]-2-ethylhexanamide

Example (i)-a B was processed as in Example (i)-a (Method 5, 6, or 7) to provide the title compound.

mp 127-128° C.;

MS (DCI/NH₃) m/e 439 (M+NH₄)⁺;

¹H NMR (DMSO-d₆, 300 MHz) δ10.2 (s, 1H), 7.9 (d, 2H), 7.8 (s, 1H), 7.5 (d, 2H), 2.3 (m, 1H), 1.6-1.2 (m, 8H), 0.9 (m, 6H).

### Example 73

### N-[4-[3,5-bis(trifluoromethyl)-1H-pyrazol-1-yl]phenyl]-4-hydroxybenzamide

Example 71 was treated with BBr₃ as described in Example 180B to provide the title compound.

mp>245° C.;

MS (DCI/NH₃) m/e 433 (M+NH₄)⁺;

¹H NMR (DMSO-d₆, 300 MHz) δ8.0 (d, 2H), 7.9 (d, 2H), 7.8 (s, 1H), 7.6 (d, 2H), 6.8 (d, 2H).

### Example 74

### N-[4-[3,5-bis(trifluoromethyl)-1H-pyrazol-1-yl]phenyl]-4-(hexyloxy)benzamide

Example (i)-a B was processed as in Example (i)-a (Method 5, 6, or 7) to provide the title compound.

mp 153-155° C.;

MS (DCI/NH₃) m/e 517 (M+NH₄)⁺;

¹H NMR (DMSO-d₆, 300 MHz) δ10.4 (s, 1H), 8.0 (m, 4H), 7.8 (s, 1H), 7.6 (d, 2H), 7.1 (d, 2H), 4.1 (t, 2H), 1.8-1.7 (m, 2H), 1.5-1.4 (m, 2H), 1.4-1.3 (m, 4H), 0.9-0.8 (m, 3H).

### Example 75

N-[4-[3,5-bis(trifluoromethyl)-1H-pyrazol-1-yl]phenyl]-3-methylbenzamide

Example (i)-a B was processed as in Example (i)-a (Method 5, 6, or 7) to provide the title compound.

mp 137-139° C.;

MS (DCI/NH₃) m/e 431 (M+NH₄)⁺;

¹H NMR (DMSO-d₆, 300 MHz) δ10.5 (s, 1H), 8.0 (d, 2H), 7.9 (s, 1H), 7.8 (m, 2H), 7.6 (d, 2H), 7.4 (dd, 2H), 2.4 (s, 3H).

### Example 76

### 2-(Acetyloxy)-N-[4-[3,5-bis(trifluoromethyl)-1H-pyrazol-1-yl]phenyl]benzamide

Example (i)-a B was processed as in Example (i)-a (Method 5, 6, or 7) to provide the title compound.

mp 159-161° C.;

MS (DCI/NH₃) m/e 475 (M+NH₄)⁺;

¹H NMR (DMSO-d₆, 300 MHz) δ10.7 (s, 1H), 8.0 (d, 2H), 7.9 (s, 1H), 7.8 (dd, 1H), 7.6 (d, 2H), 7.4 (m, 1H), 7.3 (dd, 1H), 2.2 (s, 3H).

### Example 77

### N-[4-[3,5-bis(trifluoromethyl)-1H-pyrazol-1-yl]phenyl]-N′-(4-bromo-2-methylphenyl)urea

Example (i)-a B was processed as in Example (ii)-a (Method 8) to provide the title compound.

mp 230-231° C.;

MS (DCI/NH₃) m/e 474 (M+NH₄)⁺;

¹H NMR (DMSO-d₆, 300 MHz) δ9.26 (s, 1H), 9.21 (s, 1H), 7.93 (d, 2H), 7.81 (s, 1H), 7.68 (d, 2H), 7.61 (d, 2H), 7.54 (dd, 1H), 2.55 (S, 3H).

### Example 78

### N-[4-[3,5-bis(trifluoromethyl)-1H-pyrazol-1-yl]phenyl]-2,4,6-trimethylbenzamide

Example (i)-a B was processed as in Example (i)-a (Method 5, 6, or 7) to provide the title compound.

mp 203-205° C.;

MS (DCI/NH₃) m/e 459 (M+NH₄)⁺;

¹H NMR (DMSO-d₆, 300 MHz) δ10.7 (s, 1H), 8.0 (d, 2H), 7.8 (s, 1H), 7.6 (d, 2H), 6.9 (s, 2H), 2.3 (s, 3H), 2.2 (s, 6H).

### Example 79

### N-[4-[3,5-bis(trifluoromethyl)-1H-pyrazol-1-yl]phenyl]-N′-(4-chloro-3-nitrophenyl)urea

Example (i)-a B was processed as in Example (ii)-a (Method 8) to provide the title compound.

mp 207-209° C.;

MS (ESI−) m/e 492 (M−H)⁻;

¹H NMR (DMSO-d₆, 300 MHz) δ9.42 (s, 1H), 9.32 (s, 1H), 8.33 (t, 1H), 7.81 (s, 1H), 7.72-7.63 (m, 4H), 7.55 (d, 2H).

### Example 80

### N-[4-[3,5-bis(trifluoromethyl)-1H-pyrazol-1-yl]phenyl]-2-chloro-N-methylbenzamide

Example 91 was processed as in Example (iv)-a (Method 10) to provide the title compound.

MS (DCI/NH₃) m/e 465 (M+NH₄)⁺;

¹H NMR (DMSO-d₆, 100° C., 300 MHz) δ7.58 (s, 1H), 7.47 (s, 4H), 7.40-7.23 (m, 4H), 3.38 (s, 3H).

### Example 81

### N-[4-[3,5-bis(trifluoromethyl)-1H-pyrazol-1-yl]phenyl]-2-chloro-4-nitro-N-methylbenzamide

N-[4-[3,5-bis(trifluoromethyl)-1H-pyrazol-1-yl]phenyl]-2-chloro-4-nitrobenzamide was processed as Example (i)-a in Example (iv)-a (Method 10) to provide the title compound.

MS (DCI/NH₃) m/e 510 (M+NH₄)⁺;

¹H NMR (DMSO-d₆, 300 MHz) δ8.10-8.02 (m, 2H), 7.44-7.36 (m, 3H), 7.28 (d, 2H), 7.06 (s, 1H), 3.61 (s, 3H).

### Example 82

### N-[4-[3,5-bis(trifluoromethyl)-1H-pyrazol-1-yl]phenyl]-2-chlorobenzenemethanamine

Example (i)-a B was processed as in Example (vi)-a (Method 12) to provide the title compound.

mp 240° C.;

MS (DCI/NH₃) m/e 420 (M+H)⁺;

¹H NMR (DMSO-d₆, 300 MHz) δ7.70 (s, 1H), 7.54-7.47 (m, 1H), 7.45-7.41 (m, 1H), 7.38-7.32 (m, 2H), 7.25 (d, 2H), 6.90 (t, 1H), 6.67 (d, 2H), 4.40 (d, 2H).

### Example 83

### N-[4-[3,5-bis(trifluoromethyl)-1H-pyrazol-1-yl]phenyl]-1-methyl-5-nitro-1H-pyrazole-4-carboxamide

Example (i)-a B was processed as in Example (i)-a (Method 5, 6, or 7) to provide the title compound.

mp 214-216° C.;

MS (DCI/NH₃) m/e 466 (M+NH₄)⁺;

¹H NMR (DMSO-d₆, 300 MHz) δ10.83 (s, 1H), 8.03 (s, 1H), 7.89 (d, 2H), 7.87 (s, 1H), 7.63 (d, 2H).

### Example 84

### N- [4-[3,5-bis(trifluoromethyl)-1H-pyrazol-1-yl]phenyl]-4-fluorobenzenemethanamine

Example (i)-a B was processed as in Example (vi)-a (Method 12) to provide the title compound.

mp 92-94° C.;

MS (DCI/NH₃) m/e 404 (M+H)⁺ and 421 (M+NH₄)⁺;

¹H NMR (DMSO-d₆, 300 MHz) δ7.68 (s, 1H), 7.45-7.38 (m, 2H), 7.24-7.13 (m, 4H), 6.87 (t, 1H), 6.68 (d, 2H), 4.33 (d, 2H).

### Example 85

### N-[4-[3,5-bis(trifluoromethyl)-1H-pyrazol-1-yl]phenyl]-4-bromobenzamide

Example (i)-a B was processed as in Example (i)-a (Method 5, 6, or 7) to provide the title compound.

mp 205-207° C.;

MS (DCI/NH₃) m/e 495 (M+NH₄)⁺;

1H NMR (DMSO-d₆, 300 MHz) δ10.6 (s, 1H), 8.0 (d, 2H), 7.9 (d, 2H), 7.8 (s, 1H), 7.8 (d, 2H), 7.6 (d, 2H).

### Example 86

### N-[4-[3,5-bis(trifluoromethyl)-1H-pyrazol-1-yl]phenyl]-2-(dimethylamino)benzamide

Example (i)-a B was processed as in Example (i)-a (Method 5, 6, or 7) to provide the title compound.

MS (DCI/NH₃) m/e 441 (M+H)⁺;

¹H NMR (CDCl₃, 300 MHz) δ8.1 (dd, 1H), 7.85 (d, 2H), 7.51 (m, 1H), 7.48 (d, 2H), 7.35 (t, 2H), 7.07 (s, 1H), 2.87 (s, 6H).

### Example 88

### N-[4-[3,5-bis(trifluoromethyl)-1H-pyrazol-1-yl]phenyl]-3-(dimethylamino)benzamide

Example (i)-a B was processed as in Example (i)-a (Method 5, 6, or 7) to provide the title compound.

mp 180-181° C.;

MS (DCI/NH₃) m/e 443 (M+H)⁺;

¹H NMR (DMSO-d₆, 300 MHz) δ8.00 (d, 2H), 7.82 (s, 1H), 7.60 (d, 2H), 7.35 (t, 1H), 7.24-7.20 (m, 2H), 6.98-6.95 (m, 1H), 2.98 (s, 6H).

### Example 89

### N-[4-[3,5-bis(trifluoromethyl)-1H-pyrazol-1-yl]phenyl]-4-(trifluoromethyl)benzamide

Example (i)-a B was processed as in Example (i)-a (Method 5, 6, or 7) to provide the title compound.

mp 170-172° C.;

MS (DCI/NH₃) m/e 485 (M+NH₄)⁺;

¹H NMR (DMSO-d₆, 300 MHz) δ10.8 (s, 1H), 8.2 (d, 2H), 8.0 (d, 2H), 7.9 (d, 2H), 7.8 (s, 1H), 7.6 (d, 2H).

### Example 90

### N-[4-[3,5-bis(trifluoromethyl)-1H-pyrazol-1-yl]phenyl]-4-fluorobenzamide

Example (i)-a B was processed as in Example (i)-a (Method 5, 6, or 7) to provide the title compound.

mp 184-186° C.;

MS (DCI/NH₃) m/e 435 (M+NH₄)⁺;

¹H NMR (DMSO-d₆, 300 MHz) δ10.6 (s, 1H), 8.1 (m, 2H), 8.0 (d, 2H), 7.8 (s, 1H), 7.6 (d, 2H), 7.4 (m, 2H).

### Example 91

### N-[4-[3,5-bis(trifluoromethyl)-1H-pyrazol-1-yl]phenyl]-2-chlorobenzamide

Example (i)-a B was processed as in Example (i)-a (Method 5, 6, or 7) to provide the title compound.

mp 155-157° C.;

MS (DCI/NH₃) m/e 451 (M+NH₄)⁺;

¹H NMR (DMSO-d₆, 300 MHz) δ10.9 (s, 1H), 8.0 (d, 2H), 7.8 (s, 1H), 7.6 (d, 2H), 7.6-7.4 (m, 4H).

### Example 92

### N-[4-[3,5-bis(trifluoromethyl)-1H-pyrazol-1-yl]phenyl]benzamide

Example (i)-a B was processed as in Example (i)-a (Method 5, 6, or 7) to provide the title compound.

mp 177-178° C.;

MS (DCI/NH₃) m/e 417 (M+NH₄)⁺;

¹H NMR (DMSO-d₆, 300 MHz) δ8.0-7.9 (m, 4H), 7.6 (m, 3H), 7.5 (m, 3H).

### Example 93

### N-[4-[3,5-bis(trifluoromethyl)-1H-pyrazol-1-yl]phenyl]-4-nitrobenzamide

Example (i)-a B was processed as in Example (i)-a (Method 5, 6, or 7) to provide the title compound.

mp 185-188° C.;

MS (DCI/NH₃) m/e 462 (M+NH₄)⁺;

¹H NMR (CDCl₃, 300 MHz) δ8.42 (d, 2H), 8.08 (d, 2H), 7.99 (br s, 1H), 7.85 (d, 2H), 7.56 (d, 2H), 7.09 (s, 1H).

### Example 94

### 4-[3,5-Bis(trifluoromethyl)-1H-pyrazol-1-yl]-N-(4-fluorophenyl)benzenemethanamine

Example (vii)-a A was processed as in Example (vii)-a (Method 13) to provide the title compound.

mp 102-103° C.;

MS (DCI/NH₃) m/e 421 (M+NH₄)⁺;

¹H NMR (DMSO-d₆, 300 MHz) δ7.83 (s, 1H), 7.57 (s, 4H), 6.9 (t, 2H), 6.55 (m, 2H), 6.3 (t, 1H), 4.37 (d, 2H).

### Example 95

### 3-[4-[[[3,5-bis(trifluoromethyl)-1H-pyrazol-1-yl]phenyl]methyl]amino]benzonitrile

Example (vii)-a A was processed as in Example (vii)-a (Method 13) to provide the title compound.

mp 129-130° C.;

MS (DCI/NH₃) m/e 428 (M+NH₄)⁺;

¹H NMR (DMSO-d₆, 300 MHz) δ7.83 (s, 1H), 7.57 (m, 4H), 7.45 (d, 2H), 7.4 (t, 1H), 6.68 (d, 2H), 4.48 (d, 2H).

### Example 96

### N-[4-[3,5-bis(trifluoromethyl)-1H-pyrazol-1-yl]phenyl]-2-methylbenzamide

Example (i)-a B was processed as in Example (i)-a (Method 5, 6, or 7) to provide the title compound.

mp 165-167° C.;

MS (DCI/NH₃) m/e 431 (M+NH₄)⁺;

¹H NMR (DMSO-d₆, 300 MHz) δ10.67 (br s, 1H), 7.96 (d, 2H), 7.82 (s, 1H), 7.60 (d, 2H), 7.53 (m, 4H), 2.41 (s, 3H).

### Example 97

### (E)-N-[4-[3,5-bis(trifluoromethyl)-1H-pyrazol-1-yl]phenylmethylene]-2,4-difluorobenzenamine

Example (vii)-a A was processed as in Example (viii)-a (Method 13) to provide the title compound as a byproduct with Example 108.

MS (DCI/NH₃) m/e 420 (M+NH₄)⁺;

¹H NMR (DMSO-d₆, 300 MHz) δ8.8 (s, 1H), 8.2 (d, 2H), 7.9 (s, 1H), 7.8 (d, 2H), 7.5-7.4 (m, 2H), 7.2 (m, 1H).

### Example 98

### N-[4-[3,5-bis(trifluoromethyl)-1H-pyrazol-1-yl]phenyl]-3-4-dimethoxybenzamide

Example (i)-a B was processed as in Example (i)-a (Method 5, 6, or 7) to provide the title compound.

mp 189-191° C.;

MS(DCI/NH₃) 477 (M+NH₄)⁺;

¹H NMR (DMSO-d₆, 300 MHz) δ10.40 (br s, 1H), 7.99 (d, 2H), 7.83 (s, 1H), 7.67-7.55 (m, 3H), 7.12 (d, 2H), 3.86 (s, 3H), 3.85 (s, 3H).

### Example 99

### N-[4-[3,5-bis(trifluoromethyl)-1H-pyrazol-1-yl]phenyl]cyclopentanepropanamide

Example (i)-a B was processed as in Example (i)-a (Method 5, 6, or 7) to provide the title compound.

mp 130-132° C.;

MS (DCI/NH₃) 437 (M+NH₄)⁺;

¹H NMR (DMSO-d₆, 300 MHz) δ10.24 (br s, 1H), 7.80 (s, 1H), 7.79 (d, 2H), 7.53 (d, 2H), 2.37 (t, 2H), 1.81-1.73 (m, 3H), 1.66-1.48 (m, 8H).

### Example 100

### N-[4-[3,5-bis (trifluoromethyl)-1H-pyrazol-1-yl]phenyl]-4-methylbenzamide

Example (i)-a B was processed as in Example (i)-a (Method 5, 6, or 7) to provide the title compound.

mp 225-226° C.;

MS (DCI/NH₃) 431 (M+NH₄)⁺;

¹H NMR (DMSO-d₆, 300 MHz) δ10.50 (br s, 1H), 8.01 (d, 2H), 7.91 (d, 2H), 7.83 (s, 1H), 7.61 (d, 2H), 7.37 (d, 2H), 2.40 (s, 3H).

### Example 101

### N-[4-[3,5-bis(trifluoromethyl)-1H-pyrazol-1-yl]phenyl]-3-(trifluoromethyl)benzamide

Example (i)-a B was processed as in Example (i)-a (Method 5, 6, or 7) to provide the title compound.

mp 143-145° C.;

MS(DCI/NH₃) 485 (M+NH₄)⁺;

¹H NMR (DMSO-d₆, 300 MHz) δ10.80 (br s, 1H), 8.30 (m, 2H), 8.01 (d, 2H), 7.99 (s, 1H), 7.85-7.80 (m, 2H), 7.65 (d, 2H).

### Example 102

### N-[4-[3,5-bis(trifluoromethyl)-1H-pyrazol-1-yl]phenyl]-3-methyl-2-butenamide

Example (i)-a B was processed as in Example (i)-a (Method 5, 6, or 7) to provide the title compound.

mp 150-152° C.;

MS (DCI/NH₃) 395 (M+NH₄)⁺;

¹H NMR (DMSO-d₆, 300 MHz) δ10.08 (br s, 1H), 7.76 (s, 1H), 7.72 (d, 2H), 7.45 (d, 2H), 5.83 (s, 1H), 2.09 (s, 3H), 1.81 (s, 3H).

### Example 103

### N-[4-[3,5-bis(trifluoromethyl)-1H-pyrazol-1-yl]phenyl]-2-hydroxybenzamide

Example 143 was treated with BBr₃ as described in Example 180B to provide the title compound.

mp 173-175° C.;

MS (DCI/NH₃) m/e 433 (M+NH₄)⁺;

¹H NMR (DMSO-d₆, 300 MHz) δ11.5 (br s, 1H), 10.6 (s, 1H), 8.0-7.9 (m, 3H), 7.8 (s, 1H), 7.6 (d, 2H), 7.4 (m, 1H), 7.1-7.0 (m, 2H).

### Example 104

### N-[4-[3,5-bis(trifluoromethyl)-1H-pyrazol-1-yl]phenyl]-3-hydroxybenzamide

Example 145 was treated with BBr₃ as described in Example 180B to provide the title compound.

mp 221-223° C.;

MS (DCI/NH₃) m/e 433 (M+NH₄)⁺;

¹H NMR (DMSO-d₆, 300 MHz) δ10.5 (br s, 1H), 9.8 (s, 1H), 8.0 (d, 2H), 7.8 (s, 1H), 7.6 (d, 2H), 7.4-7.3 (m, 3H), 7.0 (m, 1H).

### Example 105

### N-[4-[3,5-bis(trifluoromethyl)-1H-pyrazol-1-yl]phenyl]-2,4-dimethyl-5-thiazolecarboxamide

Example (i)-a B was processed as in Example (i)-a (Method 5, 6, or 7) to provide the title compound.

mp 158-159° C.;

MS (DCI/NH₃) m/e 435 (M+NH₄)⁺;

¹H NMR (DMSO-d₆, 300 MHz) δ10.42 (s, 1H), 7.88 (d, 2H), 7.82 (s, 1H), 7.60 (d, 2H), 2.68 (s, 3H), 2.57 (s, 3H).

### Example 106

### N-[4-[3,5-bis(trifluoromethyl)-1H-pyrazol-1-yl]phenyl]-3-pyridinecarboxamide

Example (i)-a B was processed as in Example (i)-a (Method 5, 6, or 7) to provide the title compound.

mp 185-188° C.;

MS (DCI/NH₃) m/e 401 (M+H)⁺; ¹H NMR (DMSO-d₆, 300 MHz) 6 10.78 (br s, 1H), 9.14 (d, 1H), 8.80 (dd, 1H), 8.32 (dt, 1H,), 8.01 (d, 2H), 7.83 (s, 1H), 7.65-7.58 (m, 3H).

### Example 107

### N-[4-[3,5-bis(trifluoromethyl)-1H-pyrazol-1-yl]phenyl]-4-(hydroxymethyl)benzamide

To a solution of carboxylic acid methyl ester, Examplel42, in toluene was added 1.2 equivalent of DIBAl-H (1.5 M solution in toluene) at −78° C. After stirring at −78° C. for 1 h, 1 equivalent more of DIBAl-H was added to consume all the starting material. Then the reaction mixtured was quenched with methanol followed by 1N NaOH. After stirring for 30 min, the reaction mixture was partitioned between ether and brine. The organic layer was separated, dried and concentrated to give crude material which was purified by normal phase HPLC (20:80, acetone:hexane). The desired product was collected in approximately 15% yield. mp 213-214° C.;

MS (ESI−) m/e 428 (M−H)⁻;

¹H NMR (DMSO-d₆, 300 MHz) δ10.52 (s, 1H), 8.01 (d, 2H), 7.96 (d, 2H), 7.81 (s, 1H), 7.6 (d, 2H), 7.5 (d, 2H), 5.35 (t, 1H), 4.6 (d, 2H).

### Example 108

### 4-[3,5-bis(trifluoromethyl)-1H-pyrazol-1-yl]-N-(2,4-difluorophenyl)benzenemethanamine

Example (vii)-a A was processed as in Example (vii)-a (Method 13) to provide the title compound and Example 97 as a byproduct.

MS (DCI/NH₃) m/e 422 (M+NH₄)⁺;

¹H NMR (DMSO-d₆, 300 MHz) δ7.8 (s, 1H), 7.6 (s, 4H), 7.1 (m, 1H), 6.8 (m, 1H), 6.6 (m, 1H), 6.2 (m, 1H), 4.4 (d, 2H).

### Example 109

### N-[4-[3,5-bis(trifluoromethyl)-1H-pyrazol-1-yl]phenyl]-4-(methylsulfonyl)benzamide

Example (i)-a B was processed as in Example (i)-a (Method 5, 6, or 7) to provide the title compound.

mp 216-218° C.;

MS(DCI/NH₃) 495 (M+NH₄);

¹H NMR (DMSO-d₆, 300 MHz) δ8.20 (d, 2H), 8.12 (d, 2H), 8.02 (d, 2H), 7.83 (s, 1H), 7.64 (d, 2H), 3.35 (s, 3H).

### Example 110

### N-[4-[3,5-bis(trifluoromethyl)-1H-pyrazol-1-yl]phenyl]-2-iodobenzamide

Example (i)-a B was processed as in Example (i)-a (Method 5, 6, or 7) to provide the title compound.

mp 180-182° C.;

MS(DCI/NH₃) 543 (M+NH₄)⁺;

¹H NMR (DMSO-d₆, 300 MHz) δ10.80 (br s, 1H), 7.97 (d, 1H), 7.93 (d, 2H), 7.83 (s, 1H), 7.62 (d, 2H), 7.55-7.50 (m, 2H), 7.30-7.21 (m, 1H).

### Example 111

### N-[4-[3,5-bis(trifluoromethyl)-1H-pyrazol-1-yl]phenyl]-4-heptybenzamide

Example (i)-a B was processed as in Example (i)-a (Method 5, 6, or 7) to provide the title compound.

mp 143-145° C.;

MS (DCI/NH₃) 515 (M+NH₄)⁺;

¹H NMR (DMSO-d₆, 300 MHz) δ10.51 (br s, 1H), 8.01 (d, 2H), 7.94 (d, 2H), 7.85 (s, 1H), 7.60 (d, 2H), 7.39 (d, 2H), 2.67 (t, 2H), 1.6 (m, 2H), 1.35-1.20 (m, 8H), 0.86 (t, 3H).

### Example 113

### N-[4-[3,5-bis(trifluoromethyl)-1H-pyrazol-1-yl]phenyl]-2-furancarboxamide

Example (i)-a B was processed as in Example (i)-a (Method 5, 6, or 7) to provide the title compound.

mp 179-182° C.;

MS (DCI/NH₃) m/e 407 (M+NH₄)⁺;

¹H NMR (DMSO-d₆, 300 MHz) δ10.54 (br s, 1H), 7.99 (d, 2H), 7.98 (d, 1H), 7.83 (s, 1H), 7.60 (d, 2H), 6.75-6.71 (m, 1H).

### Example 114

### N-[4-[3,5-bis(trifluoromethyl)-1H-pyrazol-1-yl]phenyl]-2-fluorobenzamide

Example (i)-a B was processed as in Example (i)-a (Method 5, 6, or 7) to provide the title compound.

mp 188-190° C.;

MS (DCI/NH₃) 435 (M+NH₄)⁺;

¹H NMR (DMSO-d₆, 300 MHz) δ10.80 (br s, 1H), 7.94 (d, 2H), 7.83 (s, 1H), 7.71 (t, 1H), 7.62 (d, 2H), 7.65-7.59 (m, 1H), 7.36 (q, 2H).

### Example 115

### N-[4-[3,5-bis(trifluoromethyl)-1H-pyrazol-1-yl]phenyl]-N′-methyl-1,2-benzenedicarboxamide

2-[[[4-[3,5-bis(trifluoromethyl)-1H-pyrazol-1-yl]phenyl]amino]carbonyl]benzoic acid (0.02 g, 0.045 mmol) in thionylchloride (1 mL) was heated to reflux for 3 h. The excess thionylchloride was removed under reduced pressure.

To the acid chloride (0.023 mmol) in CH₂Cl₂ (1 mL) was added methylamine hydrochloride (4.6 mg, 0.067 mmol) followed by triethylamine (0.019 mL, 0.14 mmol). After stirring at room temperature over night, the reaction mixture was diluted with ether and washed with 1N HCl, saturated NaHCO₃ and brine. The solvent was removed, and the crude material was purified on silica gel column, eluting with 20% acetone/hexane to give the title compound.

MS (DCI/NH₃) m/e 474 (M+NH₄)⁺;

¹H NMR (CDCl₃, 300 MHz) δ9.98 (s, 1H), 7.98 (d, 1H), 7.87 (d, 2H), 7.58 (m, 2H), 7.48 (m, 3H), 7.05 (s, 1H), 6.18 (bs, 1H), 3.01 (m, 3H).

### Example 116

### N-[4-[3,5-bis(trifluoromethyl)-1H-pyrazol-1-yl]phenyl]-4-pyridinecarboxamide

Example (i)-a B was processed as in Example (i)-a (Method 5, 6, or 7) to provide the title compound.

mp 156-157° C.;

MS (DCI/NH₃) m/e 401 (M+H)⁺;

¹H NMR (DMSO-d₆, 300 MHz) δ10.83 (br s, 1H), 8.82 (d, 2H), 8.01 (d, 2H), 7.89 (d, 2H), 7.84 (s, 1H), 7.65 (d, 2H).

### Example 117

### N-[4-[3,5-bis(trifluoromethyl)-1H-pyrazol-1-yl]phenyl]-4-chloro-2-nitrobenzamide

Example (i)-a B was processed as in Example (i)-a (Method 5, 6, or 7) to provide the title compound.

mp 163-166° C.;

MS(DCI/NH₃) 496 (M+NH₄)⁺;

¹H NMR (DMSO-d₆, 300 MHz) δ11.10 (br s, 1H), 8.57 (s, d 1H), 8.37 (dd, 1H), 7.92 (d, 3H), 7.84 (s, 1H), 7.65 (d, 2H).

### Example 118

### N-[4-[3,5-bis(trifluoromethyl)-1H-pyrazol-1-yl]phenyl]-4-cinnolinecarboxamide

Example (i)-a B was processed as in Example (i)-a (Method 5, 6, or 7) to provide the title compound.

MS (DCI/NH₃) m/e 452 (M+H)⁺;

¹H NMR (DMSO-d₆, 300 MHz) δ11.30 (br s, 1H), 9.68 (s, 1H), 8.63 (d, 1H), 8.32 (d, 1H), 8.11-7.98 (m, 1H), 8.02 (d, 2H), 7.85 (s, 1H), 7.70 (d, 2H).

### Example 119

### 4-Acetyl-N-[4-[3,5-bis(trifluoromethyl)-1H-pyrazol-1-yl]phenyl]benzamide

Example (i)-a B was processed as in Example (i)-a (Method 5, 6, or 7) to provide the title compound.

mp 203-204° C.;

MS (DCI/NH₃) m/e 459 (M+NH₄)⁺;

¹H NMR (DMSO-d₆, 300 MHz) δ10.8 (s, 1H), 8.1 (s, 4H), 8.0 (d, 2H), 7.8 (s, 1H), 7.6 (d, 2H), 2.6 (s, 3H).

### Example 120

### 1,1-Dimethylethyl 4-[[[4-[3,5-bis(trifluoromethyl)-1H-pyrazol-1-yl]phenyl]amino]carbonyl]-1-piperidinecarboxylate

Example (i)-a B was processed as in Example (i)-a (Method 5, 6, or 7) to provide the title compound.

MS (DCI/NH₃) m/e 524 (M+NH₄)⁺;

¹H NMR (DMSO-d₆, 300 MHz) δ10.29 (br s, 1H), 7.84 (d, 2H), 7.73 (s, 1H), 7.56 (d, 2H), 2.90-2.70 (m, 3H), 2.63-2.50 (m, 2H), 1.90-1.80 (m, 2H), 1.63-1.40 (m, 2H), 1.44 (s, 9H).

### Example 121

### N-[4-[3,5-bis(trifluoromethyl)-1H-pyrazol-1-yl]phenyl]-2-pyridinecarboxamide

Example (i)-a B was processed as in Example (i)-a (Method 5, 6, or 7) to provide the title compound.

MS (DCI/NH₃) m/e 401 (M+H)⁺;

¹H NMR (DMSO-d₆, 300 MHz) δ11.03 (br s, 1H), 8.78 (dd, 1H), 8.21-8.07 (m, 2H), 8.16 (d, 2H), 7.83 (s, 1H), 7.74-7.69 (m, 1H), 7.63 (d, 2H).

### Example 122

### N-[4-[3,5-bis(trifluoromethyl)-1H-pyrazol-1-yl]phenyl]-4-(diethylamino)benzamide

Example (i)-a B was processed as in Example (i)-a (Method 5, 6, or 7) to provide the title compound.

MS (DCI/NH₃) m/e 471 (M+H)⁺;

¹H NMR (DMSO-d₆, 300 MHz) δ10.15 (br s, 1H), 7.99 (d, 2H), 7.87 (s, 1H), 7.83 (d, 2H), 7.56 (d, 2H), 6.74 (d, 2H), 3.43 (q, 4H), 1.13 (t, 6H).

### Example 123

### N-[4-[3,5-bis(trifluoromethyl)-1H-pyrazol-1-yl]phenyl]cyclopentanecarboxmide

Example (i)-a B was processed as in Example (i)-a (Method 5, 6, or 7) to provide the title compound.

mp 165-168° C.;

MS (DCI/NH₃) m/e 409 (M+NH₄)⁺;

¹H NMR (DMSO-d₆, 300 MHz) δ10.22 (br s, 1H), 7.80 (d, 2H), 7.80 (s, 1H), 7.53 (d, 2H), 2.84-2.76 (m, 1H), 1.89-1.54 (m, 8H).

### Example 124

### N-[4-[3,5-bis(trifluoromethyl)-1H-pyrazol-1-yl]phenyl]cyclohexanecarboxmide

Example (i)-a B was processed as in Example (i)-a (Method 5, 6, or 7) to provide the title compound.

mp 171-173° C.;

MS (DCI/NH₃) m/e 423 (M+NH₄)⁺;

¹H NMR (DMSO-d₆, 300 MHz) δ10.23 (br s, 1H), 7.89 (d, 2H), 7.86 (s, 1H), 7.60 (d, 2H), 2.48-2.41 (m, 1H), 1.95-1.25 (m, 10H).

### Example 125

### N-[4-[3,5-bis(trifluoromethyl)-1H-pyrazol-1-yl]phenyl]-4-piperidinecarboxamide

To a stirred solution of the Boc-amine, Example 120, (165 mg, 0.323 mmol) in methylene chloride (3.0 mL) was added trifluoroacetic acid (0.250 mL, 3.25 mmol). The resulting solution was stirred at 23° C. for 2 hours at which point the reaction mixture was poured into saturated sodium bicarbonate solution (50 mL). The aqueous layer was extracted with ethyl acetate (2×50 mL). The combined organics were dried over sodium sulfate and concentrated. The crude residue was purified by flash column chromatography using 95% methylene chloride/5% methanol. Concentration of the approriate fractions afforded 45 mg, 34% yield of Example 125 as a white solid.

mp 156-159° C.;

MS (DCI/NH₃) m/e 407 (M+H)⁺;

¹H NMR (DMSO-d₆, 300 MHz) δ10.20 (br s, 1H), 7.81 (d, 2H), 7.80 (s, 1H), 7.53 (d, 2H), 3.05-2.97 (m, 2H), 2.48-2.40 (m, 2H), 1.79-1.70 (m, 2H), 1.60-1.45 (m, 2H).

### Example 126

### N-[4-[3,5-bis(trifluoromethyl)-1H-pyrazol-1-yl]phenyl]-3-(methylsulfonyl)benzamide

The procedure in J. Org. Chem. 1991, 56, 4974, hereby incorporated by reference, was followed. Briefly, to a solution of Na₂SO₃ (0.63 g, 5.0 mmol) and NaHCO₃ (1.26 g, 15 mmol) in water (5 mL) was slowly added 3-chlorosulfonylbenzoic acid (1.1 g, 5.0 mmol). The reaction mixture was heated to 75° C. for 1 hours, and then chloroacetic acid (0.71 g, 7.5 mmol) was added, followed by NaOH (0.3 g, 7.5 mmol). The resulting mixture was heated to 105° C. for 24 hours. After cooling to room temperature, the reaction was diluted with water and acidified with 1N HCl to pH 2.The solid was filtered, washed and dried to give 680 mg of the product in 75% yield.

¹H NMR (DMSO-d₆, 300MHz) δ8.4 (s, 1H), 8.25 (d, 1H), 8.17 (d, 1H), 7.8 (t, 1H), 3.35 (s, 3H);

MS (DCI/NH₃) m/e 218 (M+NH₄)⁺.

Example (i)-a B was processed as in Example (i)-a (Method 5, 6, or 7) with the benzoic acid prepared as described above to provide the title compound.

mp 194-195° C.;

MS (DCI/NH₃) m/e 495 (M+NH₄)⁺;

¹H NMR (DMSO-d₆, 300 MHz) δ8.5 (s, 1H), 8.32 (d, 1H), 8.17 (d, 1H), 8.0 (d, 2H), 7.86 (t, 1H), 7.84 (s, 1H), 7.65 (d, 2H), 3.3 (s, 3H).

### Example 127

### N-[4-[3,5-bis(trifluoromethyl)-1H-pyrazol-1-yl]phenyl]-2-(trifluoromethyl)benzamide

Example (i)-a B was processed as in Example (i)-a (Method 5, 6, or 7) to provide the title compound.

mp 133-135° C.;

MS (DCI/NH₃) m/e 485 (M+NH₄)⁺;

¹H NMR (DMSO-d₆, 300 MHz) δ10.86 (s, 1H), 7.93-7.67 (m, 8H), 7.57 (d, 1H).

### Example 128

### 3-[[[4-[3,5-bis(trifluoromethyl)-1H-pyrazol-1-yl]phenyl]methyl]amino]benzonitrile

Example (vii)-a A was processed as in Example (vii)-a (Method 13) to provide the title compound.

MS (DCI/NH₃) m/e 428 (M+NH₄)⁺;

¹H NMR (DMSO-d₆, 300 MHz) δ7.8 (s, 1H), 7.6 (m, 4H), 7.2 (m, 1H), 6.9 (m, 4H), 4.4 (d, 2H).

### Example 129

### Methyl 3-[[[4-[3,5-bis(trifluoromethyl)-1H-pyrazol-1-yl]phenyl]amino]carbonyl]benzoate

Example (i)-a B was processed as in Example (i)-a (Method 5, 6, or 7) to provide the title compound.

mp 174-175° C.;

MS (DCI/NH₃) m/e 475 (M+NH₄)⁺;

¹H NMR (DMSO-d₆, 300 MHz) 10.8 (s, 1H), 8.56 (s, 1H), 8.27 (d, 1H), 8.2 (d, 1H), 8.02 (d, 2H), 7.85 (s, 1H), 7.73 (t, 1H), 7.63 (d, 2H), 3.94 (s, 3H).

### Example 130

### N-[4-[3,5-bis(trifluoromethyl)-1H-pyrazol-1-yl]phenyl]-3-chlorobenzamide

Example (i)-a B was processed as in Example (i)-a (Method 5, 6, or 7) to provide the title compound.

mp 142-144° C.;

MS (DCI/NH₃) m/e 451 (M+NH₄)⁺;

¹H NMR (DMSO-d₆, 300 MHz) δ10.66 (s, 1H), 8.05-7.87 (m, 5H), 7.82 (s, 1H), 7.73-7.51 (m, 3H).

### Example 131

### N-[4-[3,5-bis(trifluoromethyl)-1H-pyrazol-1-yl]phenyl]-2-thiophenecarboxamide

Example (i)-a B was processed as in Example (i)-a (Method 5, 6, or 7) to provide the title compound.

mp>230° C.;

MS (DCI/NH₃) m/e 423 (M+NH₄)⁺;

¹H NMR (DMSO-d₆, 300 MHz) δ10.54 (s, 1H), 8.07 (d, 1H), 7.95 (d, 2H), 7.90 (d, 1H), 7.82 (s, 1H), 7.62 (d, 2H), 7.26 (td, 1H).

### Example 132

### (E)-3-[2-[4-[3,5-bis(trifluoromethyl)-1H-pyrazol-1-yl]phenyl]ethenyl]benzonitrile

Example (vii)-a A was processed as in Example (ix)-a (Method 14) to provide the title compound.

mp 116-117° C.;

MS (DCI/NH₃) m/e 425 (M+NH₄)⁺;

hu 1H NMR (DMSO-d₆, 300 MHz) δ8.14 (s, 1H), 7.97 (d, 1H), 7.84 (s, 1H), 7.83 (d, 2H), 7.76 (d, 1H), 7.66 (d, 2H), 7.63 (t, 1H), 7.57 (d, 1H), 7.45 (d, 1H).

### Example 133

### N-[4-[3,5-bis(trifluoromethyl)-1H-pyrazol-1-yl]phenyl]-1,4-benzenedicarboxamide

A reaction of carboxylic acid methyl ester (50 mg), Example 142, and 1M NH₃ in methanol (5 mL) in a sealed tube was stirred at 60° C. for 3 days. After cooling to room temperature, the solid precipitated out from the reaction mixture was filtered, washed with ether and dried to give the desired product in 35% yield.

mp 290-291° C.;

MS (DCI/NH₃) m/e 460 (M+NH₄)⁺;

¹H NMR (DMSO-d₆, 300 MHz) δ8.15 (s, 1H), 8.04 (s, 4H), 8.01 (d, 2H), 7.84 (s, 1H), 7.63 (d, 2H), 7.57 (s, 1H).

### Example 134

### N-[4-[3,5-bis(trifluoromethyl)-1H-pyrazol-1-yl]phenyl]-3,5-dinitrobenzamide

Example (i)-a B was processed as in Example (i)-a (Method 5, 6, or 7) to provide the title compound.

mp>230° C.;

MS (DCI/NH₃) m/e 506 (M+NH₄)⁺;

¹H NMR (DMSO-d₆, 300 MHz) δ11.17 (s, 1H), 9.20 (d, 2H), 9.03 (t, 1H), 8.03 (d, 2H), 7.85 (s, 1H), 7.70 (d, 2H).

### Example 135

### N-[4-[3,5-bis(trifluoromethyl)-1H-pyrazol-1-yl]phenyl]-2,4-difluorobenzamide

Example (i)-a B was processed as in Example (i)-a (Method 5, 6, or 7) to provide the title compound.

mp 126-128° C.;

MS (DCI/NH₃) m/e 453 (M+NH₄)⁺;

¹H NMR (DMSO-d₆, 300 MHz) δ10.79 (s, 1H), 7.92 (d, 2H), 7.84 (s, 1H), 7.80 (t, 1H), 7.62 (d, 2H), 7.48 (t, 1H), 7.26 (t, 1H).

### Example 136

### N-[4-[3,5-bis(trifluoromethyl)-1H-pyrazol-1-yl]phenyl]-2-nitrobenzamide

Example (i)-a B was processed as in Example (i)-a (Method 5, 6, or 7) to provide the title compound.

mp 174-176° C.;

MS (DCI/NH₃) m/e 462 (M+NH₄)⁺;

¹H NMR (DMSO-d₆, 300 MHz) δ11.05 (s, 1H), 8.20 (dd, 1H), 7.93-7.75 (m, 6H), 7.63 (d, 2H).

### Example 137

### N-[4-[3,5-bis(trifluoromethyl)-1H-pyrazol-1-yl]phenyl]-3-cyanobenzamide

Example (i)-a B was processed as in Example (i)-a (Method 5, 6, or 7) to provide the title compound.

mp 162-163° C.;

MS (DCI/NH₃) m/e 442 (M+NH₄)⁺;

¹H NMR (DMSO-d₆, 300 MHz) δ10.75 (s, 1H), 8.44 (s, 1H), 8.26 (d, 1H), 8.11 (d, 1H), 8.00 (d, 2H), 7.83 (s, 1H), 7.79 (t, 1H), 7.64 (d, 2H).

### Example 138

### N-[4-[3,5-bis(trifluoromethyl)-1H-pyrazol-1-yl]phenyl]-1,3-benzenedicarboxamide

Example 129 was processed as described in Example 133 to provide the title compound.

mp 244-245° C.;

MS (DCI/NH₃) m/e 460 (M+NH₄)⁺;

¹H NMR (DMSO-d₆, 300 MHz) δ8.47 (s, 1H), 8.1 (s, 1H), 8.1 (d, 2H), 8.01 (d, 2H), 7.82 (s, 1H), 7.65 (t, 1H), 7.62 (d, 2H), 7.52 (s, 1H).

### Example 139

### (Z)-3-[2-[4-[3,5-bis(trifluoromethyl)-1H-pyrazol-1-yl]phenyl]ethenyl]benzonitrile

Example (vii)-a A was processed as in Example (ix)-a (Method 14) to provide the title compound.

MS (DCI/NH₃) m/e 425 (M+NH₄)⁺;

¹H NMR (DMSO-d₆, 300 MHz) δ7.82 (s, 1H), 7.72 (d, 1H), 7.62 (s, 1H), 7.54 (d, 2H), 7.51 (d, 1H), 7.48 (t, 1H), 7.4 (d, 2H), 6.91 (d, 1H), 6.81 (d, 1H).

### Example 140

### N-[4-[3,5-bis(trifluoromethyl)-1H-pyrazol-1-yl]phenyl]-3-nitrobenzamide

Example (i)-a B was processed as in Example (i)-a (Method 5, 6, or 7) to provide the title compound.

mp 203-204° C.;

MS (DCI/NH₃) m/e 462 (M+NH₄)⁺;

¹H NMR (DMSO-d₆, 300 MHz) δ10.89 (s, 1H), 8.82 (t, 1H), 8.48-8.41 (m, 2H), 8.02 (d, 2H), 7.88 (d, 1H), 7.83 (d, 1H), 7.64 (d, 2H).

### Example 141

### 3-(aminosulfonyl)-N-[4-[3,5-bis(trifluoromethyl)-1H-pyrazol-1-yl]phenyl]benzamide

To a solution of Example (i)-a B (112 mg, 0.380 mmol) and N-methylmorpholine (0.50 mL) in dichloromethane (3 nmL) was added 3-(chlorosulfony)lbenzoyl chloride (109 mg, 0.455 mmol). The resulting solution was stirred at 23° C. for 3 hours at which point a solution of saturated ammonia in methanol (2 mL) was added. The resulting white solid was filtered and washed with hexane to provide 80 mg (40%) of the desired compound.

mp 177-178° C.;

MS(DCI/NH₃) 496 (M+H)⁺;

¹H NMR (DMSO-d₆, 300 MHz) δ8.41 (s, 1H), 8.21 (d, 1H), 8.06-7.99 (m, 1H), 8.00 (d, 2H), 7.84 (s, 1H), 7.78 (t, 1H), 7.64 (d, 2H), 7.52 (br s, 2H).

### Example 142

### methyl 4-[[[4-[3,5-bis(trifluoromethyl)-1H-pyrazol-1-yl]phenyl]amino]carbonyl]benzoate

Example (i)-a B was processed as in Example (i)-a (Method 5, 6, or 7) to provide the title compound.

mp 195-196° C.;

MS (DCI/NH₃) m/e 475 (M+NH₄)⁺;

¹H NMR (DMSO-d₆, 300 MHz) δ8.12 (m, 4H), 8.0 (d, 2H), 7.84 (s, 1H), 7.63 (d, 2H), 3.92 (s, 3H).

### Example 143

### N-[4-[3,5-bis(trifluoromethyl)-1H-pyrazol-1-yl]phenyl]-2-methoxybenzamide

Example (i)-a B was processed as in Example (i)-a (Method 5, 6, or 7) to provide the title compound.

mp 100-102° C.;

MS (DCI/NH₃) m/e 447 (M+NH₄)⁺;

¹H NMR (DMSO-d₆, 300 MHz) δ10.47 (s, 1H), 7.95 (d, 2H), 7.82 (s, 1H), 7.60 (d, 2H), 7.63 (dd, 1H), 7.53 (dt, 1H), 7.20 (d, 1H), 7.07 (t, 1H), 3.91 (s, 3H).

### Example 144

### N-[4-[3,5-bis(trifluoromethyl)-1H-pyrazol-1-yl]phenyl]-3-bromobenzamide

Example (i)-a B was processed as in Example (i)-a (Method 5, 6, or 7) to provide the title compound.

mp 158-160° C.;

MS (DCI/NH₃) m/e 497 (M+NH₄)⁺;

¹H NMR (DMSO-d₆, 300 MHz) δ10.67 (s, 1H), 8.17 (t, 1H), 8.01 (d, 2H), 8.02-7.86 (m, 1H), 7.82 (s, 1H), 7.84-7.82 (m, 1H), 7.62 (d, 2H), 7.54 (t, 1H).

### Example 145

### N-[4-[3,5-bis(trifluoromethyl)-1H-pyrazol-1-yl]phenyl]-3-methoxybenzamide

Example (i)-a B was processed as in Example (i)-a (Method 5, 6, or 7) to provide the title compound.

mp 169-172° C.;

MS (DCI/NH₃) m/e 447 (M+NH₄)⁺;

¹H NMR (DMSO-d₆, 300 MHz) δ10.55 (s, 1H), 8.02 (d, 2H), 7.82 (s, 1H), 7.72 (d, 1H), 7.58 (m, 3H), 7.38 (dd, 1H), 7.18 (dd, 1H), 3.86 (s, 3H).

### Example 146

### N-[4-[3,5-bis(trifluoromethyl)-1H-pyrazol-1-yl]phenyl]-3-fluorobenzamide

Example (i)-a B was processed as in Example (i)-a (Method 5, 6, or 7) to provide the title compound.

mp 148-151° C.;

MS (DCI/NH₃) m/e 435 (M+NH₄)⁺;

¹H NMR (DMSO-d₆, 300 MHz) δ10.64 (s, 1H), 8.0 (d, 2H), 8.05-8.0 (m, 1H), 7.84 (s, 1H), 7.85-7.78 (m, 1H), 7.74-7.43 (m, 2H), 7.62 (d, 2H).

### Example 147

### N-[4-[3,5-bis(trifluoromethyl)-1H-pyrazol-1-yl]phenyl]-2-bromobenzamide

Example (i)-a B was processed as in Example (i)-a (Method 5, 6, or 7) to provide the title compound.

mp 182-184° C.;

MS (DCI/NH₃) m/e 495 (M+NH₄)⁺;

¹H NMR (DMSO-d₆, 300 MHz) δ10.55 (s, 1H), 7.92 (d, 2H), 7.83 (s, 1H), 7.75 (d, 1H), 7.64-7.58 (m, 3H), 7.52 (td, 1H), 7.46 (dd, 1H).

### Example 148

### N-[4-[3,5-bis(trifluoromethyl)-1H-pyrazol-1-yl]phenyl]-1,3-benzodioxole-5-carboxamide

Example (i)-a B was processed as in Example (i)-a (Method 5, 6, or 7) to provide the title compound.

mp 222-224° C.;

MS (DCI/NH₃) m/e 461 (M+NH₄)⁺;

¹H NMR (DMSO-d₆, 300 MHz) δ10.39 (s, 1H), 7.98 (d, 2H), 7.82 (s, 1H), 7.60 (m, 2H), 7.54 (d, 1H), 7.09 (d, 2H), 6.18 (s, 2H).

### Example 149

### N-[4-[3,5-bis(trifluoromethyl)-1H-pyrazol-1-yl]phenyl]-2,6-dichloro-3-pyridinecarboxamide

Example (i)-a B was processed as in Example (i)-a (Method 5, 6, or 7) to provide the title compound.

mp 115-117° C.;

MS (DCI/NH₃) m/e 486 (M+NH₄)⁺;

¹H NMR (DMSO-d₆, 300 MHz) δ8.25 (d, 1H), 7.90 (d, 2H), 7.84 (s, 1H), 7.78 (d, 1H), 7.66 (d, 2H).

### Example 150

### N-[4-[3,5-bis(trifluoromethyl)-1H-pyrazol-1-yl]phenyl]-2-chloro-3-pyridinecarboxamide

Example (i)-a B was processed as in Example (i)-a (Method 5, 6, or 7) to provide the title compound.

mp 145-147° C.;

MS (DCI/NH₃) m/c 452 (M+NH₄)⁺;

¹H NMR (DMSO-d₆, 300 MHz) δ11.03 (s, 1H), 8.57 (dd, 1H), 8.16 (dd, 1H), 7.91 (d, 2H), 7.82 (s, 1H), 7.64 (d, 2H), 7.60 (dd, 1H).

### Example 151

### N-[4-[3,5-bis(trifluoromethyl)-1H-pyrazol-1-yl]phenyl]-2-chloro-6-methyl-3-pyridinecarboxamide

Example (i)-a B was processed as in Example (i)-a (Method 5, 6, or 7) to provide the title compound.

mp 155-156° C.;

MS (DCI/NH₃) m/e 466 (M+NH₄)⁺;

¹H NMR (DMSO-d₆, 300 MHz) δ10.96 (s, 1H), 8.02 (d, 1H), 7.90 (d, 2H), 7.83 (s, 1H), 7.62 (d, 2H), 7.44 (d, 1H), 2.55 (s, 3H).

### Example 152

### N-[4-[3,5-bis(trifluoromethyl)-1H-pyrazol-1-yl]phenyl]-4-fluoro-γ-oxobenzenebutanamide

Example (i)-a B was processed as in Example (i)-a (Method 5, 6, or 7) to provide the title compound.

mp 156-158° C.;

MS (DCI/NH₃) m/e 474 (M+H)⁺;

¹H NMR (DMSO-d₆, 300 MHz) δ8.04 (dd, 2H), 7.91 (s, 1H), 7.70 (d, 2H), 7.43 (d, 2H), 7.16 (t, 2H), 7.05 (s, 1H), 3.45 (t, 2H), 2.85 (t, 2H).

### Example 153

N-[4-[3,5-bis(trifluoromethyl)-1H-pyrazol-1-yl]phenyl]-1,2,3,4-tetrahydro-2-naphthalenecarboxamide

Example (i)-a B was processed as in Example (i)-a (Method 5, 6, or 7) to provide the title compound.

mp 199-201° C.;

MS (DCI/NH₃) m/e 471 (M+NH₄)⁺;

¹H NMR (DMSO-d₆, 300 MHz) δ10.35 (s, 1H), 7.84 (d, 2H), 7.80 (s, 1H), 7.56 (d, 2H), 7.11 (s, 4H), 2.97-2.72 (m, 7H).

### Example 154

### (E)-1-[4-[2-(2-chlorophenyl)ethenyl]phenyl]-3,5-bis(trifluoromethyl)-1H-pyrazole

Example (vii)-a A was processed as in Example (ix)-a (Method 14) to provide the title compound.

mp 81-82° C.;

MS (DCI/NH₃) m/e 434 (M+NH₄)⁺;

¹H NMR (DMSO-d₆, 300 MHz) δ7.92 (d, 1H) 7.87 (d, 2H), 7.86 (s, 1H), 7.65 (d, 2H), 7.6 (d, 1H), 7.53 (d, 1H), 7.44 (d, 1H), 7.43 (t, 1H), 7.36 (t, 1H).

### Example 155

### N-[4-[3,5-bis(trifluoromethyl)-1H-pyrazol-1-yl]phenyl]-2-(4-chlorophenoxy)-2-methylpropanamide

Example (i)-a B was processed as in Example (i)-a (Method 5, 6, or 7) to provide the title compound.

MS (DCI/NH₃) m/e 509 (M+NH₄)⁺;

¹H NMR (DMSO-d₆, 300 MHz) δ10.37 (s, 1H), 7.92 (d, 2H), 7.80 (s, 1H), 7.56 (d, 2H), 7.37 (d, 2H), 6.96 (d, 2H), 1.56 (s, 6H).

### Example 156

### N-[4-[3,5-bis(trifluoromethyl-1H-pyrazol-1-yl]phenyl]acetamide

Example (i)-a B was processed as in Example (i)-a (Method 5, 6, or 7) to provide the title compound.

mp 169-170° C.;

MS (DCI/NH₃) m/e 355 (M+NH₄)⁺;

¹H NMR (DMSO-d₆, 300 MHz) δ10.27 (s, 1H), 7.78 (d, 2H), 7.79 (s, 1H), 7.55 (d, 2H), 2.11 (s, 3H).

### Example 157

### 4-[[[4-[3,5-bis(trifluoromethyl)-1H-pyrazol-1-yl]phenyl]amino]carbonyl]benzoic acid

A solution of carboxylic acid methyl ester, Example 142, and 2.5 equivalent of NaOH in ethanol was stirred at 80° C. for 3 hours Then the reaction mixture was diluted with water and acidified with 1N HCl to give the precipitated product.

mp 282-283° C.;

MS (DCI/NH₃) m/e 461 (M+NH₄)⁺;

¹H NMR (DMSO-D₆, 300 MHz) δ10.75 (s, 1H), 8.09 (s, 4H), 8.02 (d, 2H), 7.84 (s, 1H), 7.63 (d, 2H).

### Example 158

### phenylmethyl N-[4-[3,5-bis(trifluoromethyl)-1H-pyrazol-1-yl]phenyl]amino]-4-oxobutyl]carbamate

Example (i)-a B was processed as in Example (i)-a (Method 5, 6, or 7) to provide the title compound.

MS (DCI/NH₃) m/e 532 (M+NH₄)⁺;

¹H NMR (DMSO-d₆, 300 MHz) δ10.23 (s, 1H), 7.78 (d, 2H), 8.00 (d, 1H), 7.52 (d, 2H), 7.34 (s, 1H), 7.40-7.25 (m, 4H), 5.00 (s, 2H), 3.08 (q, 2H), 2.38 (t, 2H), 1.78 (quintet, 2H).

### Example 159

### 3-[[[4-[3,5-bis(trifluoromethyl)-1H-pyrazol-1-yl]phenyl]amino]carbonyl]benzoic acid

A solution of Example 129 and NaOH (2.5 equivalents) in ethanol at 80° C. was stirred for 3 hours, diluted with water, acidified with 1M HCl, filtered and dried under vacuum to provide the title compound.

mp 244-245° C.;

MS (DCI/NH₃) m/e 461 (M+NH₄)⁺;

¹H NMR (DMSO-D₆, 300 MHz) δ10.78 (s, 1H), 8.55 (s, 1H), 8.23 (d, 1H), 8.17 (d, 1H), 8.02 (d, 2H), 7.84 (s, 1H), 7.7 (t, 1H), 7.63 (d, 2H).

### Example 160

### N-[4-[3,5-bis(trifluoromethyl)-1H-pyrazol-1-yl]phenyl]-2,6-difluorobenzamide

Example (i)-c B was processed as in Example (i)-c (Method 5, 6, or 7) to provide the title compound.

mp 127-128° C.;

MS (DCI/NH₃) m/e 453 (M+NH₄)⁺;

¹H NMR (DMSO-D₆, 300 MHz) δ10.38 (s, 1H), 7.86 (d, 1H), 7.78 (s, 1H), 7.69 (t, 1H), 7.59 (d, 1H), 7.52 (t, 1H), 7.45 (t, 1H), 7.15 (t, 2H).

### Example 161

### N-[4-[3,5-bis(trifluoromethyl)-1H-pyrazol-1-yl]phenyl]-3-bromo-2-thiophenecarboxamide

Example (i)-a B was processed as in Example (i)-a (Method 5, 6, or 7) to provide the title compound.

mp 139-140° C.;

MS (DCI/NH₃) m/e 503 (M+NH₄)⁺;

¹H NMR (DMSO-d₆, 300 MHz) δ7.92 (d, 1H), 7.90 (d, 2H), 7.83 (s, 1H), 7.64 (d, 2H), 7.26 (d, 1H).

### Example 162

### N-[4-[3,5-bis(trifluoromethyl)-1H-pyrazol-1-yl]phenyl]-3-methyl-2-thiophenecarboxamide

Example (i)-a B was processed as in Example (i)-a (Method 5, 6, or 7) to provide the title compound.

mp 185-188° C.;

MS (DCI/NH₃) m/e 437 (M+NH₄)⁺;

¹H NMR (DMSO-d₆, 300 MHz) δ10.30 (s, 1H), 7.90 (d, 2H), 7.80 (s, 1H), 7.71 (d, 1H), 7.58 (d, 2H), 7.06 (d, 1H), 2.43 (s, 3H).

### Example 163

### 2-amino-N-[4-[3,5-bis(trifluoromethyl)-1H-pyrazol-1-yl]phenyl]benzamide

Example 163 was prepared from Example 136 using the reduction procedure described in Example 59.

mp 204-206° C.;

MS (DCI/NH₃) m/e 415 (M+H)⁺;

¹H NMR (DMSO-d₆, 300 MHz) δ10.32 (s, 1H), 7.95 (d, 2H), 7.82 (s, 1H), 7.67 (d, 1H), 7.58 (d, 2H), 7.23 (t, 1H), 6.78 (d, 1H), 6.62 (t, 1H), 6.37 (s, 2H).

### Example 164

### N-[4-[3,5-bis(trifluoromethyl)-1H-pyrazol-1-yl]phenyl]-2-fluoro-3-pyridinecarboxamide

Example (i)-a B was processed as in Example (i)-a (Method 5, 6, or 7) to provide the title compound.

mp 110-112° C.;

MS (DCI/NH₃) m/e 436 (M+NH₄)⁺;

¹H NMR (DMSO-d₆, 300 MHz) δ10.94 (s, 1H), 8.42 (d, 1H), 8.31 (dd, 1H), 7.92 (d, 2H), 7.82 (s, 1H), 7.64 (d, 2H), 7.55 (dd, 1H).

### Example 165

### N-[4-[3,5-bis(trifluoromethyl)-1H-pyrazol-1-yl]phenyl]-3-chloro-4-(methylsulfonyl)-2-thiophenecarboxamide

Example (i)-a B was processed as in Example (i)-a (Method 5, 6, or 7) to provide the title compound.

mp 210-212° C.;

MS (DCI/NH₃) m/e 535 (M+NH₄)⁺;

¹H NMR (DMSO-d₆, 300 MHz) δ10.84 (s, 1H), 9.94 (s, 1H), 7.82 (d, 2H), 7.75 (s, 1H), 7.55 (d, 2H), 3.30 (s, 3H).

### Example 166

### N-[4-[3,5-bis(trifluoromethyl)-1H-pyrazol-1-yl]phenyl]-1H-pyrrole-2-carboxamide

Example (i)-a B was processed as in Example (i)-a (Method 5, 6, or 7) to provide the title compound.

mp>240° C.;

MS (DCI/NH₃) m/e 406 (M+NH₄)⁺;

¹H NMR (DMSO-d₆, 300 MHz) δ7.96 (d, 2H), 7.82 (s, 1H), 7.58 (d, 2H), 7.13 (s, 1H), 7.02 (s, 1H), 6.20 (s, 1H).

### Example 167

### N-[4-[3,5-bis(trifluoromethyl)-1H-pyrazol-1-yl]phenyl]-3,6-dichloro-2-pyridinecarboxamide

Example (i)-a B was processed as in Example (i)-a (Method 5, 6, or 7) to provide the title compound.

mp 118-119° C.;

MS (DCI/NH₃) m/e 486 (M+NH₄)⁺;

¹H NMR (DMSO-d₆, 300 MHz) δ11.11 (s, 1H), 8.24 (d, 1H), 7.92 (d, 2H), 7.82 (s, 1H), 7.78 (d, 1H), 7.63 (d, 2H).

### Example 168

### N-[4-[3,5-bis(trifluoromethyl)-1H-pyrazol-1-yl]phenyl]-2-(2-nitrophenoxy)acetamide

Example (i)-a B was processed as in Example (i)-a (Method 5, 6, or 7) to provide the title compound.

mp 170-173° C.;

MS (DCI/NH₃) m/e 492 (M+NH₄)⁺;

¹H NMR (DMSO-d₆, 300 MHz) δ7.78 (d, 2H), 7.22 (d, 2H), 7.05 (s, 1H), 7.03 (d, 1H), 6.70 (d, 2H), 6.26 (d, 1H), 5.72 (s, 1H), 3.92 (s, 2H).

### Example 169

### N-[4-[3,5-bis(trifluoromethyl)-1H-pyrazol-1-yl]phenyl]-4-chlorobenzeneacetamide

Example (i)-c B was processed as in Example (i)-c (Method 5, 6, or 7) to provide the title compound.

mp 122-124° C.;

MS (DCI/NH₃) m/e 465 (M+NH₄)⁺;

¹H NMR (DMSO-d₆, 300 MHz) δ9.5 (s, 1H), 7.9 (m, 2H), 7.6 (m, 1H), 7.5 (d, 1H), 7.4 (m, 3H), 7.2 (d, 2H), 3.5 (s, 2H).

### Example 170

### N-[4-[3,5-bis(trifluoromethyl)-1H-pyrazol-1-yl]phenyl]-1H-indole-2-acetamide

Example (i)-a B was processed as in Example (i)-a (Method 5, 6, or 7) to provide the title compound.

mp 176-178° C.;

MS (DCI/NH₃) m/e 470 (M+NH₄)⁺;

¹H NMR (DMSO-d₆, 300 MHz) δ10.92 (s, 1H), 10.44 (s, 1H), 7.84 (s, 1H), 7.81 (d, 2H), 7.63 (d, 1H), 7.53 (d, 2H), 7.36 (d, 1H), 7.28 (d, 1H), 7.08 (td, 1H), 6.98 (td, 1H), 3.78 (s, 2H).

### Example 171

### (E)-N-[4-[3,5-bis(trifluoromethyl)-1H-pyrazol-1-yl]phenyl]-3-(2-thienyl)-2-propenamide

Example (i)-a B was processed as in Example (i)-a (Method 5, 6, or 7) to provide the title compound.

mp 216-218° C.;

MS (DCI/NH₃) m/e 449 (M+NH₄)⁺;

¹H NMR (DMSO-d₆, 300 MHz) δ7.95 (d, 1H), 7.90 (d, 2H), 7.82 (s, 1H), 7.64 (t, 1H), 7.63 (d, 1H), 7.58 (d, 2H), 7.42 (d, 1H), 6.68 (d, 1H).

### Example 172

### N-[4-[3,5-Bis(trifluoromethyl)-1H-pyrazol-1-yl]phenyl]pyazinecarboxamide

Example (i)-a B was processed as in Example (i)-a (Method 5, 6, or 7) to provide the title compound.

mp 183-185° C.;

MS (DCI/NH₃) m/e 419 (M+NH₄)⁺;

¹H NMR (DMSO-d₆, 300 MHz) δ9.34 (d, 1H), 8.98 (d, 1H), 8.84 (dd, 1H), 8.15 (d, 2H), 7.83 (s, 1H), 7.64 (d, 2H).

### Example 173

### 1,1-Dimethylethyl [[4-[4-[3,5-bis(trifluoromethyl)-1H-pyrazol-1-yl]phenyl]amino]-4-oxobutyl]carbamate

Example (i)-a B was processed as in Example (i)-a (Method 5, 6, or 7) to provide the title compound.

MS (DCI/NH₃) m/e 481 (M+H)⁺;

¹H NMR (DMSO-d₆, 300 MHz) δ7.80 (d, 2H), 7.95 (s, 1H), 7.55 (d, 2H), 6.82 (t, 1H), 2.98 (q, 2H), 2.34 (t, 2H), 1.71 (quintet, 2H), 1.38 (s, 9H).

### Example 174

### 1-Acetyl-N-[4-[3,5-bis(trifluoromethyl)-1H-pyrazol-1-yl]phenyl]-4-piperidinecarboxamide

Example (i)-a B was processed as in Example (i)-a (Method 5, 6, or 7) to provide the title compound.

mp 94-95° C.;

MS (DCI/NH₃) m/e 466 (M+NH₄)⁺;

¹H NMR (DMSO-d₆, 300 MHz) δ10.26 (s, 1H), 7.82 (d, 2H), 7.78 (s, 1H), 7.53 (d, 2H), 4.41 (d, 1H), 3.87 (d, 1H), 3.08 (t, 1H), 2.68-2.55 (m, 2H), 2.01 (s, 3H), 1.92-1.78 (m, 2H), 1.71-1.55 (m, 2H).

### Example 175

### N-[4-[3,5-Bis(trifluoromethyl)-1H-pyrazol-1-yl]phenyl]butanamide

Example (i)-a B was processed as in Example (i)-a (Method 5, 6, or 7) to provide the title compound.

MS (DCI/NH₃) m/e 383 (M+NH₄)⁺;

¹H NMR (DMSO-d₆, 300 MHz) δ10.26 (s, 1H), 7.82 (d, 2H), 7.80 (s, 1H), 7.53 (d, 2H), 2.51-2.44 (t, 2H), 1.57 (sextet, 2H), 0.91 (t, 3H).

### Example 176

### N-[4-[3,5-Bis(trifluoromethyl)-1H-pyrazol-1-yl]phenyl]-4-chloro-2-methoxybenzamide

Example (i)-a B was processed as in Example (i)-a (Method 5, 6, or 7) to provide the title compound.

mp 178-180° C.;

MS (DCI/NH₃) m/e 481 (M+NH₄)⁺;

¹H NMR (DMSO-d₆, 300 MHz) δ10.5 (s, 1H), 8.0 (d, 2H), 7.8 (s, 1H), 7.7 (d, 1H), 7.6 (d, 2H), 7.3 (d, 1H), 7.2 (dd, 1H), 3.3 (s, 3H).

### Example 177

### N-[4-[3,5-bis(trifluoromethyl)-1H-pyrazol-1-yl]phenyl]-α-methyl-4-(2-thienylcarbonyl)benzeneacetamide

Example (i)-a B was processed as in Example (i)-a (Method 5, 6, or 7) to provide the title compound.

mp<80° C.;

MS (DCI/NH₃) m/e 555 (M+NH₄)⁺;

¹H NMR (DMSO-d₆, 300 MHz) δ8.11 (dd, 1H), 7.85 (dd, 2H), 7.83 (dd, 2H), 7.79 (s, 1H), 7.75 (dd, 1H), 7.60 (d, 2H), 7.55 (d, 2H), 7.28 (dd, 1H), 4.01 (q, 1H), 1.5 (d, 3H).

### Example 178

### N-[4-[3,5-bis(trifluoromethyl)-1H-pyrazol-1-yl]phenyl]-α-methyl-4-(2-thienylcarbonyl)benzeneacetamide

Example (i)-a B was processed as in Example (i)-a (Method 5, 6, or 7) to provide the title compound.

mp<100° C.;

MS (DCI/NH₃) m/e 555 (M+NH₄)⁺;

¹H NMR (DMSO-d₆, 300 MHz) δ10.54 (s, 1H), 8.11 (dd, 1H) 7.85 (dd, 2H), 7.83 (dd, 2H), 7.81 (s, 1H), 7.75 (dd, 1H), 7.60 (d, 2H), 7.55 (d, 2H), 7.28 (dd, 1H), 4.01 (q, 1H), 1.5 (d, 3H).

### Example 179

### N-[4-[3,5-bis(trifluoromethyl)-1H-pyrazol-1-yl]phenyl]-2-methoxy-4-(methythio)benzamide

Example (i)-a B was processed as in Example (i)-a (Method 5, 6, or 7) to provide the title compound.

mp 164-165° C.;

MS (DCI/NH₃) m/e 493 (M+NH₄)⁺;

¹H NMR (DMSO-d₆, 300 MHz) δ10.35 (s, 1H), 7.95 (d, 2H), 7.82 (s, 1H), 7.65 (d, 1H), 7.59 (d, 2H), 7.03 (d, 1H), 6.96 (dd, 1H), 3.95 (s, 3H), 2.55 (s, 3H).

### Example 180

### N-[4-[3,5-bis(trifluoromethyl)-1H-pyrazol-1-yl]phenyl]-4-hydroxy-3-nitrobenzamide

### Example 180A

Example (i)-a B and 3-nitro-4-methoxybenzoic acid were processed as in Example (i)-a (Method 5, 6, or 7) to provide the desired compound.

### Example 180B

### N-[4-[3,5-bis(trifluoromethyl)-1H-pyrazol-1-yl]phenyl]-4-hydroxy-3-nitrobenzamide

A solution of example 180A (1.0 mmol) in toluene (3 mnL) at −78° C. was treated dropwise with BBr₃ (1.0M in toluene, 1.5 equivalents for each hydroxyl), stirred at −78 ° C. for 2 hours and at room temperature for 16 hours, recooled to −78 ° C., treated with methanol (1 mL), warmed to room temperature, and concentrated. The residue was filtered through a MgSO₄/silica gel plug with 20% acetone in hexanes and further purified by HPLC eluting with 20% acetone in hexanes.

mp 193-194° C.;

MS (DCI/NH₃) m/e 478 (M+NH₄)⁺;

¹H NMR (DMSO-d₆, 300 MHz) δ10.58 (s, 1H), 8.58 (s, 1H), 8.17 (d, 1H), 7.98 (d, 2H), 7.82 (s, 1H), 7.62 (d, 2H), 7.25 (d, 1H).

### Example 181

### N-[4-[3,5-bis(trifluoromethyl)-1H-pyrazol-1-yl]phenyl]-3,4-dihydroxybenzamide

Example (i)-a B and 3,4-dimethoxybenzoic acid were processed as in examples (i)-a (Method 5, 6, or 7) and 180B to provide the title compound.

mp 233-235° C.;

MS (DCI/NH₃) m/e 449 (M+NH₄)⁺;

¹H NMR (DMSO-d₆, 300 MHz) δ8.0 (d, 2H), 7.8 (s, 1H), 7.6 (d, 2H), 7.4 (m, 2H), 6.8 (d, 1H).

### Example 182

### N-[4-[3,5-bis(trifluoromethyl)-1H-pyrazol-1-yl]phenyl]-2-hydroxy-6-methoxybenzamide

Example (i)-a B and 2,6-dimethoxybenzoic acid were processed as in examples (i)-a (Method 5, 6, or 7) and 180B (using 1.5 equivalents of 1.0M BBr₃ in toluene) to provide the title compound.

mp 114-116° C.;

MS (DCI/NH₃) m/e 446 (M+NH₄)⁺;

¹H NMR (DMSO-d₆, 300 MHz) δ10.6 (s, 1H), 10.3 (s, 1H), 8.0 (d, 2H), 7.8 (s, 1H), 7.6 (d, 2H), 7.2 (t, 1H), 6.6 (d, 1H), 6.6 (d, 1H), 3.8 (s, 3H).

### Example 183

### N-[4-[3,5-bis(trifluoromethyl)-1H-pyrazol-1-yl]phenyl]-2,4-bis(trifluoromethyl)benzamide

Example (i)-a B was processed as in Example (i)-a (Method 5, 6, or 7) to provide the title compound.

mp 200-201° C.;

MS (DCI/NH₃) m/e 553 (M+NH₄)⁺;

¹H NMR (DMSO-d₆, 300 MHz) δ8.28 (d, 1H), 8.25 (s, 1H), 8.10 (d, 1H), 7.88 (d, 2H), 7.84 (s, 1H), 7.64 (d, 2H).

### Example 184

### N-[4-[3,5-bis(trifluoromethyl)-1H-pyrazol-1-yl]phenyl]-5-methyl-4-isoxazolecarboxamide

Example (i)-a B was processed as in Example (i)-a (Method 5, 6, or 7) to provide the title compound.

mp 163-167° C.;

MS (DCI/NH₃) m/e 422 (M+NH₄)⁺;

¹H NMR (DMSO-d₆, 300 MHz) δ10.97 (s, 1H), 8.01 (d, 2H), 7.80 (s, 1H), 7.61 (d, 2H), 6.69 (s, 1H), 2.50 (s, 3H).

### Example 185

### 4-[3,5-Bis(trifluoromethyl)-1H-pyrazol-1-yl]-N-(2-chlorophenyl)benzamide

Example (v)-a C was processed as in Example (v)-a (Method 11) to provide the title compound.

mp 162-164° C.;

MS (DCI/NH₃) m/e 451 (M+NH₄)⁺;

¹H NMR (DMSO-d₆, 300 MHz) δ10.4 (s, 1H), 8.2 (d, 2H), 7.9 (s, 1H), 7.8 (d, 2H), 7.6 (m, 2H), 7.5-7.3 (m, 2H).

### Example 186

### 4-[3,5-Bis(trifluoromethyl)-1H-pyrazol-1-yl]-N-(3-cyanophenyl)benzamide

Example (v)-a C was processed as in Example (v)-a (Method 11) to provide the title compound.

mp 195-196° C.;

MS (DCI/NH₃) m/e 442 (M+NH₄)⁺;

¹H NMR (DMSO-d₆, 300 MHz) δ10.8 (s, 1H), 8.3 (s, 1H), 8.2 (d, 2H), 8.1 (m, 1H), 7.9 (s, 1H), 7.8 (d, 2H), 7.6 (d, 1H), 7.6 (d, 1H).

### Example 187

### 4-[3,5-Bis(trifluoromethyl)-1H-pyrazol-1-yl]-N-(2,4-difluorophenyl)benzamide

Example (v)-a C was processed as in Example (v)-a (Method 11) to provide the title compound.

mp 176-177° C.;

MS (DCI/NH₃) m/e 453 (M+NH₄)⁺;

¹H NMR (DMSO-d_{6,} 300 MHz) δ10.4 (s, 1H), 8.2 (d, 2H), 7.9 (s, 1H), 7.8 (d, 2H), 7.6 (m, 1H), 7.4 (m, 1H), 7.2 (m, 1H).

### Example 188

### 4-[3,5-Bis(trifluoromethyl)-1H-pyrazol-1-yl]-N-(2-cyanophenyl)benzamide

Example (v)-a C was processed as in Example (v)-a (Method 11) to provide the title compound.

mp 203-205° C.;

MS (DCI/NH₃) m/e 442 (M+NH₄)⁺;

¹H NMR (DMSO-d₆, 300 MHz) δ10.88 (s, 1H), 8.22 (d, 2H), 7.93 (s, 1H), 7.93 (d, 1H), 7.85 (d, 2H), 7.78 (dt, 1H), 7.63 (d, 1H), 7.46 (dt, 1H).

### Example 189

### 3,5-dimethyl-N-[4-(3,5-dimethyl-1H-1,2,4-triazol-1-yl)phenyl]-4-isoxazolecarboxamide

Example (xxv)-a B was processed as in Example (i)-a (Method 5, 6, or 7) to provide the title compound.

mp 170-171° C.;

MS (DCI/NH₃) m/e 312 (M+H)⁺;

¹H NMR (CDCl₃, 300 MHz) δ7.79 (d, 2H), 7.45 (d, 2H), 7.3 (s, 1H), 2.7 (s, 3H), 2.54 (s, 3H), 2.48 (s, 3H), 2.42 (s, 3H).

### Example 190

### 4-[3,5-Bis(trifluoromethyl)-1H-pyrazol-1-yl]-N-(2-nitrophenyl)benzamide

Example (v)-a C was processed as in Example (v)-a (Method 11) to provide the title compound.

mp 173-175° C.;

MS (DCI/NH₃) m/e 462 (M+NH₄)⁺;

¹H NMR (DMSO-d₆, 300 MHz) δ11.0 (s, 1H), 8.2 (d, 2H), 8.0 (d, 1H), 7.9 (s, 1H), 7.8 (d, 2H), 7.7 (m, 2H), 7.4 (m, 1H).

### Example 191

### 4-[3,5-Bis(trifluoromethyl)-1H-pyrazol-1-yl]-N-(2,6-difluorophenyl)benzamide

Example (v)-a C was processed as in Example (v)-a (Method 11) to provide the title compound.

mp 187-188° C.;

MS (DCI/NH₃) m/e 453 (M+NH₄)⁺; ¹H NMR (DMSO-d₆, 300 MHz) δ10.4 (s, 1H), 8.2 (d, 2H), 7.9 (s, 1H), 7.8 (d, 2H), 7.4 (m, 1H), 7.3 (d, 1H), 7.2 (d, 1H).

### Example 192

### 4-[3,5-Bis(trifluoromethyl)-1H-pyrazol-1-yl]-N-(2-bromophenyl)benzamide

Example (v)-a C was processed as in Example (v)-a (Method 11) to provide the title compound.

mp 163-165° C.;

MS (DCI/NH₃) m/e 496 (M+NH₄)⁺;

¹H NMR (DMSO-d₆, 300 MHz) δ10.33 (s, 1H), 8.2 (d, 2H), 7.89 (s, 1H), 7.83 (d, 2H), 7.74 (dd, 1H), 7.58 (dd, 1H), 7.45 (dt, 1H), 7.26 (dt, 1H).

### Example 193

### 4-[3,5-Bis(trifluoromethyl)-1H-pyrazol-1-yl]-N-(4-cyanophenyl)benzamide

Example (v)-a C was processed as in Example (v)-a (Method 11) to provide the title compound.

mp 192-193° C.;

MS (DCI/NH₃) m/e 442 (M+NH₄)⁺;

¹H NMR (DMSO-d₆, 300 MHz) δ10.87 (s, 1H), 8.17 (d, 2H), 8.0 (d, 2H), 7.92 (s, 1H), 7.86 (d, 2H), 7.82 (d, 2H).

### Example 194

### 4-[3,5-Bis(trifluoromethyl)-1H-pyrazol-1-yl]-N-(4-pyridinyl)benzamide

Example (v)-a C was processed as in Example (v)-a (Method 11) to provide the title compound.

mp 234-235° C.;

MS (DCI/NH₃) m/e 401 (M+H)⁺;

¹H NMR (DMSO-d₆, 300 MHz) δ11.77 (s, 1H), 8.78 (d, 2H), 8.34 (d, 2H), 8.25 (d, 2H), 7.94 (s, 1H), 7.9 (d, 2H).

### Example 195

### N-[4-[3,5-bis(trifluoromethyl)-1H-pyrazol-1-yl]phenyl]-4-fluoro-3-(trifluoromethyl)benzamide

Example (i)-a B was processed as in Example (i)-a (Method 5, 6, or 7) to provide the title compound.

mp 142-144° C.;

MS (ESI−) m/e 485 (M−H)⁻;

¹H NMR (DMSO-d_{6,} 300 MHz) δ10.7 (s, 1H, ), 8.45 (m, 2H), 7.95 (d, 2H), 7.85 (s, 1H), 7.75 (m, 1H), 7.65 (d, 2H).

### Example 196

### N-[2-(aminocarbonyl)phenyl]-4-[3,5-bis(trifluoromethyl)-1H-pyrazol-1-yl]benzamide

Example (v)-a C was processed as in Example (v)-a (Method 11) to provide the title compound.

mp 195-196° C.;

MS (DCI/NH₃) m/e 460 (M+NH₄)⁺;

¹H NMR (DMSO-d₆, 300 MHz) δ13.14 (s, 1H), 8.7 (d, 1H), 8.46 (s, 1H), 8.15 (d, 2H), 7.9 (m, 5H), 7.62 (t, 1H), 7.22 (t, 1H).

### Example 197

### N-[3-[3,5-bis(trifluoromethyl)-1H-pyrazol-1-yl]phenyl]-4-chlorobenzeneacetamide

Example (i)-b B was processed as in Example (i)-b (Method 5, 6, or 7) to provide the title compound.

mp 209-211° C.;

MS (DCI/NH₃) m/e 465 (M+NH₄)⁺;

¹H NMR (DMSO-d₆, 300 MHz) δ10.6 (s, 1H), 8.0 (t, 1H), 7.8 (s, 1H), 7.7 (d, 1H), 7.6 (t, 1H), 7.4-7.2 (m, 5H), 3.7 (s, 2H).

### Example 198

### N-[3-[3,5-bis(trifluoromethyl)-1H-pyrazol-1-yl]phenyl]-2,3-dichlorobenzamide

Example (i)-b B was processed as in Example (i)-b (Method 5, 6, or 7) to provide the title compound.

mp 119-121° C.;

MS (DCI/NH₃) m/e 485 (M+NH₄)⁺;

¹H NMR (DMSO-d₆, 300 MHz) δ11.0 (s, 1H), 8.1 (t, 1H), 7.9 (s, 1H), 7.8 (dd, 2H), 7.6 (m, 2H), 7.5 (t, 1H), 7.4 (d, 1H).

### Example 199

### N-[3-[3,5-bis(trifluoromethyl)-1H-pyrazol-1-yl]phenyl]-2-chloro-5-nitrobenzamide

Example (i)-b B was processed as in Example (i)-b (Method 5, 6, or 7) to provide the title compound.

mp 147-152° C.;

MS (DCI/NH₃) m/e 496 (M+NH₄)⁺;

¹H NMR (DMSO-d₆, 300 MHz) δ11.1 (s, 1H), 8.6 (d, 1H), 8.4 (dd, 1H), 8.1 (s, 1H), 7.9-7.8 (m, 3H), 7.6 (t, 1H), 7.2 (d, 1H).

### Example 200

### N-[4-[3,5-bis(trifluoromethyl)-1H-pyrazol-1-yl]phenyl]-4-fluoro-3-nitrobenzamide

Example (i)-a B was processed as in Example (i)-a (Method 5, 6, or 7) to provide the title compound.

mp 186-187° C.;

MS (DCI/NH₃) m/e 480 (M+NH₄)⁺;

¹H NMR (DMSO-d₆, 300 MHz) δ10.84 (s, 1H), 8.78 (dd, 1H), 8.42 (m, 1H), 8.0 (d, 2H), 7.82 (s, 1H), 7.81 (dd, 1H), 7.64 (d, 2H).

### Example 201

### N-[4-[3,5-bis(trifluoromethyl)-1H-pyrazol-1-yl]phenyl]-4-fluoro-2-(trifluoromethyl)benzamide

Example (i)-a B was processed as in Example (i)-a (Method 5, 6, or 7) to provide the title compound.

mp 178-179° C.;

MS (DCI/NH₃) m/e 503 (M+NH₄)⁺;

¹H NMR (DMSO-d₆, 300 MHz) δ10.96 (s, 1H), 7.91 (d, 1H), 7.89 (d, 2H), 7.85 (dd, 1H), 7.83 (s, 1H), 7.72 (dt, 1H), 7.62 (d, 2H).

### Example 202

### N-[3-[3,5-bis(trifluoromethyl)-1H-pyrazol-1-yl]phenyl]-4-fluorobenzamide

Example (i)-b B was processed as in Example (i)-b (Method 5, 6, or 7) to provide the title compound.

mp 150-152° C.;

MS (DCI/NH₃) m/e 435 (M+NH₄)⁺;

¹H NMR (DMSO-d₆, 300 MHz) δ10.6 (s, 1H), 8.2 (t, 1H), 8.1 (m, 2H), 8.0 (d, 1H), 7.9 (s, 1H), 7.6 (t, 1H), 7.5-7.3 (m, 3H).

### Example 203

### N-[3-[3,5-bis(trifluoromethyl)-1H-pyrazol-1-yl]phenyl]-2,4-difluorobenzamide

Example (i)-b B was processed as in Example (i)-b (Method 5, 6, or 7) to provide the title compound.

mp 133-134° C.;

MS (DCI/NH₃) m/e 453 (M+NH₄)⁺;

¹H NMR (DMSO-d₆, 300 MHz) δ7.85 (d, 1H), 7.85 (d, 1H), 7.78 (t, 1H), 7.62 (t, 1H), 7.45 (dt, 1H), 7.38 (d, 1H), 7.25 (dt, 1H).

### Example 204

### N-[3-[3,5-bis(trifluoromethyl)-1H-pyrazol-1-yl]phenyl]-3-cyanobenzamide

Example (i)-b B was processed as in Example (i)-b (Method 5, 6, or 7) to provide the title compound.

mp 160-161° C.;

MS (DCI/NH₃) m/e 442 (M+NH₄)⁺;

¹H NMR (DMSO-d₆, 300 MHz) δ10.75 (s, 1H), 8.44 (s, 1H), 8.27 (d, 1H), 8.15 (s, 1H), 8.1 (d, 1H), 7.98 (d, 1H), 7.88 (s, 1H), 7.78 (t, 1H), 7.63 (t, 1H), 7.4 (d, 1H).

### Example 205

### N-[4-[3,5-bis(trifluoromethyl)-1H-pyrazol-1-yl]phenyl]-2-bromo-3-nitrobenzamide

Example (i)-a B was processed as in Example (i)-a (Method 5, 6, or 7) to provide the title compound.

mp 213-214° C.;

MS (DCI/NH₃) m/e 541 (M+NH₄)⁺;

¹H NMR (DMSO-d₆, 300 MHz) δ11.6 (s, 1H), 8.12 (dd, 1H), 7.86 (dd, 1H), 7.86 (d, 2H), 7.83 (s, 1H), 7.77 (t, 1H), 7.64 (d, 2H).

### Example 206

### N-[4-[3,5-bis(trifluoromethyl)-1H-pyrazol-1-yl]phenyl]-2-chloro-4-fluorobenzamide

Example (i)-a B was processed as in Example (i)-a (Method 5, 6, or 7) to provide the title compound.

mp 155-157° C.;

MS (DCI/NH₃) 469 (M+H)⁺;

¹H NMR (DMSO-d₆, 300 MHz) δ7.92 (d, 2H), 7.83 (s, 1H), 7.90-7.60 (m, 2H), 7.63 (d, 2H), 7.40 (dt, 1H).

### Example 207

### N-[4-[3,5-bis(trifluoromethyl)-1H-pyrazol-1-yl]phenyl]-2-chloro-4-(methylsulfonyl)benzamide

Example (i)-a B was processed as in Example (i)-a (Method 5, 6, or 7) to provide the title compound.

mp 219-220° C.;

MS (DCI/NH₃) 529 (M+NH₄)⁺;

¹H NMR (DMSO-d₆, 300 MHz) δ11.08 (br s, 1H), 8.16 (d, 1H), 8.05-7.95 (m, 2H), 7.96 (d, 2H), 7.84 (s, 1H), 7.65 (d, 2H), 2.38 (s, 3H).

### Example 208

### N-[4-[3,5-bis(trifluoromethyl)-1H-pyrazol-1-yl]phenyl]-2,5-dichlorobenzamide

Example (i)-a B was processed as in Example (i)-a (Method 5, 6, or 7) to provide the title compound.

mp 154-156° C.;

MS(DCI/NH₃) 485 (M+NH₄)⁺;

¹H NMR (DMSO-d₆, 300 MHz) δ10.99 (br s, 1H), 7.98 (d, 2H), 7.87 (s, 2H), 7.72-7.65 (m, 4H).

### Example 209

### N-[4-[3,5-bis(trifluoromethyl)-1H-pyrazol-1-yl]phenyl]-2,3-difluorobenzamide

Example (i)-a B was processed as in Example (i)-a (Method 5, 6, or 7) to provide the title compound.

mp 147-149° C.;

MS (DCI/NH₃) m/e 453 (M+NH₄)⁺;

¹H NMR (DMSO-d₆, 300 MHz) δ10.9 (s, 1H), 7.9 (d, 2H), 7.8 (s, 1H), 7.7 (m, 1H), 7.6 (d, 2H), 7.5 (m, 1H), 7.4 (m, 1H).

### Example 210

### N-[4-[3,5-bis(trifluoromethyl)-1H-pyrazol-1-yl]phenyl]-3-chloro-4-fluorobenzamide

Example (i)-a B was processed as in Example (i)-a (Method 5, 6, or 7) to provide the title compound.

mp 138-139° C.;

MS (DCI/NH₃) m/e 469 (M+NH₄)⁺;

¹H NMR (DMSO-d₆, 300 MHz) δ10.5 (s, 1H), 8.1 (dd, 1H), 7.9 (m, 1H), 7.8 (d, 2H), 7.7 (s, 1H), 7.5 (d, 2H), 7.4 (m, 1H).

### Example 211

### N-[4-[3,5-bis(trifluoromethyl)-1H-pyrazol-1-yl]phenyl]-2,5-difluorobenzamide

Example (i)-a B was processed as in Example (i)-a (Method 5, 6, or 7) to provide the title compound.

mp 168-169° C.;

MS (DCI/NH₃) m/e 453 (M+NH₄)⁺;

¹H NMR (DMSO-d₆, 300 MHz) δ10.8 (s, 1H), 7.9 (d, 2H), 7.8 (s, 1H), 7.6 (d, 2H), 7.5-7.4 (m, 3H).

### Example 212

### N-[4-[3,5-bis(trifluoromethyl)-1H-pyrazol-1-yl]phenyl]-2-chloro-6-fluorobenzamide

Example (i)-a B was processed as in Example (i)-a (Method 5, 6, or 7) to provide the title compound.

mp 165-167° C.;

MS (DCI/NH₃) m/e 469 (M+NH₄)⁺;

¹H NMR (DMSO-d₆, 300 MHz) δ11.2 (s, 1H), 7.9 (d, 2H), 7.8 (s, 1H), 7.7 (d, 2H), 7.6-7.4 (m, 3H).

### Example 213

### N-[4-[3,5-bis(trifluoromethyl)-1H-pyrazol-1-yl]phenyl]-2-fluoro-6-(trifluoromethyl)benzamide

Example (i)-a B was processed as in Example (i)-a (Method 5, 6, or 7) to provide the title compound.

mp 175-176° C.;

MS (DCI/NH₃) m/e 503 (M+NH₄)⁺;

¹H NMR (DMSO-d₆, 300 MHz) δ11.22 (s, 1H), 7.86 (s, 1H), 7.83 (d, 2H), 7.83-7.74 (m, 3H) 7.63 (d, 2H).

### Example 214

### N-[4-[3,5-bis(trifluoromethyl)-1H-pyrazol-1-yl]phenyl]-3-chloro-2-fluorobenzamide

Example (i)-a B was processed as in Example (i)-a (Method 5, 6, or 7) to provide the title compound.

mp 113-116° C.;

MS(DCI/NH₃) 469 (M+NH₄)⁺;

¹H NMR (DMSO-d₆, 300 MHz) δ10.93 (s, 1H), 7.93 (d, 2H), 7.83 (s, 1H), 7.80 (t, 1H), 7.69 (t, 1H), 7.64 (d, 2H), 7.39 (t, 1H).

### Example 215

### N-[4-[3,5-bis(trifluoromethyl)-1H-pyrazol-1-yl]phenyl]-2-chloro-4-methoxybenzamide

Example (i)-a B was processed as in Example (i)-a (Method 5, 6, or 7) to provide the title compound.

mp 140-143° C.;

MS (DCI/NH₃) m/e 481 (M+NH₄)⁺;

¹H NMR (DMSO-d₆, 300 MHz) δ10.7 (s, 1H), 7.9 (d, 2H), 7.8 (s, 1H), 7.6 (d, 2H), 7.2 (d, 1H), 7.0 (m, 2H), 3.8 (s, 3H).

### Example 216

### N-[4-[3,5-bis(trifluoromethyl)-1H-pyrazol-1-yl]phenyl]-2,6-dichloro-3-nitrobenzamide

Example (i)-a B was processed as in Example (i)-a (Method 5, 6, or 7) to provide the title compound.

mp 160-161° C.;

MS (DCI/NH₃) m/e 530 (M+NH₄)⁺;

¹H NMR (DMSO-d₆, 300 MHz) δ8.32 (d, 1H), 8.12 (d, 1H), 7.78 (d, 2H), 7.68 (s, 1H), 7.53 (d, 2H).

### Example 217

### N-[4-[3,5-bis(trifluoromethyl)-1H-pyrazol-1-yl]phenyl]-4-bromo-2-chlorobenzamide

Example (i)-a B was processed as in Example (i)-a (Method 5, 6, or 7) to provide the title compound.

mp 164-167° C.;

MS (DCI/NH₃) 531 (M+NH₄)⁺;

¹H NMR (DMSO-d₆, 300 MHz) δ10.96 (s, 1H), 7.93 (d, 1H), 7.92 (d, 2H), 7.84 (s, 1H), 7.70-7.80 (m, 1H), 7.63 (d, 2H), 7.58 (d, 1H).

### Example 218

### N-[4-[3,5-bis(trifluoromethyl)-1H-pyrazol-1-yl]phenyl]-3,4-difluorobenzamide

Example (i)-a B was processed as in Example (i)-a (Method 5, 6, or 7) to provide the title compound.

mp 183-185° C.;

MS(DCI/NH₃) 453 (M+NH₄);

¹H NMR (DMSO-d₆, 300 MHz) δ10.65 (s, 1H), 8.30-8.24 (m, 1H), 8.11-8.06 (m, 1H), 7.99 (d, 2H), 7.90-7.87 (m, 1H), 7.83 (s, 1H), 7.80-7.60 (m, 1H), 7.62 (d, 2H).

### Example 219

### N-[4-[3,5-bis(trifluoromethyl)-1H-pyrazol-1-yl]phenyl]-2-bromo-5-methoxybenzamide

Example (i)-a B was processed as in Example (i)-a (Method 5, 6, or 7) to provide the title compound.

mp 179-181° C.;

MS (DCI/NH₃) m/e 525 (M+NH₄)⁺;

¹H NMR (DMSO-d₆, 300 MHz) δ10.8 (s, 1H), 7.9 (d, 2H), 7.8 (s, 1H), 7.6 (d, 2H), 7.2 (d, 1H), 7.0 (d, 1H), 7.0 (dd, 1H), 3.8 (s, 3H).

### Example 220

### N-[4-[3,5-bis(trifluoromethyl)-1H-pyrazol-1-yl]phenyl]-4-chloro-2-hydroxybenzamide

Example (i)-a B and 2-methoxy-4-chlorobenzoic acid were processed as in examples (i)-a (Method 5, 6, or 7) and 180B to provide the title compound.

mp 200-202° C.;

MS (DCI/NH₃) m/e 467 (M+NH₄)⁺;

¹H NMR (DMSO-d₆, 300 MHz) δ8.0 (d, 2H), 7.9 (s, 1H), 7.8 (s, 1H), 7.6 (d, 2H), 7.0 (m, 2H).

### Example 221

### N-[4-[3,5-bis(trifluoromethyl)-1H-pyrazol-1-yl]phenyl]-3-bromo-4-methoxybenzamide

Example (i)-a B was processed as in Example (i)-a (Method 5, 6, or 7) to provide the title compound.

mp 197-198° C.;

MS (DCI/NH₃) m/e 525 (M+NH₄)⁺;

¹H NMR (DMSO-d₆, 300 MHz) δ10.49 (s, 1H), 8.26 (d, 1H), 8.05 (dd, 1H), 7.99 (d, 2H), 7.82 (s, 1H), 7.6 (d, 2H), 7.29 (d, 1H), 3.96 (s, 3H).

### Example 222

### N-[4-[3,5-bis(trifluoromethyl-1H-pyrazol-1-yl]phenyl]-3-bromo-4-hydroxybenzamide

Example 221 was processed as in Example 180B to provide the title compound.

mp 165-167° C.;

MS (ESI−) m/e 492 (M−H)⁻;

¹H NMR (DMSO-d₆, 300 MHz) δ10.4 (s, 1H), 8.2 (d, 1H), 7.97 (d, 2H), 7.87 (dd, 1H), 7.81 (s, 1H), 7.59 (d, 2H), 7.07 (d, 1H).

### Example 223

### N-[4-[3,5-bis(trifluoromethyl)-1H-pyrazol-1-yl]phenyl]-2-chloro-4,5-difluorobenzamide

Example (i)-a B was processed as in Example (i)-a (Method 5, 6, or 7) to provide the title compound.

mp 149-152° C.;

MS (DCI/NH₃) 487 (M+NH₄);

¹H NMR (DMSO-d₆, 300 MHz) δ10.95 (br s, 1H), 8.00-7.90 (m, 2H), 7.91 (d, 2H), 7.84 (s, 1H), 7.64 (d, 2H).

### Example 224

### N-[3-[3,5-bis(trifluoromethyl)-1H-pyrazol-1-yl]phenyl]-2,6-difluorobenzamide

Example (i)-b B was processed as in Example (i)-b (Method 5, 6, or 7) to provide the title compound.

mp 142-143° C.;

MS (DCI/NH₃) m/e 453 (M+NH₄)⁺;

¹H NMR (DMSO-d₆, 300 MHz) δ11.2 (s, 1H), 8.09 (s, 1H), 7.87 (s, 1H), 7.82 (d, 1H), 7.63 (m, 2H), 7.42 (d, 1H), 7.3 (t, 2H).

### Example 225

### N-[4-[3,5-bis(trifluoromethyl)-1H-pyrazol-1-yl]phenyl]-4-chloro-2,5-difluorobenzamide

Example (i)-a B was processed as in Example (i)-a (Method 5, 6, or 7) to provide the title compound.

mp 160-162° C.;

MS (ESI−) m/e 468 (M−H)⁻;

¹H NMR (CDCl₃, 300 MHz) δ8.52 (d, 2H), 8.0 (dd, 1H), 7.85 (d, 2H), 7.15 (s,1H), 7.40 (dd, 1H), 7.42 (d, 2H).

### Example 226

### N-[4-[3,5-bis(trifluoromethyl)-1H-pyrazol-1-yl]phenyl]-2,3,4-trifluorobenzamide

Example (i)-a B was processed as in Example (i)-a (Method 5, 6, or 7) to provide the title compound.

mp 122-124° C.;

MS (DCI/NH₃) m/e 471 (M+NH₄)⁺;

¹H NMR (DMSO-d₆, 300 MHz) δ7.9 (d, 2H), 7.8 (s, 1H), 7.6 (d, 2H), 7.6-7.4 (m, 2H).

### Example 227

### N-[4-[3,5-bis(trifluoromethyl)-1H-pyrazol-1-yl]phenyl]-3,4,5-trifluorobenzamide

Example (i)-a B was processed as in Example (i)-a (Method 5, 6, or 7) to provide the title compound.

mp 158-159° C.;

MS (DCI/NH₃) m/e 471 (M+NH₄)⁺;

¹H NMR (DMSO-d₆, 300 MHz) δ8.02-7.94 (m, 4H), 7.82 (s, 1H), 7.63 (d, 2H).

### Example 228

### N-[4-[3,5-bis(trifluoromethyl)-1H-pyrazol-1-yl]phenyl]-2,4,5-trifluorobenzamide

Example (i)-a B was processed as in Example (i)-a (Method 5, 6, or 7) to provide the title compound.

mp 109-111° C.;

MS (DCI/NH₃) m/e 471 (M+NH₄)⁺;

¹H NMR (DMSO-d₆, 300 MHz) δ10.9 (s, 1H), 7.9 (d, 2H), 7.8 (s, 1H), 7.6 (d, 2H), 7.8-7.6 (m, 2H).

### Example 229

### N-[4-[3,5-bis(trifluoromethyl)-1H-pyrazol-1-yl]phenyl]-2,4,6-trifluorobenzamide

Example (i)-a B was processed as in Example (i)-a (Method 5, 6, or 7) to provide the title compound.

mp 183-185° C.;

MS (DCI/NH₃) 471 (M+NH₄)⁺;

¹H NMR (DMSO-d₆, 300 MHz) δ11.18 (s, 1H), 7.89 (d, 2H), 7.84 (s, 1H), 7.65 (d, 2H), 7.46-7.40 (m, 2H).

### Example 230

### N-[4-[3,5-bis(trifluoromethyl)-1H-pyrazol-1-yl]phenyl]-2,6-difluoro-3-nitrobenzamide

Example (i)-a B was processed as in Example (i)-a (Method 5, 6, or 7) to provide the title compound.

mp 187-189° C.;

MS (DCI/NH₃) m/e 498 (M+NH₄)⁺;

¹H NMR (DMSO-d₆, 300 MHz) δ11.4 (s, 1H), 8.4 (m, 1H), 7.9 (d, 2H), 7.8 (s, 1H), 7.7 (d, 2H), 7.6 (m, 1H).

### Example 231

### N-[4-[3,5-bis(trifluoromethyl)-1H-pyrazol-1-yl]phenyl]-2,3,5-trifluorobenzamide

Example (i)-a B was processed as in Example (i)-a (Method 5, 6, or 7) to provide the title compound.

mp 169-170° C.;

MS (DCI/NH₃) m/e 471 (M+NH₄)⁺;

¹H NMR (DMSO-d₆, 300 MHz) δ10.99 (s, 1H), 7.91 (d, 2H), 7.83 (s, 1H), 7.88-7.78 (m, 1H), 7.64 (d, 2H), 7.58-7.48 (m, 1H).

### Example 232

### N-[4-[3,5-bis(trifluoromethyl)-1H-pyrazol-1-yl]phenyl]-2,4-dichloro-6-fluorobenzamide

Example (i)-a B was processed as in Example (i)-a (Method 5, 6, or 7) to provide the title compound.

mp 185-188° C.;

MS (DCI/NH₃) m/e 503 (M+NH₄)⁺;

¹H NMR (DMSO-d₆, 300 MHz) δ8.01 (d, 1H), 7.94-7.85 (m, 3H), 7.82 (s, 1H), 7.62 (d, 2H).

### Example 233

### N-4-[3,5-bis(trifluoromethyl)-1H-pyrazol-1-yl]phenyl-2,4-dichloro-3,5-dinitrobenzamide

Example (i)-a B was processed as in Example (i)-a (Method 5, 6, or 7) to provide the title compound.

MS (ESI−) m/e 556 (M−H)⁻;

¹H NMR (DMSO-d₆, 300 MHz) δ8.89 (s, 1H), 7.89 (d, 2H), 7.85 (s, 1H), 7.67 (d, 2H).

### Example 234

### N-[4-[3,5-bis(trifluoromethyl)-1H-pyrazol-1-yl]phenyl]-2,3,5,6-tetrafluorobenzamide

Example (i)-a B was processed as in Example (i)-a (Method 5, 6, or 7) to provide the title compound.

mp 235-237° C.;

MS (DCI/NH₃) 489 (M+NH₄)⁺;

¹H NMR (DMSO-d₆, 300 MHz) δ8.18-8.05 (m, 1H), 7.89 (d, 2H), 7.84 (s, 1H), 7.67 (d, 2H).

### Example 235

### N-[4-[3,5-bis(trifluoromethyl)-1H-pyrazol-1-yl]phenyl]-2,3,4,5-tetrafluorobenzamide

Example (i)-a B was processed as in Example (i)-a (Method 5, 6, or 7) to provide the title compound.

mp 138-140° C.;

MS (DCI/NH₃) 470 (M+H)⁺;

¹H NMR (CDCl₃, 300 MHz) δ7.85 (d, 3H), 7.45 (d, 2H), 7.10 (s, 1H).

### Example 236

### N-[4-[3,5-bis(trifluoromethyl)-1H-pyrazol-1-yl]phenyl]-4-bromo-2,3,5,6-tetrafluorobenzamide

Example (i)-a B was processed as in Example (i)-a (Method 5, 6, or 7) to provide the title compound.

mp 183-184° C.;

MS (DCI/NH₃) m/e 507 (M+NH₄)⁺;

¹H NMR (DMSO-d₆, 300 MHz) δ7.9 (s, 1H), 7.9 (d, 2H), 7.7 (d, 2H).

### Example 237

### N-[4-[3,5-bis(trifluoromethyl)-1H-pyrazol-1-yl]phenyl]-5-methyl-2-nitrobenzamide

Example (i)-a B was processed as in Example (i)-a (Method 5, 6, or 7) to provide the title compound.

mp 188-189° C.;

MS (DCI/NH₃) m/e 458 (M+H)⁺;

¹H NMR (DMSO-d₆, 300 MHz) δ8.12 (d, 1H), 7.92-7.82 (t, 3H), 7.67-7.57 (m, 4H), 3.32 (s, 3H).

### Example 238

### N-[3-[3,5-bis(trifluoromethyl)-1H-pyrazol-1-yl]phenyl]-4-cyanobenzamide

Example (i)-b B was processed as in Example (i)-b (Method 5, 6, or 7) to provide the title compound.

mp 133-134° C.;

MS (DCI/NH₃) m/e 442 (M+NH₄)⁺;

¹H NMR (DMSO-d₆, 300 MHz) δ0.8 (s, 1H), 8.2-8.0 (m, 5H), 7.9 (d, 1H), 7.8 (s, 1H), 7.6 (t, 1H), 7.4 (d, 1H).

### Example 239

### N-[4-[3,5-bis(trifluoromethyl)-1H-pyrazol-1-yl]phenyl]-3-thiophenecarboxamide

Example (i)-a B was processed as in Example (i)-a (Method 5, 6, or 7) to provide the title compound.

mp 239-240° C.;

MS (DCI/NH₃) m/e 423 (M+NH₄)⁺;

¹H NMR (DMSO-d₆, 300 MHz) δ8.4 (m, 1H), 8.0 (d, 2H), 7.8 (s, 1H), 7.7-7.6 (m, 2H), 7.6 (d, 2H).

### Example 240

### N-[4-[3,5-bis(trifluoromethyl)-1H-pyrazol-1-yl]phenyl]-5-isoxazolecarboxamide

Example (i)-a B was processed as in Example (i)-a (Method 5, 6, or 7) to provide the title compound.

mp 202-204° C.;

MS (DCI/NH₃) m/e 408 (M+NH₄)⁺;

¹H NMR (DMSO-d₆, 300 MHz) δ11.0 (s, 1H), 8.8 (d, 1H), 8.0 (d, 2H), 7.8 (s, 1H), 7.6 (d, 2H), 7.3 (d, 1H).

### Example 241

### N-[4-[3,5-bis(trifluoromethyl)-1H-pyrazol-1-yl]phenyl]tetrahydro-2-furancarboxamide

Example (i)-a B was processed as in Example (i)-a (Method 5, 6, or 7) to provide the title compound.

mp 113-116° C.;

MS (DCI/NH₃) m/e 411 (M+H)⁺;

¹H NMR (DMSO-d₆, 300 MHz) δ7.93 (d, 2H), 7.80 (s, 1H), 7.56 (d, 2H), 4.45 (dd, 1H), 4.01 (q, 1H), 3.85 (q, 1H), 2.26-2.17 (m, 1H), 2.07-1.98 (m, 1H), 1.93-1.87 (m, 2H).

### Example 242

### N-[4-[3,5-bis(trifluoromethyl)-1H-pyrazol-1-yl]phenyl]-2-pyrrolidinecarboxamide

Example 242 was prepared from Example 246 using the procedure described to prepare Example 125.

mp 82-84° C.;

MS (DCI/NH₃) m/e 393 (M+H)⁺;

¹H NMR (DMSO-d₆, 300 MHz) δ10.30 (s, 1H), 7.90 (d, 2H), 7.82 (s, 1H), 7.55 (d, 2H), 3.76-3.74 (dd, 1H), 2.93 (t, 2H), 2.14-2.01 (m, 1H), 1.88-1.75 (m, 1H), 1.69 (q, 2H).

### Example 243

### N-[4-[3,5-bis(trifluoromethyl)-1H-pyrazol-1-yl]phenyl]tetrahydro-3-furancarboxamide

Example (i)-a B was processed as in Example (i)-a (Method 5, 6, or 7) to provide the title compound.

mp 193-194° C.;

MS (DCI/NH₃) m/e 411 (M+NH₄)⁺;

¹H NMR (DMSO-d₆, 300 MHz) δ10.70 (s, 1H), 8.18-8.03 (m, 3H), 7.90-7.78 (m, 2H), 4.31-4.18 (m, 1H), 4.17-3.89 (m, 4H), 2.45-2.25 (m, 2H).

### Example 244

### N-[4-[3,5-bis(trifluoromethyl)-1H-pyrazol-1-yl]phenyl]-1,2,3-thiadiazole-5-carboxamide

Example (i)-a B was processed as in Example (i)-a (Method 5, 6, or 7) to provide the title compound.

mp 213-214° C.;

MS (DCI/NH₃) m/e 425 (M+NH₄)⁺;

¹H NMR (DMSO-d₆, 300 MHz) δ11.33 (s, 1H), 9.89 (s, 1H), 8.12 (d, 2H), 7.84 (s, 1H), 7.66 (d, 2H).

### Example 245

### N-[4-[3,5-bis(trifluoromethyl)-1H-pyrazol-1-yl]phenyl]-2-chloro-4-pyridinecarboxamide

Example (i)-a B was processed as in Example (i)-a (Method 5, 6, or 7) to provide the title compound.

mp 183-184° C.;

MS (DCI/NH₃) m/e 452 (M+NH₄)⁺;

¹H NMR (DMSO-d₆, 300 MHz) δ10.87 (s, 1H), 8.66 (dd, 1H), 8.03 (s, 1H), 7.98 (d, 1H), 7.80 (d, 2H), 7.82 (s, 1H), 7.66 (d, 2H).

### Example 246

### 1,1-Dimethylethyl 2-[[[4-[3,5-bis(trifluoromethyl)-1H-pyrazol-1-yl]phenyl]amino]carbonyl]-1-pyrrolidinecarboxylate

Example (i)-a B was processed as in Example (i)-a (Method 5, 6, or 7) to provide the title compound.

mp 70-72° C.;

MS (DCI/NH₃) m/e 493 (M+H)⁺;

¹H NMR (DMSO-d₆, 300 MHz) δ10.36 (s, 1H), 7.84-7.77 (m, 3H), 7.56 (d, 2H), 4.31-4.18 (m, 1H), 3.48-3.35 (m, 2H), 1.98-1.80 (m, 4H), 1.40 (s, 3H), 1.27 (s, 6H).

### Example 247

### N-[4-[3,5-bis(trifluoromethyl)-1H-pyrazol-1-yl]phenyl]-5-nitro-2-furancarboxamide

Example (i)-a B was processed as in Example (i)-a (Method 5, 6, or 7) to provide the title compound.

mp 198-199° C.;

MS (DCI/NH₃) m/e 452 (M+NH₄)⁺;

¹H NMR (DMSO-d₆, 300 MHz) δ10.85 (s, 1H), 7.98 (d, 2H), 7.85 (d, 1H), 7.84 (s, 1H), 7.7 (d, 1H), 7.65 (d, 2H).

### Example 248

### N-[4-[3,5-bis(trifluoromethyl)-1H-pyrazol-1-yl]phenyl]-1-methyl-1H-pyrrole-2-carboxamide

Example (i)-a B was processed as in Example (i)-a (Method 5, 6, or 7) to provide the title compound.

mp 148-149° C.;

MS (DCI/NH₃) m/e 420 (M+NH₄)⁺;

¹H NMR (DMSO-d₆, 300 MHz) δ10.0 (s, 1H), 7.9 (d, 2H), 7.8 (s, 1H), 7.6 (d, 2H), 7.1 (m, 2H), 6.1 (dd, 1H), 3.9 (s, 3H).

### Example 249

### N-[4-[3,5-bis(trifluoromethyl)-1H-pyrazol-1-yl]phenyl]-6-chloro-3-pyridinecarboxamide

Example (i)-a B was processed as in Example (i)-a (Method 5, 6, or 7) to provide the title compound.

mp 192-194° C.;

MS (DCI/NH₃) m/e 452 (M+NH₄)⁺;

¹H NMR (DMSO-d₆, 300 MHz) δ10.79 (s, 1H), 8.98 (d, 1H), 8.37 (dd, 1H), 7.97 (d, 2H), 7.82 (s, 1H), 7.73 (d, 1H), 7.64 (d, 2H).

### Example 250

### N-[4-[3,5-bis(trifluoromethyl)-1H-pyrazol-1-yl]phenyl]-4-methyl-1,2,3-thiadizole-5-carboxamide

Example (i)-a B was processed as in Example (i)-a (Method 5, 6, or 7) to provide the title compound.

mp 164-166° C.;

MS (ESI−) m/e 420 (M−H)^{+;}

¹H NMR (DMSO-d₆, 300 MHz) δ11.06 (s, 1H), 7.92 (d, 2H,), 7.84 (s, 1H), 7.66 (d, 2H), 2.84 (s, 3H).

### Example 251

### N-[4-[3,5-bis(trifluoromethyl)-1H-pyrazol-1-yl]phenyl]-5-bromo-2-furancarboxamide

Example (i)-a B was processed as in Example (i)-a (Method 5, 6, or 7) to provide the title compound.

mp 181-182° C.;

MS (DCI/NH₃) m/e 486 (M+NH₄)⁺;

¹H NMR (DMSO-d₆, 300 MHz) δ7.96 (d, 2H), 7.82 (s, 1H), 7.61 (d, 2H), 7.44 (d, 1H), 6.88 (d, 1H).

### Example 252

### N-[4-[3,5-bis(trifluoromethyl)-1H-pyrazol-1-yl]phenyl]-3-methyl-2-furancarboxamide

Example (i)-a B was processed as in Example (i)-a (Method 5, 6, or 7) to provide the title compound.

mp 119-120° C.;

MS (DCI/NH₃) m/e 421 (M+NH₄)⁺;

¹H NMR (DMSO-d₆, 300 MHz) δ10.4 (s, 1H), 8.0 (d, 2H), 7.9 (d, 1H), 7.8 (s, 1H), 7.6 (d, 2H), 6.6 (d, 1H), 2.4 (s, 3H).

### Example 253

### N-[4-[3,5-bis(trifluoromethyl)-1H-pyrazol-1-yl]phenyl]-5-chloro-2-thiophenecarboxamide

Example (i)-a B was processed as in Example (i)-a (Method 5, 6, or 7) to provide the title compound.

mp 182-184° C.;

MS (DCI/NH₃) m/e 457 (M+NH₄)⁺;

¹H NMR (DMSO-d₆, 300 MHz) δ8.0 (d, 1H), 7.9 (d, 2H), 7.8 (s, 1H), 7.6 (d, 2H), 7.3 (d, 1H).

### Example 254

### N-[4-[3,5-bis(trifluoromethyl)-1H-pyrazol-1-yl]phenyl]tetrahydro-5-oxo-2-furancarboxamide

Example (i)-a B was processed as in Example (i)-a (Method 5, 6, or 7) to provide the title compound.

mp 172-173° C.;

MS (DCI/NH₃) m/e 425 (M+NH₄)⁺;

¹H NMR (DMSO-d₆, 300 MHz) δ10.58 (s, 1H), 7.85 (d, 2H), 7.81 (s, 1H), 7.60 (d, 2H), 5.14-5.08 (m, 1H), 2.61-2.50 (m, 3H), 2.34-2.24 (m, 1H).

### Example 255

### N-[4-[3,5-bis(trifluoromethyl)-1H-pyrazol-1-yl]phenyl]-5-oxo-2-pyrrolidinecarboxamide

Example (i)-a B was processed as in Example (i)-a (Method 5, 6, or 7) to provide the title compound.

mp decomposes>250° C.;

MS (DCI/NH₃) m/e 424 (M+NH₄)⁺;

¹H NMR (DMSO-d₆, 300 MHz) δ7.94 (s, 1H), 7.86 (d, 2H), 7.81 (s, 1H), 7.58 (d, 2H), 4.27 (m, 1H), 2.38 (m, 1H), 2.2 (m, 2H), 2.05 (m, 1H).

### Example 256

### N-[4-[3,5-bis(trifluoromethyl)-1H-pyrazol-1-yl]phenyl]-5-bromo-3-pyridinecarboxamide

Example (i)-a B was processed as in Example (i)-a (Method 5, 6, or 7) to provide the title compound.

mp 155-157° C.;

MS (DCI/NH₃) m/e 489 (M+NH₄)⁺;

¹H NMR (DMSO-d₆, 300 MHz) δ10.8 (s, 1H), 9.1 (d, 1H), 8.9 (d, 1H), 8.6 (t, 1H), 8.0 (d, 2H), 7.8 (s, 1H), 7.6 (d, 2H).

### Example 257

### N-[4-[3,5-bis(trifluoromethyl)-1H-pyrazol-1-yl]phenyl]-5-nitro-3-thiophenecarboxamide

Example (i)-a B was processed as in Example (i)-a (Method 5, 6, or 7) to provide the title compound.

mp 164-165° C.;

MS (DCI/NH₃) m/e 468 (M+NH₄)⁺;

¹H NMR (DMSO-d₆, 300 MHz) δ8.7 (d, 1H), 8.6 (d, 1H), 8.0 (d, 2H), 7.8 (s, 1H), 7.6 (d, 2H).

### Example 258

### 1,1-Dimethylethyl 4-[[[4-[3,5-bis(trifluoromethyl)-1H-pyrazol-1-yl]phenyl]amino]carbonyl]-3-thiazolidinecarboxylate

Example (i)-a B was processed as in Example (i)-a (Method 5, 6, or 7) to provide the title compound.

mp 82-84° C.;

MS (DCI/NH₃) m/e 528 (M+NH₄)⁺;

¹H NMR (DMSO-d₆, 300 MHz) δ10.45 (s, 1H), 7.82 (s, 1H), 7.80 (d, 2H), 7.58 (d, 2H), 4.68-4.42 (m, 3H), 3.58-3.43 (m, 1H), 3.24-3.15 (m, 1H), 1.30 (s, 9H).

### Example 259

### N-[4-[3,5-bis(trifluoromethyl)-1H-pyrazol-1-yl]phenyl]-5-methoxy-3-thiophenecarboxamide

Example (i)-a B was processed as in Example (i)-a (Method 5, 6, or 7) to provide the title compound.

mp 213-214° C.;

MS (DCI/NH₃) m/e 453 (M+NH₄)⁺;

¹H NMR (DMSO-d₆, 300 MHz) δ10.00 (s, 1H), 8.17 (d, 1H), 7.93 (d, 2H), 7.82 (s, 1H), 7.60 (d, 2H), 6.85 (d, 1H), 3.93 (s, 3H).

### Example 260

### N-[2-[3,5-bis(trifluoromethyl)-1H-pyrazol-1-yl]-5-pyridinyl]-2-chlorobenzamide

Example (xxiv)-a B was processed as in Example (i)-a (Method 5, 6, or 7) to provide the title compound.

mp 156-159° C.;

MS (DCI/NH₃) m/e 435 (M+H)⁺;

¹H NMR (DMSO-d₆, 300 MHz) δ11.12 (s, 1H), 8.85 (d, 1H), 8.48 (dd, 1H), 7.95 (d, 1H), 7.90 (s, 1H), 7.70-7.50 (m, 4H).

### Example 261

### N-[2-[3,5-bis(trifluoromethyl)-1H-pyrazol-1-yl]-5-pyridinyl]-3-cyanobenzamide

Example (xxiv)-a B was processed as in Example (i)-a (Method 5, 6, or 7) to provide the title compound.

mp 206-208° C.;

MS (DCI/NH₃) m/e 426 (M+H)⁺;

¹H NMR (DMSO-d₆, 300 MHz) δ10.98 (br s, 1H), 8.95 (d, 1H), 8.51 (dd, 1H), 8.17 (d, 2H), 8.15 (d, 2H), 7.94 (d, 1H), 7.87 (s, 1H).

### Example 262

### N-[2-[3,5-bis(trifluoromethyl)-1H-pyrazol-1-yl]-5-pyridinyl]-2-chloro-4,5-difluorobenzamide

Example (xxiv)-a B was processed as in Example (i)-a (Method 5, 6, or 7) to provide the title compound.

mp 113-116° C.;

MS (DCI/NH₃) m/e 471 (M+H)⁺;

¹H NMR (DMSO-d₆, 300 MHz) δ11.15 (s, 1H), 8.83 (d, 1H), 8.45 (dd, 1H), 8.02-7.91 (m, 2H), 7.94 (d, 1H), 7.87 (s, 1H).

### Example 263

### N-[4-[3,5-bis(trifluoromethyl)-1H-pyrazol-1-yl]phenyl]-2,3-dibromo-5-thiophenecarboxamide

Example (i)-a B was processed as in Example (i)-a (Method 5, 6, or 7) to provide the title compound.

mp 142-144° C.;

MS (DCI/NH₃) m/e 581 (M+NH₄)⁺;

¹H NMR (DMSO-d₆, 300 MHz) δ10.66 (s, 1H), 8.13 (s, 1H), 7.95 (d, 2H), 7.85 (s, 1H), 7.65 (d, 2H).

### Example 264

### N-[4-[3,5-bis(trifluoromethyl)-1H-pyrazol-1-yl]phenyl]-3-fluoro-4-pyridinecarboxamide

The procedure described by Shi, G.; Takagishi, S.; Schlosser, M. Tetrahetron. 1994, 50, 1129-1134 and Lecomte, L.; Ndzi, B.; Queguiner, G.; Turck, A. FR. 2,686,340-A1, hereby incorporated by reference, was used. Under reduced pressure, the volatile components were stripped off from a solution of n-butyllithium (30 mL) in hexanes. At −78° C., potassium tert-butoxide (2.75 g, 25 mmol), THF (30 mL), and a precooled solution of 3-fluoropyridine (2.5 g 25 mmol, 2.18 mL) in THF (30 mL) were consecutively added to the residue with stirring until the alcoholate dissolved. After 4 hours at −78° C., the reaction mixture was poured onto fresh dry ice. After evaporation to dryness, the solid salt was treated with a small excess of 1M hydrogen chloride in diethyl ether. Then the mixture (desired product in hydrochloric salt form and KCl salt) was concentrated to give 2.0 g of a brown solid. This mixure was used in the coupling procedure described below in which a small amount of pyridine was used to neutralize the acidic salt form.

Example (i)-a B was processed as in Example (i)-a (Method 5, 6, or 7) to provide the title compound using the isofluoronicotinic acid prepared as described in the preceding paragraph.

mp 152-153° C.;

MS (DCI/NH₃) m/e 436 (M+NH₄)⁺;

¹H NMR (DMSO-d₆, 300 MHz) δ8.80 (s, 1H), 8.63 (dd, 1H), 7.83 (d, 2H), 7.84 (s, 1H), 7.77 (t, 1H), 7.64 (d, 2H).

### Example 265

### N-[4-[3,5-bis(trifluoromethyl)-1H-pyrazol-1-yl]phenyl]-1-methyl-1H-pyrazole-4-carboxamide

To a mixture of ethyl 4-pyrazolecarboxylate (100 mg, 0.71 mmol) and K₂CO₃ (108 mg, 0.78 mmol) in CH₃CN (2 mL) was added methyl iodide (67 μL, 1.07 mmol). After the reaction mixture stirred at room temperature overnight, the precipitate from the reaction was filtered and washed with ether. The filtrates were combined, washed with brine, and dried (Na₂SO₄), and concentrated to provide 80 mg of desired product as an oil:

MS (DCI/NH₃) m/e 172 (M+NH₄)⁺;

¹H NMR (DMSO-d₆, 300MHz) δ8.29 (s, 1H), 7.82 (s, 1H), 4.2 (q, 2H), 3.87 (s, 3H), 1.25 (t, 3H).

Example (i)-a B (59 mg, 0.2 mmol), ethyl-1-methyl-4-pyrazolecarboxylate (31 mg, 0.2 mmol) and NaH (95% dry) (4.8 mg, 0.2 mmol) in DMSO (2 mL) were stirred at room temperature for 2 days and then poured into ice water with stirring. The solid was filtered, washed with water and dried to give the tittle compound in 81% yield.

mp 211-212° C.;

MS (DCI/NH₃) m/e 421 (M+NH₄)⁺;

¹H NMR (DMSO-d₆, 300 MHz) δ10.14 (s, 1H), 8.37 (s, 1H), 8.05 (s, 1H), 7.94 (d, 2H), 7.83 (s, 1H), 7.59 (d, 2H), 3.93 (s, 3H).

### Example 266

### N-[4-[3,5-bis(trifluoromethyl)-1H-pyrazol-1-yl]phenyl]-3,5-dimethyl-4-isoxazolecarboxamide

Example (i)-a B was processed as in Example (i)-a (Method 5, 6, or 7) to provide the title compound.

mp 179-180° C.;

MS (DCI/NH₃) m/e 436 (M+NH₄)⁺;

¹H NMR (DMSO-d₆, 300 MHz) δ10.40 (br s, 1H), 7.88 (d, 2H), 7.83 (s, 1H), 7.63 (d, 2H), 2.58 (s, 3H), 2.36 (s, 3H).

### Example 267

### N-[4-[3,5-bis(trifluoromethyl)-1H-pyrazol-1-yl]phenyl]-5-chloro-4-methoxy-3-thiophenecarboxamide

Example (i)-a B was processed as in Example (i)-a (Method 5, 6, or 7) to provide the title compound.

mp 119-120° C.;

MS (DCI/NH₃) m/e 487 (M+NH₄)⁺;

¹H NMR (DMSO-d₆, 300 MHz) δ10.43 (s, 1H), 8.06 (s, 1H), 7.93 (d, 2H), 7.84 (s, 1H), 7.6 (d, 2H), 3.93 (s, 3H).

### Example 268

### N-[4-[3,5-bis(trifluoromethyl)-1H-pyrazol-1-yl]phenyl]-5,6-dichloro-3-pyridinecarboxamide

Example (i)-a B was processed as in Example (i)-a (Method 5, 6, or 7) to provide the title compound.

mp 175-177° C.;

MS (DCI/NH₃) m/e 486 (M+NH₄)⁺;

¹H NMR (DMSO-d₆, 300 MHz) δ9.0 (d, 1H), 8.6 (d, 1H), 8.0 (d, 2H), 7.8 (s, 1H), 7.6 (d, 2H).

### Example 269

### N-[4-[3,5-bis(trifluoromethyl)-1H-pyrazol-1-yl]phenyl]-2,6-dichloro-4-pyridinecarboxamide

Example (i)-a B was processed as in Example (i)-a (Method 5, 6, or 7) to provide the title compound.

mp 215-216° C.;

MS (DCI/NH₃) m/e 486 (M+NH₄)⁺;

¹H NMR (DMSO-d₆, 300 MHz) δ10.92 (s, 1H), 8.04 (s, 2H), 7.97 (d, 2H), 7.84 (s, 1H), 7.67 (d, 2H).

### Example 270

### N-[4-[3,5-bis(trifluoromethyl)-1H-pyrazol-1-yl]phenyl]-2,5-dichloro-3-pyridinecarboxamide

Example (i)-a B was processed as in Example (i)-a (Method 5, 6, or 7) to provide the title compound.

mp 228-229° C.;

MS (DCI/NH₃) m/e 487 (M+NH₄)⁺;

¹H NMR (DMSO-d₆, 300 MHz) δ8.70 (d, 1H), 8.45 (d, 1H), 7.88 (d, 2H), 7.83 (s, 1H), 7.64 (d, 2H).

### Example 271

### N-[4-[3,5-bis(trifluoromethyl)-1H-pyrazol-1-yl]-3-(trifluoromethyl)phenyl]-4-chlorobenzamide

Example (xi)-a B was processed as in Example (i)-a (Method 5, 6, or 7) to provide the title compound.

mp 65-67° C.;

MS (ESI−) m/e 500 (M−H)⁻;

¹H NMR (DMSO-d₆, 300 MHz) δ10.94 (s, 1H), 8.48 (d, 1H), 8.29 (dd, 1H), 8.04 (d, 2H), 7.92 (s, 1H), 7.91 (d, 1H), 7.68 (d, 2H).

### Example 272

### N-[4-[3,5-bis(trifluoromethyl)-1H-pyrazol-1-yl]-2-fluorophenyl]-2,4-difluorobenzamide

Example (xvi)-a B was processed as in Example (i)-a (Method 5, 6, or 7) to provide the title compound.

mp 66-67° C.

MS (ESI−) m/e 452 (M−H)⁻;

¹H NMR (DMSO-d₆, 300 MHz) δ9.96 (s, 1H), 7.88 (dd, 1H), 7.8 (s, 1H), 7.68 (dd, 1H), 7.58 (m, 2H), 7.35 (m, 1H), 7.17 (m, 1H);

Anal. calcd for C₁₈H₈F₉N₃O: C, 47.69; H, 1.77; N, 9.27. Found: C, 47.78; H, 1.87; N, 9.18.

### Example 273

### N-[2,4-bis[3,5-bis(trifluoromethyl)-1H-pyrazol-1-yl]phenyl]-2,4-difluorobenzamide

Example (xvii)-a B was processed as in Example (i)-a (Method 5, 6, or 7) to provide the title compound.

mp 114-115° C.

MS (ESI−) m/e 636 (M−H)⁻;

¹H NMR (DMSO-d₆, 300 MHz) δ12.04 (s, 1H), 8.21 (d, 1H), 8.06 (d, 1H), 7.98 (dd, 1H), 7.88 (s, 1H), 7.82 (s, 1H), 7.64 (dd, 1H), 7.38 (h, 1H), 7.21 (h, 1H);

Anal. calcd for C₂₃H₉F₁₄N₅O: C, 43.34; H, 1.43; N, 10.98. Found: C, 43.7; H, 1.41; N, 10.78.

### Example 274

### methyl 2-[3,5-bis(trifluoromethyl)-1H-pyrazol-1-yl]-5-[(5-bromo-2-chlorobenzoyl)amino]benzoate

Example (x)-a C was processed as in Example (i)-a (Method 5, 6, or 7) to provide the title compound.

mp 75-76° C.

MS (DCI/NH₃) m/e 589 (M+NH₄)⁺;

¹H NMR (DMSO-d₆, 300 MHz) δ11.17 (s, 1H), 8.52 (d, 1H), 8.08 (dd, 1H), 7.98 (d, 1H), 7.82-7.74 (m, 3H), 7.58 (d, 1H), 3.64 (s, 3H).

### Example 275

### N-[4-[3,5-bis(trifluoromethyl)-1H-pyrazol-1-yl]-3-(trifluoromethyl)phenyl]-3,5-dimethyl-4-isoxazolecarboxamide

Example (xi)-a B was processed as in Example (i)-a (Method 5, 6, or 7) to provide the title compound.

mp 61-63° C.;

MS (ESI−) m/e 485 (M−H)⁻;

¹H NMR (DMSO-d₆, 300 MHz) δ10.68 (s, 1H), 8.38 (d, 1H), 8.11 (dd, 1H,), 7.93 (s, 1H), 7.92 (d, 1H), 2.61 (s, 3H), 2.38 (s, 3H);

Anal. calcd for C₁₈H₁₁F₉N₄O₂: C, 44.45; H, 2.28; N, 11.52. Found: C, 44.60; H, 2.37; N, 10.91.

### Example 276

### N-[4-[3,5-bis(trifluoromethyl)-1H-pyrazol-1-yl]-3-(trifluoromethyl)phenyl]-4-methyl-1,2,3-thiadiazole-5-carboxamide

Example (xi)-a B was processed as in Example (i)-a (Method 5, 6, or 7) to provide the title compound.

mp 134-136° C.;

MS (ESI−) m/e 488 (M−H)⁻;

¹H NMR (DMSO-d₆, 300 MHz) δ11.35 (s, 1H), 8.18 (d, 1H), 8.16 (dd, 1H), 7.96 (d, 1H), 7.94 (s, 1H), 2.86 (s, 3H);

Anal. calcd for C₁₆H₈F₉N₅OS: C, 39.27; H, 1.68; N, 14.18. Found: C, 39.29; H, 1.71; N, 13.81.

### Example 277

### N-[4-[3,5-bis(trifluoromethyl)-1H-pyrazol-1-yl]-3-chlorophenyl]-3,5-dimethyl-4-isoxazolecarboxamide

Example (xiii)-a B was processed as in Example (i)-a (Method 5, 6, or 7) to provide the title compound.

mp 64-65° C.;

MS (ESI−) m/e 451 (M−H)⁻;

¹H NMR (DMSO-d₆, 300 MHz) δ10.54 (s, 1H), 8.12 (d, 1H), 7.91 (s, 1H), 7.84 (d, 1H), 7.77 (dd, 1H), 2.48 (s, 3H), 2.36 (s, 3H);

Anal. calcd for C₁₇H₁₁ClF₆N₄O₂: C, 45.09; H, 2.44; N, 12.37. Found: C, 45.26; H, 2.5; N, 11.98.

### Example 278

### N-[4-[3,5-bis(trifluoromethyl)-1H-pyrazol-1-yl]-2-(trifluoromethyl)phenyl]-3,5-dimethyl-4-isoxazolecarboxamide

Example (xii)-a B was processed as in Example (i)-a (Method 5, 6, or 7) to provide the title compound.

mp 145-146° C.;

MS (ESI−) m/e 485 (M−H)⁻;

¹H NMR (DMSO-d₆, 300 MHz) δ8.17 (d, 1H), 8.06 (dd, 1H), 7.92 (d, 2H), 7.9 (s, 1H), 2.62 (s, 3H), 2.37 (s, 3H);

Anal. calcd for C₁₈H₁₁F₉N₄O₂: C, 44.45; H, 2.28; N, 11.52. Found: C, 44.39; H, 2.16; N, 11.3.

### Example 279

### N-[4-[3,5-bis(trifluoromethyl)-1H-pyrazol-1-yl]-2-methylphenyl]-4-methyl-1,2,3-thiadiazole-5-carboxamide

Example (xiv)-a B was processed as in Example (i)-a (Method 5, 6, or 7) to provide the title compound.

mp 196-197° C.;

MS (ESI−) m/e 434 (M−H)⁻;

¹H NMR (CDCl₃, 300 MHz) δ8.04 (s, 1H), 7.73 (d, 1H), 7.54 (d, 2H), 7.47 (dd, 1H), 2.88 (s, 3H), 2.33 (s, 3H);

Anal. calcd for C₁₆H₁₁F₆N₅OS: C, 44.14; H, 2.54; N, 16.08. Found: C, 44.25; H, 2.45; N, 15.97.

### Example 280

### N-[4-[3,5-bis(trifluoromethyl)-1H-pyrazol-1-yl]-2-methoxyphenyl]-4-methyl-1,2,3-thiadiazole-5-carboxamide

Example (xv)-a B was processed as in Example (i)-a (Method 5, 6, or 7) to provide the title compound.

mp 119-121° C.;

MS (ESI−) m/e 450 (M−H)⁻;

¹H NMR (DMSO-d₆, 300 MHz) δ8.14 (d, 1H); 7.94 (s, 1H), 7.75 (d, 1H), 7.23 (dd, 1H), 3.97 (s, 3H), 2.85 (s, 3H);

Anal. calcd for C₁₆H₁₁F₆N5O₂S: C, 42.57; H, 2.45; N, 15.51. Found: C, 43.19; H, 2.46; N, 14.46.

### Example 281

### 4-chloro-N-[4-[5-methyl-3-(trifluoromethyl)-1H-pyrazol-1-yl]phenyl]benzamide

Example (xix)-a B was processed as in Example (i)-a (Method 5, 6, or 7) to provide the title compound.

mp 145-146° C.;

MS (DCI/NH₃) m/e 370 (M+H)⁺;

¹H NMR (CDCl₃, 300 MHz) δ7.85 (d, 2H), 7.78 (d, 2H), 7.49 (d, 2H), 7.47 (d, 2H), 6.67 (s, 1H), 2.36 (s, 3H).

### Example 282

### 4-methyl-N-[4-[5-methyl-3-(trifluoromethyl)-1H-pyrazol-1-yl]phenyl]-1,2,3-thiadiazole-5-carboxamide

Example (xix)-a B was processed as in Example (i)-a (Method 5, 6, or 7) to provide the title compound.

mp 51-53° C.;

MS (DCI/NH₃) m/e 368 (M+H)⁺;

¹H NMR (CDCl₃, 300 MHz) δ7.85 (d, 2H), 7.78 (d, 2H), 7.49 (d, 2H), 7.47 (d, 2H), 6.67 (s, 1H), 2.36 (s, 3H).

### Example 283

### 3,5-Dimethyl-N-[4-[5-methyl-3-(trifluoromethyl)-1H-pyrazol-1-yl]phenyl]-4-isoxazolecarboxamide

Example (xix)-a B was processed as in Example (i)-a (Method 5, 6, or 7) to provide the title compound.

mp 180-181° C.;

MS (DCI/NH₃) m/e 365 (M+H)⁺;

¹H NMR (CDCl₃, 300 MHz) δ7.85 (d, 2H), 7.78 (d, 2H), 7.49 (d, 2H), 7.47 (d, 2H), 6.67 (s, 1H), 2.36 (s, 3H).

### Example 284

### 4-Chloro-N-[4-(5-methyl-1H-pyrazol-1-yl)phenyl]benzamide

Example (xxi)-a B was processed as in Example (i)-a (Method 5, 6, or 7) to provide the title compound.

mp 189-191° C.;

MS (DCI/NH₃) m/e 312 (M+H)⁺;

¹H NMR (DMSO-d₆, 300 MHz) δ10.50 (s, 1H), 8.02 (d, 2H), 7.91 (d, 2H), 7.63 (d, 2H), 7.54 (d, 1H), 7.50 (d, 2H), 6.26 (d, 1H), 2.34 (s, 3H).

### Example 285

### 4-Methyl-N-[4-(5-methyl-1H-pyrazol-1-yl)phenyl]-1,2,3-thiadiazole-5-carboxamide

Example (xxi)-a B was processed as in Example (i)-a (Method 5, 6, or 7) to provide the title compound.

mp 163-164° C.;

MS (DCI/NH₃) m/e 300 (M+H)⁺;

¹H NMR (DMSO-d₆, 300 MHz) δ10.89 (s, 1H), 7.83 (d, 2H), 7.55 (d, 1H), 7.53 (d, 2H), 6.27 (d, 1H), 2.93 (s, 3H), 2.35 (s, 3H).

### Example 286

### 3,5-dimethyl-N-[4-(5-methyl-1H-pyrazol-1-yl)phenyl]-4-isoxazolecarboxamide

Example (xxi)-a B was processed as in Example (i)-a (Method 5, 6, or 7) to provide the title compound.

mp 151-152° C.;

MS (DCI/NH₃) m/e 297 (M+H)⁺;

¹H NMR (DMSO-d₆, 300 MHz) δ10.23 (s, 1H), 7.79 (d, 2H), 7.54 (d, 1H), 7.50 (d, 2H), 6.26 (dd, 1H), 2.57 (s, 3H), 2.36 (s, 3H), 2.32 (s, 3H).

### Example 287

### 3,5-dimethyl-N-[4-[3-(trifluoromethyl)-1H-pyrazol-1-yl)phenyl]-4-isoxazolecarboxamide

Example (xxii)-a B was processed as in Example (i)-a (Method 5, 6, or 7) to provide the title compound.

mp 177-178° C.;

MS (DCI/NH₃) m/e 351 (M+H)⁺;

¹H NMR (DMSO-d₆, 300 MHz) δ10.24 (s, 1H), 8.69 (d, 1H), 7.86 (dd, 4H), 7.04 (d, 1H), 2.57 (s, 3H), 2.36 (s, 3H).

### Example 288

### N-[4-[5-hydroxy-3-(trifluoromethyl)-1H-pyrazol-1-yl]phenyl]-4-methyl-1,2,3-thiadiazole-5-carboxamide

Example (xxiii)-a C was processed as in Example (i)-a (Method 5, 6, or 7) to provide the title compound.

mp 177-179° C.;

MS (ESI−) m/e 368 (M−H)⁻;

¹H NMR (DMSO-d₆, 300 MHz) δ10.87 (s, 1H), 8.82 (d, 2H), 8.67 (d, 2H), 5.82 (s, 1H), 2.84 (s, 3H).

### Example 289

### N-[4-(3,5-dimethyl-1H-1,2,4-triazol-1-yl)phenyl]-4-methyl-1,2,3-thiadiazole-5-carboxamide

Example (xxv)-a B was processed as in Example (i)-a (Method 5, 6, or 7) to provide the title compound.

mp 144-145° C.;

MS (DCI/NH₃) m/e 315 (M+H)⁺;

¹H NMR (CDCl₃, 300 MHz) δ7.74 (d, 2H), 7.28 (d, 2H), 3.01 (s, 3H), 2.51 (s, 3H), 2.42 (s, 3H).

### Example 290

### 3-amino-N-(4-(3,5-bis(trifluoromethyl)-1H-pyrazol-1-yl)phenyl)isonicotinamide

### Example 290A

### 3-nitro-nicotinic acid

3-Nitro-4-methylpyridine (2 g, 14 mmol) in water (200 mL) was refluxed while a saturated solution of potassium permanganate (4.43 g, 28 mmol) in water (20 mL) was added dropwise over a 4 hour period. At the end of addition, the solution was refluxed for another two hours. The solution was filtered while hot, the brown manganese dioxide filter cake was extracted twice with hot water, and the filtrates were combined and concentrated in vacuo. Then the solid was redissolved with a minimum amount of water, and concentrated hydrochloric acid was added to acidify the solution to pH=3. The acidic solution was concentrated in vacuo to give 800 mg of brown product (carboxylic acid salt and KCl salt). The product mixture was carried for next step without further purification. Reference: Kataoka, M.; Morisawa, Y.; Kitano, N. J. Med. Chem. 1976, 30(4), 483-487.

### Example 290B

### 3-nitro-N-(4-(3,5-bis(trifluoromethyl)-1H-pyrazol-1-yl)phenyl)isonicotinamide

Example (i)-a B and Example 290A were processed as in Example (i)-a (Method 5, 6, or 7) to provide the title compound.

mp 207-209° C.;

MS (DCI) m/e (M+H)⁺;

¹H NMR (DMSO-d₆, 300 MHz) δ11.16 (s, 1H), 9.40 (s, 1H), 9.10 (d, 1H, J=6 Hz), 7.94 (d, 1H, J=6 Hz), 7.85 (d, 2H, J=9 Hz), 7.84 (s, 1H), 7.65 (d, 2H, J=9 Hz).

### Example 290

### 3-amino-N-(4-(3,5-bis(trifluoromethyl)-1H-pyrazol-1-yl)phenyl)isonicotinamide

To a slurry 5% Pd-C (11 mg) in ethyl acetate (10 mL) was added Example 290B (110 mg, 0.247 mmol). The resulting mixture was hydrogenated at 4 atm pressure at room temperature for 18 hours. After purging the reaction with nitrogen and filtering the mixture through a plug of diatomaceous earth, the solution was concentrated in vacuo and purified with 10 g of silica-gel using ethyl acetate: hexanes (v/v; 3:7) to afford the title compound as a white powder (80 mg, 77% yield).

mp 101-102° C.;

MS (DCI/NH₃) m/e 416 (M+H)⁺;

¹H NMR (DMSO-d₆, 300 MHz) δ10.55 (s, 1H), 8.87 (s, 1H), 7.94 (d, 2H), 7.86-7.82 (m, 2H), 7.61 (d, 2H), 7.54 (d, 1H), 6.40 (s, 2H).

### Example 291

### N-(4-(3,5-bis(trifluoromethyl)-1H-pyrazol-1-yl)phenyl)-3-chloro-5-methoxyisonicotinamide

Example (i)-a B was processed as described in Example (i)-a (Method 5, 6, or 7) to provide the title compound.

mp 187-188° C.;

MS (DCI/NH₃) m/e 482 (M+NH₄)⁺;

¹H NMR (DMSO-d₆, 300 MHz) δ10.81 (s, 1H), 7.98 (d, 2H), 7.85 (s, 1H), 7.65 (d, 2H), 7.58 (s, 1H), 7.35 (s, 1H), 3.95 (s, 3H).

### Example 292

### N-(6-(3,5-bis(trifluoromethyl)-1H-pyrazol-1-yl)-3-pyridinyl)-2-fluorobenzamide

### Example 292A

### 5-[3,5-bis(trifluoromethyl)-1H-pyrazol-1-yl]-2-nitropyridine

To a cold slurry (0° C.) of sodium hydride (95%, 160 mg, 6.67 mmol) in dimethylformamide (10 mL) was added 3,5-bis(trifluoromethyl)pyrazole (1.12 g, 5.50 mmol). The resulting suspension was stirred for 30 minutes. A solution of 2-chloro-4-nitropyridine (867 mg, 5.5 mmol) was added. The resulting mixture was heated at reflux for 12 hours, then cooled to room temperature. The mixture was poured into saturated sodium chloride solution (100 mL). The aqueous mixture was extracted with ethyl acetate (3×100 mL) and the combined organic layers were dried over sodium sulfate, filtered and concentrated. The residue was purified by flash column chromatography using 20% ethyl acetate/hexane to afford a yellow oil (1.78 g, 99% yield).

MS (DCI/NH₃) m/e 326 (M+H)⁺;

¹H NMR (DMSO-d₆, 300 MHz) δ9.39 (d, 1H), 8.86 (dd, 1H), 8.21 (d, 1H), 8.02 (s, 1H).

### Example 292B

### 5-[3,5-bis(trifluoromethyl)-1H-pyrazol-1-yl]-2-pyridinylamine

To a a slurry of 10% palladium on carbon (192 mg) in ethyl acetate (90 mL) under a nitrogen atmosphere was added a solution of Example 292A (1.77 g, 5.43 mmol) in ethyl acetate (10 mL). A hydrogen balloon was placed on the reaction flask and the reaction mixture was maintained under a hydrogen atmosphere for 20 hours. The reaction flask was purged with nitrogen and then the catalyst was filtered off through a diatomaceous earth/silica gel plug to afford an oil (1.20 g, 75% yield).

MS (DCI/NH₃) m/e 297 (M+H)⁺;

¹H NMR (DMSO-d₆, 300 MHz) δ7.82 (d, 1H), 7.72 (s, 1H), 7.43 (d, 1H), 7.15 (dd, 1H), 5.90 (s, 2H).

### Example 292

### N-(6-(3,5-bis(trifluoromethyl)-1H-pyrazol-1-yl)-3-pyridinyl)-2-fluorobenzamide

Example 292B was processed as described in Method 5, 6, or 7 to provide the title compound.

mp 164-165° C.;

MS (DCI/NH₃) m/e 419 (M+H)⁺;

¹H NMR (DMSO-d₆, 300 MHz) δ10.96 (s, 1H), 8.88 (d, 1H), 8.45 (dd, 1H), 7.92 (d, 1H), 7.87 (s, 1H), 7.77 (m, 1H), 7.66 (m, 1H), 7.40 (m, 2H).

### Example 293

### methyl 2-(3,5-bis(trifluoromethyl)-1H-pyrazol-1-yl)-5-((2-fluorobenzoyl)amino)benzoate

### Example 293A

### methyl 2-[3,5-bis(trifluoromethyl-1H-pyrazol-1-yl]-5-nitrobenzoate

3,5-Bis(trifluoromethyl)pyrazole (1.02 g, 5 mmol) in DMF (5 mL) was added to a mixture of NaH (23 mg, 5 mmol, 95%) in DMF (20 mL). The mixture turned brown in 5 minutes and was stirred at room temperature for one hour. Then methyl 2-fluoro-5-nitrobenzoate (1.0 g, 5.0 mmol) in DMF (10 mL) was added to the solution drop wise via syringe. Upon finishing the addition, the solution was heated to 45° C. for 10 hours. Then it was cooled to room temperature, diluted with water (20 mL) and extracted with ethyl acetate (3×20 mL). The combined organic portions were washed with 1N HCl (2×20 mL), dried over Na₂SO₄, filtered and concentrated in vacuo. This crude product was chromatographed over silica gel, using ethyl acetate:hexanes (2:8 to 3:7) in sequence. The fractions were collected and concentrated in vacuo to give the title compound (1.5 g, 79% yield) as a brown oil.

Deutsch, J.; Niclas, H. J. Synth. Commun.1991, 21(4), 505-513.

MS (DCI/NH₃) m/e 371 (M+NH₄)⁺;

¹H NMR (DMSO-d₆, 300 MHz) δ8.76-8.64 (m, 2H) 8.17 (d, 1H, J=6 Hz), 7.94 (s, 1H).

### Example 293B

### methyl 5-amino-2-[3,5-bis(trifluoromethyl)-1H-pyrazol-1-yl]benzoate

The nitro group of Example 293A was reduced with iron powder and ammonium chloride as described in Example 355B.

mp 45-47° C.;

MS (DCI/NH₃) m/e 371 (M+NH₄)⁺;

¹H NMR (DMSO-d₆, 300 MHz) δ7.6 (s, H), 7.20 (d, 1H, J=6 Hz), 6.76 (dd, 1H, J=9.3 Hz), 5.92 (s, 2H), 3.46 (s, 3H).

### Example 293

### methyl 2-(3,5-bis(trifluoromethyl)-1H-pyrazol-1-yl)-5-((2-fluorobenzoyl)amino)benzoate

Example 293 B was processed as in Method 5 or 6, or 7 to provide the title compound.

MS (DCI/NH₃) m/e 493 (M+NH₄)⁺;

¹H NMR (DMSO-d₆, 300 MHz) δ8.54 (d, 1H), 8.16 (dd, 1H), 7.80 (d, 1H), 7.76 (td, 1H), 7.69-7.60 (m, 1H), 7.45-7.35 (m, 2H), 3.65 (s, 3H).

### Example 294

### 4-(aminomethyl)-N-(4-(3,5-bis(trifluoromethyl)-1H-pyrazol-1-yl)phenyl)-2-chlorobenzamide

### Example 294A

### 4-cyano-2-chlorobenzoic acid

Under a nitrogen atmosphere, Zn(CN)₂ (58 mg, 0.50 mmol) and 4-bromo-2-chlorobenzoic acid (200 mg, 0.90 mmol) were added to dry dimethylformamide (5 mL), followed by tetrakis(triphenylphosphine)palladium(0) (43 mg, 0.036 mmol). The resulting yellow slurry was heated to 80° C. overnight. After it was cooled to room temperature, it was diluted with ethyl ether (20 mL), and washed with water (2×10 mL). Then the ethereal portion was collected, dried with Na₂SO₄, filtered and concentrated in vacuo to give 2-chloro-4-cyanobenzoic acid (60 mg, 37% yield) as a white solid.

Reference: Magidson, O. J.; Trawin, A. I. Chem Ber. 1936, 69, 537-544.

### Example 294B

### N-{4-[3,5-bis(trifluoromethyl)-1H-pyrazol-1-yl]phenyl}-2-chloro-4-cyanobenzamide

Example (i)-a B and Example 294A were processed as in Example (i)-a (Method 5, 6, or 7) to provide the title compound.

MS (DCI/NH₃) m/e 476 (M+NH₄)⁺

¹H NMR (DMSO-d₆, 300 MHz) δ8.03 (s, 1H), 8.0 (d, 1H), 7.9 (m, 3H), 7.8 (s, 1H), 7.6 (d, 2H).

### Example 294

### 4-(aminomethyl)-N-(4-(3,5-bis(trifluoromethyl)-1H-pyrazol-1-yl)phenyl)-2-chlorobenzamide

To a solution of Example 294B (40 mg, 0.087 mmol) in methanol (10 mL) was added colboltous chloride hexahydrate (23 mg, 0.17 mmol) and sodium borohydride (34 mg, 0.9 mmol) in portions at 0° C. with stirring. After 4 hours at 0° C., the black slurry was acidified with 1N hydrochloric acid solution until the solid was totally dissolved. After removal of methanol in vacuo, the aqueous layer was made alkaline with NaOH solution and extracted with ethyl acetate (3×20 mL). The combine organic layers were washed with saturated sodium chloride solution (2×20 mL) and dried over Na₂SO₄, filtered and concentrated in vacuo to give the title compound (30 mg,75% yield) as a pale yellow powder. Reference: Suzuki, Y.; Miyaji, Y.; Imai, Z. Tetrahedron Lett, 1969, 4555-4558.

mp 90-93° C.;

MS (DCI/NH₃) m/e 480 (M+NH₄)⁺;

¹H NMR (DMSO-d₆, 300 MHz) δ7.94 (d, 2H), 7.84 (s, 1H), 7.66-7.55 (m, 4H), 7.42 (d, 1H), 4.39 (s, 2H), 3.90 (s, 2H).

### Example 295

### N-(4-(3,5-bis(trifluoromethyl)-1H-pyrazol-1-yl)phenyl)-2-methylacrylamide

Example (i)-a B and was processed as in Example (i)-a (Method 5, 6, or 7) to provide the title compound.

mp 160-162° C.;

MS (DCI/NH₃) m/e 364 (M+H)⁺;

¹H NMR (DMSO-d₆, 300 MHz) δ10.1 (s, 1H), 7.9 (d, 2H), 7.8 (s, 1H), 7.6 (d, 2H), 5.8 (d, 1H), 5.6 (d, 1H), 2.5 (s, 3H).

### Example 296

### N-(4-(3,5-bis(trifluoromethyl)-1H-pyrazol-1-yl)phenyl)-4-chloro-2-fluorobenzamide

Example (i)-a B was processed as in Example (i)-a (Method 5, 6, or 7) to provide the title compound.

mp 156-158° C.;

MS (ESI) m/e 486 (M+Cl)⁻; 450 (M−1)⁻;

¹HNMR (300 MHz, CDCl₃) δ8.53 (d, 1H, J=16.2 Hz), 8.16 (dd, 1H, J=8.7, 8.4 Hz), 7.84 (m, 2H), 7.52 (m, 2H), 7.35 (dd, 1H, J=8.4, 1.8 Hz), 7.27 (dd, 1H, J=12.0, 1.8 Hz), 7.08 (s, 1H).

Anal. Calcd for C₁₈H₉ClF₇N₃O: C, 47.86; H, 2.01; N, 9.30. Found: C, 48.14; H, 2.05; N, 9.11.

### Example 297

### N-(5-(3,5-bis(trifluoromethyl)-1H-pyrazol-1-yl)-2-pyridinyl)-2-fluorobenzamide

### Example 297A

### 5-chloro-2-nitropyridine

To cold (0° C.) concentrated sulfuric acid (80 mL) was added 30% hydrogen peroxide (40 mL). To this solution was added 2-amino-5-chloropyridine (4.00 g, 31.11 mmol). The solution became lime colored within 30 minutes. The reaction mixture was allowed to warm to room temperature and stirred for 20 hours. The reaction mixture was then poured into ice water and a white precipitate formed. This solid was filtered and dried in vacuo to afford 5-chloro-2-nitropyridine (3.10 g, 63% yield).

MS (DCI/NH₃) m/e 129 (M+H for aniline)⁺;

¹H NMR (DMSO-d₆, 300 MHz) δ8.78 (m, 1H), 8.37 (m, 2H).

### Example 297B

### 5-[3,5-bis(trifluoromethyl)-1H-pyrazol-1-yl]-2-nitropyridine

To a cold slurry (0° C.) of sodium hydride (95%, 439 mg, 18.30 mmol) in dimethylformamide 3.0 mL) was added 3,5-bis(trifluoromethyl)pyrazole (2.50 g, 12.30 mmol). The resulting suspension was stirred for 30 minutes. A solution of 5-chloro-2-nitropyridine (1.90 g, 12.00 mmol) was added. The resulting mixture was heated at reflux for 24 hours, then cooled to room temperature. The mixture was poured into saturated sodium chloride solution (100 mL). The aqueous mixture was extracted with ethyl acetate (3×100 mL) and the combined organic layers were dried over sodium sulfate, filtered and concentrated. The residue was purified by flash column chromatography using 20% ethyl acetate/hexane to afford a yellow oil (1.17 g, 30% yield).

MS (DCI/NH₃) nme 297 (M+1 for aniline)⁺;

¹H NMR (DMSO-d₆, 300 MHz) δ9.38 (d, 1H), 8.88 (dd, 1H), 8.21 (d, 1H), 8.02 (s, 1H).

### Example 297C

### 5-[3,5-bis(trifluoromethyl)-1H-pyrazol-1-yl]-2-pyridinamine

To a solution of Example 297B (183 mg, 0.54 mmol) in acetic acid (3.0 mL) was added zinc powder (71 mg, 1.10 mmol). The resulting mixture was heated at 70° C. for one hour. The reaction mixture was cooled and poured into saturated sodium bicarbonate solution (100 mL). The aqueous layer was extracted with ethyl acetate (3×100 mL). The combined organic layers were dried over sodium sulfate filtered and concentrated to a crude oil which was used in the next step without further purification.

MS (DCI/NH₃) m/e 297 (M+H)⁺;

¹H NMR (DMSO-d₆, 300 MHz) δ7.81 (d, 1H), 7.74 (s, 1H), 7.44 (d, 1H), 7.15 (dd, 1H), 5.93 (s, 2H).

### Example 297

### N-(5-(3,5-bis(trifluoromethyl)-1H-pyrazol-1-yl)-2-pyridinyl)-2-fluorobenzamide

Example 297C was processed as in Example (i)-a (Method 5, 6, or 7) to provide the title compound.

mp 153-155° C.;

MS (DCI/NH₃) m/e 419 (M+H)⁺;

¹H NMR (DMSO-d₆, 300 MHz) δ10.95 (s, 1H), 8.88 (d, 1H), 8.47 (dd, 1H), 7.92 (d, 1H), 7.87(s, 1H), 7.77 (m, 1H), 7.66 (m, 1H), 7.40 (m, 2H).

### Example 298

### N-{3-amino-4-[3,5-bis(trifluoromethyl)-1H-pyrazol-1-yl]phenyl}-2-fluorobenzamide

### Example 298A

### 2-[3,5-bis(trifluoromethyl)-1H-pyrazol-1-yl]-5-nitrobenzoic acid

To a cold (0° C.) slurry of potassium hydride (35%, 1.09 g, 9.55 mmol) in tetrahydrofuran (20.0 mL) was added 3,5-bis(trifluoromethyl)pyrazole (1.56 g, 7.64 mmol) in portions over 15 min. The resulting mixture was stirred at 0° C. for 30 min., then solid 2-fluoro-4-nitrobenzoic acid (708 mg, 3.82 mmol) was added. The mixture was heated at reflux for 20 hours, cooled, then poured into 1N HCl solution (100 mL). The aqueous layer was extracted with ethyl acetate (3×100 mL). The combined organic layers were dried over sodium sulfate, filtered and concentrated. The crude oil was used in the next step without purification (450 mg, 34%).

MS (DCI/NH₃) m/e 357 (M+NH₄)⁺(for corresponding aniline);

¹H NMR (DMSO-d₆, 300 MHz) δ8.72 (d, 1H), 8.60 (dd, 1H), 8.09 (dd, 1H), 7.88 (s, 1H).

### Example 298B

### 2-(trimethylsilyl)ethyl 2-[3,5-bis(trifluoromethyl)-1H-pyrazol-1-yl]-5-nitrophenylcarbamate

A mixture of Example 298A (421 mg, 1.23 mmol), triethylamine (1.0 mL, 6.15 mmol), diphenylphosphorylazide (0.40 mL, 1.85 mmol) and P-trimethylsilylethanol (0.88 mL, 6.15 mmol) in toluene was heated at 70° C. for 20 hours. The reaction mixture was cooled and concentrated in vacuo. Purification of the crude residue with flash chromatography eluting with 10% ethyl acetate/hexane affored the title compound (230 mg, 39% yield) as a yellow oil.

MS (DCI/NH₃) m/e 502 (M+NH₄)⁺;

¹H NMR (DMSO-d₆, 300 MHz) δ9.82 (s, 1H), 8.81 (d, 1H), 8.03 (dd, 1H), 7.85 (s, 1H), 7.75 (d, 1H), 4.09 (t, 2H), 0.95 (t, 2H), 0.02 (s, 9H).

### Example 298C

### 2-(trimethylsilyl ethyl 5-amino-2-[3,5-bis(trifluoromethyl)-1H-pyrazol-1-yl]phenylcarbamate

Example 298B was reduced using general hydrogenation method described in method 4.

¹H NMR (DMSO-d₆, 300 MHz) δ8.71 (s, 1H), 7.60 (s, 1H), 6.99 (dd, 1H), 6.38 (dd, 1H), 5.68 (s, 2H), 4.03 (t, 2H), 0.91 (t, 2H), 0.02 (s, 9H).

### Example 298D

### 2-(trimethylsilyl)ethyl 2-[3,5-bis(trifluoromethyl)-1H-pyrazol-1-yl]-5-[(2-fluorobenzoyl)amino]phenylcarbamate

Example 298C was processed as in Example (i)-a (Method 5, 6, or 7) to provide the title compound.

MS (DCI/NH₃) m/e 594 (M+NH₄)⁺;

¹H NMR (DMSO-d₆, 300 MHz) δ10.78 (s, 1H), 9.24 (s, 1H), 8.25 (d, 1H), 7.74 (s, 1H), 7.65 (m, 3H), 7.38 (m, 3H), 4.05 (t, 2H), 0.95 (t, 2H), 0.02 (s, 9H).

### Example 298

### N-{3-amino-4-[3,5-bis(trifluoromethyl)-1H-pyrazol-1-yl]phenyl}-2-fluorobenzamide

A mixture of Example 298D (49 mg, 0.085 mmol) and tetrabutylammonium fluoride (01.5 mL, 015 mmol) in tetrahydrofuran (1.0 mL) and DMSO (1.0 mL) was heated at 80° C. for 48 hours. The reaction mixture was cooled and purified directly by flash chromatography using 40% ethyl acetate/hexane affording the title compound as an oil (37 mg, 99% yield).

MS (DCI/NH₃) nme 450 (M+NH₄)⁺;

¹H NMR (DMSO-d₆, 300 MHz) δ7.71 (s, 1H), 7.70-7.75 (m, 3H), 7.40-7.30 (m, 2H), 7.15 (d, 1H), 6.85 (dd, 1H), 5.38 (s, 2H).

### Example 299

### N-(4-(3,5-bis(trifluoromethyl)-1H-pyrazol-1-yl)-3-cyanophenyl)-2-fluorobenzamide

### Example 299A

### 5-amino-2-[3,5-bis(trifluoromethyl)-1H-pyrazol-1-yl]benzonitrile

Sodium hydride (95%, 130 mg, 5.39 mmol) was combined with dimethylformamide (20 mL) under a nitrogen atmosphere. To this slurry was added a solution of 3,5-bis(trifluoromethyl)pyrazole (1 g, 4.9 mmol) in dimethylformamide (5 mL). The mixture turned brown in 5 minutes and was stirred at room temperature for one hour. Then 2-fluoro-5-nitro-benzonitrile (814 mg, 4.9 mmol) in dimethylformamide (10 mL) was added to the solution drop wise by syringe. Upon finishing addition, the solution was heated to 45° C. for 10 hours. Then it was cooled to room temperature, diluted with H₂O (20 mL) and extracted with ethyl acetate (3×20 mL). The combined organic portions were washed with 1N HCl (2×20 mL), dried over Na₂SO₄, filtered and concentrated in vacuo. This crude product, obtained as a brown oil (1.4 g, 84% yield), was used without additional purification.

### Example 299B

### 2-[3,5-bis(trifluoromethyl)-1H-pyrazol-1-yl]-5-nitrobenzonitrile

The nitro group of Example 299A was reduced with iron powder and ammonium chloride as described in Example 355B.

mp 124-126° C.;

MS (DCI/NH₃) m/e 338 (M+NH₄)⁺;

¹H NMR (DMSO-d₆, 300 MHz) δ9.31 (s, 1H), 7.71 (d, 1H), 7.05 (d, 1H), 6.94 (dd, 1H).

### Example 299

### N-(4-(3,5-bis(trifluoromethyl)-1H-pyrazol-1-yl)-3-cyanophenyl)-2-fluorobenzamide

Example 299B was processed as in Example (i)-a (Method 5, 6, or 7) to provide the title compound.

mp 132-134° C.;

MS (DCI/NH₃) m/e 460 (M+NH₄)⁺;

¹H NMR (DMSO-d₆, 300 MHz) δ11.15 (s, 1H), 8.58 (d, 1H), 8.47 (d, 1H), 8.20 (dd, 1H), 8.04 (s, 1H), 7.47-8.28 (m, 4H).

### Example 300

### N-{4-[5-cyano-3-(trifluoromethyl)-1H-pyrazol-1-yl]phenyl}-2-fluorobenzamide

### Example 300A

### 2,2,2-trifluoroacetaldehyde N-(4-nitrophenyl)hydrazone

A 1 L round bottom flask equipped with a stir bar and a 250 mL pressure equalizing dropping funnel was charged with trifluoroacetic acid (10.0 mL, 130 mmol) and ether (350 mL). To this cold solution (0° C.) solution was added lithium aluminum hydride (1 M soln. in ether, 100 mL, 100 mmol) via the dropping funnel over 20 min. The resulting solution was stirred at 0° C. for 1 h. The reaction was quenched by the addition of methanol (10 mL), followed by water (10 mL), then concentrated HCl (17 mL). The ether layer was extracted with water (300 mL), then dried over sodium sulfate, filtered and concentrated. The crude material was used in the next step without further purification. A mixture of the trifluoroacetaldehyde thus produced (ca. 130 mmol), 4-nitrophenylhydrazine (15.02 g, 98.04 mmol), ethanol (250 mL) and concentrated HCl (5.0 mL) were heated to 100° C. for 2 hours. The reaction was cooled, approximately 90% of the ethanol was removed in vacuo, and then ether (350 mL) was added. The ether layer was washed with saturated sodium bicarbonate solution (300 mL), then dried over sodium sulfate, filtered and concentrated to a crude orange solid (22.8 g, 99%) which was pure enough to use in the next step.

MS (DCI/NH₃) m/e 251 (M+NH₄)⁺;

¹H NMR (DMSO-d₆, 300 MHz) δ11.78 (s, 1H), 8.20 (d, 2H), 7.55 (q, 1H), 7.19 (d, 2H).

### Example 300B

### 2,2,2-trifluoro-N-(4-nitrophenyl)ethanehydrazonoyl chloride

To a solution of Example 300A (7.4 g, 0.031 mol) in DMF (30 mL) was added a solution of N-chlorosuccininmide (4.38 g, 0.033 mol, 1.05 eq) in DMF (15 mL) dropwise at 0° C. After addition, the resulting dark green mixture was stirred at room temperature for two hours. The reaction mixture was then poured into an ice water bath with stirring. A light brown solid formed after about 30 minutes at which point the solid was filtered, and dried in a vacuum oven at 40° C. for 12 hours to give 11 g of an orange solid which was pure enough to use in the next step.

MS (DCI/NH₃) m/e 285 (M+NH₄)⁺;

¹H NMR (DMSO-d₆, 300 MHz) δ11.30 (s, 1H), 8.24 (d, 2H), 7.44 (d, 2H).

### Example 300C

### 1-(4-nitrophenyl)-3-(trifluoromethyl)-1H-pyrazole-5-carbonitrile

To a 250 mL round bottom flask charged with Example 300B (8.81 g, 33.75 mmol) at room temperature was added toluene (68 mL) followed by 2-chloroacrylonitrile (5.4 mL, 67.5 mmol), then triethylamine (10.35 mL, 74.25 mmol). The resulting dark reaction mixture was heated to 80° C. for 1 hour. The reaction mixture was cooled and diluted with ethyl acetate (200 mL). The organic layer was washed with 1 N hydrochloric acid solution (150 mL), dried over sodium sulfate, filtered and concentrated. The crude residue was purified by flash chromatography using 10% ethyl acetate/90% hexane affording the title compound as a yellow oil (4.94 g, 58% yield).

MS (DCI/NH₃) m/e 270 (M+NH₄)⁺ (For the corresponding aniline produced in the analysis.)

¹H NMR (DMSO-d₆, 300 MHz) δ8.53 (d, 2H), 8.20 (s, 1H), 8.13 (d, 2H).

### Example 300D

### 1-(4-aminophenyl)-3-(trifluoromethyl)-1H-pyrazole-5-carbonitrile

To a 50 mL round bottom flask was Example 300C (1 g, 3.5 mmol), ammonium chloride (138 mg, 2.8 mmol, 0.8 eq), iron powder (1.59 g, 28 mmol, 8 eq) and a mixture of ethanol: H₂O (3:1, 32 mL). The mixture was heated to reflux for 2 hours. After it was cooled to room temperature, the mixture was passed through a diatomaceous earth pad and the filtrate was concentrated in vacuo. The resulting solid was redissolved in dichloromethane (30 mL) and washed with NaHCO₃ solution (30 mL). The dichloromethane portion was dried with Na₂SO₄ and concentrated in vacuo to give the amine (800 mg, 91%) as a crude brown solid which was pure enough to use in the next step.

MS (DCI/NH₃) m/e 252 (M+NH₄)⁺;

¹H NMR (DMSO-d₆, 300 MHz) δ7.96 (s, 1H), 7.38 (d, 2H), 6.70 (d, 2H), 5.71 (s, 2H).

### Example 300

### N-{4-[5-cyano-3-(trifluoromethyl)-1H-pyrazol-1-yl]phenyl}-3-fluoroisonicotinamide

Example 300D was processed as in Example (i)-a (Method 5, 6, or 7) to provide the title compound.

mp 161-162° C.;

MS (DCI/NH₃) m/e 393 (M+NH₄)⁺;

¹H NMR (DMSO-d₆, 300 MHz) δ11.05 (s, 1H), 8.79 (s, 1H), 8.62 (d, 1H), 8.07 (s, 1H), 7.96 (d, 2H), 7.81 (d, 2H), 7.75 (t, 1H);

³C NMR(DMSO-d₆, 75 MHz) δ161.3, 155.0 (d, J=259 Hz), 146.4, 142.0 (q, J=39 Hz), 139.9, 139.0 (d, J=23 Hz), 133.1, 131.2 (d, J=14 Hz), 124.9, 123.2, 120.5 (q, J=262 Hz), 120.5, 116.7, 114.7, 109.9.

Anal. calcd for C₁₇H₉F₄N₅O: C, 54.40; H, 2.41; N, 18.66. Found: C, 54.47; H, 2.52; N, 18.49.

### Example 301

### 2-fluoro-N-(4-(5-(2-furyl)-3-(trifluoromethyl)-1H-pyrazol-1-yl)phenyl)benzamide

### Example 301A

### 5-(2-furyl)-1-(4-nitrophenyl)-3-(trifluoromethyl)-1H-pyrazole

4-Nitrophenylhydrazine (7.75 g, 50.5 mmol) in a mixture of absolute ethanol (75 mL) and concentrated HCl (40 mL) was treated with 4,4,4-trifluoro-1-(2-furyl)-1,3-butanedione (12.5 g, 60.6 mmol) and heated to reflux for 2 hours. The reaction mixture was cooled to room temperature and diluted with hexanes/ethyl acetate (600 mL of a 1:1 mixture). The layers were separated, and the organic layer was washed with 1.0 N HCl (3×100 mL) and then saturated brine solution. The resultant mixture was dried over Na₂SO₄, and concentrated in vacuo. Purification using silica gel chromatography (97:3 hexanes/ethyl acetate gradient to 95:5 hexanes/ethyl acetate) yielded a white amorphous solid (14.7 g, 90% yield).

¹HNMR (300 MHz, CDCl₃) δ8.33 (dt, 2H, J=9.3, 2.7 Hz), 7.62 (dt, 2H, J=9.0, 2.7 Hz), 7.45 (dd, 1H, J=1.5, 0.6 Hz), 6.92 (s, 1H), 6.47 (dd, 1H, J=3.6, 1.5 Hz), 6.39 (dd, 1H, J=3.3, 0.6 Hz).

### Example 301B

### 4-[5-(2-furyl)-3-(trifluoromethyl)-1H-pyrazol-1-yl]aniline

A solution of Example 301A (1.2 g, 3.7 mmol) in isopropanol (80 mL) was treated with 10% Pd/C (400 mg) and placed under a hydrogen atmosphere (balloon). After 1.75 hours the reaction was complete and the mixture was filtered through a plug of diatomaceous earth. Concentration in vacuo was followed by purification using silica gel chromatography (6:1 hexanes/ethyl acetate) yielding a white amorphous solid (1.0 g, 92% yield).

MS (ESI+) m/e 294 (M+1)⁺;

¹HNMR (300 MHz, CD₃OD) δ7.55 (dd, 1H, J=1.8, 0.9 Hz), 7.13-7.08 (m, 2H), 6.95 (s, 1H), 6.79 (m, 2H), 6.39 (dd, 1H, J=3.3, 1.8 Hz), 5.93 (d, 1H, J=3.6 Hz).

Example 3012-fluoro-N-(4-(5-(2-furyl)-3-(trifluoromethyl)-1H-pyrazol-1-yl)phenyl benzamide

Example 301B was processed as in Example (i)-a (Method 5, 6, or 7) to provide the title compound.

mp 171-172° C.;

MS (DCI/NH₃) m/e 433 (M+NH₄)⁺; 416 (M+H)⁺;

¹H NMR (DMSO-d₆, 300 MHz) δ7.91 (m, 2H), 7.76 (dt, 1H), 7.62-7.54 (m, 2H), 7.45 (ddd, 2H), 7.33 (dt, 1H), 7.27 (ddd, 1H), 7.02 (s, 1H), 6.45(dd, 1H), 6.15(dd, 1H);

¹³C NMR (DMSO-d₆, 75 MHz) δ145.1, 144.2, 141.2, 138.1, 136.6, 134.4, 134.3, 131.32, 131.29, 127.9, 125.8, 125.7, 122.0, 117.5, 117.2, 112.6, 111.6, 104.5.

### Example 302

### N-(4-(5-cyano-3-(trifluoromethyl)-1H-pyrazol-1-yl)phenyl)-4-methyl-1,2,3-thiadiazole-5-carboxamide

Example 300D was processed as in Example (i)-a (Method 5, 6, or 7) to provide the title compound.

mp 193-195° C.;

MS (DCI/NH₃) m/e 396 (M+NH₄)⁺;

¹H NMR (DMSO-d₆, 300 MHz) δ11.05 (s, 1H), 8.07 (s, 1H), 7.94 (d, 2H), 7.82 (d, 2H), 2.84 (s, 3H).

### Example 303

### N-(4-(5-cyano-3-(trifluoromethyl)-1H-pyrazol-1-yl)phenyl)isonicotinamide

Example 300D was processed as in Example (i)-a (Method 5, 6, or 7) to provide the title compound.

mp 145-146° C.;

MS (DCI/NH₃) m/e 358 (M+1)⁺;

¹H NMR (DMSO-d₆, 300 MHz) δ10.85 (s, 1H), 8.83 (d, 2H), 8.09 (s, 1H), 8.04 (d, 2H), 7.90 (d, 2H), 7.82 (d, 2H).

### Example 304

### N-(4-(5-cyano-3-(trifluoromethyl)-1H-pyrazol-1-yl)phenyl)-3-fluoroisonicotinamide

Example 300D was processed as in Example (i)-a (Method 5, 6, or 7) to provide the title compound.

mp 161-162° C.;

MS (DCI/NH₃) m/e 393 (M+NH₄)⁺;

¹H NMR (DMSO-d₆, 300 MHz) δ11.05 (s, 1H), 8.79 (s, 1H), 8.62 (d, 1H), 8.07 (s, 1H), 7.96 (d, 2H), 7.81 (d, 2H), 7.75 (t, 1H);

¹³C NMR(DMSO-d₆, 75 MHz) δ161.3, 155.0 (d, J=259 Hz), 146.4, 142.0 (q, J=39 Hz), 139.9, 139.0 (d, J=23 Hz), 133.1, 131.2 (d, J=14 Hz), 124.9, 123.2, 120.5 (q, J=262 Hz), 120.5, 116.7, 114.7, 109.9.

Anal. calcd for C₁₇H₉F₄N₅O: C, 54.40; H, 2.41; N, 18.66. Found: C, 54.47; H, 2.52; N, 18.49.

### Example 305

### N-(4-(5-acetyl-3-(trifluoromethyl)-1H-pyrazol-1-yl)phenyl)-2-fluorobenzamide

### Example 305A

### 1-(4-nitrophenyl)-3-(trifluoromethyl)-1H-pyrazole-5-carboxylic acid

Example 301A (3.7 g, 11.6 mmol) in a mixture of tert-butanol (65 mL) and 0.5 N NaOH (35 mL) was treated with KMnO₄ (4.5 g, 28.5 mmol) and heated at 75° C. for 1 hour. The mixture was cooled to ambient temperature, and the second portion of KMnO₄ (4.5 g, 28.5 mmol) was added. After stirring for an additional 1 hour at 75° C., the reaction mixture was cooled to ambient temperature and filtered through a thick plug of diatomaceous earth. The diatomaceous earth was washed with water (3×100 mL). The combined washes were concentrated to 50% of the original volume and acidified to pH=3 with 50% HCl solution. Next, the mixture was extracted with ethyl acetate (3×100 mL) and the combined extracts were dried over Na₂SO₄, and concentrated in vacuo. Purification using silica gel chromatography (75:20:5 hexanes/ethyl acetate/acetic acid gradient to 55:35:10 hexanes/ethyl acetate/acetic acid) yielded a white amorphous solid (1.8 g, 51% yield) along with 1.3 g of the corresponding ketoacid intermediate. The ketoacid intermediate was resubmitted to the conditions above to produce additional carboxylic acid (300 mg, yield after resubmission 60%, unoptimized).

MS (ESI−) m/e 300 (M−1)⁻;

¹HNMR (300 MHz, DMSO-d₆) δ8.32 (d, 2H, J=8.8 Hz), 7.84 (d, 2H, J=8.8 Hz), 7.16 (s, 1H).

### Example 305B

### N-methoxy-N-methyl-1-(4-nitrophenyl)-3-(trifluoromethyl)-1H-pyrazole-5-carboxamide

Example 305A (1.8 g, 5.9 mmol) in CH₂Cl₂ (25 mL, 0.2 M) was treated with N,O-dimethylhydroxylamine hydrochloride (674 mg, 6.9 mmol), EDC (1.1 g, 5.9 mmol), 4-methylmorpholine (1.6 mL, 14.6 mmol), and 1-hydroxybenzotriazole hydrate (742 mg, 5.5 mmol). The mixture was stirred for 14 hours, then washed with 10% aqueous NaHSO₄. The organic layer was dried over NASO₄, and concentrated in vacuo. Purification using silica gel chromatography (2:1 hexanes/ethyl acetate) yielded a white foam (1.8 g, 83% yield). MS (ESI+) m/e 345 (M+1)⁺;

¹HNMR (300 MHz, CDCl₃) δ8.35 (ddd, 2H, J=8.7,3.0,1.8 Hz), 7.68 (ddd, 2H, J=9.3, 2.7, 2.1 Hz), 7.08 (s, 1H), 3.64 (s, 3H), 3.31 (s, 3H).

### Example 305C

### 1-(4-aminophenyl)-N-methoxy-N-methyl-3-(trifluoromethyl)-1H-pyrazole-5-carboxamide

Example 305B (1.2 g, 3.5 mmol) in an ethanol/water mixture (36 mL, 2:1 ratio respectively) was treated with iron powder (1.2 g) and ammonium chloride (120 mg). The mixture was heated at 80° C. for 35 minutes. The mixture was diluted with ethyl acetate (20 mL) and filtered through a thin plug of diatomaceous earth and concentrated in vacuo. Purification using silica gel chromatography (1:1 hexanes/ethyl acetate gradient to 1:2 hexanes/ethyl acetate) yielded a white foam (1.0 g, 94% yield).

MS (ESI+) m/e 315 (M+1)⁺;

¹HNMR (300 MHz, CD₃OD) δ7.16 (ddd, 2H, J=8.7, 3.0, 2.1 Hz), 7.02 (s, 1H), 6.74 (ddd, 2H, J=9.0, 3.0, 2.4 Hz), 3.58 (s, 3H), 3.20 (s, 3H).

### Example 305D

### 1-[1-(4-aminophenyl)-3-(trifluoromethyl)-1H-pyrazol-5-yl]-1-ethanone

Example 305C (56 mg, 0.18 mmol) in THF (2 mL) was slowly added to methyllithium (330 μL of a 1.4 M solution in diethyl ether, 0.46 mmol) at 0° C. The reaction was stirred at 0° C. for five minutes then 10% aqueous NaHSO₄ was added. The aqueous layer was back extracted with ethyl acetate (3×5 mL) and the combined extracts were concentrated in vacuo. The mixture was purified by silica gel chromatography (1:1 hexanes/ethyl acetate) to yield a white foam (36 mg, 75% yield). MS (ESI+) m/e 270 (M+1)⁺;

¹HNMR (300 MHz, DMSO-d₆) δ7.72 (s, 1H), 7.06 (ddd, 2H, J=8.4, 3.0, 2.1 Hz), 6.58 (ddd, 2H, J=8.7, 3.0, 2.1 Hz), 5.47 (s, 2H), 2.49 (s, 3H).

### Example 305

### N-(4-(5-acetyl-3-(trifluoromethyl)-1H-pyrazol-1-yl)phenyl)-2-fluorobenzamide

Example 305 D was processed as in Example (i)-a (Method 5, 6, or 7) to provide the title compound.

mp 188-189° C.;

MS (ESI+) m/e 393 (M+1)⁺;

¹HNMR (400 MHz, DMSO-d₆) δ10.93 (s, 1H), 8.78 (d, 1H, J=1.6 Hz), 8.62 (dd, 1H, J=4.8, 1.2 Hz), 7.86 (s, 1H), 7.82 (ddd, 2H, J=8.8, 6.8, 2.8 Hz), 7.74 (t, 1H, J=5.3 Hz), 7.50 (ddd, 2H, J=8.8, 7.2, 3.2 Hz), 2.57 (s, 3H);

¹³CNMR (100 MHz, DMSO-d₆) δ187.8, 161.0, 156.3, 153.7, 146.4, 141.2, 141.0, 140.6, 139.0, 138.9, 135.4, 131.4, 131.2, 126.5, 123.2, 122.2, 119.7, 119.6, 111.1, 28.8;

Anal. calcd for C₁₈H₁₂F₄N₄O₂: C, 55.1 1; H, 3.08; N, 14.28. Found: C, 55.05; H, 3.33; N, 13.71.

### Example 306

### N-(4-(5-cyano-3-(trifluoromethyl)-1H-pyrazol-1-yl)phenyl)-2-fluoronicotinamide

Example 300D was processed as in Example (i)-a (Method 5, 6, or 7) to provide the title compound.

mp 150-151° C.;

MS (DCI/NH₃) m/e 393 (M+NH₄)⁺;

¹H NMR (DMSO-d₆, 300 MHz) δ10.96 (s, 1H), 8.44 (d, 1H), 8.30 (td, 1H), 8.09 (s, 1H), 7.97 (d, 2H), 7.82 (d, 2H), 7.59-7.52 (m, 1H).

### Example 307

### N-(4-(5-cyano-3-(trifluoromethyl)-1H-pyrazol-1-yl)phenyl)-2,3,5-trifluorobenzamide

Example 300D was processed as in Example (i)-a (Method 5, 6, or 7) to provide the title compound.

MS (DCI/NH₃) m/e 428 (M+NH4)⁺;

¹H NMR (DMSO-d₆, 300 MHz) δ10.99 (s, 1H), 8.09 (s, 1H), 7.96 (d, 2H), 7.83 (s, 1H), 7.82 (d, 1H), 7.9-7.50 (m, 1H).

### Example 308

### 2-fluoro-N-(4-(5-(2-thienyl)-3-(trifluoromethyl)-1H-pyrazol-1-yl)phenyl)benzamide

### Example 308A

### 1-(4-nitrophenyl)-5-(2-thienyl)-3-(trifluoromethyl)-1H-pyrazole

Sodium methoxide (0.46 g, 8.52 mmol) in diethyl ether (15 mL) was added dropwise to methyl trifluoroacetate (0.82 mL mL, 7.79 mmol) in diethyl ether (10 mL). 2-Acetyl-thiophene (1 g, 7.79 mmol) in diethyl ether (10 mL) was subsequently added, and the mixture was heated to reflux for 16 hours. After cooling to room temperature, the mixture was concentrated to dryness. The crude intermediate was taken into ethanol (20 mL), and then concentrated HCl (5 mL) and 4-nitrophenylhydrazine (1.21 g, 7.79 mmol) were added followed by heating to reflux for 16 hours. The mixture was freed of solvent and used without additional purification.

MS (DCI/NH₃) m/e 310 (M+H)⁺ (For aniline produced under analysis conditions.);

¹H NMR (DMSO-d₆, 300 MHz) δ8.38 (d, 2H), 7.78 (d, 2H), 7.72 (dd, 1H), 7.40 (s, 1H), 7.21 (dd, 1H), 7.13 (dd, 1H).

### Example 308B

### 4-[5-(2-thienyl)-3-(trifluoromethyl)-1H-pyrazol-1-yl]aniline

The above intermediate was taken into ethanol/water (50 mL, 3:1/v:v), iron powder (3.04 g, 54.53 mmol) and ammonium chloride (0.41 g, 7.79 mmol) were added, and the mixture was heated to reflux for 1 hour. The mixture was filtered through diatomaceous earth (5 g) and freed of solvent. The product was purified by silica gel chromatography (37 g) eluting with 50% acetone in hexanes (v:v). Yield (0.36 g, 15% for three steps).

MS (DCI/NH₃) m/e 310 (M+H)⁺.

### Example 308

### 2-fluoro-N-(4-(5-(2-thienyl)-3-(trifluoromethyl)-1H-pyrazol-1-yl)phenyl)benzamide

Example 308B was processed as in Example (i)-a (Method 5, 6, or 7) to provide the title compound.

mp 154-156° C.;

MS (DCI/NH₃) m/e 449 (M+NH₄)⁺; 432 (M+H)⁺;

¹H NMR (DMSO-d₆, 300 MHz) δ10.75 (s, 1H), 7.9 (d, 2H), 7.75-7.65 (m, 1H), 7.7 (dd, 1H), 7.65-7.55 (m, 1H), 7.5 (d, 2H), 7.45-7.30 (m, 2H), 7.3 (s, 1H), 7.25 (dd, 1H), 7.1 (m, 1H).

### Example 309

### 2-fluoro-N-(4-(5-(methylsulfanyl)-3-(trifluoromethyl)-1H-pyrazol-1-yl)phenyl)benzamide

### Examples 309A-1 and 309A-2

### 5-(methylsulfanyl)-1-(4-nitrophenyl)-3-(trifluoromethyl)-1H-pyrazole

### and

### 1-(4-nitrophenyl)-3-(trifluoromethyl)-1H-pyrazol-5-ol

S-Methyl 4,4,4-trifluoro-3-oxothiobutyrate (2.76 mL, 0.02 mol) and p-nitrophenylhydrazine (3.06 g, 0.02 mol) were dissolved in ethanol 18 mL and 4M HCl/dioxane (18 mL). The solution was refluxed overnight. After cooling the reaction mixture to room temperature, it was partitioned between ether and water. The ether layer was extracted with saturated aquesous NaHCO₃ (3x), washed with brine, dried over Na₂SO₄ and evaporated to give 5-(methylsulfanyl)-1-(4-nitrophenyl)-3-(trifluoromethyl)-1H-pyrazole (Example 309A-1, 0.61 g, 10% yield). The NaHCO₃ extractions were combined and washed with ether. The NaHCO₃ solution was then acidified with 1N HCl to pH≈5 and extracted with ether (3x). The ether extracts were combined, dried over Na₂SO₄ and concentrated to give 1-(4-nitrophenyl)-3-(trifluoromethyl)-1H-pyrazol-5-ol (Example 309A-2, 4.02 g, 74% yield).

Example 309A- 1:

MS (DCI/NH₃) m/e 304 (M+H)⁺;

¹H NMR (DMSO-d₆, 300 MHz) δ8.44 (d, 2H), 7.94 (d, 2H), 7.18 (s, 1H), 2.6 (s, 3H).

Example 309A-2:

MS (DCI/NH₃) m/e 291 (M+NH₄)⁺;

¹H NMR (DMSO-d₆, 300 MHz) δ8.39 (d, 2H), 8.1 (d, 2H), 6.0 (s, 1H).

### Example 309B

### 4-[5-(methylsulfanyl)-3-(trifluoromethyl)-1H-pyrazol-1-yl]aniline

Example 309A-1was reduced with Fe powder as described previously to gave the title compound in 75% yield.

MS (DCI/NH₃) m/e 291(M+NH₄)⁺;

¹H NMR (DMSO-d₆, 300 MHz) δ7.12 (d, 2H), 6.9 (s, 1H), 6.63 (d, 2H), 5.55 (s, 2H), 2.5 (s, 3H).

### Example 309

### 2-fluoro-N-(4-(5-(methylsulfanyl)-3-(trifluoromethyl)-1H-pyrazol-1-yl)phenyl)benzamide

Example 309B was processed as in Example (i)-a (Method 5, 6, or 7) to provide the title compound.

mp 162-163° C.;

MS (DCI/NH₃) m/e 413 (M+NH₄)⁺;

¹H NMR (DMSO-d₆, 300 MHz) δ10.7 (s, 1H), 7.91 (d, 2H), 7.7 (t, 1H), 7.6 (m, 1H), 7.55 (d, 2H), 7.37 (m, 2H), 7.02 (s, 1H), 2.53 (s, 3H).

### 310

### 2-fluoro-N-(4-(5-(3-pyridinyl)-3-(trifluoromethyl)-1H-pyrazol-1-yl)phenyl)benzamide

### Example 310A

### 4-[5-(3-pyridinyl)-3-(trifluoromethyl)-1H-pyrazol-1-yl]aniline

Sodium methoxide (0.46 g, 8.52 mmol) in diethyl ether (15 mL) was added dropwise to methyl trifluoroacetate (0.83 mL, 7.85 mmol) in diethyl ether (10 mL). 2-Acetyl-pyridine (0.88 mL, 7.85 mmol) in diethyl ether (10 mL) was then added, and the mixture was heated to reflux for 16 hours. The mixture was freed of solvent and then redissolved in ethanol (20 mL). Concentrated HCl (5 mL) and 4-nitrophenylhydrazine (1.21 g, 7.85 mmol) were added, and the mixture was heated to reflux for 16 hours. A solvent change to ethanol/water (50 mL, 3:1/v:v) was performed, iron powder (3.04 g, 54.53 mmol) and ammonium chloride (0.41 g, 7.79 mmol) were added, and the mixture was heated to reflux for 1 hour. The reaction mixture was filtered through diatomaceous earth (5 g), and the filtrate was concentrated to dryness. The product was purified by silica gel chromatography (75 mL silica gel) eluting with 50% acetone in hexanes (v:v). Yield (0.57 g, 22% for three steps).

MS (DCI/NH₃) m/e 305 (M+H)⁺;

¹H NMR (DMSO-d₆, 300 MHz) δ9.10 (mn, 1H), 8.59 (dd, 1H), 8.26 (dt, 1H), 7.70 (s, 1H), 7.49 (m, 1H), 7.28 (d, 2H), 6.67 (d, 2H), 5.60 (s, 2H),

### Example 310

### 2-fluoro-N-(4-(5-(3-pyridinyl)-3-(trifluoromethyl)-1H-pyrazol-1-yl)phenyl)benzamide

Example 310B was processed as in Example (i)-a (Method 5, 6, or 7) to provide the title compound.

mp 138-140° C.;

MS (DCI/NH₃) m/e 427 (M+H)⁺;

¹H NMR (DMSO-d₆, 300 MHz) δ10.7 (s, 1H), 8.6-8.6 (m, 2H), 7.8 (d, 2H), 7.7-7.65 (m, 2H), 7.65-7.55 (m, 1H), 7.45-7.3 (m, 6H).

### Example 311

### 3-fluoro-N-(4-(5-(2-thienyl)-3-(trifluoromethyl)-1H-pyrazol-1-yl)phenyl)isonicotinamide

Example 308B was processed as in Example (i)-a (Method 5, 6, or 7) to provide the title compound.

mp 153-155° C.;

MS (DCI/NH₃) m/e 433 (M+H)⁺;

¹H NMR (DMSO-d₆, 300 MHz) δ11.0 (s, 1H), 8.8 (s, 1H), 8.6 (d, 1H), 7.9 (d, 2H), 7.75 (t, 1H), 7.6 (d, 1H), 7.5 (d, 2H), 7.3 (s, 1H), 7.25 (d, 1H), 7.1 (m, 1H).

### Example 312

### N-(4-(5-methoxy-3-(trifluoromethyl)-1H-pyrazol-1-yl)phenyl)isonicotinamide

### Example 312A

### methyl 1-(4-nitrophenyl)-3-(trifluoromethyl)-1H-pyrazol-5-yl ether

To a mixture of Example 309A-2 (0.776 g, 2.84 mmol) and K₂CO₃ (0.94 g, 6.8 mmol) in acetonitrile (10 mL) was added dimethyl sulfate (0.32 mL, 3.4 mmol). Then the reaction mixture was stirred at room temperature overnight. The reaction mixture was diluted with ether and washed with brine. After evaporation of the solvent, the crude was passed through short silica gel plug eluting with methylene chloride to give the title compound (0.71 g, 88% yield).

MS (DCI/NH₃) m/e 305 (M+NH₄)⁺;

¹H NMR (DMSO-d₆, 300 MHz) δ8.39 (d, 2H), 8.03 (d, 2H), 6.59 (s, 1H), 4.08 (s, 3H).

### Example 312B

### 4-[5-methoxy-3-(trifluoromethyl)-1H-pyrazol-1-yl]aniline Reduction of the nitro group of Example 312A with Fe powder gave the title compound in 82% yield.

MS (DCI/NH₃) m/e 275 (M+NH₄)⁺;

¹H NMR (DMSO-d₆, 300 MHz) δ7.18 (d, 2H), 6.61 (d, 2H), 6.36 (s, 1H), 5.41 (s, 2H), 3.94 (s, 3H).

### Example 312

### N-(4-(5-methoxy-3-(trifluoromethyl)-1H-pyrazol-1-yl)phenyl)isonicotinamide

Example 312B was processed as in Example (i)-a (Method 5, 6, or 7) to provide the title compound.

mp 181-182° C.;

MS (DCI/NH₃) m/e 380 (M+NH₄)⁺;

¹H NMR (DMSO-d₆, 300 MHz) δ10.61 (s, 1H), 8.8 (d, 2H), 7.94 (d, 2H), 7.89 (d, 2H), 7.66 (d, 2H), 6.49 (s, 1H), 4.02 (s, 3H).

### Example 313

### 2-fluoro-N-(4-(5-methoxy-3-(trifluoromethyl)-1H-pyrazol-1-yl)phenyl)benzamide

Example 312B was processed as in Example (i)-a (Method 5, 6, or 7) to provide the title compound.

mp 167-168° C.;

MS (DCI/NH₃) m/e 397 (M+NH₄)⁺;

¹H NMR (DMSO-d₆, 300 MHz) δ10.61 (s, 1H), 7.86 (d, 2H), 7.69 (t, 1H), 7.64 (d, 2H), 7.6 (m, 1H), 7.35 (m, 2H), 6.46 (s, 1H), 4.01 (s, 3H).

### Example 314

### N-(4-(5-methoxy-3-(trifluoromethyl)-1H-pyrazol-1-yl)phenyl)-4-methyl-1,2,3-thiadiazole-5-carboxamide

Example 312B was processed as in Example (i)-a (Method 5, 6, or 7) to provide the title compound.

mp 159-160° C.;

MS (DCI/NH₃) m/e 401 (M+NH₄)⁺;

¹H NMR (DMSO-d₆, 300 MHz) δ10.92 (s, 1H), 7.84 (d, 2H), 7.66 (d, 2H), 6.49 (s, 1H), 4.01 (s, 3H), 2.84 (s, 3H).

### Example 315

### N-(4-(5-acetyl-3-(trifluoromethyl)-1H-pyrazol-1-yl)phenyl)-2-fluoronicotinamide

Example 305 D was processed as in Example (i)-a (Method 5, 6, or 7) to provide the title compound.

mp 188-189° C.;

MS (ESI+) m/e 393 (M+1)⁺;

¹HNMR (400 MHz, DMSO-d₆) δ10.93 (s, 1H), 8.78 (d, 1H, J=1.6 Hz), 8.62 (dd, 1H, J=4.8, 1.2 Hz), 7.86 (s, 1H), 7.82 (ddd, 2H, J=8.8, 6.8, 2.8 Hz), 7.74 (t, 1H, J=5.3 Hz), 7.50 (ddd, 2H, J=8.8, 7.2, 3.2 Hz), 2.57 (s, 3H);

¹³CNMR (100 MHz, DMSO-D₆) δ187.8, 161.0, 156.3, 153.7, 146.4, 141.2, 141.0, 140.6, 139.0, 138.9, 135.4, 131.4, 131.2, 126.5, 123.2, 122.2, 119.7, 119.6, 111.1, 28.8;

Anal. calcd for C₁₈H₁₂F₄N₄O₂: C, 55.11; H, 3.08; N, 14.28. Found: C, 55.05; H, 3.33; N, 13.71.

### Example 316

### 2-fluoro-N-(4-(5-(methylsulfanyl)-3-(trifluoromethyl)-1H-pyrazol-1-yl)phenyl)nicotinamide

Example 309B was processed as in Example (i)-a (Method 5, 6, or 7) to provide the title compound.

mp 167-168° C.;

MS (DCI/NH₃) m/e 414 (M+NH₄)⁺;

¹H NMR (DMSO-d₆, 300 MHz) δ10.88 (s, 1H), 8.43 (m, 1H), 8.3 (m, 1H), 7.9 (d, 2H), 7.58 (d, 2H), 7.55 (m, 1H), 7.04 (s, 1H), 2.55 (s, 3H).

### Example 317

### 2-fluoro-N-(4-(5-methoxy-3-(trifluoromethyl)-1H-pyrazol-1-yl)phenyl)nicotinamide

Example 312B was processed as in Example (i)-a (Method 5, 6, or 7) to provide the title compound.

mp 153-154° C.;

MS (DCI/NH₃) m/e 398 (M+NH₄)⁺;

¹H NMR (DMSO-d₆, 300 MHz) δ10.78 (s, 1H), 8.42 (m, 1H), 8.29 (m, 1H), 7.86 (d, 2H), 7.66 (d, 2H), 7.53 (m, 1H), 6.48 (s, 1H), 4.0 (s, 3H).

### Example 318

### 3-fluoro-N-(4-(3-(4-pyridinyl)-5-(trifluoromethyl)-1H-pyrazol-1-yl)phenyl)isonicotinamide

### Example 318A

### 4-[3-(4-pyridinyl)-5-(trifluoromethyl)-1H-pyrazol-1-yl]aniline

Sodium methoxide (2.6 g, 48.1 mmol) in diethyl ether (30 mL) was added dropwise to methyl trifluoroacetate (4.1 mL, 40.8 mmol) in diethyl ether (10 mL). 4-Acetylpyridine (4.58 mL, 41.3 mmol) in diethyl ether (10 mL) was then added, and the mixture was heated to reflux for 16 hours. The mixture was freed of solvent and then redissolved in ethanol (200 mL). Concentrated HCl (41 mL) and 4-nitrophenylhydrazine (6.3 g, 41.2 mmol) were added, and the mixture was heated to reflux for 16 hours. A solvent change to ethanol/water (250 mL, 3:1/v:v) was performed, iron powder (10 g, 179 mmol) and ammonium chloride (2.5 g, 47.2 mmol) were added, and the mixture was heated to reflux for 1 hour. The reaction mixture was filtered through diatomaceous earth (50 g), and the filtrate was concentrated to dryness. The product was purified by silica gel chromatography (200 mL silica gel) eluting with 50% acetone in hexanes (v:v). Yield (2.26 g, 18% for three steps).

MS (DCI/NH₃) m/e 305 (M+H)⁺

¹H NMR (DMSO-d₆, 300 MHz) δ8.70 (d, 2H), 7.90 (d, 2H), 7.80 (s, 1H), 7.15 (d, 2H), 6.65 (d, 2H), 5.60 (s, 2H).

### Example 318

### 3-fluoro-N-(4-(3-(4-pyridinyl)-5-(trifluoromethyl)-1H-pyrazol-1-yl)phenyl)isonicotinamide

Example 318A was processed as in Example (i)-a (Method 5, 6, or 7) to provide the title compound.

mp 148-152° C.;

MS (DCI/NH₃) m/e 428 (M+H)⁺;

¹H NMR (DMSO-d₆, 300 MHz) δ11.05 (s, 1H), 8.8 (d, 1H), 8.7 (d, 2H), 8.4 (dd, 1H), 7.9-7.8 (m, 5H), 7.75 (t, 1H), 7.65 (d, 2H);

¹³C NMR (DMSO-d₆, 75 MHz) δ161.2, 156.7, 153.3, 150.4, 150.3, 148.6, 146.4, 146.3, 139.6, 139.1, 138.8, 138.3, 134.0, 126.7, 124.8, 132.2, 121.2, 120.2, 119.8, 117.6, 114.1, 107.7, 107.6;

Anal. calcd for C₂₁H₁₃F₄N₅O: C, 59.02; H, 3.06; N,16.38. Found: C, 58.82; H, 3.20; N, 16.44.

Example 319

### N-(4-(5-ethoxy-3-(trifluoromethyl)-1H-pyrazol-1-yl)phenyl)-2-fluoronicotinamide

### Example 319A

### 4-[5-ethoxy-3-(trifluoromethyl)-1H-pyrazol-1-yl]aniline

Example 309A-2 was alkylated as described in Example 312A (substituting ethyl bromide for dimethyl sulfate). Yield, 65%. Subsequent reduction with iron powder supplied the aniline. Yield, 71%. MS (DCI/NH₃) m/e 289 (M+NH₄)⁺;

¹H NMR (DMSO-d₆, 300 MHz) δ7.18 (d, 2H), 6.62 (d, 2H), 6.34 (s, 1H), 5.4 (s, 2H), 4.23 (q, 2H), 1.34 (t, 3H).

### Example 319

### N-(4-(5-ethoxy-3-(trifluoromethyl)-1H-pyrazol-1-yl)phenyl)-2-fluoronicotinamide

Example 319A was processed as in Example (i)-a (Method 5, 6, or 7) to provide the title compound.

mp 152-153° C.;

MS (DCI/NH₃) m/e 412 (M+NH₄)⁺;

¹H NMR (DMSO-d₆, 300 MHz) δ10.3 (s, 1H), 8.92 (m, 1H), 8.28 (m, 1H), 7.85 (d, 2H), 7.67 (d, 2H), 7.54 (m, 1H), 6.48 (s, 1H), 4.3 (q, 2H), 1.38 (t, 3H).

### Example 320

### 3-fluoro-N-(4-(5-(methylsulfanyl)-3-(trifluoromethyl)-1H-pyrazol-1-yl)phenyl)isonicotinamide

Example 309B was processed as in Example (i)-a (Method 5, 6, or 7) to provide the title compound.

mp 142-143° C.;

MS (DCI/NH₃) m/e 414 (M+NH₄)⁺;

¹H NMR (DMSO-d₆, 300 MHz) δ8.79 (s, 1H), 8.62 (d, 1H), 7.9 (d, 2H), 7.76 (t, 1H), 7.59 (d, 2H), 7.04 (s, 1H), 2.58 (s, 3H).

### Example 321

### 3-fluoro-N-(4-(5-methoxy-3-(trifluoromethyl)-1H-pyrazol-1-yl)phenyl)isonicotinamide

Example 312B was processed as in Example (i)-a (Method 5, 6, or 7) to provide the title compound.

mp 160-161° C.;

MS (DCI/NH₃) m/e 398 (M+NH₄)⁺;

¹H NMR (DMSO-d₆, 300 MHz) δ8.79 (s, 1H), 8.61 (d, 1H), 7.76 (d, 2H), 7.74 (t, 1H), 7.68 (d, 2H), 6.49 (s, 1H), 4.01 (s, 3H).

### Example 322

### N-(4-(5-(difluoromethoxy)-3-(trifluoromethyl)-1H-pyrazol-1-yl)phenyl)isonicotinamide

### Example 322A

### 5-(difluoromethoxy)-1-(4-nitrophenyl)-3-(trifluoromethyl)-1H-pyrazole

To a solution of Example 309A-2 (1.00 g, 3.66 mmol) in dry dimethylformamide (10 mL) was added potassium carbonate (1.42 g, 10.3 mmol). This mixture was heated for 5 minutes at 80° C., then chlorodifluoromethane was bubbled through the reaction mixture for 30 minutes will maintaining the temperature at 80° C. Introduction of chlorodifluoromethane was stopped after 30 min, then the reaction mixture was heated an additional 30 min. The reaction mixture was partitioned between ether and water. The ether layer was separated and washed with brine, dried over sodium sulfate, filtered and concentrated to give the crude difluoromethoxyether (0.90 g, 76% yield) which was sufficiently pure to use in the next step.

MS (DCI/NH₃) m/e 311 (M+NH₄)⁺ (For corresponding aniline produced by analysis.);

¹H NMR (DMSO-d₆, 300 MHz) δ8.44 (d,2H), 7.98 (d, 2H), 7.2-7.68 (t, 1H), 6.94 (s, 1H).

### Example 322B

### 4-[5-(difluoromethoxy)-3-(trifluoromethyl)-1H-pyrazol-1-yl]aniline

A mixture of the Example 322A (0.90 g, 2.79 mmol), iron powder (1.25 g) and ammonium chloride (0.125 g) in ethanol (12 mL) and water (4 mL) was heated to 100° C. for 30 minutes. Then the reaction mixture was cooled to room temperature and partitioned between ether and brine. The ether layer was dried over sodium sulfate and concentrated to give the crude amine, which was dissolved in methylene chloride and passed through a short silica gel plug to give the pure amine (0.80 g, 97% yield).

MS (DCI/NH₃) m/e 311 (M+NH₄)⁺;

¹H NMR (DMSO-d₆, 300 MHz) δ7.18 (d, 2H), 7.11-7.59 (t, 1H), 6.73 (s, 1H), 6.65 (d, 2H), 5.55 (s, 2H).

### Example 322

### N-(4-(5-(difluoromethoxy)-3-(trifluoromethyl)-1H-pyrazol-1-yl)phenyl)isonicotinamide

Example 322B was processed as in Example (i)-a (Method 5, 6, or 7) to provide the title compound.

mp 154-155° C.;

MS (DCI/NH₃) m/e 416 (M+NH₄)⁺;

¹H NMR (DMSO-d₆, 300 MHz) δ8.8 (d, 2H), 7.99 (d, 2H), 7.88 (d, 2H), 7.65 (d, 2H), 7.16-7.64 (t, 1H), 6.84 (s, 1H).

### Example 323

### N-(4-(5-(difluoromethoxy)-3-(trifluoromethyl)-1H-pyrazol-1-yl)phenyl)-4-methyl-1,2,3-thiadiazole-5-carboxamide

Example 322B was processed as in Example (i)-a (Method 5, 6, or 7) to provide the title compound.

mp 122-123° C.;

MS (DCI/NH₃) m/e 437 (M+NH₄)⁺;

¹H NMR (DMSO-d₆, 300 MHz) δ7.89 (d, 2H), 7.65 (d, 2H), 7.16-7.64 (t, 1H), 6.84 (s, 1H), 2.84 (s, 3H).

### Example 324

### N-(4-(5-(difluoromethoxy)-3-(trifluoromethyl)-1H-pyrazol-1-yl)phenyl)-2-fluoronicotinamide

Example 322B was processed as in Example (i)-a (Method 5, 6, or 7) to provide the title compound.

mp 149-150° C.;

MS (DCI/NH₃) m/e 434 (M+NH₄)⁺;

¹H NMR (DMSO-d₆, 300 MHz) δ8.44 (m, 1H), 8.28 (m, 1H), 7.9 (d, 2H), 7.65 (d, 2H), 7.55 (m, 1H), 7.16-7.64 (t, 1H), 6.84 (s, 1H).

### Example 325

### N-(4-(5-chloro-3-(trifluoromethyl)-1H-pyrazol-1-yl)phenyl)-2-fluoronicotinamide

### Example 325A

### 5-chloro-1-(4-nitrophenyl)-3-(trifluoromethyl)-1H-pyrazole

A slurry of Example 309A-2 (4.63 g, 16.9 mmol) in phenylphosphinic dichloride (11.5 mL, 81.1 mmol) was heated to 145° C. for 48 hours in a sealed tube with stirring. The reaction mixture was cooled to room temperature and carefully poured into saturated sodium bicarbonate solution (300 mL). The aqueous layer was further basified with 1 N NaOH (50 mL). The aqueous layer was extracted with ether (2×300 mL). The combined organic layers were dried over sodium sulfate, filtered and concentrated. The crude oil was purified with 93% hexane/7% ethyl acetate to afford the title compound as a light yellow oil (3.10 g, 63% yield).

MS (DCI/NH₃) m/e 279 (M+NH₄)⁺ (for the corresponding aniline produced in the analysis);

¹H NMR (DMSO-d₆, 300 MHz) δ8.47 (d, 2H), 8.02 (d, 2H), 7.44 (s, 1H).

### Example 325B

### 4-[5-chloro-3-(trifluoromethyl)-1H-pyrazol-1-yl]aniline

The nitro group of Example 325A was reduced with by the iron reduction procedure described previously.

MS (DCI/NH₃) m/e 279 (M+NH₄)⁺;

¹H NMR (DMSO-d₆, 300 MHz) δ7.19 (s, 1H), 7.18 (d, 2H), 6.65 (d, 2H), 5.62 (s, 2H).

### Example 325

### N-(4-(5-chloro-3-(trifluoromethyl)-1H-pyrazol-1-yl)phenyl)-2-fluoronicotinamide

Example 325B was processed as in Example (i)-a (Method 5, 6, or 7) to provide the title compound.

MS (DCI/NH₃) m/e 402 (M+NH₄)⁺;

¹H NMR (DMSO-d₆, 300 MHz) δ10.91 (s, 1H), 8.45 (d, 1H), 8.30 (dt, 1H), 7.93 (d, 2H), 7.63 (d, 2H), 7.57 (dt, 1H), 7.31 (s, 1H).

### Example 326

### 2-fluoro-N-(4-(5-nitro-3-(trifluoromethyl)-1H-pyrazol-1-yl)phenyl)benzamide

### Example 326A

### tert-butyl 1-(4-nitrophenyl)-3-(trifluoromethyl)-1H-pyrazol-5-ylcarbamate

To a solution of Example 305A (2.11 g, 6.96 mmol) in toluene (40.0 mL) was added triethylamine (1.5 mL, 10.4 mmol) followed by diphenylphosphorylazide (2.25 mL, 10.4 mmol) and tert-butanol (4.7 mL, 48.7 mmol). The resulting mixture was heated at 80° C. for 20 hours. The reaction mixture was cooled and concentrated. The crude oil was chromatographed with 25% ethyl acetate/75% hexane to afford the title compound as a thick yellow oil (2.59 g, 99% yield).

MS (DCI/NH₃) m/e 373 (M+H)⁺;

¹H NMR (DMSO-d₆, 300 MHz) δ9.79 (s, 1H), 8.45 (d, 2H), 7.88 (d, 2H), 6.88 (s, 1H), 1.32 (s, 9H).

### Example 326B

### tert-butyl 1-(4-aminophenyl)-3-(trifluoromethyl)-1H-pyrazol-5-ylcarbamate

The nitro group of Example 326A was reduced with by the iron reduction procedure described previously.

MS (DCI/NH₃) m/e 343 (M+H)⁺;

¹H NMR (DMSO-d₆, 300 MHz) δ9.16 (s, 1H), 7.10 (d, 2H), 6.69 (s, 1H), 6.64 (d, 2H), 1.33 (s, 9H).

### Example 326C

### tert-butyl 1-{4-[(2-fluorobenzoyl)amino]phenyl}-3-(trifluoromethyl)-1H-pyrazol-5-ylcarbamate

Example 326B was processed as in Example (i)-a (Method 5, 6, or 7) to provide the title compound.

MS (DCI/NH₃) m/e 465 (M+H)⁺;

¹H NMR (DMSO-d₆, 300 MHz) δ10.66 (s, 1H), 9.43 (s, 1H), 8.10 (dt, 1H), 7.89 (d, 2H), 7.70 (dt, 1H), 7.51 (d, 2H), 7.50-7.29 (m, 2H), 6.79 (s, 1H), 1.34 (s, 9H).

### Example 326

### 2-fluoro-N-(4-(5-nitro-3-(trifluoromethyl)-1H-pyrazol-1-yl)phenyl)benzamide

To an ice cold (0° C.) flask containing Example 326C (25 mg, 0.054 mmol) was added sulfuric acid (1 mL). This mixture was stirred at room temperature for 30 min. 30% Hydrogen peroxide (0.5 mL) solution was then added and the resulting mixture was stirred at room temperature for 20 hours. The mixture was poured into saturated sodium bicarbonate solution (30 mL), and the aqueous layer was extracted with ethyl acetate (3×30 mL) The combined organic layers was dried over sodium sulfate, filtered and concentrated. The crude oil was purified by flash column chromatography with 10% ethyl acetate/90% hexane to afford the title compound as an oil (7 mg, 33% yield).

MS (DCI/NH₃) m/e 412 (M+NH₄)⁺;

¹H NMR (DMSO-d₆, 300 MHz) δ10.76 (s, 1H), 8.15 (s, 1H), 7.91 (d, 2H), 7.75-7.58 (m, 3H), 7.67 (d, 2H), 7.43- 7.27 (m, 2H).

### Example 327

### N-(4-(5-(difluoromethoxy)-3-(trifluoromethyl)-1H-pyrazol-1-yl)phenyl)-3-fluoroisonicotinamide

Example 322B was processed as in Example (i)-a (Method 5, 6, or 7) to provide the title compound.

mp 135-136° C.;

MS (DCI/NH₃) m/e 434 (M+NH₄)⁺;

¹H NMR (DMSO-d₆, 300 MHz) δ10.98 (s, 1H), 8.79 (s, 1H), 8.62 (d, 1H), 7.9 (d, 2H), 7.74 (t, 1H), 7.66 (d, 2H), 7.16-7.66 (t, 1H), 6.85 (s, 1H).

### Example 328

### N-(4-(5-chloro-3-(trifluoromethyl)-1H-pyrazol-1-yl)phenyl)-3-fluoroisonicotinamide

Example 325B was processed as in Example (i)-a (Method 5, 6, or 7) to provide the title compound.

mp 128-129° C.;

MS (DCI/NH₃) m/e 412 (M+NH₄)⁺;

¹H NMR (DMSO-d₆, 300 MHz) δ11.03 (s, 1H), 8.80 (s, 1H), 8.63 (d, 1H), 7.93 (d, 2H), 7.75 (t, 1H), 7.67 (d, 2H), 7.32 (s, 1H);

¹³C NMR (DMSO-d₆, 75 MHz) δ161.2, 154.5, 146.4, 141.8, 139.4, 139.0, 132.6, 131.2, 129.1, 126.4, 123.3, 121.0, 120.3, 105.0;

Anal. calcd for C₁₆H₉ClF₄N₄O: C, 49.95; H, 2.35; N, 14.56. Found: C, 50.07; H, 2.46; N, 14.47.

### Example 329

### N-(4-(5-(difluoromethyl)-3-(3-pyridinyl)-1H-pyrazol-1-yl)phenyl)-3-fluoroisonicotinamide

### Example 329A

### nicotinaldehyde N-(4-nitrophenyl)hydrazone

3-Pyridine carboxaldehyde (3.69 mL, 0.04 mol), p-nitrophenylhydrazine (6 g, 0.04 mol), 1 drop of acetic acid, and ethanol (150 mL) were combined. The slurry was heated at 100° C. for 12 hours with stirring. After it was cooled to room temperature, the yellow solid was filtered and dried to give the title compound (9.12 g 96%) which was pure enough to use in the next step.

MS (DCI/NH₃) m/e 243 (M+1)⁺;

¹H NMR (DMSO-d₆, 300 MHz) δ11.45 (s, 1H), 8.89 (d, 1H), 8.59 (d, 1H), 8.57-8.52 (m, 1H), 8.16 (d, 2H), 8.08 (s, 1H), 7.78 (dt, 1H), 7.21 (d, 2H).

### Example 329B

### N-(4-nitrophenyl)-3-pyridinecarbohydrazonoyl chloride

To a solution of the Example 329A (6 g, 0.025 mol) in DMF (10 mL) at 0° C. was added a solution of N-chlorosuccinimide (3.45 g, 0.026 mol, 1.05 eq) in N,N-dimethylformamide (15 mL) dropwise over 30 minutes. After addition, the resulting dark green mixture was stirred at room temperature for two hours. Then it was poured into ice water with stirring. The resulting light brown solid was filtered, and dried in a vacuum oven at 40° C. for 12 hours to give the title compound (4.9 g, 71% yield) as an orange solid which was used in the next step without additional purification.

MS (DCI/NH₃) m/e 277 (M+1)⁺;

¹H NMR (DMSO-d₆, 300 MHz) δ10.91 (s, 1H), 9.17 (d, 1H), 8.68 (dd, 1H), 8.32 (dd, 1H), 8.20 (d, 2H), 7.61-7.53 (m, 3H).

### Example 329C

### methyl 1-(4-nitrophenyl)-3-(3-pyridinyl)-1H-pyrazole-5-carboxylate

Example 329B (2.0 g, 7.2 mmol), methyl α-chloroacrylate (1.5 g, 10.8 mmol, 1.5 eq), toluene (15 mnL), and triethylamine (2.5 mL, 18 mmol, 2.5 eq) were combined and heated at 80° C. for 8 hours. After cooling to room temperature, the mixture was diluted with ethyl acetate (30 mL), and washed with 1N HCl (30 mL), and saturated NaCl solution (30 mL). The organic portion was dried over Na₂SO₄, filtered, and concentrated in vacuo. This dark brown crude oil was purified by flash chromatography, using ethyl acetate-hexane (v/v, 3:7) to give the pyrazole (650 mg, 28%) as a brown oil.

MS (DCI/NH₃) m/e 243 (M+1)⁺;

¹H NMR (DMSO-d₆, 300 MHz) δ9.19 (d, 1H), 8.60 (dd, 1H), 8.39 (d, 2H), 8.33 (dd, 1H), 7.94 (d, 2H), 7.88 (s, 1H), 7.50 (dd, 1H).

### Example 329D

### [1-(4-nitrophenyl)-3-(3-pyridinyl)-1H-pyrazol-5-yl]methanol

To a −78° C. solution of Example 329C (650 mg, 2.0 mmol) in THF (30 mL) was added DIBAL (1M soln in hexane, 6.7 mL, 6.7 mmol) dropwise with stirring. After two hours at −78° C., the mixture was warmed to 0° C. and stirred an additional two hours. After it was quenched with potassium sodium tartrate solution (30 mL), the resulting mixture was stirred at room temperature overnight. Then the reaction mixture was diluted with ethyl acetate (20 mL) and washed with brine solution (20 mL). The organic layer was dried over NASO₄, filtered and concentrated vacuo. The crude product was purified by flash chromatography, with ethyl acetate-hexane (v/v, 75:25) to give the alcohol (370 mg,60%) as an oil.

MS (DCI/NH₃) m/e 297 (M+1)⁺;

¹H NMR (DMSO-d₆, 300 MHz) δ9.13 (d, 1H), 8.59 (dd, 1H), 8.42 (d, 2H), 8.29 (dd, 1H), 8.09 (d, 2H), 7.50 (dd, 1H), 7.19 (s, 1H), 5.81 (t, 1H), 4.67 (d, 2H).

### Example 329E

### 1-(4-nitrophenyl)-3-(3-pyridinyl)-1H-pyrazole-5-carbaldehyde

Under an argon atmosphere, solid tetrapropylammoniumperruthenate (21 mg, 0.06 mmol) was added in one portion to a solution of Example 329D (350 mg, 1.2 mmol) dissolved in dichloromethane (5 mL) and acetonitrile (0.5 mL). N-methylmorpholine N-oxide (208 mg, 1.8 mmol) was then added followed by flame dried powdered molecular sieves (1 g). The resulting black mixture was stirred at room temperature for 18 hours. The mixture was diluted with 10 mL of dichloromethane and filtered through a short silica gel plug with ethyl acetate-hexane (v/v, 7:3) to afford the aldehyde (180 mg, 53% yield) as an oil.

MS (DCI/NH₃) m/e 295 (M+1)⁺;

¹H NMR (DMSO-d₆, 300 MHz) δ9.93 (s, 1H), 9.19 (d, 1H), 8.63 (dd, 1H), 8.43 (d, 2H), 8.36 (dd, 1H), 8.04 (d, 2H), 7.99 (s, 1H), 7.52 (dd, 1H).

### Example 329F

### 3-[5-(1,3-dithiolan-2-yl)-1-(4-nitrophenyl)-1H-pyrazol-3-yl]pyridine

Example 329E (150 mg, 0.51 mmol), a catalytic amount of p-toluenesulfonic acid (3 mg), 1,2-ethanediol (0.04 mL, 0.51 mmol) and toluene (50 mL) were combined and refluxed for 4 hours in a Dean-Stark apparatus. Then the solution was cooled to room temperature. The toluene solution was washed with NaHCO₃ solution (20 mL) and brine (20 mL). The organic layer was separated, dried with Na₂SO₄, filtered and concentrated in vacuo to give the dithiane (135 mg, 72%) as a crude yellow solid which was pure enough to use in the next step.

MS (DCI/NH₃) m/e 371 (M+1)⁺;

¹H NMR (DMSO-d₆, 300 MHz) δ9.11 (d, 1H), 8.57 (dd, 1H), 8.43 (d, 2H), 8.27 (dd, 1H), 7.97 (d, 2H), 7.48 (dd, 1H), 7.29 (s, 1H), 6.03 (s, 1H), 3.48-3.36 (m, 2H), 3.10-3.04 (m, 2H).

### Example 329G

### 3-[5-(difluoromethyl)-1-(4-nitrophenyl)-1H-pyrazol-3-yl]pyridine

To a cold (0° C.) solution of 1,3-dibromo-5,5-dimethylhydantoin (302 mg, 1.06 mmol) in anhydrous dichloromethane (5 mL) under argon atmosphere was added HF-pyridine (0.2 mL, 0.88 mmol), followed by Example 329F (130 mg, 0.35 mmol). The resulting red solution was stirred at 0° C. for 45 minutes, then diluted with dichloromethane (10 mL) and quenched with NaHCO₃ solution (10 mL). The organic layer was separated and washed with more NaHCO₃ solution (10 mL) dried with NASO₄, filtered and concentrated in vacuo to give 100 mg of black crude material. This crude product was purified by HPLC with ethyl acetate-hexane (v/v, 6:4) to give the difluoromethane (40 mg, 46%) as an oil.

See Reference: Katzenellenbogen, J. A.; Sondej, S. C. J. Org. Chem. 1986, 51(18), 3508-3513.

MS (DCI/NH₃) m/e 317 (M+1)⁺;

¹H NMR (DMSO-d₆, 300 MHz) δ9.19 (d, 1H), 8.62 (dd, 1H), 8.46 (d, 2H), 8.33 (dt, 1H), 7.96 (d, 2H), 7.65 (s, 1H), 7.52 (dd, 1H), 7.49 (s, 1H).

### Example 329H

### 4-[5-(difluoromethyl)-3-(3-pyridinyl)-1H-pyrazol-1-yl]aniline

The title compound was prepared by iron powder and ammonium chloride reduction as previously described. The product was used in the subsequent step without additional purification or charactherization.

### Example 329

### N-(4-(5-(difluoromethyl)-3-(3-pyridinyl)-1H-pyrazol-1-yl)phenyl)-3-fluoroisonicotinamide

Example 329H was processed as in Example (i)-a (Method 5, 6, or 7) to provide the title compound.

mp 176-178° C.;

MS (ESI−) m/e 408 (M−1)⁻;

¹H NMR (DMSO-d₆, 300 MHz) δ9.14 (d, 1H), 8.8 (d, 1H), 8.64-8.58 (m, 2H), 8.48 (dd, 1H), 8.29 (dt, 1H), 7.92 (d, 2H), 7.75 (t, 1H), 7.65 (d, 2H), 7.53-7.46 (m, 2H), 3.72 (t, 1H).

### Example 330

### N-(4-(5-cyano-3-(2-pyridinyl)-1H-pyrazol-1-yl)phenyl)-3-fluoroisonicotinamide

### Example 330A

### N-(4-nitrophenyl)-2-pyridinecarbohydrazonoyl chloride

This compound was obtained from 2-pyridinecarboxaldehyde 4-nitrophenylhydrazone in 84% yield using the methodology described in the preparation of Example 329A.

MS (DCI) m/e 277 (M+1)⁺;

¹H NMR (DMSO-d₆, 300 MHz) δ10.93 (s, 1H), 8.68 (d, 1H), 8.21 (d, 2H), 7.93 (td, 1H), 7.58 (d, 2H), 7.49 (dd, 1H), 7.24 (d, 1H).

### Example 330B

### 1-(4-nitrophenyl)-3-(2-pyridinyl)-1H-pyrazole-5-carbonitrile

The title compound was prepared from Example 330A and 2-chloroacrylonitrile in 39% yield using methodology described in the preparation of Example 329.

MS (DCI) m/e 292 (M+1)⁺;

¹H NMR (CDCl₃, 300 MHz) δ8.70 (d, 1H), 8.45 (d, 2H), 8.16-8.07 (m, 3H), 7.84 (td, 1H), 7.80 (s, 1H), 7.35 (dd, 1H).

### Example 330C

### 1-(4-aminophenyl)-3-(2-pyridinyl)-1H-pyrazole-5-carbonitrile

This material was prepared in 34% yield from Example 330B using methodology described in the preparation of Example 329.

MS (DCI) m/e 262 (M+1)⁺;

¹H NMR (DMSO-d₆, 300 MHz) δ8.66 (d, 1H), 8.03 (d, 1H), 7.92 (td, 1H), 7.82 (s, 1H), 7.43-7.36 (m, 3H), 6.61 (d, 2H), 5.68 (s, 2H).

### Example 330

### N-(4-(5-cyano-3-(2-pyridinyl)-1H-pyrazol-1-yl)phenyl)-3-fluoroisonicotinamide

Example 330C was processed as in Example (i)-a (Method 5, 6, or 7) to provide the title compound.

mp 118-119° C.;

MS (DCI/NH₃) m/e 385 (M+1)⁺;

¹H NMR (DMSO-d₆, 300 MHz) δ8.8 (d, 1H), 8.68 (dt, 1H), 8.63 (dd, 1H), 8.08 (d, 1H), 7.99-7.92 (m, 4H), 7.85 (d, 2H), 7.77 (t, 1H), 7.48-7.42 (m, 1H).

### Example 331

### N-(4-(5-cyano-3-(3-pyridinyl)-1H-pyrazol-1-yl)phenyl)-4-methyl-1,2,3-thiadiazole-5-carboxamide

### Example 331A

### 1-(4-nitrophenyl)-3-(3-pyridinyl)-1H-pyrazole-5-carbonitrile

The title compound was prepared in 32% yield using the methodology described in the preparation of Example 304 using 2-chloroacrylonitrile and the chlorohydrazone previously described in the preparation of Example 329.

MS (DCI) m/e 292 (M+1)⁺;

¹H NMR (DMSO-d₆, 300 MHz) δ9.19-9.16 (m, 1H), 8.66 (d, 1H), 8.51 (d, 2H), 8.37-8.30 (m, 1H), 8.23 (s, 1H), 8.17 (d, 2H), 7.56 (dd, 1H).

### Example 331B

### 1-(4-aminophenyl)-3-(3-pyridinyl)-1H-pyrazole-5-carbonitrile

The title compound was prepared in 84% yield from the 331A using methodology described in the preparation of Example 304.

MS (DCI) m/e 262 (M+1)⁺;

¹H NMR (DMSO-d₆, 300 MHz) δ9.10 (d, 1H), 8.25 (dt, 1H), 7.95 (s, 1H), 7.50 (dd, 1H), 7.39 (d, 2H), 6.70 (d, 2H), 5.65 (s, 2H).

### Example 331

### N-(4-(5-cyano-3-(3-pyridinyl)-1H-pyrazol-1-yl)phenyl)-4-methyl-1,2,3-thiadiazole-5-carboxamide

Example 331B was processed as in Example (i)-a (Method 5, 6, or 7) to provide the title compound.

mp 215-218° C.;

MS (ESI) m/e 386 (M−1)⁻, 388 (M+1)⁺;

¹H NMR (DMSO-d₆, 300 MHz) δ9.15 (d, 1H), 8.63 (dd, 1H), 8.30 (dt, 1H), 8.10 (s, 1H), 7.95 (d, 2H), 7.85 (d, 2H), 7.54 (dd, 1H), 2.80 (s, 3H).

### Example 332

### N-(4-(5-bromo-3-(trifluoromethyl)-1H-pyrazol-1-yl)phenyl)-3-fluoroisonicotinamide

### Example 332A

### 5-bromo-1-(4-nitrophenyl)-3-(trifluoromethyl)-1H-pyrazole

Example 309A-2 (442 mg, 1.62 mmol) and phosphorous tribromide (2.63, 9.17 mmol) were heated at 160° C. for 20 hours. The reaction mixture was cooled and saturated sodium bicarbonate solution (20 mL) was added cautiously over 30 minutes. The mixture was diluted further with bicarbonate solution (100 mL). The aqueous layer was extracted with ethyl acetate (2×150 mL). The combined organic layers were dried over sodium sulfate, filtered and concentrated. The crude oil was purified by flash chromatography using 95% hexane/5% ethyl acetate affording the title compound as a dark brown oil (467 mg, 86% yield).

MS (DCI/NH₃) m/e 325 (M+NH₄)⁺ (for aniline produced in analysis);

¹H NMR (DMSO-d₆, 300 MHz) δ8.45 (d, 2H), 7.98 (d, 2H), 7.43 (s, 1H).

### Example 332B

### 4-[5-bromo-3-(trifluoromethyl)-1H-pyrazol-1-yl]aniline

The title compound was prepared by iron powder and ammonium chloride reduction as previously described. The product was used in the subsequent step without additional purification or charactherization.

MS (DCI/NH₃) m/e 323 (M+NH₄)⁺;

¹H NMR (DMSO-d₆, 300 MHz) δ7.16 (d, 2H), 7.14 (s, 1H), 6.67 (d, 2H), 5.60 (s, 2H).

### Example 332

### N-(4-(5-bromo-3-(trifluoromethyl)-1H-pyrazol-1-yl)phenyl)-3-fluoroisonicotinamide

Example 332B was processed as in Example (i)-a (Method 5, 6, or 7) to provide the title compound.

mp 143-144° C.;

MS (DCI/NH₃) m/e 446 (M+NH₄)⁺;

¹H NMR (DMSO-d₆, 300 MHz) δ11.03 (s, 1H), 8.80 (s, 1H), 8.63 (d, 1H), 7.92 (d, 2H), 7.76 (t, 1H), 7.64 (d, 2H), 7.33 (s, 1H);

Anal. calcd for C₁₆H₉BrF₄N₄O: C, 44.77; H, 2.11; N, 12.95. Found: C, 44.43; H, 2.11; N, 12.95.

### Example 333

### 3-fluoro-N-(4-(5-nitro-3-(trifluoromethyl)-1H-pyrazol-1-yl)phenyl)isonicotinamide

### Example 333A

### tert-butyl 1-[4-(isonicotinoylamino)phenyl]-3-(trifluoromethyl)-1H-pyrazol-5-ylcarbamate

Example 326B was processed as in Example (i)-a (Method 5, 6, or 7) to provide the title compound.

MS (DCI/NH₃) m/e 483 (M+NH₄)⁺;

¹H NMR (DMSO-d₆, 300 MHz) δ10.95 (s, 1H), 9.46 (s, 1H), 8.79 (s, 1H), 8.62 (d, 1H), 7.87 (d, 2H), 7.74 (t, 1H), 7.54 (d, 2H), 6.80 (s, 1H), 1.33 (s, 9H).

### Example 333

### 3-fluoro-N-(4-(5-nitro-3-(trifluoromethyl)-1H-pyrazol-1-yl)phenyl)isonicotinamide

A solution of Example 333A (100 mg, 0.215 mmol) in trifluoroacetic acid (2 mL) and methylene chloride (2 mL) was stirred at room temperature for 30 min. The solvent was removed in vacuo and the residue was dissolved in acetonitrile (1 mL). Sodium nitrate (300 mg) and copper sulfate were mixed together in a separate flask with acetonitrile (2 mL) and water (1 mL). The amine solution was added slowly over 5 minutes, then the resulting mixture was allowed to stir for 15 minutes. The reaction mixture was poured into saturated sodium bicarbonate solution (50 mL). The aqueous layer was extracted with ethyl acetate (2×50 mL). The combined organic layers were dried over sodium sulfate, filtered and concentrated. The residue was purified by flash chromatography with 50% ethyl acetate/50% hexane to obtain the title compound (8 mg, 9% yield).

mp 188-190° C.;

MS (DCI/NH₃) m/e 413 (M+NH₄)⁺;

¹H NMR (DMSO-d₆, 300 MHz) δ11.01 (s, 1H), 8.80 (s, 1H), 8.63 (d, 1H), 8.14 (s, 1H), 7.89 (d, 2H), 7.76 (t, 1H), 7.69 (d, 2H);

Anal. calcd for C₁₆H₉F₄N₅O₃: C, 48.61; H, 2.29; N, 17.71. Found: C, 48.89; H, 2.37; N, 17.38.

### Example 334

### 3-fluoro-N-{4-[5-nitro-3-(3-pyridinyl)-1H-pyrazol-1-yl]phenyl}isonicotinamide

### Example 334A

### 1-(4-nitrophenyl)-3-(3-pyridinyl)-1H-pyrazol-5-amine

3-Oxo-3-pyridin-3-yl-propionitrile (3.57 g, 24.4 mmol)[Chem. Abstr.; 60; 10689d; 1964] and p-nitrophenylhydrazine (3.74 g, 24.4 mmol) were dissolved in ethanol (100 mL), treated with 4N HCl in dioxane (61 mL) and refluxed for 2 hours. After cooling to ambient temperature and evaporation to dryness, the residue was partitioned between ethyl acetate and 1N sodium bicarbonate solution. After removal of the aqueous phase, the organic layer was dried over MgSO₄ and concentrated in vacuo to provide 5.57 g (19.8 mmol, 81%) of crude product. Silica gel chromatography of the crude product eluting with hexanes-acetone (4 step gradient from 6:1 to 1:1) provided 3.59 g (12.8 mmol, 52.5%) of pure product as an oil.

MS (ESI−) m/e 280 (M−H)⁻;

¹H NMR (DMSO-d₆, 300 MHz) δ9.02 (dd, J=0.5, 2 Hz, 1H), 8.54 (dd, J=2.5 Hz, 1H), 8.37 (dm, J=9 Hz, 2H), 8.17 (dt, J=8.2 Hz, 1H), 8.04 (dm, J=9 Hz, 2H), 7.46 (dd, J=5.8 Hz, 1H), 6.11 (s, 1H), 5.92 (s, 2H).

### Example 334B

### [4-nitro-[3-(3-pyridyl)-5-(bis-Boc-amino)-1H-pyrazol-1-yl]benzene

Example 334A (0.66 g, 2.35 mmol) was dissolved into dioxane (5 mL) and treated with di-t-butyldicarbonate (0.62 g, 2.82 mmol) and a catalytic amount of 4-(dimethylamino)pyridine at 60° C. for 1 day. Additional di-t-butyldicarbonate (0.62 g, 2.82 mmol) and 4-(dimethylamino)pyridine were introduced at 60° C. for 3 hours to completely consume the starting material. The solvent was evaporated in vacuo and the residue was purified by chromatography on silica gel (Biotage 40S) eluting with hexanes-acetone (step gradient 9:1 to 2:1) to provide 537 mg (1.41 mmol, 48%) of pure product as the bis-Boc material (some mono-Boc product was sometimes also present and was combined with the bis-Boc product for the subsequent reactions).

MS (ESI−) m/e 380 (M−H)⁻; MS (ESI+) m/e 482 (M+H)⁺;

¹H NMR (DMSO-d₆, 300 MHz) δ9.14 (d, J=2 Hz, 1H), 8.62 (dd, J=2.5 Hz, 1H), 8.52 (dm, J=9 Hz, 2H), 8.29 (dt, J=8.2 Hz, 1H), 7.78 (dm, J=9 Hz, 2H), 7.53 (dd, J=5.8 Hz, 1H), 7.33 (s, 1H), 1.29 (s, 18H).

### Example 334C

### [4-nitro-[3-(3-pyridyl)-5-(bis-Boc-amino)-1H-pyrazol-1-yl]]benzene

Example 334B (525 mg, 1.11 mmol) was dissolved in ethanol (10 mL) and water (0.5 mL) and reduced with iron and ammonium chloride as described previously to provide 375 mg (0.83 mmol, 75%) as a mixture of mono and bis-Boc protected product which was used directly in the subsequent amide coupling reactions. MS (ESI+) m/e 352(M+H)⁺(mono-Boc);

(ESI−) m/e 452(M+H)⁺(bis-Boc);

¹H NMR (DMSO-d₆, 300 MHz) δ9.04 (d, J=2 Hz, 0.33H), 9.02 (d, J=2 Hz, 0.67H), 8.44 (s, 0.67H), 8.55-8.50 (m, 1H), 8.62-8.14 (m, 1H), 7.47-7.42 (m, 1H), 7.15 (dm, J=9 Hz, 1.33H), 7.06 (dm, J=9 Hz, 0.67H), 7.04 (s, 0.33H), 6.82 (s, 0.67H), 6.67-6.62 (m, 2H), 5.43 (s, 0.67H), 5.36 (s, 1.33H), 1.30-1.38 (m, 12H).

### Example 334D

### N-[4-[3-(3-pyridyl)-5-(bis-Boc-amino)-1H-pyrazol-1-yl]phenyl]-3-fluoropyridin-4-yl-carboxamide

Example 334C was processed as in Example (i)-a (Method 5, 6, or 7) to provide 530 mg of product as a mixture of mono and bis Boc protected substances.

MS (ESI−) m/e 473(M−H)⁻;

¹H NMR (DMSO-d₆, 300 MHz) δ9.23 (s, 0.4H), 9.09 (d, J=2 Hz, 0.6H), 9.06 (d, J=2 Hz, 0.4H), 8.78 (s, 1H), 8.62 (d, J=5 Hz, 1H), 8.58-8.54 (m, 1H), 8.26-8.20 (m, 1H), 7.92-7.84 (m, 2H), 7.76-7.73 (m, 1H), 7.59 (dm, J=9 Hz, 0.8H), 7.52-7.44 (m, 2.2H), 7.18 (s, 0.6H), 6.93 (s, 0.4H), 5.98 (s, 0.4H);

### Example 334E

### N-[4-[3-(3-pyridyl)-5-amino-1H-pyrazol-1-yl]phenyl]-3-fluoropyridin-4-yl-carboxamide

Example 334E (530 mg) was treated with 4N HCl in dioxane (20 mL) for 1 hour. The excess reagent and solvent were removed by evaporation in vacuo, and the residue (0.60 g) was used without purification.

MS (ESI−) m/e 373(M−H)⁻; 409(M+Cl)⁻;

¹H NMR (DMSO-d₆, 300 MHz) δ10.92 (s, 1H), 9.15 (s, 1H), 8.79 (s, 1H), 8.72 (d, J=6 Hz, 1H), 8.67-8.62 (m, 2H), 7.89-7.85 (m, 2H), 7.74 (t, J=5 Hz, 1H), 7.66 (dm, J=9 Hz, 2H), 6.14 (s, 1H);

### Example 334

### 3-fluoro-N-{4-[5-nitro-3-(3-pyridinyl)-1H-pyrazol-1-yl]phenyl}isonicotinamide

Example 334E (54 mg, 0.14 mmol) in 10% H₂SO₄ (1 mL) was added dropwise to NaNO₂ (400 mg) in water (2 mL) at 50° C. The outgassing of the reaction stopped after approximately 15 minutes. The reaction was cooled to ambient temperature and diluted with 1N NaHCO₃ solution. The product was extracted into ethyl acetate, the ethyl acetate layer was washed with water (2x) and dried over MgSO₄. After evaporation of the solvent, the residue was purified by chromatography on silica gel (2 g Alltech Extract-Clean™ silica) by elution with hexanes-ethyl acetate (1:2) to provide 17 mg (0.042 mmol, 30%) of the title compound as an off-white solid.

mp 213-215° C.;

MS (ESI−) m/e 403(M−H)⁻; 439(M+Cl)⁻;

¹H NMR (DMSO-d₆, 300 MHz) δ11.02 (s, 1H) 9.19 (d, 1H, J=2 Hz), 8.82 (s, 1H), 8.62-8.66 (m, 2H), 8.35 (dt, 1H, J=8.2 Hz), 8.22 (s, 1H), 7.89 (d, 2H, J=9 Hz), 7.77 (t, 1H, J=5 Hz), 7.69 (d, 2H, J=9 Hz), 7.53 (dd, 1H, J=5.8 Hz).

### Example 335

### N-(4-(5-bromo-3-(trifluoromethyl)-1H-pyrazol-1-yl)phenyl)-4-methyl-1,2,3-thiadiazole-5—

Example 332B was processed as in Example (i)-a (Method 5, 6, or 7) to provide the title compound.

mp 145-147° C.;

MS (DCI/NH₃) m/e 451 (M+NH₄)⁺;

¹H NMR (DMSO-d₆, 300 MHz) δ11.02 (s, 1H), 7.90 (d, 2H), 7.64 (d, 2H), 7.32 (s, 1H), 2.84 (s, 3H);

Anal. calcd for C₁₄H₉BrF₃N₅OS: C, 48.61; H, 2.29; N, 17.71. Found: C, 48.89; H, 2.37; N, 17.38.

### Example 336 N-{4-[5-chloro-3-(trifluoromethyl)-1H-pyrazol-1-yl]phenyl}-3-fluoroisonicotinamide

### Example 336A

### 1-(4-nitrophenyl)-3-(trifluoromethyl)-1H-pyrazol-5-amine

To a cold (0° C.) mixture of 2,2,2-trifluoro-N-(4-nitrophenyl)ethanehydrazonoyl chloride (161 mg, 0.60 mmol) and 5-aminotetrazole (51 mg, 0.60 mmol) in ethanol was added triethylamine (0.180 mL, 1.27 mmol). The resulting mixture was stirred at room temperature for one hour then heated to reflux for 4 hours. The reaction mixture was cooled and poured into saturated sodium bicarbonate solution (50 mL) and then extracted with ethyl acetate (3×50 mL). The combined organic layers were dried over sodium sulfate, filtered and concentrated. The crude residue was purified using flash chromatography with 50% ethyl acetate/50% hexane to afford the title compound as an oil (95 mg, 58% yield).

MS (DCI/NH₃) m/e 274 (M+1)⁺;

¹H NMR (DMSO-d₆, 300 MHz) δ8.41 (d, 2H), 7.91 (d, 2H), 7.32 (s, 2H).

### Example 336B

### 4-[5-chloro-3-(trifluoromethyl)-1H-pyrazol-1-yl]aniline

A solution of Example 336A (90 mg, 0.329 mmol) in acetonitrile (1 mL) was added to to a cold solution (0° C.) of copper chloride (66 mg, 0.49 mmol) and t-butyl nitrite (0.058 mL, 0.49 mmol) in acetonitrile (2 mL). The resulting mixture was stirred for 30 minutes, then poured into brine (50 mL). The aqueous layer was extracted with ethyl acetate (3×50 mL). The combined organic layers were dried over sodium sulfate, filtered and concentrated. The crude residue was purified by flash chromatography with 10% ethyl acetate/90% hexane to afford the chlorotriazole as an oil (70 mg, 73% yield). MS (DCI/NH₃) m/e 280 (M+NH₄,)⁺ (For aniline produced by the analysis.); ¹H NMR (DMSO-d₆, 300 MHz) δ8.50 (d, 2H), 8.07 (d, 2 H). This nitro compound was subjected to the usual iron reduction conditions and used without purification in the next step. TLC analysis indicated that the reaction was complete.

### Example 336

### N-{4-[5-chloro-3-(trifluoromethyl)-1H-pyrazol-1-yl]phenyl}-3-fluoroisonicotinamide

mp 167-170° C.;

MS (DCI/NH₃) m/e 386 (M+H)⁺;

¹H NMR (DMSO-d₆, 300 MHz) δ11.08 (s, 1H), 8.80 (s, 1H), 8.63 (d, 1H), 7.95 (d, 2H), 7.76 (d, 2H), 7.75 (s, 1H).

### N-(4-(5-chloro-3-(trifluoromethyl)-1H-1,2,4-triazol-1-yl)phenyl)-3-fluoroisonicotinamide

Example 325B was processed as in Example (i)-a (Method 5, 6, or 7) to provide the title compound.

mp 167-170° C.;

MS (DCI/NH₃) m/e 386 (M+H)⁺;

¹H NMR (DMSO-d₆, 300 MHz) δ11.08 (s, 1H), 8.80 (s, 1H), 8.63 (d, 1H), 7.95 (d, 2H), 7.76 (d, 2H), 7.75 (s, 1H).

### Example 337

### 4-methyl-N-(4-(5-nitro-3-(3-pyridinyl)-1H-pyrazol-1-yl)phenyl)-1,2,3-thiadiazole-5—

### Example 337A

### tert-butyl 1-(4-{[(4-methyl-1,2,3-thiadiazol-5-yl)carbonyl]amino}phenyl)-3-(3-pyridinyl)-1H-pyrazol-5-ylcarbamate

The aniline used to prepare Example 334 (510 mg, 1.45 mmol) was was processed as in Example (i)-a (Method 5, 6, or 7) to provide 580 mg (1.21 mmol, 84%) of product that was used directly in the next step.

MS (ESI−) m/e 476(M−H)⁻ (mono-Boc); 576(M−H)⁻ (bis-Boc);

¹H NMR (DMSO-d₆, 300 MHz) δ10.96 (s, 0.4H), 10.93 (s, 0.6H), 9.97 (s, 0.6H), 9.10 (D, J=2 HZ, 0.4H), 9.06(d, J=2 Hz, 0.6H), 8.61-8.54 (m, 1.4H), 8.26-8.20 (m, 1H), 7.88 (d, J=9 Hz, 0.8H), 7.83 (d, J=9 Hz, 1.2H), 7.59 (d, J=9 Hz, 1.2H), 7.52-7.45 (m, 1.4H), 7.42-7.37 (m, 0.4H), 7.18 (s, 0.4H), 6.93 (s, 0.6H), 2.83 (s, 3H).

### Example 337B

### N-{4-[5-amino-3-(3-pyridinyl)-1H-pyrazol-1-yl]phenyl}-4-methyl-1,2,3-thiadiazole-5-carboxamide

Example 337A (580 mg, 1.21 mmol) was treated with 4N HCl in dioxane (20 mL) for 1 hour. The excess reagent and solvent were evaporated in vacuo to provide 0.71 mg of solid. The solid was partitioned between ethyl acetate and 1N sodium bicarbonate solution, and the organic layer was further washed with water (2x) and dried over MgSO₄ to provide 363 mg (0.96 mmol, 79%) of product.

MS (ESI+) m/e 378(M+H)⁺;

¹H NMR (DMSO-d₆, 300 MHz) δ10.91 (s, 1H), 8.96 (d, J=2 Hz, 1H), 8.51 (d, J=5 Hz, 1H), 8.12 (d, J=8 Hz, 1H), 7.83 (d, J=9 Hz, 2H), 7.67 (d, J=9 Hz, 2H),7.42 (dd, J=5.8 Hz, 1H, 6.00 (s, 1H), 5.54 (s, 2H), 2.83 (s, 3H).

### Example 337

### N-[4-[3-(3-pyridyl)-5-nitro-1H-pyrazol-1-yl]phenyl]-4-methylthiadiazol-5-yl-carboxamide

Example 337B (145 mg, 0.38 mmol) was dissolved in 10% H₂SO₄ (1.5 mL) and added in portions over 1 minute to sodium nitrite (548 mg, 7.9 mmol) in 5.5 mL water. The mixture was allowed to react with vigorous stirring for 5 minutes at 60° C. The reaction was quenched by the addition of 1N sodium bicarbonate solution, and the product was extracted into ethyl acetate followed by concentration in vacuo. Additional purification was achieved chromatographically with an Alltech Extract-Clean™ cartridge eluting with ethyl acetate to provide 30 mg (0.073 mmol, 19%) of the title compound.

mp 208-210° C.;

MS (ESI−) m/e 406(M−H)⁻;

¹H NMR (DMSO-d₆, 300 MHz) δ9.18 (d, 1H, J=2 Hz), 8.63 (dd, 1H, J=2.5 Hz), 8.33 (dt, 1H, J=8.2 Hz), 8.20 (s, 1H), 7.87 (d, 2H, J=9 Hz), 7.69 (d, 2H, J=9 Hz), 7.53 (dd, 1H, J=5.8 Hz), 2.86 (s, 3H).

### Example 338

### N-(4-(5-cyano-3-(1,3-thiazol-2-yl)-1H-pyrazol-1-yl)phenyl)-3-fluoroisonicotinamide

### Example 338A

### 1,3-thiazole-2-carbaldehyde N-(4-nitrophenyl)hydrazone

The hydrazone was prepared from 4-nitrophenylhydrazine and 2-thiazolecarboxaldehyde in 88% yield using the methodology described in the preparation of Example 300A.

MS (DCI) m/e 249 (M+1)⁺;

¹H NMR (DMSO-d₆, 300 MHz) δ8.21 (s, 1H), 8.09 (d, 2H), 7.88 (d, 1H), 7.69 (d, 1H), 7.09 (d, 2H).

### Example 338B

### N-(4-nitrophenyl)-1,3-thiazole-2-carbohydrazonoyl chloride

The chlorohydrazone was prepared in 88% yield from the hydrazone prepared above using methodology described in the preparation of Example 300B.

¹H MS (DCI) m/e 283 (M+1)⁺;

¹H NMR (DMSO-d₆, 300 MHz) δ11.10 (s, 1H), 8.24 (d, 2H), 7.96 (d, 1H), 7.92 (d, 1H), 7.48 (d, 2H).

### Example 338C

### 1-(4-nitrophenyl)-3-(1,3-thiazol-2-yl)-1H-pyrazole-5-carbonitrile

The title compound was prepared in 15% yield using the chlorohydrazone prepared above and the reagents and methodology described in the preparation of Example 300C.

MS (DCI) m/e 298 (M+1)⁺;

¹H NMR (DMSO-d₆, 300 MHz) δ8.51 (d, 2H), 8.15 (d, 2H), 8.09 (s, 1H), 8.04 (d, 1H), 7.97 (d, 1H).

### Example 338D

### 1-(4-aminophenyl)-3-(1,3-thiazol-2-yl)-1H-pyrazole-5-carbonitrile

The compound was prepared in 36% yield from the nitrophenyl compound prepared above using the methodology described in the preparation of Example 300D.

MS (ESI) m/e 268 (M+1)⁺, 266 (M−1)⁻.

¹H NMR (DMSO-d₆, 300 MHz) δ7.97 (d, 1H), 7.83 (d, 1H), 7.81 (s, 1H),7.38 (d, 2H), 6.70 (d, 2H), 5.68 (s, 2H).

### Example 338

### N-(4-(5-cyano-3-(1,3-thiazol-2-yl)-1H-pyrazol-1-yl)phenyl)-3-fluoroisonicotinamide

Example 338D was processed as in Example (i)-a (Method 5, 6, or 7) to provide the title compound.

mp 210-211° C.;

MS (ESI) m/e 389 (M−1)⁻, 391 (M+1)⁺;

¹H NMR (DMSO-d₆, 300 MHz) δ11.04 (s, 1H), 8.80 (d, 1H), 8.63 (dd, 1H), 8.00 (d, 1H), 7.97 (d, 2H), 7.89 (d, 1H), 7.84 (d, 2H), 7.76 (t, 1H).

### Example 339

### N-(4-(3-(5-bromo-3-pyridinyl)-5-(difluoromethoxy)-1H-pyrazol-1-yl)phenyl)-3-fluoroisonicotinamide

### Example 339A

### 3-(5-bromo-3-pyridinyl)-1-(4-nitrophenyl)-1H-pyrazol-5-ol

Condensation of methyl 5-bromonicotinoylacetate and p-nitrophenylhydrazine using methodology previously described gave the title compound in quantitative yield.

MS (APCI) m/e 361 (M+H)⁺;

¹H NMR (DMSO-d₆, 300 MHz) δ9.1 (d, 1H), 8.73 (d, 1H), 8.52 (t, 1H), 8.38 (d, 2H), 8.22 (d, 2H), 6.33 (s, 1H).

### Example 339B

### 3-bromo-5-[5-(difluoromethoxy)-1-(4-nitrophenyl)-1H-pyrazol-3-yl]pyridine

This intermediate was prepared by alkylation of Example 339A in 81% yield using the procedure described in the preparation of Example 322A.

MS (DCI/NH₃) m/e 430 (M+NH₄)⁺;

¹H NMR (DMSO-d₆, 300 MHz) δ9.4 (d, 1H), 8.75 (d, 1H), 8.57 (t, 1H), 8.44 (d, 2H), 8.07 (d, 2H), 7.66-7.18 (t, 1H), 7.14 (s, 1H).

### Example 339C

### 4-[3-(5-bromo-3-pyridinyl)-5-(difluoromethoxy)-1H-pyrazol-1-yl]phenylamine

This intermediate was prepared by reduction of the above compound with iron powder in 82% yield as described in the preparation of Example 322B.

MS (DCI/NH₃) m/e 400 (M+NH₄)⁺;

¹H NMR (DMSO-d₆, 300 MHz) δ9.04 (d, 1H), 8.67 (d, 1H), 8.45 (t, 1H), 7.54-7.06 (t, 1H), 7.22 (d, 2H), 6.93 (s, 1H), 6.65 (d, 2H), 5.47 (s, 2H).

### Example 339

### N-(4-(3-(5-bromo-3-pyridinyl)-5-(difluoromethoxy)-1H-pyrazol-1-yl)phenyl)-3-fluoroisonicotinamide

Example 339C was processed as in Example (i)-a (Method 5, 6, or 7) to provide the title compound.

mp 179-180° C.;

MS (DCI/NH₃) m/e 506 (M+H)⁺;

¹H NMR (DMSO-d₆, 300 MHz) δ10.94 (s, 1H), 9.1 (d, 1H), 8.8 (s, 1H), 8.72 (d, 1H), 8.62 (d, 1H), 8.51 (t, 1H), 7.9 (d, 2H), 7.74 (t, 1H), 7.71 (d, 2H), 7.13-7.61 (t, 1H), 7.05 (s, 1H).

### Example 340

### N-(4-(5-cyano-3-(1,3-thiazol-2-yl)-1H-pyrazol-1-yl)phenyl)-4-methyl-1,2,3-thiadiazole-5-carboxamide

Example 338D was processed as in Example (i)-a (Method 5, 6, or 7) to provide the title compound.

mp 230° C.;

MS (ESI−) m/e 392 (M−1)⁻;

¹H NMR (DMSO-d₆, 300 MHz) δ8.10 (d, 1H), 7.96 (d, 2H), 7.97 (s, 1H), 7.89 (d, 1H), 7.85 (d, 2H), 2.86 (d, 3H).

### Example 341

### N-(4-(5-cyano-3-(1,3-thiazol-2-yl)-1H-pyrazol-1-yl)phenyl)-3-fluoroisonicotinamide

Example 338D was processed as in Example (i)-a (Method 5, 6, or 7) to provide the title compound.

mp 162-163° C.;

MS (DCI/NH₃) m/e 434 (M+1)⁺;

¹H NMR (DMSO-d₆, 300 MHz) δ11.06 (s, 1H), 8.80 (d, 1H), 8.63 (dd, 1H), 8.00 (d, 1H), 7.93 (d, 2H), 7.87 (d, 1H), 7.78 (t, 1H), 7.66 (d, 2H), 7.62 (s, 1H);

IR (KBr) cm⁻¹ 3277, 3102, 1653, 1604, 1541, 1516, 1416, 1389, 1325, 1295, 1247, 1168, 1125, 990, 934, 844, 726.

### Example 342

### 4-methyl-N-(4-(3-(1,3-thiazol-2-yl)-5-(trifluoromethyl)-1H-pyrazol-1-yl)phenyl)-1,2,3-thiadiazole-5-carboxamide

### Example 342A

### 4,4,4-trifluoro-1-(1,3-thiazol-2-yl)-1,3-butanedione, sodium salt

To a slurry of sodium methoxide (5.2 g, 96 mmol) in ethyl ether (250 mL) under nitrogen was added methyl trifluoroacetate (9.66 mL, 96 mmol) slowly with stirring. The resulting white slurry was stirred at room temperature for 30 minutes. It was cooled to 0° C. and 2-acetylthiazole (8.28 mL, 80 mmol) was added dropwise to the mixture. This slurry became a clear solution upon addition of 2-acetylthiazole. Then the mixture was heated to reflux for 1 hour. This resulting reddish slurry was cooled to room temperature and ethyl ether was removed in vacuo to give the diketone product (17.80 g, quantitative) as an off white solid. This crude product was not further purified before the next step.

MS (ESI) m/e 222 (M−1)⁻;

¹H NMR (DMSO-d₆, 300 MHz) δ7.88 (d, 1H), 7.84 (d, 1H), 6.39 (s, 1H).

### Example 342B

### 2-[5-(trifluoromethyl)-1H-pyrazol-3-yl]-1,3-thiazole

Example 342A (4.8 g, 22 mmol), anhydrous hydrazine (1.28 mL, 26.4 mmol), and dry toluene (100 mL) were combined and heated to reflux for 3 hours. The reaction mixture was cooled to room temperature and the toluene was removed in vacuo. This crude material was purified by flash chromatography, eluting with ethyl acetate-hexanes (v/v, 3:7) to give the desired pyrazole product (1.8 g, 38%).

MS (DCI/NH₃) m/e 220 (M+1)⁺;

¹H NMR (DMSO-d₆, 300 MHz) δ10.52 (s, 1H), 8.02 (d, 1H), 7.49 (d, 1H), 7.25 (s, 1H), 6.98 (s, 1H).

### Example 342C

### 2-[1-(4-nitrophenyl)-5-(trifluoromethyl)-1H-pyrazol-3-yl]-1,3-thiazole

To a cooled (0° C.) slurry of sodium hydride (95%, 432 mg, 17 mmol) and dry DMF (20 mL) was added dropwise Example 342B (3.4 g, 16 mmol) in dry DMF (5 mL). The resulting mixture was stirred for 10 minutes, 4-fluoronitrobenzene (1.80 mL, 17 mmol) was also added dropwise to the reaction mixture at 0° C. After addition, the mixture was heated to reflux for 3 hours. After the reaction was complete, the reaction mixture was cooled to room temperature, partitioned between 30 mL of ethyl acetate (30 mL) and water (20 mL). The organic layer was separated, dried with Na₂SO₄, filtered and concentrated in vacuo to give a mixture of regioisomers (5 g, 91%, 2:1 mixture of regioisomers). This crude material was not purified before next iron reduction step.

MS (ESI) m/e 341 (M+1)⁺;

Compound 1: 1H NMR (DMSO-d₆, 300 MHz) δ8.36 (d, 2H), 7.97 (d, 1H), 7.91 (d, 1H), 7.82 (d, 2H), 7.66 (s, 1H);

Compound 2: 1H NMR (DMSO-d₆, 300 MHz) δ8.47 (d, 2H), 8.02 (d, 1H), 7.95 (d, 2H), 7.89 (d, 1H), 7.73 (s, 1H).

### Example 342D

### 4-[3-(1,3-thiazol-2-yl)-5-(trifluoromethyl)-1H-pyrazol-1-yl]aniline

Iron powder (5.75 g, 103 mmol), ammonium chloride (595 mg, 12 mmol), Example 342C (isomeric mixture from previous step, 5 g, 15 mmol) and ethanol-H₂O (4:1, 50 mL) were combined. This resulting black mixture was heated to reflux for 8 hours. The reaction mixture was cooled to room temperature, passed through a diatomaceous earth pad and a silica gel plug, eluting with ethyl alcohol. After the desired fractions was combined and concentrated in vacuo, the residue was diluted with dichloromethane (20 mL) and washed with NaHCO₃ (20 mL×2). The organic portion was dried with Na₂SO₄, filtered and concentrated in vacuo. This brown crude product was purified by flash chromatography, eluting with ethyl acetate-hexanes (v/v, 2:8) to give the desired product as a pale white solid (1.5 g, 33% yield).

MS (ESI) m/e 311 (M+1)⁺, 309 (M−1)⁻;

¹H NMR (DMSO-d₆, 300 MHz) δ7.97 (d, 1H), 7.82 (d, 1H), 7.49 (s, 1H), 7.19 (d, 2H), 6.65 (d, 2H), 5.65 (s, 2H).

### Example 342

### 4-methyl-N-(4-(3-(1,3-thiazol-2-yl)-5-(trifluoromethyl)-1H-pyrazol-1-yl)phenyl)-1,2,3-thiadiazole-5-carboxamide

Example 342D was processed as in Example (i)-a (Method 5, 6, or 7) to provide the title compound.

mp 162-163° C.;

MS (ESI) m/e 437 (M+1)⁺, 435 (M−1)⁻;

¹H NMR (DMSO-d₆, 300 MHz) δ11.04 (s, 1H), 8.00 (d, 1H), 7.92 (d, 2H), 7.86 (d, 1H), 7.65 (d, 2H), 7.62 (s, 1H), 2.86 (s, 3H).

### Example 343

### N-(4-(3-(2,4-dimethyl-1,3-thiazol-5-yl)-5-(trifluoromethyl)-1H-pyrazol-1-yl)phenyl)-3-fluoroisonicotinamide

### Example 343A

### 4-[3-(2,4-dimethyl-1,3-thiazol-5-yl)-5-(trifluoromethyl)-1H-pyrazol-1-yl]aniline

A mixture of sodium methoxide (2.10 g, 38.65 mmol), methyl trifluoroacetate (3.90 mL, 38.65 mmol) and 5-acetyl-2,4-dimethylthiazole (5.0 g, 32.2 mmol) in ether (150 mL) was heated at reflux for 16 hours. The reaction mixture was cooled and ether was removed in vacuo. Ethanol (100 mL), 4-nitrophenylhydrazine (4.92 g, 32.2 mmol) and concentrated HCl (10 mL) were added and the resulting mixture was heated to reflux for 16 hours. The reaction mixture was cooled and iron powder (12.5 g, 225 mmol) was added and the mixture was heated at reflux for 2 hours. The reaction mixture was cooled and poured in saturated sodium bicarbonate solution (200 mL). The aqueous layer was extracted with ethyl acetate (3×150 mL). The combined organic extracts were dried over sodium sulfate, filtered and concentrated. The residue was purified three times by flash chromatography using 15% isopropanol/85% hexane to afford (150 mg, 1.4% yield) the desired product.

¹H NMR (DMSO-d₆, 300 MHz) δ7.28 (s, 1H), 7.14 (d, 2H), 6.64 (d, 2H), 5.60 (s, 2H), 2.61 (s, 3H), 2.51 (s, 3H).

### Example 343

### N-(4-(3-(2,4-dimethyl-1,3-thiazol-5-yl)-5-(trifluoromethyl)-1H-pyrazol-1-yl)phenyl)-3-fluoroisonicotinamide

Example 342D was processed as in Example (i)-a (Method 5, 6, or 7) to provide the title compound.

mp 194-195° C.;

MS (DCI/NH₃) m/e 462 (M+H)⁺; ¹H NMR (DMSO-d6, 300 MHz) 8 11.01 (s, 1H), 8.80 (s, 1H), 8.62 (d, 1H), 7.91 (d, 2H), 7.75 (t, 1H), 7.60 (d, 2H), 7.41 (s, 1H), 2.63 (s, 3H), 2.51 (s, 3H).

### Example 344

### 3-fluoro-N-(4-(5-(1-methyl-1H-pyrrol-3-yl)-3-(trifluoromethyl)-1H-pyrazol-1-yl)phenyl)isonicotinamide

### Example 344A

### 4-[5-(1-methyl-1H-pyrrol-3-yl)-3-(trifluoromethyl)-1H-pyrazol-1-yl]phenylamine

3-Acetyl-1-methyl pyrrole (5 g, 40.65 mmol), sodium methoxide (2.6 g, 48.15 mmol) and methyl trifluoroacetate (4.9 mL, 48.15 mmol) were combined with diethyl ether (200 mL). The mixture was heated to reflux for 2 hours. After cooling to room temperature, solvent was removed. Hydrazine monohydrate (2.16 mL, 44.58 mmol) and toluene (150 mL) were added, and the reaction mixture was heated to reflux for 16 hours. Upon cooling to room temperature, solvent was once again removed. The crude material was dissolved in dimethylformamide (100 mL) and cooled to 0° C. This solution was added dropwise to a mixture of sodium hydride (60% in mineral oil, 1.79 g, 44.72 mmol) in dimethylformamide (30 mL). After stirring at 0° C. for 30 minutes, 1-fluoro-4-nitrobenzene (4.3 mL, 40.65 mmol) was added. The resulting mixture was warmed to 90° C. for 16 hours. After cooling to 0° C., the reaction was quenched with water (5 mL). The quenched mixture was partitioned between ethyl acetate (100 mL) and water (100 mL). The aqueous layer was back extracted with ethyl acetate (2×100 mL). The organic layers were combined, washed with brine (2×100 mL), dried over magnesium sulfate and concentrated to dryness. Iron powder (15.6 g, 0.28 mol), ammonium chloride (2.26 g, 40.65 mmol) and a mixture of ethanol/water (200 mL, 3:1/v:v) were added to the crude intermediate. The mixture was heated to reflux for 1 hour. After cooling to ambient temperature, the reaction mixture was passed through a pad of diatomaceous earth (20 g). The filtrate was concentrated to dryness. The crude product was purified by silica gel chromatography eluting with 40% acetone in hexanes (v:v). Fractions containing the desired product were combined and freed of solvent (2.11 g, 17% yield).

MS (DCI/NH₃) m/e 307 (M+H)⁺;

¹H NMR (DMSO-d₆, 300 MHz) δ7.01 (d, 2H), 7.95 (s, 1H), 6.72 (t, 1H), 6.60 (d, 2H), 6.37 (t, 1H), 5.85 (m, 1H), 5.53 (s, 2H), 3.53 (s, 3H)

### Example 344

### 3-fluoro-N-(4-(5-(1-methyl-1H-pyrrol-3-yl)-3-(trifluoromethyl)-1H-pyrazol-1-yl)phenyl)isonicotinamide

Example 344A was processed as in Example (i)-a (Method 5, 6, or 7) to provide the title compound.

mp 171-173° C.;

MS (DCI/NH₃) m/e 430 (M+H)⁺;

¹H NMR (DMSO-d₆, 300 MHz) δ8.80 (d, 1H), 8.60 (dd, 1H), 7.85 (d, 2H), 7.45 (d, 2H), 7.22 (s, 1H), 6.91 (s, 1H), 6.75 (m, 1H), 6.70 (t, 1H), 5.80 (m, 1H), 3.55 (s, 3H).

### Example 345

### 3-fluoro-N-(4-(3-tetrahydro-2-furanyl-5-(trifluoromethyl)-1H-pyrazol-1-yl)phenyl)isonicotinamide

### Example 345A

### 3-(2-furyl)-5-(trifluoromethyl)-1H-pyrazole

4,4,4-Trifluoro-1-(2-furyl)-1,3-butanedione (0.9 g, 4.39 mmol) and hydrazine monohydrate (0.19 mL, 4.82 mmol) were combined in toluene (10 mL) and refluxed overnight. After cooling to room temperature, solvent was removed in vacuo. The product (0.77 g, 87% crude yield) was used without further purification.

¹H NMR (DMSO-d₆, 300 MHz) δ7.85 (m, 1H), 7.00 (s, 1H), 6.95 (d, 1H), 6.67 (m, 1H).

### Example 345B

### 3-(2-furyl)-1-(4-nitrophenyl)-5-(trifluoromethyl)-1H-pyrazole

To a mixture of sodium hydride (60% in mineral oil, 0.193 g, 4.83 mmol) and dimethylformamide (10 mL) under nitrogen at 0° C. was added dropwise the Example 345A (0.89 g, 4.41 mmol) dissolved in dimethylformamide (5 mL) over a period of 10 minutes. Then 1-fluoro-4-nitrobenzene (0.47 mL, 4.43 mmol) was added dropwise, and the resulting mixture was heated to 100° C. for 3 hours. The reaction mixture was cooled and partitioned between water (20 mL) and ethyl acetate (30 mL). The aqueous layer was further washed with ethyl acetate (2×20 mL). The organic washes were combined and dried over MgSO₄. Solvent was removed, and the crude product was loaded onto a filter cake (70 mL silica gel and 10 g anhydrous magnesium sulfate), and the product eluted with 50% acetone in hexanes (v:v). Fractions containing the desired product and the regioisomer were combined and concentrated in vacuo. The two isomers were separated by HPLC (silica gel, YMC) eluting with 10% ethyl acetate in hexanes. The regioisomers were present in a 1:2 ratio with the desired material being the minor constituent. Overall yield: 0.35 g (26%) of the desired product.

MS (DCI/NH₃) m/e 324 (M+H)⁺;

¹H NMR (DMSO-d₆, 300 MHz) δ8.45 (d, 2H), 7.92 (d, 2H), 7.83 (m, 1H), 7.62 (s, 1H), 7.05 (m, 1H), 6.67 (m, 1H).

### Example 345C

### (+) 4-[3-tetrahydro-2-furanyl-5-(trifluoromethyl)-1H-pyrazol-1-yl]phenylamine

A solution of the above compound and 10% palladium on carbon in methanol containgin one drop of concentrated hydrochloric acid was hydrogenated at 4 atm at room temperature for 18 hours, filtered through a short silica gel plug, and concentrated to provide the desire compound.

MS (DCI/NH₃) m/e 298 (M+H)⁺.

### Example 345D

### 3-fluoro-N-(4-(3-tetrahydro-2-furanyl-5-(trifluoromethyl)-1H-pyrazol-1-yl)phenyl)isonicotinamide

Example 345C was processed as in Example (i)-a (Method 5, 6, or 7) to provide the title compound.

mp 116-118° C.;

MS (DCI/NH₃) m/e 421 (M+H)⁺;

¹H NMR (DMSO-d₆, 300 MHz) δ11.00 (s, 1H), 8.80 (d, 1H), 8.62 (dd, 1H), 7.9 (d, 2H), 7.85 (t, 1H), 7.52 (d, 2H), 7.10 (s, 1H), 4.95-4.90 (m, 1H), 3.97-3.89 (m, 1H), 3.81-3.73 (m, 1H), 2.30-2.21 (m, 1H), 2.27-1.90 (m, 3H).

### Example 346

### 3-chloro-N-(4-(5-chloro-3-(trifluoromethyl)-1H-pyrazol-1-yl)phenyl)isonicotinamide

Example 325B was processed as in Example (i)-a (Method 5, 6, or 7) to provide the title compound using 3-chloroisonicotinic acid prepared as described in the reference below.

Reference: Lecomte, L.; Ndzi, B.; Queguiner, G.; Turck, A. FR. 2,686,340-A1.

mp 184-185° C.;

MS (DCI/NH₃) m/e 418 (M+NH₄)⁺;

¹H NMR (DMSO-d₆, 300 MHz) δ11.06 (s, 1H), 8.83 (s, 1H), 8.71 (d, 1H), 7.92 (d, 2H), 7.73 (d, 1H), 7.66 (d, 2H), 7.32 (s, 1H).

### Example 347

### N-(4-(5-chloro-3-(1,3-thiazol-2-yl)-1H-pyrazol-1-yl)phenyl)-3-fluoroisonicotinamide

### Example 347A

### methyl 3-oxo-3-(1,3-thiazol-2-yl)propanoate

To a cold solution (−78° C.) of diisopropylamine (7.5 mL, 51.82 mmol) in diethyl ether (200 mL) was added n-BuLi (2.5 M in hexane, 18.0 mL, 45 mmol). The resulting solution was stirred at −78° C. for 30 minutes at which point neat 2-acetylthiazole (5.07 g, 39.87 mmol) was added. The resulting solution was stirred for one hour at −78° C. and neat methyl cyanoformate (4.7 mL, 59.81 mmol) was added and the resulting mixture was stirred at −78° C. for 3 hours. The reaction mixture was then warmed to room temperature over a period of one hour. The reaction was quenched by the addition of water (150 mL). The layers were separated. The aqueous layer was acidified to pH 1, then extracted with ether (150 mL). The organic layer was dried over sodium sulfate, filtered and concentrated to give the title compound as an oil (7.37 g, 99% yield).

MS (DCI/NH₃) m/e 186 (M+H)⁺;

¹H NMR (DMSO-d₆, 300 MHz) δ8.29 (d, 1H), 8.18 (d, 1H), 4.21 (s, 2H).

### Example 347B

### 1-(4-nitrophenyl)-3-(1,3-thiazol-2-yl)-1H-pyrazol-5-ol

A mixture of Example 347A (7.32 g, 39.6 mmol), 4-nitrophenylhydrazine (6.65 g, 43.5 mmol), concentrated HCl (15 mL) and water (15 mL) in dioxane (200 mL) was heated at reflux for 4 hours. The reaction mixture was cooled to room temperature and approximately 75% of the solvent was removed in vacuo. The reaction mixture was diluted with brine (200 mL) and the aqueous mixture was extracted with ethyl acetate (2×200 mL). The combined organic layers were dried over sodium sulfate, filtered and concentrated to a crude orange solid (7.32 g, 64% yield) which was pure enough for the next step.

MS (DCI/NH₃) m/e 306 (M+NH₄)⁺;

¹H NMR (DMSO-d₆, 300 MHz) δ8.40 (d, 2H), 8.17 (d, 2H), 7.92 (d, 1H), 7.79 (d, 1H), 6.10 (s, 1H).

### Example 347C

### 2-[5-chloro-1-(4-nitrophenyl)-1H-pyrazol-3-yl]-1,3-thiazole

A mixture of the Example 347B (938 mg, 3.25 mmol) and phenylphosphinic dichloride (5.0 mL, 35.3 mmol) was heated at 150° C. for 24 hours. The reaction mixture was cooled and poured slowly into saturated sodium bicarbonate solution (150 mL). The aqueous layer was extracted with ether (3×150 mL). The combined organic layers were dried over sodium sulfate, filtered and concentrated. The crude residue was purified by flash chromatography with 90% hexane/10% ethyl acetate affording the title compound as a yellow oil (215 mg, 22% yield).

MS (DCI/NH₃) m/e 307 (M+H)⁺;

¹H NMR (DMSO-d₆, 300 MHz) δ8.46 (d, 2H), 8.04 (d, 2H), 7.99 (d, 1H), 7.86 (d, 1H), 7.29 (s, 1H).

### Example 347D

### 4-[5-chloro-3-(1,3-thiazol-2-yl)-1H-pyrazol-1-yl]phenylamine

The nitro group of Example 347C was reduced with iron as described previously.

MS (DCI/NH₃) m/e 277 (M+H)⁺;

¹H NMR (DMSO-d₆, 300 MHz) δ7.92 (d, 1H), 7.78 (d, 1H), 7.21 (d, 2H), 7.06 (s, 1H), 6.68 (d, 2H), 5.58 (s, 2H).

### Example 347

### N-(4-(5-chloro-3-(1,3-thiazol-2-yl)-1H-pyrazol-1-yl)phenyl)-3-fluoroisonicotinamide

Example 347D was processed as in Example (i)-a (Method 5, 6, or 7) to provide the title compound.

mp 194-195° C.;

MS (DCI/NH₃) m/e 400 (M+H)⁺;

¹H NMR (DMSO-d₆, 300 MHz) δ10.98 (s, 1H), 8.79 (s, 1H), 8.63 (d, 1H), 7.95 (d, 1H), 7.93 (d, 2H), 7.81 (d, 1H), 7.75 (t, 1H), 7.68 (d, 2H), 7.17 (s, 1H);

Anal. calcd for C₁₈H₁₁ClFN₅OS: C, 54.07; H, 2.77; N, 17.51. Found: C, 53.90; H, 3.05; N, 17.00.

### Example 348

### N-(4-(5-bromo-3-(trifluoromethyl)-1H-pyrazol-1-yl)phenyl)-2,3-difluorobenzamide

Example 332B was processed as in Example (i)-a (Method 5, 6, or 7) to provide the title compound.

mp 109-112° C.;

MS (DCI/NH₃) m/e 446 (M+H)⁺;

¹H NMR (DMSO-d₆, 300 MHz) δ7.91 (d, 2H), 7.70-7.60 (m, 1H), 7.61 (d, 2H), 7.58-7.51 (m, 1H), 7.41-7.33 (m, 1H), 7.30 (s, 1H);

Anal. calcd for C₁₇H₉F₅N₃OBr: C, 45.30; H, 2.12; N, 9.32. Found: C, 45.09; H, 2.3; N, 9.07.

### Example 349

### N-(4-(5-bromo-3-(trifluoromethyl)-1H-pyrazol-1-yl)phenyl)-3-chloroisonicotinamide

Example 332B was processed as in Example (i)-a (Method 5, 6, or 7) to provide the title compound.

mp 79-82° C.;

MS (DCI/NH₃) m/e 445 (M+H)⁺;

¹H NMR (DMSO-d₆, 300 MHz) δ8.81 (s, 1H), 8.70 (d, 1H), 7.90 (d, 2H), 7.72 (d, 1H), 7.62 (d, 2H, J=9 Hz), 7.30 (s, 1H).

### Example 350

### 2-chloro-N-(4-(5-cyano-3-(trifluoromethyl)-1H-pyrazol-1-yl)phenyl)benzamide

Example 300D was processed as in Example (i)-a (Method 5, 6, or 7) to provide the title compound.

mp 162-163° C.;

MS (ESI) m/e 391 (M+1)⁺, 389 (M−1)⁻;

¹H NMR (DMSO-d₆, 300 MHz) δ10.91 (s, 1H), 8.09 (s, 1H), 7.98 (d, 2H), 7.79 (d, 2H), 7.67-7.46 (m, 4H);

IR (KBr) cm⁻¹ 3279, 3144, 2241, 1659, 1606, 1516, 1474, 1413, 1377, 1325, 1238, 1200, 1152, 1099,972,827,751.

### Example 351

### 3-chloro-N-(4-(5-cyano-3-(trifluoromethyl)-1H-pyrazol-1-yl)phenyl)isonicotinamide

Example 300D was processed as in Example (i)-a (Method 5, 6, or 7) to provide the title compound.

mp 221-222° C.;

MS (ESI−) m/e 374 (M−1)⁻;

¹H NMR (DMSO-d₆, 300 MHz) δ11.06 (s, 1H), 8.8 (d, 1H), 8.63 (dd, 1H), 8.08 (s, 1H), 7.97 (d, 2H), 7.82 (d, 2H), 7.76 (t, 1H);

IR (KBr) cm⁻¹ 3188, 3132, 3046, 2244, 1694, 1609, 1557, 1513, 1475, 1417, 1326, 1242, 1153, 1129, 1101, 972, 843.

### Example 352

### N-(4-(5-(difluoromethoxy)-3-(trifluoromethyl)-1H-pyrazol-1-yl)phenyl)-2-fluorobenzamide

Example 322B was processed as in Example (i)-a (Method 5, 6, or 7) to provide the title compound.

mp 130-131° C.;

MS (DCI/NH₃) m/e 433 (M+NH₄)⁺;

¹H NMR (DMSO-d₆, 300 MHz) δ10.7 (s, 1H), 7.92 (d, 2H), 7.7 (t, 1H), 7.61 (d, 2H), 7.6 (m, 1H), 7.32-7.41 (m, 2H), 7.15-7.65 (t, 1H), 6.82 (s, 1H).

### Example 353

### 2-chloro-N-(4-(5-(difluoromethoxy)-3-(trifluoromethyl)-1H-pyrazol-1-yl)phenyl)benzamide

Example 322B was processed as in Example (i)-a (Method 5, 6, or 7) to provide the title compound.

mp 122-123° C.;

MS (DCI/NH₃) m/e 449 (M+NH₄)⁺;

¹H NMR (DMSO-d₆, 300 MHz) δ10.82 (s, 1H), 7.92 (d, 2H), 7.62 (d, 2H), 7.59-7.66 (m, 2H), 7.45-7.57 (m, 2H), 7.16-7.64 (t, 1H), 6.85 (s, 1H).

### Example 354

### N-(4-(5-(difluoromethoxy)-3-(trifluoromethyl)-1H-pyrazol-1-yl)phenyl)-2,3-difluorobenzamide

Example 322B was processed as in Example (i)-a (Method 5, 6, or 7) to provide the title compound.

mp 154-155° C.;

MS (DCI/NH₃) m/e 451 (M+NH₄)⁺;

¹H NMR (DMSO-d₆, 300 MHz) δ10.69 (s, 1H), 7.79 (d, 2H), 7.53 (d, 3H), 7.4 (t, 1H), 7.2 (m, 1H), 7.09-7.45 (t, 1H), 6.69 (s, 1H).

### Example 355

### N-(4-(5-chloro-3-(3-furyl)-1H-pyrazol-1-yl)phenyl)-4-methyl-1,2,3-thiadiazole-5-carboxamide

### Example 355A

### 3-(3-furyl)-1-(4-nitrophenyl)-1H-pyrazol-5-ol

Ethyl β-oxo-3-furanpropionate (2 g, 10.9 mmol) in ethanol (100 mL) was added p-nitrophenylhydrazine (1.77 g, 11.6 mmol) and 4M HCl in dioxane. The mixture was heated to reflux for 3 hours. Upon cooling to room temperatur, the solvent was removed and the crude product was used in the next step without further purification.

MS (APCI) m/e 270 (M−H)⁻;

¹H NMR (DMSO-d₆, 300 MHz) δ8.35 (d, 2H), 8.20 (s, 1H), 8.13 (d, 2H), 7.75 (t, 1H), 6.88 (d, 1H), 6.90 (s, 1H).

### Example 355B

### 4-[5-chloro-3-(3-furyl)-1H-pyrazol-1-yl]aniline

Example 355A (1.0 g, 3.7 mmol) was added to phenylphosphonic dichloride (5 mL) in a sealed tube. The mixture was heated to 120° C. (oil bath) for 5 hours. Upon cooling to room temperature, the mixture was poured over a period of 30 minutes into an ice cold saturated aqueous solution of NaHCO₃ (100 mL). The resulting mixture was extracted with ethyl acetate (3×100 mL). The organic layers were combined and passed through a filter cake (100 mL silica gel and 15 g annhydrous magnesium) eluting with ethyl acetate. Solvent was removed leaving the product as a brown oil.

MS (DCI/NH₃) m/e 260 (M+H)⁺ (For aniline produced under analysis conditions.)

¹H NMR (DMSO-d₆, 300 MHz) δ8.4 (d, 2H), 8.2 (s, 1H), 8.0 (d, 2H), 7.8 (t, 1H), 7.1 (s, 1H), 6.9 (m, 1H)

The crude product was redissolved in ethanol/water (20 mL, 3:1/v:v). Iron powder (1.5 g, 27.3 mmol) and ammonium chloride (0.206 g, 3.89 mmol) were added, and the mixture was warmed to reflux for 1 hour. After cooling to room temperature, solvent was removed, and the residue was passed through a filter cake (100 mL silica gel and 15 g annhydrous magnesium sulfate) eluting with 20% acetone in hexanes (v:v). Fractions containing the desired product were combined, and solvent was removed leaving the product as a off white solid. (0.42 g, 44% overall yield).

MS (DCI/NH₃) m/e 260 (M+H)⁺;

¹H NMR (DMSO-d₆, 300 MHz) δ7.15 (d, 2H), 6.81 (s, 1H), 6.65 (d, 2H), 5.50 (s, 2H).

### Example 355

### N-(4-(5-chloro-3-(3-furyl)-1H-pyrazol-1-yl)phenyl)-4-methyl-1,2,3-thiadiazole-5-carboxamide

Example 355B was processed as in Example (i)-a (Method 5, 6, or 7) to provide the title compound.

mp 150-152° C.;

MS (DCI/NH₃) m/e 386 (M+H)⁺;

¹H NMR (DMSO-d₆, 300 MHz) δ8.17 (m, 1H), 7.89 (d, 2H), 7.76 (t, 1H), 7.62 (d, 2H), 6.96 (s, (1H), 6.86 (m, 1H), 2.84 (s, 3H).

### Example 356

### N-(4-(5-chloro-3-(3-furyl)-1H-pyrazol-1-yl)phenyl)-3-fluoroisonicotinamide

Example 355B was processed as in Example (i)-a (Method 5, 6, or 7) to provide the title compound.

mp 165-166° C.;

MS (DCI/NH₃) m/e 383 (M+H)⁺;

¹H NMR (DMSO-d₆, 300 MHz) δ10.98 (s, 1H), 8.80 (s, 1H), 8.63 (d, 1H), 8.18 (s, 1H), 7.90 (d, 2H), 7.77-7.74 (m, 2H), 7.65 (d, 2H), 6.97 (s, 1H), 6.87 (m, 1H).

### Example 357

### N-(4-(5-cyano-3-tetrahydro-2-furanyl-1H-pyrazol-1-yl)phenyl)-3-fluoroisonicotinamide

### Example 357A

### (+)-N′-(4-nitrophenyl)tetrahydro-2-furancarbohydrazide

A mixture of 4-nitrophenylhydrazine (719 mg, 4.70 mmol), tetrahydro-2-furoic acid (818 mg, 7.05 mmol), dimethylaminopyridine (860 mg, 7.05 mmol) and 1-(3-dimethylaminopropyl)-3-ethylcarbodiimide hydrochloride (1.34 g, 7.05 mmol) in methylene chloride (20 mL) was stirred at room temperature for 24 hours. The reaction mixture was diluted with ethyl acetate (150 mL) and the organic mixture was washed with 1N HCl solution (150 mL) and saturated sodium bicarbonate solution (150 mnL). The organic layer was dried over sodium sulfate, filtered and concentrated to a crude solid which was pure enough to use in the next step (1.20 g, 99% yield).

MS (DCI/NH₃) m/e 269 (M+NH₄)⁺;

¹H NMR (DMSO-d₆, 300 MHz) δ10.05 (s, 1H), 9.00 (s, 1H), 8.06 (d, 2H), 6.71 (d, 2H), 4.43 (dd, 1H), 3.95 (m, 1H), 3.80 (m, 1H), 2.20 (m, 1H), 2.01-1.80 (m, 3H).

### Example 357B

### N-(4-nitrophenyl)tetrahydro-2-furancarbohydrazonoyl chloride

A mixture of Example 357A (1.15 g, 4.58 mmol), triphenylphosphine (1.80 g, 6.87 mmol), and carbon tetrachloride (0.70 mL, 6.87 mmol) in methylene chloride (10 mL) and acetonitrile (5 mL) was stirred at room temperature for 20 hours. The reaction mixture was concentrated and purified by flash chromatography using 15% ethyl acetate/85% hexane to afford the title compound (420 mg, 34% yield) as an oil.

MS (DCI/NH₃) m/e 287 (M+NH₄)⁺;

¹H NMR (DMSO-d₆, 300 MHz) δ10.45 (s, 1H), 8.17 (d, 2H), 7.34 (d, 2H), 4.74 (t, 1H), 3.92-3.79 (m, 2H), 2.25-1.88 (m, 4H).

### Example 357C

### 1-(4-nitrophenyl)-3-tetrahydro-3-furanyl-1H-pyrazole-5-carbonitrile

A mixture of the Example 357C (207 mg, 0.768 mmol), 2-chloroacrylonitrile (100 mg, 1.15 mmol) and triethylamine (0.225 mL, 1.61 mmol) in toluene (5 mL) was heated to 70° C. for 2 hours. The reaction mixture was concentrated and purified by flash chromatography using 10% ethyl acetate/90% hexane to afford the title compound (155 mg, 71% yield) as an oil.

MS (DCI/NH₃) m/e 285 (M+H)⁺;

¹H NMR (DMSO-d₆, 300 MHz) δ8.48 (d, 2H), 8.07 (d, 2H), 7.56 (s, 1H), 4.99 (t, 1H), 3.92 (m, 1H), 3.80 (m, 1H), 2.27 (m, 1H), 2.00 (m, 3H).

### Example 357D

### 1-(4-aminophenyl)-3-tetrahydro-3-furanyl-1H-pyrazole-5-carbonitrile

The nitro group of Example 357C was reduced with iron as described previously.

MS (DCI/NH₃) m/e 255 (M+H)⁺;

¹H NMR (DMSO-d₆, 300 MHz) δ7.27 (d, 2H), 7.26 (s, 1H), 6.67 (d, 2H), 5.57 (s, 2H), 4.91 (t, 1H), 3.90 (m, 1H), 3.78 (m, 1H), 2.25 (m, 1H), 1.98 (m, 3H).

### Example 357

### N-(4-(5-cyano-3-tetrahydro-2-furanyl- lH-pyrazol- 1-yl)phenyl)-3-fluoroisonicotinamide

Example 357D was processed as in Example (i)-a (Method 5, 6, or 7) to provide the title compound.

mp 160-162° C.;

MS (DCI/NH₃) m/e 395 (M +NH₄)⁺;

¹H NMR (DMSO-d₆, 300 MHz) δ11.01 (s, 1H), 8.80 (s, 1H), 8.62 (d, 1H), 7.93 (d, 2H), 7.75 (t, 1H), 7.74 (d, 2H), 7.43 (s, 1H), 4.96 (t, 1H), 3.93 (m, 1H), 3.80 (m, 1H), 2.30 (m, 1H), 1.99 (m, 3H).

### Example 358

### N-(4-(5-(difluoromethoxy)-3-(1-methyl-1H-pyrrol-3-yl)-1H-pyrazol-1-yl)phenyl)-3-fluoroisonicotinamide

### Example 358A

### methyl 3-(1-methyl-1H-pyrrol-3-yl)-3-oxopropanoate

To a −78° C. solution of lithium hexamethyldisilazide (2 mL, 2 mmol) in tetrahydrofuran (5 mL) was added 3-acetyl-1-methylpyrrole (0.24 mL, 2 mmol). The reaction was warmed to 0° C. and stirred for 1 hour. The reaction mixture was again cooled to −78° C., and methylcyanoformate (0.19 mL, 2.4 mmol) was added. After stirring for 1 hour at −78° C., the reaction was slowly allowed to warm to room temperature. Then the reaction mixture was partitioned between ether and 1 N HCl. The organic layer was washed with saturated aqueous NaHCO₃ and brine, dried over NaSO₄, and concentrated to give crude material. Purification by HPLC (silica gel; acetone-hexane, 20:80) provided the desired product (0.18 g, 50% yield). MS (DCI/NH₃) m/e 199 (M+NH₄)⁺;

¹H NMR (DMSO-d₆, 300 MHz) δ7.64 (t, 1H), 6.84 (dd, 1H), 6.47 (dd, 1H), 5.45 (s, 1H), 3.67 (s, 3H), 3.66 (s, 3H).

### Example 358B

### 3-(1-methyl-1H-pyrrol-3-yl)-1-(4-nitrophenyl)-1H-pyrazol-5-ol

Condensation of of the P-ketoester prepared above with p-nitrophenylhydrazine using conditions previously described furnished the hydroxypyrazole in 64% yield.

MS (DCI/NH₃) m/e 302 (M+NH₄)⁺;

¹H NMR (DMSO-d₆, 300 MHz) δ8.34 (d, 2H), 8.17 (d, 2H), 7.3 (bt, 1H), 6.87 (bt, 1H), 6.49 (bt, 1H), 5.22 (bs, 1H), 3.68 (bs, 3H).

### Example 358C

### 5-(difluoromethoxy)-3-(1-methyl-1H-pyrrol-3-yl)-1-(4-nitrophenyl)-1H-pyrazole

The difluoromethoxy ether was prepared using alkylation conditions analogous to those described in the preparation of Example 322A in 59% yield.

MS (DCI/NH₃) m/e 335 (M+H)⁺;

¹H NMR (DMSO-d₆, 300 MHz) δ8.49 (d, 2H), 7.97 (d, 2H), 7.64-7.16 (t, 1H), 7.25 (t, 1H), 6.78 (t, 1H), 6.48 (s, 1H) 6.42 (dd, 1H), 3.65 (s, 3H).

### Example 358D

### 4-[5-(difluoromethoxy)-3-(1-methyl-1H-pyrrol-3-yl)-1H-pyrazol-1-yl]aniline

The aniline was prepared using the iron powder reduction conditions described in the preparation of 322B in 93% yield.

MS (DCI/NH₃) m/e 305 (M+H)⁺;

¹H NMR (DMSO-d₆, 300 MHz) δ7.51-7.03 (t, 1H), 7.15 (d, 2H), 7.09 (t, 1H), 6.7 (t, 1H), 6.63 (d, 2H), 6.3 (dd, 1H), 6.2 (s, 1H), 5.33 (s, 2H), 3.63 (s, 3H).

### Example 358

### N-(4-(5-(difluoromethoxy)-3-(1-methyl-1H-pyrrol-3-yl)-1H-pyrazol-1-yl)phenyl)-3-fluoroisonicotinamide

Example 358D was processed as in Example (i)-a (Method 5, 6, or 7) to provide the title compound.

mp 135-136° C.;

MS (DCI/NH₃) m/e 428 (M+NH₄)⁺;

¹H NMR (DMSO-d₆, 300 MHz) δ8.79 (s, 1H), 8.62 (d, 1H), 7.85 (d, 2H), 7.74 (t, 1H), 7.62 (d, 2H), 7.17 (t, 1H), 7.1-7.58 (t, 1H), 6.74 (t, 1H), 6.37 (dd, 1H), 6.34 (s, 1H), 3.65 (s, 3H).

### Example 359

### N-(4-(5-(difluoromethoxy)-3-(3-furyl)-1H-pyrazol-1-yl)phenyl)-3-fluoroisonicotinamide

### Example 359A

Example 355A (1 g, 3.89 mmol) and KCO₃ (1.53 g, 11.1 mmol) were combined in dimethylformamide (10 mL) and heated to 50° C. Chlorodifluoromethane was bubbled into the reaction mixture for 45 minutes. The mixture was then cooled to room temperature and partition between saturated NaCl solution (50 mL) and diethyl ether (50 mL). The organic layer was separated, the aqueous layer was washed again with diethyl ether (2×50 mL). The combined organic layers were dried over MgSO₄ and concentrated in vacuo. The residue was passed through a silica gel cake (150 mL) eluting with 20% acetone in hexanes and then concentrated in vacuo to provide the desired product.

MS (DCI/NH₃) m/e 292 (M+H)⁺ (For the aniline produced under the analysis conditions.);

¹H NMR (DMSO-d₆, 300 MHz) δ8.42 (d, 2H), 8.29 (s, 1H), 7.99 (d, 2H), 7.80 (t, 1H), 7.42 (t, 1H), 6.92 (s, 1H), 6.70 (s, 1H).

The crude product was redissolved in 20 mL of ethanol/water (3:1/v:v). Iron powder (1.5 g, 27.27 mmol) and ammonium chloride (0.206 g, 3.89 mmol) were added and the mixture was warmed to reflux for 1 hour. Upon cooling to room temperature, solvent was removed in vacuo, and the residue was loaded onto a filter cake (100 mL silica gel and 15 g anhydrous magnesium sulfate) and then eluted with 50% acetone in hexanes (v:v). Fractions containing the desired product were combined and solvent removed in vacuo leaving the product as an off white solid (0.52 g, 48% overall yield).

MS (DCI/NH₃) m/e 292 (M+H)⁺.

### Example 359

### N-(4-(5-(difluoromethoxy)-3-(3-furyl)-1H-pyrazol-1-yl)phenyl)-3-fluoroisonicotinamide

Example 359A was processed as in Example (i)-a (Method 5, 6, or 7) to provide the title compound.

mp 172-174° C.;

MS (DCI/NH₃) m/e 415 (M+H)⁺;

¹H NMR (DMSO-d₆, 300 MHz) δ8.77 (m, 1H), 8.60 (m, 1H), 8.20 (m, 1H), 7.87 (d, 2H), 7.75 (m, 1H), 7.73 (m, 1H), 7.63 (d, 2H), 7.35 (t, 1H), 6.89 (m, 1H), 6.55 (s, 1H).

### Example 360

### N-(4-(5-(difluoromethoxy)-3-(1-methyl-1H-pyrrol-2-yl)-1H-pyrazol-1-yl)phenyl)-3-fluoroisonicotinamide

### Example 360A

### ethyl 3-(1-methyl-1H-pyrrol-2-yl)-3-oxopropanoate

1-Methyl-2-pyrrolecarboxylic acid (1.25 g, 10 mmol) was heated to reflux in thionyl chloride (10 mL) for 2 hours. The excess thionyl chloride was removed under vacuum. Ethyl malonate (2.64 g, 20 mmol) in tetrahydrofuran (50 mL, containing 1 mg of 2,2′-bipyridyl as an indicator) was cooled to −70° C. n-Butyllithium (2.5 M solution in hexane) was added slowly until the pink color persisted for several minutes. After stirring for 5 minutes, 1-methyl-2-pyrrolecarboxylic acid chloride in tetrahydrofuran (6 mL) was then added dropwise . The reaction was stirred at −70° C. for 30 minutes and slowly warmed to room temperature for 2 hours. The reaction mixture was partitioned between ether and 1 N HCl. The organic layer was washed with saturated aqueous NaHCO₃ and brine, dried over Na₂SO₄ and concentrated to give the crude beta-ketoester (0.65 g, 33% yield).

MS (DCI/NH₃) m/e 213 (M+NH₄)⁺;

¹H NMR (DMSO-d₆, 300 MHz) δ7.2 (t, 1H), 7.11 (dd, 1H), 6.15 (dd, 1H), 4.1 (q, 2H), 3.89 (s, 2H), 3.84 (s, 3H), 1.18 (t, 3H).

### Example 360B

### 3-(1-methyl-1H-pyrrol-2-yl)-1-(4-nitrophenyl)-1H-pyrazol-5-ol

Condensation of of the beta-ketoester prepared above with p-nitrophenylhydrazine using conditions previously described furnished the hydroxypyrazole in 44% yield.

MS (DCI/NH₃) m/e 302 (M+NH₄)⁺;

¹H NMR (DMSO-d₆, 300 MHz) δ8.34 (d, 2H), 8.17 (bd, 2H), 6.85 (bs, 1H), 6.49 (bs, 1H), 6.04 (bs, 1H), 5.8 (bs, 1H), 3.95 (bs, 3H).

### Example 360C

### 5-(difluoromethoxy)-3-(1-methyl-1H-pyrrol-2-yl)-1-(4-nitrophenyl)-1H-pyrazole

The difluoromethoxy ether was prepared using alkylation conditions analogous to those described in the preparation of Example 322A in 23% yield.

MS (DCI/NH₃) m/e 335 (M+H)⁺;

¹H NMR (DMSO-d₆, 300 MHz) δ8.4 (d, 2H), 8.1 (d, 2H), 7.68-7.2 (t, 1H), 6.9 (t, 1H), 6.63 (s, 1H), 6.48 (dd, 1H) 6.08 (dd, 1H), 3.96 (s, 3H).

### Example 360D

### 4-[5-(difluoromethoxy)-3-(1-methyl-1H-pyrrol-2-yl)-1H-pyrazol-1-yl]aniline

The aniline was prepared using the iron powder reduction conditions described in the preparation of 322B in quantitative yield.

MS (DCI/NH₃) m/e 305 (M+H)⁺;

¹H NMR (DMSO-d₆, 300 MHz) 667 7.66-7.18 (t, 1H), 7.2 (d, 2H), 6.8 (t, 1H), 6.64 (d, 2H), 6.4 (dd, 1H), 6.36 (s, 1H), 6.03 (dd, 1H), 5.39 (s, 2H), 3.87 (s, 3H).

### Example 360

### N-(4-(5-(difluoromethoxy)-3-(1-methyl-1H-pyrrol-2-yl)-1H-pyrazol-1-yl)phenyl)-3-fluoroisonicotinamide

Example 360D was processed as in Example (i)-a (Method 5, 6, or 7) to provide the title compound.

mp 154-155° C.;

MS (DCI/NH₃) m/e 428 (M+NH₄)⁺;

¹H NMR (DMSO-d₆, 300 MHz) δ10.9 (s, 1H), 8.8 (s, 1H), 8.62 (d, 1H), 7.87 (d, 2H), 7.74 (t, 1H), 7.68 (d, 2H), 7.14-7.62 (t, 1H), 6.84 (t, 1H), 6.06 (t, 1H), 4.49 (s, 1H), 4.48 (t, 1H), 3.92 (s, 3H).

What is claimed is:

1. A compound having Formula I 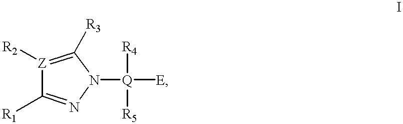
or a pharmaceutically acceptable salt or prodrug thereof, where R₁ and R₃ are independently selected from
(1) hydrogen,
(2) aryl,
(3) perfluoroalkyl of one to fifteen carbons,
(4) halo,
(5) —CN,
(6) —NO₂,
(7) —OH,
(8) —OG where G is a hydroxyl protecting group,
(9) —CO₂R₆ where R₆ is selected from
(a) hydrogen,
(b) cycloalkyl of three to twelve carbons,
(c) aryl,
(d) aryl substituted with 1, 2, 3, 4, or 5 substituents independently selected from
(i) alkyl of one to fifteen carbons,
(ii) alkoxy of one to fifteen carbons,
(iii) thioalkoxy of one to fifteen carbons,
(iv) halo,
(v) —NO₂, and
(vi) —N₃,
(e) a carboxy protecting group,
(f) alkyl of one to fifteen carbons,
(g) alkyl of one to fifteen carbons substituted with 1, 2, or 3, or 4 substituents independently selected from
(i) alkoxy of one to fifteen carbons,
(ii) thioalkoxy of one to fifteen carbons,
(iii) aryl,
(iv) aryl substituted with 1, 2, 3, 4, or 5 substituents independently selected from alkyl of one to fifteen carbons,
alkoxy of one to fifteen carbons,
thioalkoxy of one to fifteen carbons,
halo,
—NO₂, and
—N₃,
(v) cycloalkyl of three to twelve carbons, and
(vi) halo,
(h) alkenyl of three to fifteen carbons,
provided that a carbon of a carbon-carbon double bond is not attached directly to oxygen,
(i) alkynyl of three to fifteen carbons,
provided that a carbon of a carbon-carbon triple bond is not attached directly to oxygen, and
(j) cycloalkyl of three to twelve carbons,
(10) —L₁NR₇R₈ where L₁ is selected from
(a) a covalent bond,
(b) —X′C(X)— where X and X′ are independently O or S,
(c) —C(X)—, and
(d) —NR₆— and
R₇ and R₈ are independently selected from
(a) hydrogen,
(b) alkanoyl where the alkyl part is one to fifteen carbons,
(c) alkoxycarbonyl where the alkyl part is one to fifteen carbons,
(d) alkoxycarbonyl where the alkyl part is one to fifteen carbons and is substituted with 1 or 2 substituents selected from the group consisting of aryl,
(e) cycloalkyl of three to twelve carbons,
(f) aryl,
(g) aryl substituted with 1, 2, 3, 4, or 5 substituents independently selected from
(i) alkyl of one to fifteen carbons,
(ii) alkoxy of one to fifteen carbons,
(iii) thioalkoxy of one to fifteen carbons,
(iv) halo,
(v) —NO₂, and
(vi) —N₃,
(h) —OR₆,
provided that only one of R₇ or R₈ is -OR₆,
(i) a nitrogen protecting group,
(j) alkyl of one to fifteen carbons,
(k) alkyl of one to fifteen carbons substituted with 1, 2, or 3, or 4 substituents independently selected from
(i) alkoxy of one to fifteen carbons,
(ii) thioalkoxy of one to fifteen carbons,
(iii) aryl,
(iv) aryl substituted with 1, 2, 3, 4, or 5 substituents independently selected from alkyl of one to fifteen carbons,
alkoxy of one to fifteen carbons,
thioalkoxy of one to fifteen carbons,
halo,
—NO₂, and
—N₃,
(v) cycloalkyl of three to fifteen carbons,
(vi) halo,
(vii) —CO₂R₆, and
(viii) —OH,
(l) alkenyl of three to fifteen carbons,
provided that a carbon of a carbon-carbon double bond is not attached directly to nitrogen,
(m) alkynyl of three to fifteen carbons,
provided that a carbon of a carbon-carbon triple bond is not attached directly to nitrogen,
(n) —SO₂-alkyl, and
(o) cycloalkyl of three to twelve carbons, or
R₇ and R₈ together with the nitrogen atom to which they are attached form a ring selected from
(i) aziridine,
(ii) azetidine,
(iii) pyrrolidine,
(iv) piperidine,
(v) piperazine,
(vi) morpholine,
(vii) thiomorpholine, and
(viii) thiomorpholine sulfone
where (i)-(viii) can be optionally substituted with 1, 2, or 3 substituents selected from the group consisting of alkyl of one to fifteen carbons,
(11) —L₂R₉ where L₂ is selected from
(a) —L₁—,
(b) —O—, and
(c) —S(O)t— where t is 0, 1, or 2 and
R₉ is selected from
(a) cycloalkyl of three to twelve carbons,
(b) aryl
(c) aryl substituted with 1, 2, 3, 4, or 5 substituents independently selected from
(i) alkyl of one to fifteen carbons,
(ii) alkoxy of one to fifteen carbons,
(iii) thioalkoxy of one to fifteen carbons,
(iv) halo,
(v) —NO₂, and
(vi) —N₃,
(d) alkyl of one to fifteen carbons,
(e) heterocycle,
(f) alkenyl of two to fifteen carbons, and
(e) alkyl of one to fifteen carbons substituted with 1, 2, or 3, or 4 substituents independently selected from
(i) alkenyl of two to fifteen carbons,
(ii) alkoxy of one to fifteen carbons,
(iii) —CN,
(iv) —CO₂R₆,
(v) —OH,
provided that no two —OH groups are attached to the same carbon,
(vi) thioalkoxy of one to fifteen carbons,
(vii) alkynyl of two to fifteen carbons,
(viii) aryl,
(ix) aryl substituted with 1, 2, 3, 4, or 5 substituents independently selected from alkyl of one to fifteen carbons,
alkoxy of one to fifteen carbons,
thioalkoxy of one to fifteen carbons,
halo,
—NO₂, and
—N₃,
(x) cycloalkyl of three to twelve carbons, and
(xi) halo,
(xii) —NR₇R₈,
(xiii) heterocycle, and
(xiv) heterocycle substituted with 1, 2, or 3, or 4 substituents independently selected from
alkyl of one to fifteen carbons,
alkoxy of one to fifteen carbons,
thioalkoxy of one to fifteen carbons,
halo,
—NO₂, and
—N₃,
(12) alkyl of one to fifteen carbons substituted with 1, 2, 3, 4, or 5 halo substituents,
(13) alkyl of one to fifteen carbons,
(14) alkenyl of two to fifteen carbons,
(15) alkynyl of two to fifteen carbons
where (13)-(15) can be optionally substituted with
(a) (═X),
(b) alkanoyloxy where the alkyl part is one to fifteen carbons,
(c) alkoxy of one to fifteen carbons,
(d) alkoxy of one to fifteen carbons substituted with 1, 2, 3, 4, or 5 substituents selected from the group consisting of halo,
(e) thioalkoxy of one to fifteen carbons,
(f) perfluoroalkoxy of one to fifteen carbons,
(g) —N₃,
(h) —NO₂,
(i) —CN,
(j) —OH,
(k) —OG
(l) cycloalkyl of three to twelve carbons,
(m) halo,
(n) —CO₂R₆,
(o) —L₁NR₇R₈, and
(p) —L₂R₉,
(16) —L₂-heterocycle, and
(17) —L₂-heterocycle where the heterocycle is substituted with 1, 2, 3 or 4 substituents independently selected from
(a) alkyl of one to fifteen carbons,
(b) perfluoroalkyl of one to fifteen carbons,
(c) alkoxy of one to fifteen carbons,
(d) thioalkoxy of one to fifteen carbons,
(e) halo, and
(f) —NO₂,
(18) —NR_{X}C(O)NR_{Y}R_{Z} where R_{X}, R_{Y} and R_{Z} are independently selected from
(a) hydrogen and
(b) alkyl of one to fifteen carbons,
(19) —C(═NR_{X})NR_{Y}R_{Z},
(20) —NR_{X}C(═NR_{X′})NR_{Y}R_{Z} where R_{X}, R_{Y} and R_{Z} are defined previously and R_{X′} is selected from
(a) hydrogen and
(b) alkyl of one to fifteen carbons,
(21) —NR_{X}C(O)OR_{W}, where R_{W} is selected from
(a) alkyl of one to fifteen carbons and
(b) alkenyl of three to fifteen carbons,
provided that a carbon of a carbon-carbon double bond is not attached directly to oxygen, and
(22) —OC(O)NR₇R₈;
Z is nitrogen or carbon;
R₂ is absent or is selected from
(1) hydrogen,
(2) —CO₂R₆,
(3) alkyl of one to fifteen carbons,
(4) —C(O)R₆ where R₆ is selected from
(a) alkyl of one to fifteen carbons,
(b) aryl, and
(c) heterocycle,
(5) —C(O)NR_{7′}R_{8′} where R_{7′} and R_{8′} are independently selected from
(a) hydrogen,
(b) alkyl of one to fifteen carbons, or
R_{7′} and R_{8′} together with the nitrogen to which they are attached form a ring selected from
(i) piperidine,
(ii) piperazine,
(iii) morpholine,
(iv) thiomorpholine, and
(v) thiomorpholine sulfone
(6) perfluoroalkyl of one to fifteen carbons,
(7) cycloalkyl of three to ten carbons,
(8) alkyl of one to fifteen carbons substituted with 1, 2, 3, 4, or 5 substituents selected from the group conststing of halo,
(9) alkyl of one to fifteen carbons substituted with
(a) —CN,
(b) —OH,
provided that no two —OH groups are attached to the same carbon,
(c) (═X), and
(d) —CO₂R₆, and
(10) halogen;
provided that when X is nitrogen, R₂ is absent;
Q is aryl or heterocycle where, when Q is phenyl, the phenyl is 2-, 3-, or 4- substituted by E relative to the position of attachment of the pyrazole or 1,2,4-triazole ring to the phenyl ring;
R₄ and R₅ are independently selected from
(1) hydrogen,
(2) alkyl of one to fifteen carbons,
(3) alkyl of one to fifteen carbons substituted with 1, 2, 3, 4, or 5 halo substituents,
(4) alkyl of one to fifteen carbons substituted with
(a) —CN,
(b) —CO₂R₆,
(c) —L₁NR₇R₈, and
(d) —L₂R₉,
(5) perfluoroalkyl of one to fifteen carbons,
(6) —CN,
(7) —CO₂R₆,
(8) —L₁NR₇R₈,
(9) —L₂R₉,
(10) alkoxy of one to fifteen carbons,
(11) thioalkoxy of one to fifteen carbons,
(12) halo,
(13) —C(═NR₆)NR₇R₈,
(14) —NR₁₂(═NR₆)NR₇R₈ where R₆, R₇, and R₈ are defined previously and R₁₂ is selected from
(a) hydrogen,
(b) cycloalkyl of three to twelve carbons,
(c) aryl,
(d) alkyl of one to fifteen carbons, and
(e) alkyl of one to fifteen carbons substituted with 1, 2, or 3, or 4 substituents independently selected from
(i) alkenyl of two to fifteen carbons,
(ii) alkoxy of one to fifteen carbons,
(iii) thioalkoxy of one to fifteen carbons,
(iv) alkynyl of two to fifteen carbons, and
(v) aryl,
(15) —L₂-heterocycle, and
(16) —L₂-heterocycle where the heterocycle is substituted with 1, 2, 3, or 4 substituents independently selected from
(a) alkyl of one to fifteen carbons,
(b) perfluoroalkyl of one to fifteen carbons,
(c) alkoxy of one to fifteen carbons,
(d) thioalkoxy of one to fifteen carbons,
(e) halo,
(f) —N₃, and
(g) —NO₂;
E is
(1) —L₃-B where L₃ is selected from
(a) a covalent bond,
(b) alkenylene of two to six carbons in the Z or E configuration,
(c) alkynylene of two to six carbons,
(d) —C(X)—,
(e) —N═N—,
(f) —NR₇—,
(g) —N(R₇)C(O)N(R₈)—,
(h) —N(R₇)SO₂N(R₈)—,
(i) —X—,
(j) —(CH₂)ₘO—,
(k) —O(CH₂)ₘ—,
(l) —N(R₇)C(X)—,
(m) —C(X)N(R₇)—,
(n) —S(O)ₜ(CH₂)ₘ—,
(o) —(CH₂)ₘS(O)ₜ—,
(p) —NR₇(CH₂)ₘ—,
(q) —(CH₂)ₘNR₇—,
(r) —NR₇S(O)ₜ—,
(s) —S(O)ₜNR₇—,
(t) —N═C(H)—,
(u) —C(H)═N—,
(v) —ON═CH—,
(w) —CH═NO—
where (g)-(w) are drawn with their left ends attached to Q,
(x) —N(R₇)C(O)N(R₁₀)(R₁₁)— where R₁₀ and R₁₁ together with the nitrogen atom to which they are attached form a ring selected from
(i) morpholine,
(ii) thiomorpholine,
(iii) thiomorpholine sulfone, and
(iv) piperidine
where (i)-(iv) are attached to Q through the nitrogen to which is attached R₇ and to B through a carbon in the ring,
(y) —N(R₇)SO₂N(R₁₀)(R₁₁)—, and
(z) —N(R₇)C(O)N(R₁₀)(R₁₁)— and B is selected from
(a) alkyl of one to fifteen carbons,
(b) alkenyl of three to fifteen carbons in the E or Z configuration, provided that a carbon of a carbon-carbon double bond is not directly attached to L₃ when L₃ is other than a covalent bond,
(c) alkynyl of three to fifteen carbons, provided that a carbon of a carbon-carbon triple bond is not directly attached to L₃ when L₃ is other than a covalent bond
where (a), (b) and (c), can be optionally substituted with 1, 2, 3, or 4 substituents independently selected from
(i) 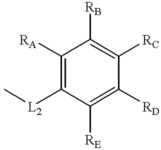
where L₂ is defined previously and R_{A}, R_{B}, R_{C}, R_{D}, and R_{E} are independently selected from hydrogen,
alkanoyl where the alkyl part is one to fifteen carbons,
alkanoyloxy where the alkyl part is one to fifteen carbons,
alkoxy of one to fifteen carbons,
thioalkoxy of one to fifteen carbons,
alkoxy of one to fifteen carbons substituted with 1, 2, 3, 4, or 5 substituents selected from the group consisting of halo,
perfluoroalkyl of one to fifteen carbons,
perfluoroalkoxy of one to fifteen carbons,
—N₃,
—NO₂,
—CN,
—OH,
—OG,
cycloalkyl of three to fifteen carbons,
halo,
—CO₂R₆
—L₁NR₇R₈
—L₂R₉ alkyl of one to fifteen carbons,
alkyl of one to fifteen carbons substituted with 1, 2, 3, 4, or 5 substituents independently selected from (═X),
alkanoyloxy where the alkyl part is one to fifteen carbons,
alkoxy of one to fifteen carbons,
thioalkoxy of one to fifteen carbons,
alkoxy of one to fifteen carbons substituted with 1, 2, 3,4, or 5 halo substituents,
perfluoroalkoxy of one to fifteen carbons,
—N₃,
—NO₂,
—CN,
—OH,
provided that no two —OH groups are attached to the same carbon,
—OG,
cycloalkyl of three to fifteen carbons,
halo,
—CO₂R₆,
—L₁NR₇R₈, and
—L₂R₉,
—L₂-heterocycle, and
—L₂-heterocycle where the heterocycle is substituted with 1, 2, 3, or 4 substituents independently selected from alkyl of one to fifteen carbons,
perfluoroalkyl of one to fifteen carbons,
alkoxy of one to fifteen carbons,
thioalkoxy of one to fifteen carbons,
halo,
—NR_{X}C(O)NR_{Y}R_{Z},
—C(═NR_{X})R_{Y}R_{Z},
—NO₂, and
—N₃,
(ii) (═X)
(iii) alkanoyloxy where the alkyl part is one to fifteen carbons,
(iv) alkoxy of one to fifteen carbons,
(v) alkoxy of one to fifteen carbons substituted with 1, 2, 3, 4, or 5 substituents selected from the group consisting of halo,
(vi) thioalkoxy of one to fifteen carbons,
(vii) perfluoroalkoxy of one to fifteen carbons,
(viii) —N₃,
(ix) —NO₂,
(x) —CN,
(xi) —OH,
provided that no two —OH groups are attached to the same carbon,
(xii) —OG,
(xiii) cycloalkyl of three to fifteen carbons,
(xiv) halo,
(xv) —CO₂R₆,
(xvi) —L₁NR₇R₈,
(xvii) perfluoroalkyl of one to fifteen carbons,
(xviii) —L₂-heterocycle, and
(xix) —L₂-heterocycle where the heterocycle is substituted with 1, 2, 3, or 4 substituents independently selected from (═X),
alkanoyl where the alkyl part is one to fifteen carbons,
alkanoyloxy where the alkyl part is one to fifteen carbons,
alkoxy of one to fifteen carbons,
alkoxy of one to fifteen carbons substituted with 1, 2, 3, 4, or 5 substituents selected from the group consisting of halo,
thioalkoxy of one to fifteen carbons,
perfluoroalkyl of one to fifteen carbons,
perfluoroalkoxy of one to fifteen carbons,
—N₃,
—-NO₂,
—CN,
—OH,
provided that no two —OH groups are attached to the same carbon,
—OG,
cycloalkyl of three to fifteen carbons,
halo,
—CO₂R₆,
—L₁NR₇R₈, and
—L₂R₉,
(d) cycloalkyl of three to twelve carbons,
(e) cycloalkenyl of four to twelve carbons,
provided that a carbon of a carbon-carbon-double bond is not attached directly to L₃ when L₃ is other than a covalent bond
where (d) and (e) can be optionally substituted with 1, 2, 3, 4, or 5 substituents independently selected from
(i) alkyl of one to fifteen carbons,
(ii) aryl,
(iii) alkoxy of one to fifteen carbons,
(iv) thioalkoxy of one to fifteen carbons,
(v) halo,
(vi) —OH,
provided that no two —OH groups are attached to the same carbon,
(vii) oxo,
(viii) perfluoroalkyl,
(ix) heterocycle, and
(x) heterocycle substituted with 1, 2, 3, 4, or 5 substituents independently selected from alkyl of one to fifteen carbons,
perfluoroalkyl of one to fifteen carbons,
alkoxy of one to fifteen carbons,
thioalkoxy of one to fifteen carbons,
halo,
—NO₂, and
—N₃,
(f) 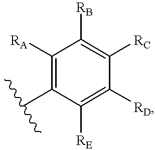
provided that when R₁ and R₃ are both perfluoroalkyl of one carbon, Z is carbon, R₂ is hydrogen, Q is phenyl that is 4-substituted by E relative to the position of attachment of the pyrazole ring to the phenyl group, R₄ and R₅ are hydrogen, E is —L₃-B, L₃ is —N(R₇)C(X)—, R₇ is hydrogen, X is oxygen, and R_{A}, R_{B}, R_{D}, and R_{E} are hydrogen, R_{C} is other than chloro, and
(g) heterocycle where the heterocycle can be optionally substituted with 1, 2, 3, or 4 substituents independently selected from
(i) (═X),
(ii) alkanoyl where the alkyl part is one to fifteen carbons,
(iii) alkanoyloxy where the alkyl part is one to fifteen carbons,
(iv) alkoxy of one to fifteen carbons,
(v) alkoxy of one to fifteen carbons substituted with 1, 2, 3, 4, or 5 substituents selected from the group consisting of halo,
(vi) halo,
(vii) thioalkoxy of one to fifteen carbons,
(viii) perfluoroalkyl of one to fifteen carbons,
(ix) perfluoroalkoxy of one to fifteen carbons,
(x) —N₃,
(xi) —NO₂,
(xii) —CN,
(xiii) —OH,
provided that no two —OH groups are attached to the same carbon,
(xiv) —OG,
(xv) cycloalkyl of three to fifteen carbons,
(xvi) halo,
(xvii) —CO₂R₆,
(xviii) alkyl optionally substituted with —OH,
(xix) —L₁NR₇R₈, and
(xx) —L₂R₉,
provided that when R₁ and R₃ are perfluoroalkyl of one carbon, Z is carbon, R₂ is hydrogen, Q is phenyl that is 4-substituted by E relative to the position of attachment of the pyrazole ring to the phenyl group, R₄ and R₅ are hydrogen, E is —L₃-B, L₃ is —N(R₇)C(X)—, R₇ is hydrogen, X is oxygen, and B is a 1,2,3-thiadiazolyl ring attached to L₃ through the 5-position of the ring, the substituent at the 4-position of the 1,2,3-thiadiazolyl ring is other than alkyl of one carbon, and
further provided that when R₁ and R₃ are perfluoroalkyl of one carbon, Z is carbon, R₂ is hydrogen, Q is phenyl that is 4-substituted by E relative to the position of attachment of the pyrazole ring to the phenyl group, R4 and R₅ are hydrogen, E is —L₃-B, L₃ is —N(R₇)C(X)—, R₇ is hydrogen, X is oxygen, and B is an isoxazole ring attached to L₃ through the 4-position of the ring, the substituents at the 3- and 5- positions of the isoxazole ring are not both alkyl of one carbon or
(2) 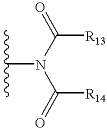
where R₁₃ and R₁₄ are independently selected from
(a) hydrogen,
(b) alkyl of one to fifteen carbons,
(c) alkenyl of three to fifteen carbons in the E or Z configuration, provided that a carbon of a carbon-carbon double bond is not attached directly to the C(═O) group,
(d) alkynyl of three to fifteen carbons, provided that a a carbon-carbon triple bond is not directly attached to the C(═O) group
where (b), (c), and (d) can be optionally substituted with 1, 2, 3, or 4 substituents independently selected from
(i) 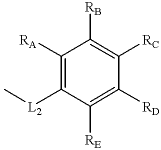
(ii) (═X),
(iii) alkanoyloxy where the alkyl part is one to fifteen carbons,
(iv) alkoxy of one to fifteen carbons,
(v) alkoxy of one to fifteen carbons substituted with 1, 2, 3, 4, or 5 substituents selected from the group consisting of halo,
(vi) thioalkoxy of one to fifteen carbons,
(vii) perfluoroalkoxy of one to fifteen carbons,
(viii) —N₃,
(ix) —NO₂,
(x) —CN,
(xi) —OH,
provided that no two —OH groups are attached to the same carbon,
(xii) —OG,
(xiii) cycloalkyl of three to fifteen carbons,
(xiv) halo,
(xv) —CO₂R₆,
(xvi) —L₁NR₇R₈,
(xvii) perfluoroalkyl of one to fifteen carbons,
(xviii) —L₂-heterocycle, and
(xix) —L₂-heterocycle where the heterocycle is substituted with 1, 2, 3,or 4 substituents independently selected from (═X),
alkanoyl where the alkyl part is one to fifteen carbons,
alkanoyloxy where the alkyl part is one to fifteen carbons,
alkoxy of one to fifteen carbons,
alkoxy of one to fifteen carbons substituted with 1, 2, 3, 4, or 5 substituents selected from the group consisting of halo,
thioalkoxy of one to fifteen carbons,
perfluoroalkyl of one to fifteen carbons,
perfluoroalkoxy of one to fifteen carbons,
—N₃,
—NO₂,
—CN,
—OH,
provided that no two —OH groups are attached to the same carbon,
—OG,
cycloalkyl of three to fifteen carbons,
halo,
—CO₂R₆,
—L₁NR₇R₈,
—L₂R₉,
(e) cycloalkyl of three to twelve carbons,
(f) cycloalkenyl of four to twelve carbons,
provided that a carbon of a carbon-carbon double bond is not attached directly to the C(═O) group
where (e) and (f) can be optionally substituted with 1, 2, 3, 4, or 5 substituents independently selected from
(i) alkyl of one to fifteen carbons,
(ii) aryl,
(iii) alkoxy of one to fifteen carbons,
(iv) thioalkoxy of one to fifteen carbons,
(v) halo,
(vi) —OH,
provided that no two —OH groups are attached to the same carbon,
(vii) heterocycle, and
(viii) heterocycle substituted with 1, 2, 3, 4, or 5 substituents independently selected from alkyl of one to fifteen carbons,
perfluoroalkyl of one to fifteen carbons,
alkoxy of one to fifteen carbons,
thioalkoxy of one to fifteen carbons,
halo,
—NO₂, and
—N₃,
(g) heterocycle, and
(h) heterocycle substituted with 1, 2, 3, or 4 substituents independently selected from
(i) (═X),
(ii) alkanoyl where the alkyl part is one to fifteen carbons,
(iii) alkanoyloxy where the alkyl part is one to fifteen carbons,
(iv) alkoxy of one to fifteen carbons,
(v) alkoxy of one to fifteen carbons substituted with 1, 2, 3, 4, or 5 substituents selected from the group consisting of halo,
(vi) thioalkoxy of one to fifteen carbons,
(vii) perfluoroalkyl of one to fifteen carbons,
(viii) perfluoroalkoxy of one to fifteen carbons,
(ix) —N₃,
(x) —NO₂,
(xi) —CN,
(xii) —OH,
provided that no two —OH groups are attached to the same carbon,
(xiii) —OG,
(xiv) cycloalkyl of three to fifteen carbons,
(xv) halo,
(xvi) —CO₂R₆,
(xvii) —L₁NR₇R₈,
(xviii) —L₂R₉,
provided that at least one of R₁₃ and R₁₄ is other than hydrogen, or R₁₃ and R₁₄ together with the nitrogen to which they are attached form a ring selected from
(a) succinimidyl,
(b) maleimidyl,
(c) glutarimidyl,
(d) phthalimidyl,
(e) naphthalimidyl,
(f) 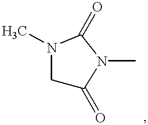
(g) 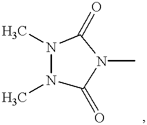
(h) 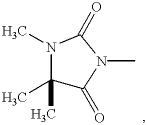
(i) 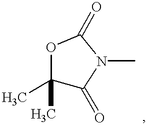
(j) 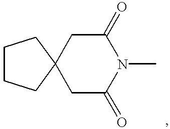
(k) 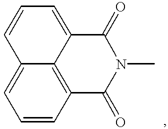
(l) 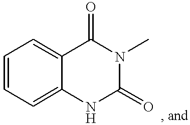
(m) 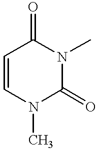
where (a)-(m) can be optionally substituted with 1, 2, 3, 4, or 5 substituents selected from halo and —L₂R₉.

2. A compound according to claim 1 of Formula 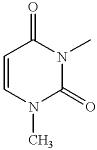
or a pharmaceutically acceptable salt or prodrug thereof, where
Z is carbon, R₂ is hydrogen, and R₁, R₃, R₄, R₅, and E are defined above.

3. A compound according to claim 2 where R₁ is perfluoroalkyl of one to fifteen carbons and R₄ and R₅ are hydrogen.

4. A compound according to claim 3 where L₃ is —N(R₇)C(X)—, R₇ is hydrogen, and W is O.

5. A compound according to claim 4 selected from
N-[4-[3,5-bis(trifluoromethyl)-1H-pyrazol-1-yl]phenyl]-cyclopropanecarboxamide,
N-[4-[3,5-bis(trifluoromethyl)-1H-pyrazol-1-yl]phenyl]-2,2,3,3-tetramethylcyclopropanecarboxamide,
N-[4-[3,5-bis(trifluoromethyl)-1H-pyrazol-1-yl]phenyl]-2,2-dichloro-1-methylcyclopropanecarboxamide,
N-[4-[3,5 -bis(trifluoromethyl)-1H-pyrazol-1-yl]phenyl]-2-oxo-6-pentyl-2H-pyran-3-carboxamide,
N-[4-[3,5-bis(trifluoromethyl)-1H-pyrazol-1-yl]phenyl]-1-cyclohexene-1-carboxamide,
N-[4-[3,5-bis(trifluoromethyl)-1H-pyrazol-1-yl]phenyl]-2-methylcyclopropanecarboxamide,
N-[4-[3,5-bis(trifluoromethyl)-1H-pyrazol-1-yl]phenyl]-5-(3,5-dichlorophenoxy)-2-furancarboxamide,
N-[4-[3,5-bis(trifluoromethyl)-1H-pyrazol-1-yl]phenyl]-1-methyl-2-cyclohexene-1-carboxamide,
N-[4-[3,5-bis(trifluoromethyl)-1H-pyrazol-1-yl]phenyl]-1-cyclopentene-1-carboxamide,
N-[4-[3,5-bis(trifluoromethyl)-1H-pyrazol-1-yl]phenyl]-3-methoxycyclohexanecarboxamide,
N-[4-[3,5-bis(trifluoromethyl)-1H-pyrazol-1-yl]phenyl]-2-butynamide,
N-[4-[3,5-bis(trifluoromethyl)-1H-pyrazol-1-yl]phenyl]-3-furancarboxamide,
N-[4-[3,5-bis(trifluoromethyl)-1H-pyrazol-1-yl]phenyl]-2-methyl-3-nitrobenzamide,
N-[4-[3,5-bis(trifluoromethyl)-1H-pyrazol-1-yl]phenyl]-1-hydroxycyclopropanecarboxamide,
N-[4-[3,5-bis(trifluoromethyl)-1H-pyrazol-1-yl]phenyl]cycloheptanecarboxamide,
N-[4-[3,5-bis(trifluoromethyl)-1H-pyrazol-1-yl]phenyl]-2-benzofurancarboxamide,
N-[4-[3,5-bis(trifluoromethyl)-1H-pyrazol-1-yl]phenyl]-5-fluoro-1H-indole-2-carboxamide,
(E)-N-[4-[3,5-bis(trifluoromethyl)-1H-pyrazol-1-yl]phenyl]-3-(2-chlorophenyl)-2-propenamide,
2-benzoyl-N-[4-[3,5-bis(trifluoromethyl)-1H-pyrazol-1-yl]phenyl]benzamide,
3a(S)-(3aα,4β,6aα)-N-[4-[3,5-bis(trifluoromethyl)-1H-pyrazol-1-yl]-phenyl]hexahydro-2-oxo-1H-thieno[3,4-d]imidazole-4-pentanamide,
N-[4-[3,5-bis(trifluoromethyl)-1H-pyrazol-1-yl]phenyl]-3-iodobenzamide,
exo-N-[4-[3,5-bis(trifluoromethyl)-1H-pyrazol-1-yl]phenyl]bicyclo[2.2.1]hept-5-ene-2-carboxamide,
N-[4-[3,5-bis(trifluoromethyl)-1H-pyrazol-1-yl]phenyl]-2-methylcyclohexanecarboxamide,
(R)-phenylmethyl [1-[[[4-[3,5-bis(trifluoromethyl)-1H-pyrazol-1-yl]phenyl]amino]carbonyl]propyl]carbamate,
N-[4-[3,5-bis(trifluoromethyl)-1H-pyrazol-1-yl]phenyl]-3-cyclohexene-1-carboxamide,
N-[4-[3,5-bis(trifluoromethyl)-1H-pyrazol-1-yl]phenyl]-1-methylcyclopropanecarboxamide,
N-[4-[3,5-bis(trifluoromethyl)-1H-pyrazol-1-yl]phenyl]-5-methyl-2-thiophenecarboxamide,
N-[4-[3,5-bis(trifluoromethyl)-1H-pyrazol-1-yl]phenyl]-4-(1H-pyrrol-1-yl)benzamide,
N-[4-[3,5-bis(trifluoromethyl)-1H-pyrazol-1-yl]phenyl]-7-methoxy-2-benzofurancarboxamide,
N-[4-[3,5-bis(trifluoromethyl)-1H-pyrazol-1-yl]phenyl]-3-(hydroxymethyl)benzamide,
N-[4-[3,5-bis(trifluoromethyl)-1H-pyrazol-1-yl]phenyl]-2-cyanoacetamide,
N-[4-[3,5-bis(trifluoromethyl)-1H-pyrazol-1-yl]phenyl]-2-cyclohexane-1-carboxamide,
N-[4-[3,5-bis(trifluoromethyl)-1H-pyrazol-1-yl]phenyl]-4-methylcyclohexanecarboxamide,
(R)-N-[4-[3,5-bis(trifluoromethyl)-1H-pyrazol-1-yl]phenyl]-α-methoxy-α-(trifluoromethyl)benzeneacetamide,
N-[4-[3,5-bis(trifluoromethyl)-1H-pyrazol-1-yl]phenyl]heptanamide,
N-[4-[3,5-bis(trifluoromethyl)-1H-pyrazol-1-yl]phenyl]-2-phenoxybenzamide,
3-Amino-N-[4-[3,5-bis(trifluoromethyl)-1H-pyrazol-1-yl]phenyl]benzamide,
4-Amino-N-[4-[3,5-bis(trifluoromethyl)-1H-pyrazol-1-yl]phenyl]benzanmide,
4-Azido-N-[4-[3,5-bis(trifluoromethyl)-1H-pyrazol-1-yl]phenyl]benzamide,
N-[4-[3,5-bis(trifluoromethyl)-1H-pyrazol-1-yl]-2-thiopheneacetamide,
N-[3,5-bis(trifluoromethyl)-1H-pyrazol-1-yl]phenyl]-1-tricyclo [3.3.1.1^{3,7}]-decanecarboxmide,
N-[4-[3,5-bis(trifluoromethyl)-1H-pyrazol-1-yl]phenyl]-N²-[(1,1-dimethylethoxy)carbonyl]-L-asparagine, phenylmethyl ester,
1,1-dimethylethyl [7-[[4-[3,5-bis(trifluoromethyl)-1H-pyrazol-1-yl]phenyl]amino]-
N-[4-[3,5-bis(trifluoromethyl)-1H-pyrazol-1-yl]phenyl]-3-(methylthio)propanamide,
N-[4-([3,5-bis(trifluoromethyl)-1H-pyrazol-1-yl]phenyl]-1-naphthylenecarboxamide,
N-[4-[3,5-bis(trifluoromethyl)-1H-pyrazol-1-yl]phenyl]-4-cyanobenzamide,
(trans)-N-[4-[3,5-bis(trifluoromethyl)-1H-pyrazol-1-yl]phenyl]-2-phenylcyclopropanecarboxamide,
N-[4-[3,5-bis(trifluoromethyl)-1H-pyrazol-1-yl]phenyl]-4-iodobenzamide,
N-[4-[3,5-bis(trifluoromethyl)-1H-pyrazol-1-yl]phenyl]-3-chloropropanamide,
N-[4-[3,5-bis(trifluoromethyl)-1H-pyrazol-1-yl]phenyl]-4-methoxybenzamide,
N-[4-[3,5-bis(trifluoromethyl)-1H-pyrazol-1-yl]phenyl]-2-ethylhexanamide,
N-[4-[3,5-bis(trifluoromethyl)-1H-pyrazol-1-yl]phenyl]-4-hydroxybenzamide,
N-[4-[3,5-bis(trifluoromethyl)-1H-pyrazol-1-yl]phenyl]-4-(hexyloxy)benzamide,
N-[4-[3,5-bis(trifluoromethyl)-1H-pyrazol-1-yl]phenyl]-3-methylbenzamide,
2-(acetyloxy)-N-[4-[3,5-bis(trifluoromethyl)-1H-pyrazol-1-yl]phenyl]benzamide,
N-[4-[3,5-bis(trifluoromethyl)-1H-pyrazol-1-yl]phenyl]-2,4,6-trimethylbenzamide,
N-[4-[3,5-bis(trifluoromethyl)-1H-pyrazol-1-yl]phenyl]-1-methyl-5-nitro-1H-pyrazole-4-carboxamide,
N-[4-[3,5-bis(trifluoromethyl)-1H-pyrazol-1-yl]phenyl]-4-bromobenzamide,
N-[4-[3,5-bis(trifluoromethyl)-1H-pyrazol-1-yl]phenyl]-2-(dimethylamino)benzamide,
N-[4-[3,5-bis(trifluoromethyl)-1H-pyrazol-1-yl]phenyl]-3-(dimethylamino)benzamide,
N-[4-[3,5-bis(trifluoromethyl)-1H-pyrazol-1-yl]phenyl]-4-(trifluoromethyl)benzamide,
N-[4-[3,5-bis(trifluoromethyl)-1H-pyrazol-1-yl]phenyl]-4-fluorobenzamide,
N-[4-[3,5-bis(trifluoromethyl)-1H-pyrazol-1-yl]phenyl]-2-chlorobenzamide,
N-[4-[3,5-bis(trifluoromethyl)-1H-pyrazol-1-yl]phenyl]benzamide,
N-[4-[3,5-bis(trifluoromethyl)-1H-pyrazol-1-yl]phenyl]-4-nitrobenzamide,
N-[4-[3,5-bis(trifluoromethyl)-1H-pyrazol-1-yl]phenyl]-2-methylbenzamide,
N-[4-[3,5-bis(trifluoromethyl)-1H-pyrazol-1-yl]phenyl]-3,4-dimethoxybenzamide,
N-[4-[3,5-bis(trifluoromethyl)-1H-pyrazol-1-yl]phenyl]cyclopentanepropanamide,
N-[4-[3,5-bis(trifluoromethyl)-1H-pyrazol-1-yl]phenyl]-4-methylbenzamide,
N-[4-[3,5-bis(trifluoromethyl)-1H-pyrazol-1-yl]phenyl]-3-(trifluoromethyl)benzamide,
N-[4-[3,5-bis(trifluoromethyl)-1H-pyrazol-1-yl]phenyl]-3-methyl-2-butenamide,
N-[4-[3,5-bis(trifluoromethyl)-1H-pyrazol-1-yl]phenyl]-2-hydroxybenzamide,
N-[4-[3,5-bis(trifluoromethyl)-1H-pyrazol-1-yl]phenyl]-3-hydroxybenzamide,
N-[4-[3,5-bis(trifluoromethyl)-1H-pyrazol-1-yl]phenyl]-2,4-dimethyl-5-thiazolecarboxamide,
N-[4-[3,5-bis(trifluoromethyl)-1H-pyrazol-1-yl]phenyl]-3-pyridinecarboxamide,
N-[4-[3,5-bis(trifluoromethyl)-1H-pyrazol-1-yl]phenyl]-4-(hydroxymethyl)benzamide,
N-[4-[3,5-bis(trifluoromethyl)-1H-pyrazol-1-yl]phenyl]-4-(methylsulfonyl)benzamide,
N-[4-[3,5-bis(trifluoromethyl)-1H-pyrazol-1-yl]phenyl]-2-iodobenzamide,
N-[4-[3,5-bis(trifluoromethyl)-1H-pyrazol-1-yl]phenyl]-4-heptybenzamide,
N-[4-[3,5-bis(trifluoromethyl)-1H-pyrazol-1-yl]phenyl]-2-furancarboxamide,
N-[4-[3,5-bis(trifluoromethyl)-1H-pyrazol-1-yl]phenyl]-2-fluorobenzamide,
N-[4-[3,5-bis(trifluoromethyl)-1H-pyrazol-1-yl]phenyl]-N′-methyl-1,2-benzenedicarboxamide,
N-[4-[3,5-bis(trifluoromethyl)-1H-pyrazol-1-yl]phenyl]-4-pyridinecarboxamide,
N-[4-[3,5-bis(trifluoromethyl)-1H-pyrazol-1-yl]phenyl]-4-chloro-2-nitrobenzamide,
N-[4-[3,5-bis(trifluoromethyl)-1H-pyrazol-1-yl]phenyl]-4-cinnolinecarboxamide,
4-acetyl-N-[4-[3,5-bis(trifluoromethyl)-1H-pyrazol-1-yl]phenyl]benzamide,
1,1-dimethylethyl 4-[[[4-[3,5-bis(trifluoromethyl)-1H-pyrazol-1-yl]phenyl]-amino]carbonyl]-1-piperidinecarboxylate,
N-[4-[3,5-bis(trifluoromethyl)-1H-pyrazol-1-yl]phenyl]-2-pyridinecarboxamide,
N-[4-[3,5-bis(trifluoromethyl)-1H-pyrazol-1-yl]phenyl]-4-(diethylamino)benzamide,
N-[4-[3,5-bis(trifluoromethyl)-1H-pyrazol-1-yl]phenyl]cyclopentanecarboxamide,
N-[4-[3,5-bis(trifluoromethyl)-1H-pyrazol-1-yl]phenyl]cyclohexanecarboxamide,
N-[4-[3,5-bis(trifluoromethyl)-1H-pyrazol-1-yl]phenyl]-4-piperidinecarboxamide,
N-[4-[3,5-bis(trifluoromethyl)-1H-pyrazol-1-yl]phenyl]-3-(methylsulfonyl)benzamide,
N-[4-[3,5-bis(trifluoromethyl)-1H-pyrazol-1-yl]phenyl]-2-(trifluoromethyl)benzamide,
methyl 3-[[[4-[3,5-bis(trifluoromethyl)-1H-pyrazol-1-yl]phenyl]amino]-carbonyl]benzoate,
N-[4-[3,5-bis(trifluoromethyl)-1H-pyrazol-1-yl]phenyl]-3-chlorobenzamide,
N-[4-[3,5-bis(trifluoromethyl)-1H-pyrazol-1-yl]phenyl]-2-thiophenecarboxamide,
N-[4-[3,5-bis(trifluoromethyl)-1H-pyrazol-1-yl]phenyl]-1,4-benzenedicarboxamide,
N-[4-[3,5-bis(trifluoromethyl)-1H-pyrazol-1-yl]phenyl]-3,5-dinitrobenzamide,
N-[4-[3,5-bis(trifluoromethyl)-1H-pyrazol-1-yl]phenyl]-2,4-difluorobenzamide,
N-[4-[3,5-bis(trifluoromethyl)-1H-pyrazol-1-yl]phenyl]-2-nitrobenzamide,
N-[4-[3,5-bis(trifluoromethyl)-1H-pyrazol-1-yl]phenyl]-3-cyanobenzamide,
N-[4-[3,5-bis(trifluoromethyl)-1H-pyrazol-1-yl]phenyl]-1,3-benzenedicarboxamide,
N-[4-[3,5-bis(trifluoromethyl)-1H-pyrazol-1-yl]phenyl]-3-nitrobenzamide,
3-(aminosulfonyl)-N-[4-[3,5-bis(trifluoromethyl)-1H-pyrazol-1-yl]phenyl]benzamide,
methyl 4-[[[4-[3,5-bis(trifluoromethyl)-1H-pyrazol-1-yl]phenyl]amino]-carbonyl]benzoate,
N-[4-[3,5-bis(trifluoromethyl)-1H-pyrazol-1-yl]phenyl]-2-methoxybenzamide,
N-[4-[3,5-bis(trifluoromethyl)-1H-pyrazol-1-yl]phenyl]-3-bromobenzamide,
N-[4-[3,5-bis(trifluoromethyl)-1H-pyrazol-1-yl]phenyl]-3-methoxybenzamide,
N-[4-[3,5-bis(trifluoromethyl)-1H-pyrazol-1-yl]phenyl]-3-fluorobenzamide,
N-[4-[3,5-bis(trifluoromethyl)-1H-pyrazol-1-yl]phenyl]-2-bromobenzamide,
N-[4-[3,5-bis(trifluoromethyl)-1H-pyrazol-1-yl]phenyl]-1,3-benzodioxole-5-carboxamide,
N-[4-[3,5-bis(trifluoromethyl)-1H-pyrazol-1-yl]phenyl]-2,6-dichloro-3-pyridinecarboxamide,
N-[4-[3,5-bis(trifluoromethyl)-1H-pyrazol-1-yl]phenyl]-2-chloro-3-pyridinecarboxamide,
N-[4-[3,5-bis(trifluoromethyl)-1H-pyrazol-1-yl]phenyl]-2-chloro-6-methyl-3 -, pyridinecarboxamide,
N-[4-[3,5-bis(trifluoromethyl)-1H-pyrazol-1-yl]phenyl]-4-fluoro-γ-oxobenzenebutanamide,
N-[4-[3,5-bis(trifluoromethyl)-1H-pyrazol-1-yl]phenyl]-1,2,3,4-tetrahydro-2-naphthalenecarboxamide,
N-[4-[3,5-bis(trifluoromethyl)-1H-pyrazol-1-yl]phenyl]-2-(4-chlorophenoxy)-2-methylpropanamide,
N-[4-[3,5-bis(trifluoromethyl)-1H-pyrazol-1-yl]phenyl]acetamide,
4-[[[4-[3,5-bis(trifluoromethyl)-1H-pyrazol-1-yl]phenyl]amino]carbonyl]benzoic acid,
phenylmethyl N-[4-[3,5-bis(trifluoromethyl)-1H-pyrazol-1-yl]phenyl]amino]-4-oxobutyl]carbamate,
3-[[[4-[3,5-bis(trifluoromethyl)-1H-pyrazol-1-yl]phenyl]amino]carbonyl]benzoic acid,
N-[4-[3,5-bis(trifluoromethyl)-1H-pyrazol-1-yl]phenyl]-3-bromo-2-thiophenecarboxamide,
N-[4-[3,5-bis(trifluoromethyl)-1H-pyrazol-1-yl]phenyl]-3-methyl-2-thiophenecarboxamide,
2-amino-N-[4-[3,5-bis(trifluoromethyl)-1H-pyrazol-1-yl]phenyl]benzamide,
N-[4-[3,5-bis(trifluoromethyl)-1H-pyrazol-1-yl]phenyl]-2-fluoro-3-pyridinecarboxamide,
N-[4-[3,5-bis(trifluoromethyl)-1H-pyrazol-1-yl]phenyl]-3-chloro-4-(methylsulfonyl)-2-thiophenecarboxamide,
N-[4-[3,5-bis(trifluoromethyl)-1H-pyrazol-1-yl]phenyl]-1H-pyrrole-2-carboxamide,
N-[4-[3,5-bis(trifluoromethyl)-1H-pyrazol-1-yl]phenyl]-3,6-dichloro-2-pyridinecarboxamide,
N-[4-[3,5-bis(trifluoromethyl)-1H-pyrazol-1-yl]phenyl]-2-(2-nitrophenoxy)acetamide,
N-[4-[3,5-bis(trifluoromethyl)-1H-pyrazol-1-yl]phenyl]-1H-indole-2-acetamide,
(E)-N-[4-[3,5-bis(trifluoromethyl)-1H-pyrazol-1-yl]phenyl]-3-(2-thienyl)-2-propenamide,
N-[4-[3,5-bis(trifluoromethyl)-1H-pyrazol-1-yl]phenyl]pyazinecarboxamide,
1,1-dimethylethyl [[4-[4-[3,5-bis(trifluoromethyl)-1H-pyrazol-1-yl]phenyl]amino]-4-oxobutyl]carbamate,
1-acetyl-N-[4-[3,5-bis(trifluoromethyl)-1H-pyrazol-1-yl]phenyl]-4-piperidinecarboxamide,
N-[4-[3,5-bis(trifluoromethyl)-1H-pyrazol-1-yl]phenyl]butanamide,
N-[4-[3,5-bis(trifluoromethyl)-1H-pyrazol-1-yl]phenyl]-4-chloro-2-methoxybenzamide,
N-[4-[3,5-bis(trifluoromethyl)-1H-pyrazol-1-yl]phenyl]-α-methyl-4-(2-thienylcarbonyl)benzeneacetamide,
N-[4-[3,5-bis(trifluoromethyl)-1H-pyrazol-1-yl]phenyl]-α-methyl-4-(2-thienylcarbonyl)benzeneacetamide,
N-[4-[3,5-bis(trifluoromethyl)-1H-pyrazol-1-yl]phenyl]-2-methoxy-4-(methythio)benzamide,
N-[4-[3,5-bis(trifluoromethyl)-1H-pyrazol-1-yl]phenyl]-4-hydroxy-3-nitrobenzamide,
N-[4-[3,5-bis(trifluoromethyl)-1H-pyrazol-1-yl]phenyl]-3,4-dihydroxy benzamide,
N-[4-[3,5-bis(trifluoromethyl)-1H-pyrazol-1-yl]phenyl]-2-hydroxy-6-methoxybenzamide,
N-[4-[3,5-bis(trifluoromethyl)-1H-pyrazol-1-yl]phenyl]-2,4-bis(trifluoromethyl)benzamide,
N-[4-[3,5-bis(trifluoromethyl)-1H-pyrazol-1-yl]phenyl]-5-methyl-4-isoxazolecarboxamide,
N-[4-[3,5-bis(trifluoromethyl)-1H-pyrazol-1-yl]phenyl]-4-fluoro-3-(trifluoromethyl)benzamide,
N-[4-[3,5-bis(trifluoromethyl)-1H-pyrazol-1-yl]phenyl]-4-fluoro-3-nitrobenzamide,
N-[4-[3,5-bis(trifluoromethyl)-1H-pyrazol-1-yl]phenyl]-4-fluoro-2-(trifluoromethyl)benzamide,
N-[4-[3,5-bis(trifluoromethyl)-1H-pyrazol-1-yl]phenyl]-2-bromo-3-nitrobenzamide,
N-[4-[3,5-bis(trifluoromethyl)-1H-pyrazol-1-yl]phenyl]-2-chloro-4-fluorobenzamide,
N-[4-[3,5-bis(trifluoromethyl)-1H-pyrazol-1-yl]phenyl]-2-chloro-4-(methylsulfonyl)benzamide,
N-[4-[3,5-bis(trifluoromethyl)-1H-pyrazol-1-yl]phenyl]-2,5-dichlorobenzamide,
N-[4-[3,5-bis(trifluoromethyl)-1H-pyrazol-1-yl]phenyl]-2,3-difluorobenzamide,
N-[4-[3,5-bis(trifluoromethyl)-1H-pyrazol-1-yl]phenyl]-3-chloro-4-fluorobenzamide,
N-[4-[3,5-bis(trifluoromethyl)-1H-pyrazol-1-yl]phenyl]-2,5-difluorobenzamide,
N-[4-[3,5-bis(trifluoromethyl)-1H-pyrazol-1-yl]phenyl]-2-chloro-6-fluorobenzamide,
N-[4-[3,5-bis(trifluoromethyl)-1H-pyrazol-1-yl]phenyl]-2-fluoro-6-(trifluoromethyl)benzamide,
N-[4-[3,5-bis(trifluoromethyl)-1H-pyrazol-1-yl]phenyl]-3-chloro-2-fluorobenzamide,
N-[4-[3,5-bis(trifluoromethyl)-1H-pyrazol-1-yl]phenyl]-2-chloro-4-methoxybenzamide,
N-[4-[3,5-bis(trifluoromethyl)-1H-pyrazol-1-yl]phenyl]-2,6-dichloro-3-nitrobenzamide,
N-[4-[3,5-bis(trifluoromethyl)-1H-pyrazol-1-yl]phenyl]-4-bromo-2-chlorobenzamide,
N-[4-[3,5-bis(trifluoromethyl)-1H-pyrazol-1-yl]phenyl]-3,4-difluorobenzamide,
N-[4-[3,5-bis(trifluoromethyl)-1H-pyrazol-1-yl]phenyl]-2-bromo-5-methoxybenzamide,
N-[4-[3,5-bis(trifluoromethyl)-1H-pyrazol-1-yl]phenyl]-4-chloro-2-hydroxybenzamide,
N-[4-[3,5-bis(trifluoromethyl)-1H-pyrazol-1-yl]phenyl]-3-bromo-4-methoxybenzamide,
N-[4-[3,5-bis(trifluoromethyl)-1H-pyrazol-1-yl]phenyl]-3-bromo-4-hydroxybenzamide,
N-[4-[3,5-bis(trifluoromethyl)-1H-pyrazol-1-yl]phenyl]-2-chloro-4,5-difluorobenzamide,
N-[4-[3,5-bis(trifluoromethyl)-1H-pyrazol-1-yl]phenyl]-4-chloro-2,5-difluorobenzamide,
N-[4-[3,5-bis(trifluoromethyl)-1H-pyrazol-1-yl]phenyl]-2,3,4-trifluorobenzamide,
N-[4-[3,5-bis(trifluoromethyl)-1H-pyrazol-1-yl]phenyl]-3,4,5-trifluorobenzamide,
N-[4-[3,5-bis(trifluoromethyl)-1H-pyrazol-1-yl]phenyl]-2,4,5-trifluorobenzamide,
N-[4-[3,5-bis(trifluoromethyl)-1H-pyrazol-1-yl]phenyl]-2,4,6-trifluorobenzamide,
N-[4-[3,5-bis(trifluoromethyl)-1H-pyrazol-1-yl]phenyl]-2,6-difluoro-3-nitrobenzamide,
N-[4-[3,5-bis(trifluoromethyl)-1H-pyrazol-1-yl]phenyl]-2,3,5-trifluorobenzamide,
N-[4-[3,5-bis(trifluoromethyl)-1H-pyrazol-1-yl]phenyl]-2,4-dichloro-6-fluorobenzamide,
N-4-[3,5-bis(trifluoromethyl)-1H-pyrazol-1-yl]phenyl-2,4-dichloro-3,5-dinitrobenzamide,
N-[4-[3,5-bis(trifluoromethyl)-1H-pyrazol-1-yl]phenyl]-2,3,5,6-tetrafluorobenzamide,
N-[4-[3,5-bis(trifluoromethyl)-1H-pyrazol-1-yl]phenyl]-2,3,4,5-tetrafluorobenzamide,
N-[4-[3,5-bis(trifluoromethyl)-1H-pyrazol-1-yl]phenyl]-4-bromo-2,3,5,6-tetrafluorobenzamide,
N-[4-[3,5-bis(trifluoromethyl)-1H-pyrazol-1-yl]phenyl]-5-methyl-2-nitrobenzamide,
N-[4-[3,5-bis(trifluoromethyl)-1H-pyrazol-1-yl]phenyl]-3-thiophene-carboxamide,
N-[4-[3,5-bis(trifluoromethyl)-1H-pyrazol-1-yl]phenyl]-5-isoxazolecarboxamide,
N-[4-[3,5-bis(trifluoromethyl)-1H-pyrazol-1-yl]phenyl]tetrahydro-2-furancarboxamide,
N-[4-[3,5-bis(trifluoromethyl)-1H-pyrazol-1-yl]phenyl]-2-pyrrolidinecarboxamide,
N-[4-[3,5-bis(trifluoromethyl)-1H-pyrazol-1-yl]phenyl]tetrahydro-3-furancarboxamide,
N-[4-[3,5-bis(trifluoromethyl)-1H-pyrazol-1-yl]phenyl]-1,2,3-thiadiazole-5-carboxamide,
N-[4-[3,5-bis(trifluoromethyl)-1H-pyrazol-1-yl]phenyl]-2-chloro-4-pyridinecarboxamide,
1,1-dimethylethyl 2-[[[4-[3,5-bis(trifluoromethyl)-1H-pyrazol-1-yl]phenyl]-amino]carbonyl]-1-pyrrolidinecarboxylate,
N-[4-[3,5-bis(trifluoromethyl)-1H-pyrazol-1-yl]phenyl]-5-nitro-2-furancarboxamide,
N-[4-[3,5-bis(trifluoromethyl)-1H-pyrazol-1-yl]phenyl]-1-methyl-1H-pyrrole-2-carboxamide,
N-[4-[3,5-bis(trifluoromethyl)-1H-pyrazol-1-yl]phenyl]-6-chloro-3-pyridinecarboxamide,
N-[4-[3,5-bis(trifluoromethyl)-1H-pyrazol-1-yl]phenyl]-5-bromo-2-furancarboxamide,
N-[4-[3,5-bis(trifluoromethyl)-1H-pyrazol-1-yl]phenyl]-3-methyl-2-furancarboxamide,
N-[4-[3,5-bis(trifluoromethyl)-1H-pyrazol-1-yl]phenyl]-5-chloro-2-thiophenecarboxamide,
(S)-N-[4-[3,5-bis(trifluoromethyl)-1H-pyrazol-1-yl]phenyl]tetrahydro-5-oxo-2-furancarboxamide,
N-[4-[3,5-bis(trifluoromethyl)-1H-pyrazol-1-yl]phenyl]-5-oxo-2-pyrrolidinecarboxamide,
N-[4-[3,5-bis(trifluoromethyl)-1H-pyrazol-1-yl]phenyl]-5-bromo-3-pyridinecarboxamide,
N-[4-[3,5-bis(trifluoromethyl)-1H-pyrazol-1-yl]phenyl]-5-nitro-3-thiophenecarboxamide,
1,1-dimethylethyl 4-[[[4-[3,5-bis(trifluoromethyl)-1H-pyrazol-1-yl]phenyl]-amino]carbonyl]-3-thiazolidinecarboxylate,
N-[4-[3,5-bis(trifluoromethyl)-1H-pyrazol-1-yl]phenyl]-5-methoxy-3-thiophenecarboxamide,
N-[4-[3,5-bis(trifluoromethyl)-1H-pyrazol-1-yl]phenyl]-2,3-dibromo-5-thiophenecarboxamide,
N-[4-[3,5-bis(trifluoromethyl)-1H-pyrazol-1-yl]phenyl]-3-fluoro-4-pyridinecarboxamide,
N-[4-[3,5-bis(trifluoromethyl)-1H-pyrazol-1-yl]phenyl]-1-methyl-1H-pyrazole-4-carboxamide,
N-[4-[3,5-bis(trifluoromethyl)-1H-pyrazol-1-yl]phenyl]-5-chloro-4-methoxy-3-thiophenecarboxamide,
N-[4-[3,5-bis(trifluoromethyl)-1H-pyrazol-1-yl]phenyl]-5,6-dichloro-3-pyridinecarboxamide,
N-[4-[3,5-bis(trifluoromethyl)-1H-pyrazol-1-yl]phenyl]-2,6-dichloro-4-pyridinecarboxamide,
N-[4-[3,5-bis(trifluoromethyl)-1H-pyrazol-1-yl]phenyl]-2,5-dichloro-3-pyridinecarboxamide,
3-amino-N-(4-(3,5-bis(trifluoromethyl)-1H-pyrazol-1-yl)phenyl)isonicotinamide,
N-(4-(3,5-bis(trifluoromethyl)-1H-pyrazol-1-yl)phenyl)-3-chloro-5-methoxyisonicotinamide,
4-(aminomethyl)-N-(4-(3,5-bis(trifluoromethyl)-1H-pyrazol-1-yl)phenyl)-2-chlorobenzamide,
N-(4-(3,5-bis(trifluoromethyl)-1H-pyrazol-1-yl)phenyl)-2-methylacrylamide,
N-(4-(3,5-bis(trifluoromethyl)-1H-pyrazol-1-yl)phenyl)-4-chloro-2-fluorobenzamide,
N-(4-(5-cyano-3-(trifluoromethyl)-1H-pyrazol-1-yl)phenyl)-2-fluorobenzamide,
2-fluoro-N-(4-(5-(2-furyl)-3-(trifluoromethyl)-1H-pyrazol-1-yl)phenyl)benzamide,
N-(4-(5-cyano-3-(trifluoromethyl)-1H-pyrazol-1-yl)phenyl)-4-methyl-1,2,3-thiadiazole-5-carboxamide,
N-(4-(5-cyano-3-(trifluoromethyl)-1H-pyrazol-1-yl)phenyl)isonicotinamide,
N-(4-(5-cyano-3-(trifluoromethyl)-1H-pyrazol-1-yl)phenyl)-3-fluoroisonicotinamide,
N-(4-(5-acetyl-3-(trifluoromethyl)-1H-pyrazol-1-yl)phenyl)-2-fluorobenzamide,
N-(4-(5-cyano-3-(trifluoromethyl)-1H-pyrazol-1-yl)phenyl)-2-fluoronicotinamide,
N-(4-(5-cyano-3-(trifluoromethyl)-1H-pyrazol-1-yl)phenyl)-2,3,5-trifluorobenzamide,
2-fluoro-N-(4-(5-(2-thienyl)-3-(trifluoromethyl)-1H-pyrazol-1-yl)phenyl)benzamide,
2-fluoro-N-(4-(5-(methylsulfanyl)-3-(trifluoromethyl)-1H-pyrazol-1-yl)phenyl)benzamide,
2-fluoro-N-(4-(5-(3-pyridinyl)-3-(trifluoromethyl)-1H-pyrazol-1-yl)phenyl)benzamide,
3-fluoro-N-(4-(5-(2-thienyl)-3-(trifluoromethyl)-1H-pyrazol-1-yl)phenyl)isonicotinamide,
N-(4-(5-methoxy-3-(trifluoromethyl)-1H-pyrazol-1-yl)phenyl)isonicotinamide,
2-fluoro-N-(4-(5-methoxy-3-(trifluoromethyl)-1H-pyrazol-1-yl)phenyl)benzamide,
N-(4-(5-methoxy-3-(trifluoromethyl)-1H-pyrazol-1-yl)phenyl)-4-methyl-1,2,3-thiadiazole-5-carboxamide,
N-(4-(5-acetyl-3-(trifluoromethyl)-1H-pyrazol-1-yl)phenyl)-2-fluoronicotinamide,
2-fluoro-N-(4-(5-(methylsulfanyl)-3-(trifluoromethyl)-1H-pyrazol-1-yl)phenyl)nicotinamide,
2-fluoro-N-(4-(5-methoxy-3-(trifluoromethyl)-1H-pyrazol-1-yl)phenyl)nicotinamide,
N-(4-(5-ethoxy-3-(trifluoromethyl)-1H-pyrazol-1-yl)phenyl)-2-fluoronicotinamide,
3 -fluoro-N-(4-(5-(methylsulfanyl)-3-(trifluoromethyl)-1H-pyrazol-1-yl)phenyl)isonicotinamide,
3-fluoro-N-(4-(5-methoxy-3-(trifluoromethyl)-1H-pyrazol-1-yl)phenyl)isonicotinamide,
N-(4-(5-(difluoromethoxy)-3-(trifluoromethyl)-1H-pyrazol-1-yl)phenyl)isonicotinamide,
N-(4-(5-(difluoromethoxy)-3-(trifluoromethyl)-1H-pyrazol-1-yl)phenyl)-4-methyl-1,2,3-thiadiazole-5-carboxamide,
N-(4-(5-(difluoromethoxy)-3-(trifluoromethyl)-1H-pyrazol-1-yl)phenyl)-2-fluoronicotinamide,
N-(4-(5-chloro-3-(trifluoromethyl)-1H-pyrazol-1-yl)phenyl)-2-fluoronicotinamide,
2-fluoro-N-(4-(5-nitro-3-(trifluoromethyl)-1H-pyrazol-1-yl)phenyl)benzamide,
N-(4-(5-(difluoromethoxy)-3-(trifluoromethyl)-1H-pyrazol-1-yl)phenyl)-3-fluoroisonicotinamide,
N-(4-(5-chloro-3-(trifluoromethyl)-1H-pyrazol-1-yl)phenyl)-3-fluoroisonicotinamide,
N-(4-(5-bromo-3-(trifluoromethyl)-1H-pyrazol-1-yl)phenyl)-3-fluoroisonicotinamide,
3-fluoro-N-(4-(5-nitro-3-(trifluoromethyl)-1H-pyrazol-1-yl)phenyl)isonicotinamide,
N-(4-(5-bromo-3-(trifluoromethyl)-1H-pyrazol-1-yl)phenyl)-4-methyl-1,2,3-thiadiazole-5-carboxamide,
N-(4-(5-chloro-3-(trifluoromethyl)-1H-1,2,4-triazol-1-yl)phenyl)-3-fluoroisonieotinamide,
3-fluoro-N-(4-(5-(1-methyl-1H-pyrrol-3-yl)-3-(trifluoromethyl)-1H-pyrazol-1-yl)phenyl)isonicotinamide,
3-chloro-N-(4-(5-chloro-3-(trifluoromethyl)-1H-pyrazol-1-yl)phenyl)isonicotinamide,
N-(4-(5-bromo-3-(trifluoromethyl)-1H-pyrazol-1-yl)phenyl)-2,3-difluorobenzamide,
N-(4-(5-bromo-3-(trifluoromethyl)-1H-pyrazol-1-yl)phenyl)-3-chloroisonicotinamide,
2-chloro-N-(4-(5-cyano-3-(trifluoromethyl)-1H-pyrazol-1-yl)phenyl)benzamide,
3-chloro-N-(4-(5-cyano-3-(trifluoromethyl)-1H-pyrazol-1-yl)phenyl)isonicotinamide,
N-(4-(5-(difluoromethoxy)-3-(trifluoromethyl)-1H-pyrazol-1-yl)phenyl)-2-fluorobenzamide,
2-chloro-N-(4-(5-(difluoromethoxy)-3-(trifluoromethyl)-1H-pyrazol-1-yl)phenyl)benzamide,
and
N-(4-(5-(difluoromethoxy)-3-(trifluoromethyl)-1H-pyrazol-1-yl)phenyl)-2,3-difluorobenzamide.

6. A compound according to claim 3 where L₃ is —N(R₇)C(X)—, R₇ is alkyl of one to fifteen carbons, and W is O.

7. A compound according to claim 6 selected from
N-[4-[3,5-bis(trifluoromethyl)-1H-pyrazol-1-yl]phenyl]-2-chloro-N-methylbenzamide
and
N-[4-[3,5-bis(trifluoromethyl)-1H-pyrazol-1-yl]phenyl]-2-chloro-4-nitro-N-methylbenzamide.

8. A compound according to claim 3 where L₃ is —N(R₇)C(O)N(R₈)— and R₇ and R₈ are hydrogen.

9. A compound according to claim 8 selected from
ethyl 3-[[[[4-[3,5-bis(trifluoromethyl)-1H-pyrazol-1-yl]phenyl]-amino]carbonyl]-amino]benzoate,
N-[4-[3,5-bis(trifluoromethyl)-1H-pyrazol-1-yl]phenyl]-N′-(3-cyanophenyl)urea,
N-[4-[3,5-bis(trifluoromethyl)-1H-pyrazol-1-yl]phenyl]-N′-(3-nitrophenyl)urea,
N-[4-[3,5-bis(trifluoromethyl)-1H-pyrazol-1-yl]phenyl]-N′-(4-fluorophenyl)urea,
N-[4-[3,5-bis(trifluoromethyl)-1H-pyrazol-1-yl]phenyl]-N′-[4-(trifluoromethyl)phenyl]urea,
N-[4-[3,5-bis(trifluoromethyl)-1H-pyrazol-1-yl]phenyl]-N′-(3,5-dimethylphenyl)urea,
N-[4-[3,5-bis(trifluoromethyl)-1H-pyrazol-1-yl]phenyl]-N′-phenylurea,
N-[4-[3,5-bis(trifluoromethyl)-1H-pyrazol-1-yl]phenyl]-N′-(3-chloro-2-methylphenyl)urea,
N-[4-[3,5-bis(trifluoromethyl)-1H-pyrazol-1-yl]phenyl]-N′-[4-(butyloxyphenyl)urea,
N-[4-[3,5-bis(trifluoromethyl)-1H-pyrazol-1-yl]phenyl]-N′-(2-methyl-3-nitrophenyl)urea,
N-[4-[3,5-bis(trifluoromethyl)-1H-pyrazol-1-yl]phenyl]-N′-(2-chloro-4-nitrophenyl)urea,
N-(4-acetylphenyl)-N′-[4-[3,5-bis(trifluoromethyl)-1H-pyrazol-1-yl]-phenyl]urea,
N-[4-[3,5-bis(trifluoromethyl)-1H-pyrazol-1-yl]phenyl]-N′-(4-methyl-2-nitrophenyl)urea,
N-[4-[3,5-bis(trifluoromethyl)-1H-pyrazol-1-yl]phenyl]-N′-(4-bromo-2,6-dimethylphenyl)urea,
N-[4-[3,5-bis(trifluoromethyl)-1H-pyrazol-1-yl]phenyl]-N′-heptylurea,
N-[4-[3,5-bis(trifluoromethyl)-1H-pyrazol-1-yl]phenyl]-N′-(4-chloro-2-nitrophenyl)urea,
N-[4-[3,5-bis(trifluoromethyl)-1H-pyrazol-1-yl]phenyl]-N′-2-methyl-5-nitrophenyl)urea,
N-[4-[3,5-bis(trifluoromethyl)-1H-pyrazol-1-yl]phenyl]-N′-(4-bromo-2-methylphenyl)urea,
and
N-[4-[3,5-bis(trifluoromethyl)-1H-pyrazol-1-yl]phenyl]-N′-(4-chloro-3-nitrophenyl)urea.

10. A compound according to claim 3 where L₃ is —NR₇S(O)ₜ—, t is 2, and R₇ is hydrogen.

11. A compound according to claim 10 that is
N-[4-[3,5-bis(trifluoromethyl)-1H-pyrazol-1-yl]phenyl]-3,5-difluorobenzenesulfonamide.

12. A compound according to claim 3 where L₃ is —C(X)N(R₇)—, X is O, and R₇ is hydrogen.

13. A compound according to claim 12 selected from
4-[3,5-bis(trifluoromethyl)-H-pyrazol-1-yl]-N-(4-fluorophenyl)benzamide,
4-[3,5-bis(trifluoromethyl)-1H-pyrazol-1-yl]-N-(2-chlorophenyl)benzamide,
4-[3,5-bis(trifluoromethyl)-1H-pyrazol-1-yl]-N-(3-cyanophenyl)benzamide,
4-[3,5-bis(trifluoromethyl)-1H-pyrazol-1-yl]-N-(2,4-difluorophenyl)benzamide,
4-[3,5-bis(trifluoromethyl)-1H-pyrazol-1-yl]-N-(2-cyanophenyl)benzamide,
4-[3,5-bis(trifluoromethyl)-1H-pyrazol-1-yl]-N-(2-nitrophenyl)benzamide,
4-[3,5-bis(trifluoromethyl)-H-pyrazol-1-yl]-N-(2,6-difluorophenyl)benzamide,
4-[3,5-bis(trifluoromethyl)-1H-pyrazol-1-yl]-N-(2-bromophenyl)benzamide,
4-[3,5-bis(trifluoromethyl)-1H-pyrazol-1-yl]-N-(4-cyanophenyl)benzamide,
4-[3,5-bis(trifluoromethyl)-1H-pyrazol-1-yl]-N-(4-pyridinyl)benzamide,
and
N-[2-(aminocarbonyl)phenyl]-4-[3,5-bis(trifluoromethyl)-1H-pyrazol-1-yl]benzamide.

14. A compound according to claim 3 where L₃ is —NR₇(CH₂)ₘ—, R₇ is hydrogen, and m is 1.

15. A compound according to claim 14 selected from
N-[4-[3,5-bis(trifluoromethyl)-1H-pyrazol-1-yl]phenyl]-2-chlorobenzenemethanamine
and
N-[4-[3,5-bis(trifluoromethyl)-1H-pyrazol-1-yl]phenyl]-4-fluorobenzenemethanamine.

16. A compound according to claim 3 where L₃ is —(CH₂)ₘNR₇—, R₇ is hydrogen, and m is 1.

17. A compound according to claim 16 selected from
4-[3,5-bis(trifluoromethyl)-1H-pyrazol-1-yl]-N-(4-fluorophenyl)benzenemethanamine,
3-[4-[[[3,5-bis(trifluoromethyl)-1H-pyrazol-1-yl]phenyl]methyl]amino]benzonitrile,
4-[3,5-bis(trifluoromethyl)-1H-pyrazol-1-yl]-N-(2,4-difluorophenyl)benzenemethanamine,
and
3-[[[4-[3,5-bis(trifluoromethyl)-1H-pyrazol-1-yl]phenyl]methyl]amino]benzonitrile.

18. A compound according to claim 3 where L₃ is —C(H)═N—.

19. A compound according to claim 18 that is
(E)-N-[4-[3,5-bis(trifluoromethyl)-1H-pyrazol-1-yl]phenylmethylene]-2,4-difluorobenzenamine.

20. A compound according to claim 3 where L₃ is alkenylene of two to six carbons in the Z or E configuration.

21. A compound according to claim 20 selected from
(E)-3-[2-[4-[3,5-bis(trifluoromethyl)-1H-pyrazol-1-yl]phenyl]ethenyl]benzonitrile,
(Z)-3-[2-[4-[3,5-bis(trifluoromethyl)-1H-pyrazol-1-yl]phenyl]ethenyl]benzonitrile,
and
(E)-1-[4-[2-(2-chlorophenyl)ethenyl]phenyl]-3,5-bis(trifluoromethyl)-1H-pyrazole.

22. A compound according to claim 2 where
Z is carbon, R₂ is hydrogen, and R₁, R₃, and E are defined above,
and
R₄ and R₅ are independently selected from
(1) hydrogen,
(2) alkyl of one to fifteen carbons,
(3) alkoxy of one to fifteen carbons,
(4) halo,
(5) perfluoroalkyl of one to fifteen carbons,
(6) —CO₂R₆,
(7) substituted heterocycle,
(8) —L₁NR₇R₈, and
(9) —CN.

23. A compound according to claim 22 selected from
N-[4-[3,5-bis(trifluoromethyl)-1H-pyrazol-1-yl]-3-(trifluoromethyl)phenyl]-4-chlorobenzamide,
N-[4-[3,5-bis(trifluoromethyl)-1H-pyrazol-1-yl]-2-fluorophenyl]-2,4-difluorobenzamide,
N-[2,4-bis[3,5-bis(trifluoromethyl)-1H-pyrazol-1-yl]phenyl]-2,4-difluorobenzamide,
methyl 2-[3,5-bis(trifluoromethyl)-1H-pyrazol-1-yl]-5-[(5-bromo-2-chlorobenzoyl)amino]benzoate,
N-[4-[3,5-bis(trifluoromethyl)-1H-pyrazol-1-yl]-3-(trifluoromethyl)phenyl]-3,5-dimethyl-4-isoxazolecarboxamide,
N-[4-[3,5-bis(trifluoromethyl)-1H-pyrazol-1-yl]-3-(trifluoromethyl)phenyl]-4-methyl-1,2,3-thiadiazole-5-carboxamide,
N-[4-[3,5-bis(trifluoromethyl)-1H-pyrazol-1-yl]-3-chlorophenyl]-3,5-dimethyl-4-isoxazolecarboxamide,
N-[4-[3,5-bis(trifluoromethyl)-1H-pyrazol-1-yl]-2-(trifluoromethyl)phenyl]-3,5-dimethyl-4-isoxazolecarboxamide,
N-[4-[3,5-bis(trifluoromethyl)-1H-pyrazol-1-yl]-2-methylphenyl]-4-methyl-1,2,3-thiadiazole-5-carboxamide,
N-[4-[3,5-bis(trifluoromethyl)-1H-pyrazol-1-yl]-2-methoxyphenyl]-4-methyl-1,2,3-thiadiazole-5-carboxamide
methyl 2-(3,5-bis(trifluoromethyl)-1H-pyrazol-1-yl)-5-((2-fluorobenzoyl)amino)benzoate,
N-(3-amino-4-(3,5-bis(trifluoromethyl)-1H-pyrazol-1-yl)phenyl)-2-fluorobenzamide,
and
N-(4-(3,5-bis(trifluoromethyl)-1H-pyrazol-1-yl)-3-cyanophenyl)-2-fluorobenzamide.

24. A compound according to claim 2 where
R₁ is perfluoroalkyl of one to fifteen carbons and R₃ is alkyl of one to fifteen carbons;

25. A compound according to claim 24 selected from
4-chloro-N-[4-[5-methyl-3-(trifluoromethyl)-1H-pyrazol-1-yl]phenyl]benzamide,
4-methyl-N-[4-[5-methyl-3-(trifluoromethyl)-1H-pyrazol-1-yl]phenyl]-1,2,3-thiadiazole-5-carboxamide, and
3,5-dimethyl-N-[4-[5-methyl-3-(trifluoromethyl)-1H-pyrazol-1-yl]phenyl]-4-isoxazolecarboxamide.

26. A compound according to claim 2 where
R₁ is hydrogen and R₃ is alkyl of one to fifteen carbons.

27. A compound according to claim 26 selected from
4-chloro-N-[4-(5-methyl-1H-pyrazol-1-yl)phenyl]benzamide,
4-methyl-N-[4-(5-methyl-1H-pyrazol-1-yl)phenyl]-1,2,3-thiadiazole-5-carboxamide,
and
3,5-dimethyl-N-[4-(5-methyl-1H-pyrazol-1-yl)phenyl]-4-isoxazolecarboxamide.

28. A compound according to claim 2 where
R₁ is perfluoroalkyl of one to fifteen carbons and R₃ is hydrogen;

29. A compound according to claim 28 that is
3,5-dimethyl-N-[4-[3-(trifluoromethyl)-1H-pyrazol-1-yl)phenyl]-4-isoxazolecarboxamide.

30. A compound according to claim 2 where
R₁ is perfluoroalkyl of one to fifteen carbons and R₃ is hydroxyl;

31. A compound according to claim 30 that is
N-[4-[5-hydroxy-3-(trifluoromethyl)-1H-pyrazol-1-yl]phenyl]-4-methyl-1,2,3-thiadiazole-5-carboxamide.

32. A compound according to claim 1 of formula 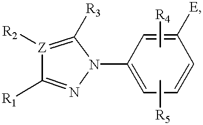
or a pharmaceutically acceptable salt or prodrug thereof, where
R₁, R₂, R₃, R₄, R₅, Z, and E are defined above.

33. A compound according to claim 32 selected from
N-[3-[3,5-bis(trifluoromethyl)-1H-pyrazol-1-yl]phenyl]-4-chlorobenzeneacetamide,
N-[3-[3,5-bis(trifluoromethyl)-1H-pyrazol-1-yl]phenyl]-2,3-dichlorobenzamide,
N-[3-[3,5-bis(trifluoromethyl)-1H-pyrazol-1-yl]phenyl]-2-chloro-5-nitrobenzamide,
N-[3-[3,5-bis(trifluoromethyl)-1H-pyrazol-1-yl]phenyl]-4-fluorobenzamide,
N-[3-[3,5-bis(trifluoromethyl)-1H-pyrazol-1-yl]phenyl]-2,4-difluorobenzamide,
N-[3-[3,5-bis(trifluoromethyl)-1H-pyrazol-1-yl]phenyl]-3-cyanobenzamide,
N-[3-[3,5-bis(trifluoromethyl)-1H-pyrazol-1-yl]phenyl]-2,6-difluorobenzamide, and
N-[3-[3,5-bis(trifluoromethyl)-1H-pyrazol-1-yl]phenyl]-4-cyanobenzamide.

34. A compound according to claim 1 of formula 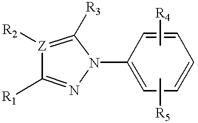
or a pharmaceutically acceptable salt or prodrug thereof, where
R₁, R₂, R₃, R₄, R₅, Z, and E are defined above.

35. A compound according to claim 34 selected from
N-[4-[3,5-bis(trifluoromethyl)-1H-pyrazol-1-yl]phenyl]-2,6-difluorobenzamide and
N-[4-[3,5-bis(trifluoromethyl)-1H-pyrazol-1-yl]phenyl]-4-chlorobenzeneacetamide.

36. A compound according to claim 1 of formula 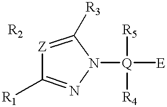
or a pharmaceutically acceptable salt or prodrug thereof, where
Q is heterocycle, and R₁, R₂, R₃, R₄, R₅, Z, and E are defined above.

37. A compound according to claim 36 selected from
N-[2-[3,5-bis(trifluoromethyl)-1H-pyrazol-1-yl]-5-pyridinyl]-2-chlorobenzamide,
N-[2-[3,5-bis(trifluoromethyl)-1H-pyrazol-1-yl]-5-pyridinyl]-3-cyanobenzamide,
N-[2-[3,5-bis(trifluoromethyl)-1H-pyrazol-1-yl]-5-pyridinyl]-2-chloro-4,5-difluorobenzamide,
N-(6-(3,5-bis(trifluoromethyl)-1H-pyrazol-1-yl)-3-pyridinyl)-2-fluorobenzamide, and
N-(5-(3,5-bis(trifluoromethyl)-1H-pyrazol-1-yl)-2-pyridinyl)-2-fluorobenzamide.

38. A compound according to claim 1 of formula 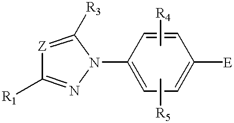
or a pharmaceutically acceptable salt or prodrug thereof, where
Z is nitrogen, and R₁, R₃, R₄, R₅, and E are defined above.

39. A compound according to claim 38 selected from
3,5-dimethyl-N-[4-(3,5-dimethyl-1H-1,2,4-triazol-1-yl)phenyl]-4-isoxazolecarboxamide and
N-[4-(3,5-dimethyl-1H-1,2,4-triazol-1-yl)phenyl]-4-methyl-1,2,3-thiadiazole-5-carboxamide.

40. A compound according to claim 2 where R₁ is —L₂-heterocycle, and the heterocycle can be optionally substituted.

41. A compound according to claim 40 selected from the group consisting of
3-fluoro-N-(4-(3-(4-pyridinyl)-5-(trifluoromethyl)-1H-pyrazol-1-yl)phenyl)isonicotinamide,
N-(4-(5-(difluoromethyl)-3-(3-pyridinyl)-1H-pyrazol-1-yl)phenyl)-3-fluoroisonicotinamide,
N-(4-(5-cyano-3-(2-pyridinyl)-1H-pyrazol-1-yl)phenyl)-3-fluoroisonicotinamide,
N-(4-(5-cyano-3-(3-pyridinyl)-1H-pyrazol-1-yl)phenyl)-4-methyl-1,2,3-thiadiazole-5-carboxamide,
3-fluoro-N-(4-(5-nitro-3-(3-pyridinyl)-1H-pyrazol-1-yl)phenyl)isonicotinamide,
4-methyl-N-(4-(5-nitro-3-(3-pyridinyl)-1H-pyrazol-1-yl)phenyl)-1,2,3-thiadiazole-5-carboxamide,
N-(4-(5-cyano-3-(1,3-thiazol-2-yl)-1H-pyrazol-1-yl)phenyl)-3-fluoroisonicotinamide,
N-(4-(3-(5-bromo-3-pyridinyl)-5-(difluoromethoxy)-1H-pyrazol-1-yl)phenyl)-3-fluoroisonicotinamide,
N-(4-(5-cyano-3-(1,3-thiazol-2-yl)-1H-pyrazol-1-yl)phenyl)-4-methyl-1,2,3-thiadiazole-5-carboxamide,
N-(4-(5-cyano-3-(1,3-thiazol-2-yl)-1H-pyrazol-1-yl)phenyl)-3-fluoroisonicotinamide,
4-methyl-N-(4-(3-(1,3-thiazol-2-yl)-5-(trifluoromethyl)-1H-pyrazol-1-yl)phenyl)-1,2,3-thiadiazole-5-carboxamide,
N-(4-(3-(2,4-dimethyl-1,3-thiazol-5-yl)-5-(trifluoromethyl)-1H-pyrazol-1-yl)phenyl)-3-fluoroisonicotinamide,
3-fluoro-N-(4-(3-tetrahydro-2-furanyl-5-(trifluoromethyl)-1H-pyrazol-1-yl)phenyl)isonicotinamide,
N-(4-(5-chloro-3-(1,3-thiazol-2-yl)-1H-pyrazol-1-yl)phenyl)-3-fluoroisonicotinamide,
N-(4-(5-chloro-3-(3-furyl)-1H-pyrazol-1-yl)phenyl)-4-methyl-1,2,3-thiadiazole-5-carboxamide,
N-(4-(5-chloro-3-(3-furyl)-1H-pyrazol-1-yl)phenyl)-3-fluoroisonicotinamide,
N-(4-(5-cyano-3-tetrahydro-2-furanyl-1H-pyrazol-1-yl)phenyl)-3-fluoroisonicotinamide,
N-(4-(5-(difluoromethoxy)-3-(1-methyl-1H-pyrrol-3-yl)-1H-pyrazol-1-yl)phenyl)-3-fluoroisonicotinamide,
N-(4-(5-(difluoromethoxy)-3-(3-furyl)-1H-pyrazol-1-yl)phenyl)-3-fluoroisonicotinamide, and
N-(4-(5-(difluoromethoxy)-3-(1-methyl-1H-pyrrol-2-yl)-1H-pyrazol-1-yl)phenyl)-3-fluoroisonicotinamide.

42. A compound selected from
N-[4-[3,5-bis(trifluoromethyl)-1H-pyrazol-1-yl]phenyl]cyclopropanecarboxamide,
N-[4-[3,5-bis(trifluoromethyl)-1H-pyrazol-1-yl]phenyl]-2,2,3,3-tetramethylcyclopropane-carboxamide,
N-[4-[3,5-bis(trifluoromethyl)-1H-pyrazol-1-yl]phenyl]-2,2-dichloro-1-methylcyclopropanecarboxamide,
N-[4-[3,5-bis(trifluoromethyl)-1H-pyrazol-1-yl]phenyl]-2-oxo-6-pentyl-2H-pyran-3-carboxamide,
N-[4-[3,5-bis(trifluoromethyl)-1H-pyrazol-1-yl]phenyl]-3,5-difluorobenzenesulfonamide,
N-[4-[3,5-bis(trifluoromethyl)-1H-pyrazol-1-yl]phenyl]-1-cyclohexene-1-carboxamide,
N-[4-[3,5-bis(trifluoromethyl)-1H-pyrazol-1-yl]phenyl]-2-methylcyclopropanecarboxamide,
N-[4-[3,5-bis(trifluoromethyl)-1H-pyrazol-1-yl]phenyl]-5-(3,5-dichlorophenoxy)-2-furancarboxamide,
N-[4-[3,5-bis(trifluoromethyl)-1H-pyrazol-1-yl]phenyl]-1-methyl-2-cyclohexene-1-carboxamide,
N-[4-[3,5-bis(trifluoromethyl)-1H-pyrazol-1-yl]phenyl]-1-cyclopentene-1-carboxamide,
N-[4-[3,5-bis(trifluoromethyl)-1H-pyrazol-1-yl]phenyl]-3-methoxycyclohexanecarboxamide,
N-[4-[3,5-bis(trifluoromethyl)-1H-pyrazol-1-yl]phenyl]-2-butynamide,
ethyl 3-[[[[4-[3,5-bis(trifluoromethyl)-1H-pyrazol-1-yl]phenyl]amino]carbonyl]-amino]benzoate,
N-[4-[3,5-bis(trifluoromethyl)-1H-pyrazol-1-yl]phenyl]-3-furancarboxamide,
N-[4-[3,5-bis(trifluoromethyl)-1H-pyrazol-1-yl]phenyl]-2-methyl-3-nitrobenzamide,
N-[4-[3,5-bis(trifluoromethyl)-1H-pyrazol-1-yl]phenyl]-N′-(3-cyanophenyl)urea,
N-[4-[3,5-bis(trifluoromethyl)-1H-pyrazol-1-yl]phenyl]-1-hydroxycyclopropanecarboxamide,
N-[4[3,5-bis(trifluoromethyl)-1H-pyrazol-1-yl]phenyl]cycloheptanecarboxamide,
N-[4-[3,5-bis(trifluoromethyl)-1H-pyrazol-1-yl]phenyl]-2-benzofurancarboxamide,
N-[4-[3,5-bis(trifluoromethyl)-1H-pyrazol-1-yl]phenyl]-5-fluoro-1H-indole-2-carboxamide,
(E)-N-[4-[3,5-bis(trifluoromethyl)-1H-pyrazol-1-yl]phenyl]-3-(2-chlorophenyl)-2-propenamide,
2-benzoyl-N-[4-[3,5-bis(trifluoromethyl)-1H-pyrazol-1-yl]phenyl]benzamide,
3a(S)-(3aoα,4β,6aα)-ν-[4-[3,5-bis(trifluoromethyl)-1H-pyrazol-1-yl]phenyl]hexahydro-2-oxo-1H-thieno[3,4-d]imidazole-4-pentanamide,
N-[4-[3,5-bis(trifluoromethyl)-1H-pyrazol-1-yl]phenyl]-3-iodobenzamide,
exo-N-[4-[3,5-bis(trifluoromethyl)-1H-pyrazol-1-yl]phenyl]bicyclo[2.2.1]hept-5-ene-2-carboxamide,
N-[4-[3,5-bis(trifluoromethyl)-1H-pyrazol-1-yl]phenyl]-2-methylcyclohexanecarboxamide,
phenylmethyl [1-[[[4-[3,5-bis(trifluoromethyl)-1H-pyrazol-1-yl]phenyl]amino]-carbonyl]propyl]carbamate,
N-[4-[3,5-bis(trifluoromethyl)-1H-pyrazol-1-yl]phenyl]-3-cyclohexene-1-carboxamide,
4-[3,5-bis(trifluoromethyl)-1H-pyrazol-1-yl]-N-(4-fluorophenyl)benzamide,
N-[4-[3,5-bis(trifluoromethyl)-1H-pyrazol-1-yl]phenyl]-N′-(3-nitrophenyl)urea,
N-[4-[3,5-bis(trifluoromethyl)-1H-pyrazol-1-yl]phenyl]-N′-(4-fluorophenyl)urea,
N-[4-[3,5-bis(trifluoromethyl)-1H-pyrazol-1-yl]phenyl]-N′-[4-(trifluoromethyl)phenyl]urea,
N-[4-[3,5-bis(trifluoromethyl)-1H-pyrazol-1-yl]phenyl]-N′-(3,5-dimethylphenyl)urea,
N-[4-[3,5-bis(trifluoromethyl)-1H-pyrazol-1-yl]phenyl]-1-methylcyclopropanecarboxamide,
N-[4-[3,5-bis(trifluoromethyl)-1H-pyrazol-1-yl]phenyl]-N′-phenylurea,
N-[4-[3,5-bis(trifluoromethyl)-1H-pyrazol-1-yl]phenyl]-N′-(3-chloro-2-methylphenyl)urea,
N-[4-[3,5-bis(trifluoromethyl)-1H-pyrazol-1-yl]phenyl]-N′-[4-(butyloxyphenyl)urea,
N-[4-[3,5-bis(trifluoromethyl)-1H-pyrazol-1-yl]phenyl]-N′-(2-methyl-3-nitrophenyl)urea,
N-[4-[3,5-bis(trifluoromethyl)-1H-pyrazol-1-yl]phenyl]-N′-(2-chloro-4-nitrophenyl)urea,
N-(4-acetylphenyl)-N′-[4-[3,5-bis(trifluoromethyl)-1H-pyrazol-1-yl]phenyl]urea,
N-[4-[3,5-bis(trifluoromethyl)-1H-pyrazol-1-yl]phenyl]-N′-(4-methyl-2-nitrophenyl)urea,
N-[4-[3,5-bis(trifluoromethyl)-1H-pyrazol-1-yl]phenyl]-5-methyl-2-thiophenecarboxamide,
N-[4-[3,5-bis(trifluoromethyl)-1H-pyrazol-1-yl]phenyl]-N′-(4-bromo-2,6-dimethylphenyl)urea,
N-[4-[3,5-bis(trifluoromethyl)-1H-pyrazol-1-yl]phenyl]-4-(1H-pyrrol-1-yl)benzamide,
N-[4-[3,5-bis(trifluoromethyl)-1H-pyrazol-1-yl]phenyl]-N′-heptylurea,
N-[4-[3,5-bis(trifluoromethyl)-1H-pyrazol-1-yl]phenyl]-N′-(4-chloro-2-nitrophenyl)urea,
N-[4-[3,5-bis(trifluoromethyl)-1H-pyrazol-1-yl]phenyl]-7-methoxy-2-benzofurancarboxamide,
N-[4-[3,5-bis(trifluoromethyl)-1H-pyrazol-1-yl]phenyl]-N′-2-methyl-5-nitrophenyl)urea,
N-[4-[3,5-bis(trifluoromethyl)-1H-pyrazol-1-yl]phenyl]-3-(hydroxymethyl)benzamide,
N-[4-[3,5-bis(trifluoromethyl)-1H-pyrazol-1-yl]phenyl]-2-cyanoacetamide,
N-[4-[3,5-bis(trifluoromethyl)-1H-pyrazol-1-yl]phenyl]-2-cyclohexane-1-carboxamide,
N-[4-[3,5-bis(trifluoromethyl)-1H-pyrazol-1-yl]phenyl]-4-methylcyclohexanecarboxamide,
N-[4-[3,5-bis(trifluoromethyl)-1H-pyrazol-1-yl]phenyl]-α-methoxy-α-(trifluoromethyl)benzeneacetamide,
N-[4-[3,5-bis(trifluoromethyl)-1H-pyrazol-1-yl]phenyl]heptanamide,
N-[4-[3,5-bis(trifluoromethyl)-1H-pyrazol-1-yl]phenyl]-2-phenoxybenzamide,
3-amino-N-[4-[3,5-bis(trifluoromethyl)-1H-pyrazol-1-yl]phenyl]benzamide,
4-amino-N-[4-[3,5-bis(trifluoromethyl)-1H-pyrazol-1-yl]phenyl]benzamide,
4-azido-N-[4-[3,5-bis(trifluoromethyl)-1H-pyrazol-1-yl]phenyl]benzamide,
N-[4-[3,5-bis(trifluoromethyl)-1H-pyrazol-1-yl]-2-thiopheneacetamide,
N-[3,5-bis(trifluoromethyl)-1H-pyrazol-1-yl]phenyl]-1-tricyclo[3.3.1.1^{3,7}]decanecarboxmide,
N-[4-[3,5-bis(trifluoromethyl)-1H-pyrazol-1-yl]phenyl]-N²-[(1,1-dimethylethoxy)carbonyl]-1-asparagine, phenylmethyl ester,
1,1-dimethylethyl [7-[[4-[3,5-bis(trifluoromethyl)-1H-pyrazol-1-yl]phenyl]amino]-7-oxoheptyl]carbamate,
N-[4-[3,5-bis(trifluoromethyl)-1H-pyrazol-1-yl]phenyl]-3-(methylthio)propanamide,
N-[4-[3,5-bis(trifluoromethyl)-1H-pyrazol-1-yl]phenyl]-1-naphthylenecarboxamide,
N-[4-[3,5-bis(trifluoromethyl)-1H-pyrazol-1-yl]phenyl]-4-cyanobenzamide,
trans-N-[4-[3,5-bis(trifluoromethyl)-1H-pyrazol-1-yl]phenyl]-2-phenylcyclopropane-carboxamide,
N-[4-[3,5-bis(trifluoromethyl)-1H-pyrazol-1-yl]phenyl]-4-iodobenzamide,
N-[4-[3,5-bis(trifluoromethyl)-1H-pyrazol-1-yl]phenyl]-3-chloropropanamide,
N-[4-[3,5-bis(trifluoromethyl)-1H-pyrazol-1-yl]phenyl]-4-methoxybenzamide,
N-[4-[3,5-bis(trifluoromethyl)-1H-pyrazol-1-yl]phenyl]-2-ethylhexanamide,
N-[4-[3,5-bis(trifluoromethyl)-1H-pyrazol-1-yl]phenyl]-4-hydroxybenzamide,
N-[4-[3,5-bis(trifluoromethyl)-1H-pyrazol-1-yl]phenyl]-4-(hexyloxy)benzamide,
N-[4-[3,5-bis(trifluoromethyl)-1H-pyrazol-1-yl]phenyl]-3-methylbenzamide,
2-(acetyloxy)-N-[4-[3,5-bis(trifluoromethyl)-1H-pyrazol-1-yl]phenyl]benzamide,
N-[4-[3,5-bis(trifluoromethyl)-1H-pyrazol-1-yl]phenyl]-N′-(4-bromo-2-methylphenyl)urea,
N-[4-[3,5-bis(trifluoromethyl)-1H-pyrazol-1-yl]phenyl]-2,4,6-trimethylbenzamide,
N-[4-[3,5-bis(trifluoromethyl)-1H-pyrazol-1-yl]phenyl]-N′-(4-chloro-3-nitrophenyl)urea,
N-[4-[3,5-bis(trifluoromethyl)-1H-pyrazol-1-yl]phenyl]-2-chloro-N-methylbenzamide,
N-[4-[3,5-bis(trifluoromethyl)-1H-pyrazol-1-yl]phenyl]-2-chloro-4-nitro-N-methylbenzamide,
N-[4-[3,5-bis(trifluoromethyl)-1H-pyrazol-1-yl]phenyl]-2-chlorobenzenemethanamine,
N-[4-[3,5-bis(trifluoromethyl)-1H-pyrazol-1-yl]phenyl]-1-methyl-5-nitro-1H-pyrazole-4-carboxamide,
N-[4-[3,5-bis(trifluoromethyl)-1H-pyrazol-1-yl]phenyl]-4-fluorobenzenemethanamine,
N-[4-[3,5-bis(trifluoromethyl)-1H-pyrazol-1-yl]phenyl]-4-bromobenzamide,
N-[4-[3,5-bis(trifluoromethyl)-1H-pyrazol-1-yl]phenyl]-2-(dimethylamino)benzamide,
N-[4-[3,5-bis(trifluoromethyl)-1H-pyrazol-1-yl]phenyl]-3-(dimethylamino)benzamide,
N-[4-[3,5-bis(trifluoromethyl)-1H-pyrazol-1-yl]phenyl]-4-(trifluoromethyl)benzamide,
N-[4-[3,5-bis(trifluoromethyl)-1H-pyrazol-1-yl]phenyl]-4-fluorobenzamide,
N-[4-[3,5-bis(trifluoromethyl)-1H-pyrazol-1-yl]phenyl]-2-chlorobenzamide,
N-[4-[3,5-bis(trifluoromethyl)-1H-pyrazol-1-yl]phenyl]benzamide,
N-[4-[3,5-bis(trifluoromethyl)-1H-pyrazol-1-yl]phenyl]-4-nitrobenzamide,
4-[3,5-bis(trifluoromethyl)-1H-pyrazol-1-yl]-N-(4-fluorophenyl)benzenemethanarnine,
3-[4-[[[3,5-bis(trifluoromethyl)-1H-pyrazol-1-yl]phenyl]methyl]amino]benzonitrile,
N-[4-[3,5-bis(trifluoromethyl)-1H-pyrazol-1-yl]phenyl]-2-methylbenzamide,
(E)-N-[4-[3,5-bis(trifluoromethyl)-1H-pyrazol-1-yl]phenylmethylene]-2,4-difluoro-benzenamnine,
N-[4-[3,5-bis(trifluoromethyl)-1H-pyrazol-1-yl]phenyl]-3-4-dimethoxybenzamide,
N-[4-[3,5-bis(trifluoromethyl)-1H-pyrazol-1-yl]phenyl]cyclopentanepropanamide,
N-[4-[3,5-bis(trifluoromethyl)-1H-pyrazol-1-yl]phenyl]-4-methylbenzamide,
N-[4-[3,5-bis(trifluoromethyl)-1H-pyrazol-1-yl]phenyl]-3-(trifluoromethyl)benzamide,
N-[4-[3,5-bis(trifluoromethyl)-1H-pyrazol-1-yl]phenyl]-3-methyl-2-butenamide,
N-[4-[3,5-bis(trifluoromethyl)-1H-pyrazol-1-yl]phenyl]-2-hydroxybenzamide,
N-[4-[3,5-bis(trifluoromethyl)-1H-pyrazol-1-yl]phenyl]-3-hydroxybenzamide,
N-[4-[3,5-bis(trifluoromethyl)-1H-pyrazol-1-yl]phenyl]-2,4-dimethyl-5-thiazolecarboxamide,
N-[4-[3,5-bis(trifluoromethyl)-1H-pyrazol-1-yl]phenyl]-3-pyridinecarboxamide,
N-[4-[3,5-bis(trifluoromethyl)-1H-pyrazol-1-yl]phenyl]-4-(hydroxymethyl)benzamide,
4-[3,5-bis(trifluoromethyl)-1H-pyrazol-1-yl]-N-(2,4-difluorophenyl)benzenemethanamine,
N-[4-[3,5-bis(trifluoromethyl)-1H-pyrazol-1-yl]phenyl]-4-(methylsulfonyl)benzamide,
N-[4-[3,5-bis(trifluoromethyl)-1H-pyrazol-1-yl]phenyl]-2-iodobenzamide,
N-[4-[3,5-bis(trifluoromethyl)-1H-pyrazol-1-yl]phenyl]-4-heptybenzamide,
N-[4-[3,5-bis(trifluoromethyl)-1H-pyrazol-1-yl]phenyl]-2-furancarboxamide,
N-[4-[3,5-bis(trifluoromethyl)-1H-pyrazol-1-yl]phenyl]-2-fluorobenzamide,
N-[4-[3,5-bis(trifluoromethyl)-1H-pyrazol-1-yl]phenyl]-N′-methyl-1,2-benzenedicarboxamide,
N-[4-[3,5-bis(trifluoromethyl)-1H-pyrazol-1-yl]phenyl]-4-pyridinecarboxamide,
N-[4-[3,5-bis(trifluoromethyl)-1H-pyrazol-1-yl]phenyl]-4-chloro-2-nitrobenzamide,
N-[4-[3,5-bis(trifluoromethyl)-1H-pyrazol-1-yl]phenyl]-4-cinnolinecarboxamide,
4-acetyl-N-[4-[3,5-bis(trifluoromethyl)-1H-pyrazol-1-yl]phenyl]benzamide,
1,1-dimethylethyl 4-[[[4-[3,5-bis(trifluoromethyl)-1H-pyrazol-1-yl]phenyl]-amino]carbonyl]-1-piperidinecarboxylate,
N-[4-[3,5-bis(trifluoromethyl)-1H-pyrazol-1-yl]phenyl]-2-pyridinecarboxamide,
N-[4-[3,5-bis(trifluoromethyl)-1H-pyrazol-1-yl]phenyl]-4-(diethylamino)benzamide,
N-[4-[3,5-bis(trifluoromethyl)-1H-pyrazol-1-yl]phenyl]cyclopentanecarboxmide,
N-[4-[3,5-bis(trifluoromethyl)-1H-pyrazol-1-yl]phenyl]cyclohexanecarboxmide,
N-[4-[3,5-bis(trifluoromethyl)-1H-pyrazol-1-yl]phenyl]-4-piperidinecarboxamide,
N-[4-[3,5-bis(trifluoromethyl)-1H-pyrazol-1-yl]phenyl]-3-(methylsulfonyl)benzamide,
N-[4-[3,5-bis(trifluoromethyl)-1H-pyrazol-1-yl]phenyl]-2-(trifluoromethyl)benzamide,
3-[[[4-[3,5-bis(trifluoromethyl)-1H-pyrazol-1-yl]phenyl]methyl]amino]benzonitrile,
methyl 3-[[[4-[3,5-bis(trifluoromethyl)-1H-pyrazol-1-yl]phenyl]amino]carbonyl]benzoate,
N-[4-[3,5-bis(trifluoromethyl)-1H-pyrazol-1-y]phenyl-3-chlorobenzamide,
N-[4-[3,5-bis(trifluoromethyl)-1H-pyrazol-1-yl]phenyl]-2-thiophenecarboxamide,
(E)-3-[2-[4-[3,5-bis(trifluoromethyl)-1H-pyrazol-1-yl]phenyl]ethenyl]benzonitrile,
N-[4-[3,5-bis(trifluoromethyl)-1H-pyrazol-1-yl]phenyl]-1,4-benzenedicarboxamide,
N-[4-[3,5-bis(trifluoromethyl)-1H-pyrazol-1-yl]phenyl]-3,5-dinitrobenzamide,
N-[4-[3,5-bis(trifluoromethyl)-1H-pyrazol-1-yl]phenyl]-2,4-difluorobenzamide,
N-[4-[3,5-bis(trifluoromethyl)-1H-pyrazol-1-yl]phenyl]-2-nitrobenzamide,
N-[4-[3,5-bis(trifluoromethyl)-1H-pyrazol-1-yl]phenyl]-3-cyanobenzamide,
N-[4-[3,5-bis(trifluoromethyl)-1H-pyrazol-1-yl]phenyl]-1,3-benzenedicarboxamide,
(Z)-3-[2-[4-[3,5-bis(trifluoromethyl)-1H-pyrazol-1-yl]phenyl]ethenyl]benzonitrile,
N-[4-[3,5-bis(trifluoromethyl)-1H-pyrazol-1-yl]phenyl]-3-nitrobenzamide,
3-(aminosulfonyl)-N-[4-[3,5-bis(trifluoromethyl)-1H-pyrazol-1-yl]phenyl]benzamide,
methyl 4-[[[4-[3,5-bis(trifluoromethyl)-1H-pyrazol-1-yl]phenyl]amino]carbonyl]benzoate,
N-[4-[3,5-bis(trifluoromethyl)-1H-pyrazol-1-yl]phenyl]-2-methoxybenzamide,
N-[4-[3,5-bis(trifluoromethyl)-1H-pyrazol-1-yl]phenyl]-3-bromobenzamide,
N-[4-[3,5-bis(trifluoromethyl)-1H-pyrazol-1-yl]phenyl]-3-methoxybenzamide,
N-[4-[3,5-bis(trifluoromethyl)-1H-pyrazol-1-yl]phenyl]-3-fluorobenzamide,
N-[4-[3,5-bis(trifluoromethyl)-1H-pyrazol-1-yl]phenyl]-2-bromobenzamide,
N-[4-[3,5-bis(trifluoromethyl)-1H-pyrazol-1-yl]phenyl]-1,3-benzodioxole-5-carboxamide,
N-[4-[3,5-bis(trifluoromethyl)-1H-pyrazol-1-yl]phenyl]-2,6-dichloro-3-pyridinecarboxamide,
N-[4-[3,5-bis(trifluoromethyl)-1H-pyrazol-1-yl]phenyl]-2-chloro-3-pyridinecarboxamide,
N-[4-[3,5-bis(trifluoromethyl)-1H-pyrazol-1-yl]phenyl]-2-chloro-6-methyl-3-pyridinecarboxamide,
N-[4-[3,5-bis(trifluoromethyl)-1H-pyrazol-1-yl]phenyl]-4-fluoro-γ-oxobenzenebutanamide,
N-[4-[3,5-bis(trifluoromethyl)-1H-pyrazol-1-yl]phenyl]-1,2,3,4-tetrahydro-2-naphthalenecarboxamide,
(E)-1-[4-[2-(2-chlorophenyl)ethenyl]phenyl]-3,5-bis(trifluoromethyl)-1H-pyrazole,
N-[4-[3,5-bis(trifluoromethyl)-1H-pyrazol-1-yl]phenyl]-2-(4-chlorophenoxy)-2-methylpropanamide,
N-[4-[3,5-bis(trifluoromethyl)-1H-pyrazol-1-yl]phenyl]acetamide,
4-[[[4-[3,5-bis(trifluoromethyl)-1H-pyrazol-1-yl]phenyl]amino]carbonyl]benzoic acid,
phenylmethyl N-[4-[3,5-bis(trifluoromethyl)-1H-pyrazol-1-yl]phenyl]amino]-4-oxobutyl]carbamate,
3-[[[4-[3,5-bis(trifluoromethyl)-1H-pyrazol-1-yl]phenyl]amino]carbonyl]benzoic acid,
N-[4-[3,5-bis(trifluoromethyl)-1H-pyrazol-1-yl]phenyl]-2,6-difluorobenzamide,
N-[4-[3,5-bis(trifluoromethyl)-1H-pyrazol-1-yl]phenyl]-3-bromo-2-thiophenecarboxamide,
N-[4-[3,5-bis(trifluoromethyl)-1H-pyrazol-1-yl]phenyl]-3-methyl-2-thiophenecarboxamide,
2-amino-N-[4-[3,5-bis(trifluoromethyl)-1H-pyrazol-1-yl]phenyl]benzamide,
N-[4-[3,5-bis(trifluoromethyl)-1H-pyrazol-1-yl]phenyl]-2-fluoro-3-pyridinecarboxamide,
N-[4-[3,5-bis(trifluoromethyl)-1H-pyrazol-1-yl]phenyl]-3-chloro-4-(methylsulfonyl)-2-thiophenecarboxamide,
N-[4-[3,5-bis(trifluoromethyl)-1H-pyrazol-1-yl]phenyl]-1H-pyrrole-2-carboxamide
N-[4-[3,5-bis(trifluoromethyl)-1H-pyrazol-1-yl]phenyl]-3,6-dichloro-2-pyridinecarboxamide
N-[4-[3,5-bis(trifluoromethyl)-1H-pyrazol-1-yl]phenyl]-2-(2-nitrophenoxy)acetamide,
N-[4-[3,5-bis(trifluoromethyl)-1H-pyrazol-1-yl]phenyl]-4-chlorobenzeneacetamide,
N-[4-[3,5-bis(trifluoromethyl)-1H-pyrazol-1-yl]phenyl]-1H-indole-2-acetamide,
(E)-N-[4-[3,5-bis(trifluoromethyl)-1H-pyrazol-1-yl]phenyl]-3-(2-thienyl)-2-propenamide,
N-[4-[3,5-bis(trifluoromethyl)-1H-pyrazol-1-yl]phenyl]pyazinecarboxamide,
1,1-dimethylethyl [[4-[4-[3,5-bis(trifluoromethyl)-1H-pyrazol-1-yl]phenyl]amino]-4-oxobutyl]carbamate,
1-acetyl-N-[4-[3,5-bis(trifluoromethyl)-1H-pyrazol-1-yl]phenyl]-4-piperidinecarboxamide,
N-[4-[3,5-bis(trifluoromethyl)-1H-pyrazol-1-yl]phenyl]butanamide,
N-[4-[3,5-bis(trifluoromethyl)-1H-pyrazol-1-yl]phenyl]-4-chloro-2-methoxybenzamide,
N-[4-[3,5-bis(trifluoromethyl)-1H-pyrazol-1-yl]phenyl]-α-methyl-4-(2-thienylcarbonyl)benzeneacetamide,
N-[4-[3,5-bis(trifluoromethyl)-1H-pyrazol-1-yl]phenyl]-α-methyl-4-(2-thienylcarbonyl)benzeneacetamide,
N-[4-[3,5-bis(trifluoromethyl)-1H-pyrazol-1-yl]phenyl]-2-methoxy-4-(methythio)benzamide,
N-[4-[3,5-bis(trifluoromethyl)-1H-pyrazol-1-yl]phenyl]-4-hydroxy-3-nitrobenzamide,
N-[4-[3,5-bis(trifluoromethyl)-1H-pyrazol-1-yl]phenyl]-3,4-dihydroxybenzamide,
N-[4-[3,5-bis(trifluoromethyl)-1H-pyrazol-1-yl]phenyl]-2-hydroxy-6-methoxybenzamide,
N-[4-[3,5-bis(trifluoromethyl)-1H-pyrazol-1-yl]phenyl]-2,4-bis(trifluoromethyl)benzamide,
N-[4-[3,5-bis(trifluoromethyl)-1H-pyrazol-1-yl]phenyl]-5-methyl-4-isoxazolecarboxamide,
4-[3,5-bis(trifluoromethyl)-1H-pyrazol-1-yl]-N-(2-chlorophenyl)benzamide,
4-[3,5-bis(trifluoromethyl)-1H-pyrazol-1-yl]-N-(3-cyanophenyl)benzamide,
4-[3,5-bis(trifluoromethyl)-1H-pyrazol-1-yl]-N-(2,4-difluorophenyl)benzamide,
4-[3,5-bis(trifluoromethyl)-1H-pyrazol-1-yl]-N-(2-cyanophenyl)benzamide,
N-[4-[5-[3,5-dimethyl-1H-1,2,4-triazol-1-yl)-3-(trifluoromethyl)-1H-pyrazol-1-yl]phenyl]-4-isoxazolecarboxamide,
4-[3,5-bis(trifluoromethyl)-1H-pyrazol-1-yl]-N-(2-nitrophenyl)benzamide,
4-[3,5-bis(trifluoromethyl)-1H-pyrazol-1-yl]-N-(2,6-difluorophenyl)benzamide,
4-[3,5-bis(trifluoromethyl)-1H-pyrazol-1-yl]-N-(2-bromophenyl)benzamide,
4-[3,5-bis(trifluoromethyl)-1H-pyrazol-1-yl]-N-(4-cyanophenyl)benzamide,
4-[3,5-bis(trifluoromethyl)-1H-pyrazol-1-yl]-N-(4-pyridinyl)benzamide,
N-[4-[3,5-bis(trifluoromethyl)-1H-pyrazol-1-yl]phenyl]-4-fluoro-3-(tnrfluoromethyl)benzamide,
N-[2-(aminocarbonyl)phenyl]-4-[3,5-bis(trifluoromethyl)-1H-pyrazol-1-yl]benzamide,
N-[3-[3,5-bis(trifluoromethyl)-1H-pyrazol-1-yl]phenyl]-4-chlorobenzeneacetamide,
N-[3-[3,5-bis(trifluoromethyl)-1H-pyrazol-1-yl]phenyl]-2,3-dichlorobenzamide,
N-[3-[3,5-bis(trifluoromethyl)-1H-pyrazol-1-yl]phenyl]-2-chloro-5-nitrobenzamide,
N-[4-[3,5-bis(trifluoromethyl)-1H-pyrazol-1-yl]phenyl]-4-fluoro-3-nitrobenzamide,
N-[4-[3,5-bis(trifluoromethyl)-1H-pyrazol-1-yl]phenyl]-4-fluoro-3-nitrobenzamide,
N-[4-[3,5-bis(trifluoromethyl)-1H-pyrazol-1-yl]phenyl]-4-fluoro-2-(trifluoromethyl)-benzamide,
N-[3-[3,5-bis(trifluoromethyl)-1H-pyrazol-1-yl]phenyl]-4-fluorobenzamide,
N-[3-[3,5-bis(trifluoromethyl)-1H-pyrazol-1-yl]phenyl]-2,4-difluorobenzamide,
N-[3-[3,5-bis(trifluoromethyl)-1H-pyrazol-1-yl]phenyl]-3-cyanobenzamide,
N-[4-[3,5-bis(trifluoromethyl)-1H-pyrazol-1-yl]phenyl]-2-bromo-3-nitrobenzamide,
N-[4-[3,5-bis(trifluoromethyl)-1H-pyrazol-1-yl]phenyl]-2-chloro-4-fluorobenzamide,
N-[4-[3,5-bis(trifluoromethyl)-1H-pyrazol-1-yl]phenyl]-2-chloro-4-(methylsulfonyl)-benzamide,
N-[4-[3,5-bis(trifluoromethyl)-1H-pyrazol-1-yl]phenyl]-2,5-dichlorobenzamide,
N-[4-[3,5-bis(trifluoromethyl)-1H-pyrazol-1-yl]phenyl]-2,3-difluorobenzamide,
N-[4-[3,5-bis(trifluoromethyl)-1H-pyrazol-1-yl]phenyl]-3-chloro-4-fluorobenzamide,
N-[4-[3,5-bis(trifluoromethyl)-1H-pyrazol-1-yl]phenyl]-2,5-difluorobenzamide,
N-[4-[3,5 -bis(trifluoromethyl)-1H-pyrazol-1-yl]phenyl]-2-chloro-6-fluorobenzamide,
N-[4-[3,5-bis(trifluoromethyl)-1H-pyrazol-1-yl]phenyl]-2-fluoro-6-(trifluoromethyl)-benzamide,
N-[4-[3,5-bis(trifluoromethyl)-1H-pyrazol-1-yl]phenyl]-3 -chloro-2-fluorobenzamide,
N-[4-[3,5-bis(trifluoromethyl)-1H-pyrazol-1-yl]phenyl]-2-chloro-4-methoxybenzamide,
N-[4-[3,5-bis(trifluoromethyl)-1H-pyrazol-1-yl]phenyl]-2,6-dichloro-3-nitrobenzamide,
N-[4-[3,5-bis(trifluoromethyl)-1H-pyrazol-1-yl]phenyl]-4-bromo-2-chlorobenzamide,
N-[4-[3,5-bis(trifluoromethyl)-1H-pyrazol-1-yl]phenyl]-3,4-difluorobenzamide,
N-[4-[3,5-bis(trifluoromethyl)-1H-pyrazol-1-yl]phenyl]-2-bromo-5-methoxybenzamide,
N-[4-[3,5-bis(trifluoromethyl)-1H-pyrazol-1-yl]phenyl]-4-chloro-2-hydroxybenzamide,
N-[4-[3,5-bis(trifluoromethyl)-1H-pyrazol-1-yl]phenyl]-3-bromo-4-methoxybenzamide,
N-[4-[3,5-bis(trifluoromethyl)-1H-pyrazol-1-yl]phenyl]-3-bromo-4-hydroxybenzamide,
N-[4-[3,5-bis(trifluoromethyl)-1H-pyrazol-1-yl]phenyl]-2-chloro-4,5-difluorobenzamide,
N-[3-[3,5-bis(trifluoromethyl)-1H-pyrazol-1-yl]phenyl]-2,6-difluorobenzamide,
N-[4-[3,5-bis(trifluoromethyl)-1H-pyrazol-1-yl]phenyl]-4-chloro-2,5-difluorobenzamide,
N-[4-[3,5-bis(trifluoromethyl)-1H-pyrazol-1-yl]phenyl]-2,3,4-trifluorobenzamide,
N-[4-[3,5-bis(trifluoromethyl)-1H-pyrazol-1-yl]phenyl]-3,4,5-trifluorobenzamide,
N-[4-[3,5-bis(trifluoromethyl)-1H-pyrazol-1-yl]phenyl]-2,4,5-trifluorobenzamide,
N-[4-[3,5-bis(trifluoromethyl)-1H-pyrazol-1-yl]phenyl]-2,4,6-trifluorobenzamide,
N-[4-[3,5-bis(trifluoromethyl)-1H-pyrazol-1-yl]phenyl]-2,6-difluoro-3-nitrobenzamide,
N-[4-[3,5-bis(trifluoromethyl)-1H-pyrazol-1-yl]phenyl]-2,3,5-trifluorobenzamide,
N-[4-[3,5-bis(trifluoromethyl)-1H-pyrazol-1-yl]phenyl]-2,4-dichloro-6-fluorobenzamide,
N-4-[3,5-bis(trifluoromethyl)-1H-pyrazol-1-yl]phenyl-2,4-dichloro-3,5-dinitrobenzamide,
N-[4-[3,5-bis(trifluoromethyl)-1H-pyrazol-1-yl]phenyl]-2,3,5,6-tetrafluorobenzamide,
N-[4-[3,5-bis(trifluoromethyl)-1H-pyrazol-1-yl]phenyl]-2,3,4,5-tetrafluorobenzamide,
N-[4-[3,5-bis(trifluoromethyl)-1H-pyrazol-1-yl]phenyl]-4-bromo-2,3,5,6-tetrafluorobenzamide,
N-[4-[3,5-bis(trifluoromethyl)-1H-pyrazol-1-yl]phenyl]-5-methyl-2-nitrobenzamide,
N-[3-[3,5-bis(trifluoromethyl)-1H-pyrazol-1-yl]phenyl]-4-cyanobenzamide,
N-[4-[3,5-bis(trifluoromethyl)-1H-pyrazol-1-yl]phenyl]-3-thiophenecarboxamide,
N-[4-[3,5-bis(trifluoromethyl)-1H-pyrazol-1-yl]phenyl]-5-isoxazolecarboxamide,
N-[4-[3,5-bis(trifluoromethyl)-1H-pyrazol-1-yl]phenyl]tetrahydro-2-furancarboxamide,
N-[4-[3,5-bis(trifluoromethyl)-1H-pyrazol-1-yl]phenyl]-2-pyrrolidinecarboxamide,
N-[4-[3,5-bis(trifluoromethyl)-1H-pyrazol-1-yl]phenyl]tetrahydro-3-furancarboxamide,
N-[4-[3,5-bis(trifluoromethyl)-1H-pyrazol-1-yl]phenyl]-1,2,3-thiadiazole-5-carboxamide,
N-[4-[3,5-bis(trifluoromethyl)-1H-pyrazol-1-yl]phenyl]-2-chloro-4-pyridinecarboxamide,
1,1-dimethylethyl 2-[[[4-[3,5-bis(trifluoromethyl)-1H-pyrazol-1-yl]phenyl]amino]carbonyl]-1-pyrrolidinecarboxylate,
N-[4-[3,5-bis(trifluoromethyl)-1H-pyrazol-1-yl]phenyl]-5-nitro-2-furancarboxamide,
N-[4-[3,5-bis(trifluoromethyl)-1H-pyrazol-1-yl]phenyl]-1-methyl-1H-pyrrole-2-carboxamide,
N-[4-[3,5 -bis(trifluoromethyl)-1H-pyrazol-1-yl]phenyl]-6-chloro-3-pyridinecarboxamide,
N-[4-[3,5-bis(trifluoromethyl)-1H-pyrazol-1-yl]phenyl]-5-bromo-2-furancarboxamide,
N-[4-[3,5-bis(trifluoromethyl)-1H-pyrazol-1-yl]phenyl]-3-methyl-2-furancarboxamide,
N-[4-[3,5-bis(trifluoromethyl)-1H-pyrazol-1-yl]phenyl]-5-chloro-2-thiophenecarboxamide,
(S)-N-[4-[3,5-bis(trifluoromethyl)-1H-pyrazol-1-yl]phenyl]tetrahydro-5-oxo-2-furan-carboxamide,
N-[4-[3,5-bis(trifluoromethyl)-1H-pyrazol-1-yl]phenyl]-5-oxo-2-pyrrolidinecarboxamide,
N-[4-[3,5-bis(trifluoromethyl)-1H-pyrazol-1-yl]phenyl]-5-bromo-3-pyridinecarboxamide,
N-[4-[3,5-bis(trifluoromethyl)-1H-pyrazol-1-yl]phenyl]-5-nitro-3-thiophenecarboxamide,
1,1-dimethylethyl 4-[[[4-[3,5-bis(trifluoromethyl)-1H-pyrazol-1-yl]phenyl]amino]-carbonyl]-3-thiazolidinecarboxylate,
N-[4-[3,5-bis(trifluoromethyl)-1H-pyrazol-1-yl]phenyl]-5-methoxy-3-thiophenecarboxamide,
N-[2-[3,5-bis(trifluoromethyl)-1H-pyrazol-1-yl]-5-pyridinyl]-2-chlorobenzamide,
N-[2-[3,5-bis(trifluoromethyl)-1H-pyrazol-1-yl]-5-pyridinyl]-3-cyanobenzamide,
N-[2-[3,5-bis(trifluoromethyl)-1H-pyrazol-1-yl]-5-pyridinyl]-2-chloro-4,5-difluorobenzamide,
N-[4-[3,5-bis(trifluoromethyl)-1H-pyrazol-1-yl]phenyl]-2,3-dibromo-5-thiophenecarboxamide,
N-[4-[3,5-bis(trifluoromethyl)-1H-pyrazol-1-yl]phenyl]-3-fluoro-4-pyridinecarboxamide,
N-[4-[3,5-bis(trifluoromethyl)-1H-pyrazol-1-yl]phenyl]-1-methyl-1H-pyrazole-4-carboxamide,
N-[4-[3,5-bis(trifluoromethyl)-1H-pyrazol-1-yl]phenyl]-5-chloro-4-methoxy-3-thiophenecarboxamide,
N-[4-[3,5-bis(trifluoromethyl)-1H-pyrazol-1-yl]phenyl]-5,6-dichloro-3-pyridinecarboxamide
N-[4-[3,5-bis(trifluoromethyl)-1H-pyrazol-1-yl]phenyl]-2,6-dichloro-4-pyridinecarboxamide,
N-[4-[3,5-bis(trifluoromethyl)-1H-pyrazol-1-yl]phenyl]-2,5-dichloro-3-pyridinecarboxamide,
N-[4-[3,5-bis(trifluoromethyl)-1H-pyrazol-1-yl]-3-(trifluoromethyl)phenyl]-4-chlorobenzamide,
N-[4-[3,5-bis(trifluoromethyl)-1H-pyrazol-1-yl]-2-fluorophenyl]-2,4-difluorobenzamide,
N-[2,4-bis[3,5-bis(trifluoromethyl)-1H-pyrazol-1-yl]phenyl]-2,4-difluorobenzamide,
methyl 2-[3,5-bis(trifluoromethyl)-1H-pyrazol-1-yl]-5-[(5-bromo-2-chlorobenzoyl)amino]benzoate,
N-[4-[3,5-bis(trifluoromethyl)-1H-pyrazol-1-yl]-3-(trifluoromethyl)phenyl]-3,5-dimethyl-4-isoxazolecarboxamide,
N-[4-[3,5-bis(trifluoromethyl)-1H-pyrazol-1-yl]-3-(trifluoromethyl)phenyl]-4-methyl-1,2,3-thiadiazole-5-carboxamide,
N-[4-[3,5-bis(trifluoromethyl)-1H-pyrazol-1-yl]-3-chlorophenyl]-3,5-dimethyl-4-isoxazolecarboxamide,
N-[4-[3,5-bis(trifluoromethyl)-1H-pyrazol-1-yl]-2-(trifluoromethyl)phenyl]-3,5-dimethyl-4-isoxazolecarboxamide,
N-[4-[3,5-bis(trifluoromethyl)-1H-pyrazol-1-yl]-2-methylphenyl]-4-methyl-1,2,3-thiadiazole-5-carboxamide,
N-[4-[3,5-bis(trifluoromethyl)-1H-pyrazol-1-yl]-2-methoxyphenyl]-4-methyl-1,2,3-thiadiazole-5-carboxamide,
4-chloro-N-[4-[5-methyl-3-(trifluoromethyl)-1H-pyrazol-1-yl]phenyl]benzamide,
4-methyl-N-[4-[5-methyl-3-(trifluoromethyl)-1H-pyrazol-1-yl]phenyl]-1,2,3-thiadiazole-5-carboxamide,
3,5-dimethyl-N-[4-[5-methyl-3-(trifluoromethyl)-1H-pyrazol-1-yl]phenyl]-4-isoxazolecarboxamide,
4-chloro-N-[4-(5-methyl-1H-pyrazol-1-yl)phenyl]benzamide,
4-methyl-N-[4-(5-methyl-1H-pyrazol-1-yl)phenyl]-1,2,3-thiadiazole-5-carboxamide,
3,5-dimethyl-N-[4-(5-methyl-1H-pyrazol-1-yl)phenyl]-4-isoxazolecarboxamide,
3,5-dimethyl-N-[4-[3-(trifluoromethyl)-1H-pyrazol-1-yl)phenyl]-4-isoxazolecarboxamide,
N-[4-[5-hydroxy-3-(trifluoromethyl)-1H-pyrazol-1-yl]phenyl]-4-methyl-1,2,3-thiadiazole-5-carboxamide,
N-[4-[5-(3,5-dimethyl-1H-1,2,4-triazol-1-yl)-3-(trifluoromethyl)-1H-pyrazol-1-yl]phenyl]-1,2,3-thiadoazole-5-carboxamide,
3-amino-N-(4-(3,5-bis(trifluoromethyl)-1H-pyrazol-1-yl)phenyl)isonicotinamide,
N-(4-(3,5-bis(trifluoromethyl)-1H-pyrazol-1-yl)phenyl)-3-chloro-5-methoxyisonicotinamide,
N-(6- (3,5-bis(trifluoromethyl)-1H-pyrazol-1-yl)-3-pyridinyl)-2-fluorobenzamide,
methyl 2-(3,5-bis(trifluoromethyl)-1H-pyrazol-1-yl)-5-((2-fluorobenzoyl)amino)benzoate,
4-(aminomethyl)-N-(4-(3,5-bis(trifluoromethyl)-1H-pyrazol-1-yl)phenyl)-2-chlorobenzamide,
N-(4-(3,5-bis(trifluoromethyl)-1H-pyrazol-1-yl)phenyl)-2-methylacrylamide,
N-(4-(3,5-bis(trifluoromethyl)-1H-pyrazol-1-yl)phenyl)-4-chloro-2-fluorobenzamide,
N-(5-(3,5-bis(trifluoromethyl)-1H-pyrazol-1-yl)-2-pyridinyl)-2-fluorobenzamide,
N-(3-amino-4-(3,5-bis(trifluoromethyl)-1H-pyrazol-1-yl)phenyl)-2-fluorobenzamide,
N-(4-(3,5-bis(trifluoromethyl)-1H-pyrazol-1-yl)-3-cyanophenyl)-2-fluorobenzamide,
N-(4-(5-cyano-3-(trifluoromethyl)-1H-pyrazol-1-yl)phenyl)-2-fluorobenzamide,
2-fluoro-N-(4-(5-(2-furyl)-3-(trifluoromethyl)-1H-pyrazol-1-yl)phenyl)benzamide,
N-(4-(5-cyano-3-(trifluoromethyl)-1H-pyrazol-1-yl)phenyl)-4-methyl-1,2,3-thiadiazole-5-carboxamide,
N-(4-(5-cyano-3-(trifluoromethyl)-1H-pyrazol-1-yl)phenyl)isonicotinamide,
N-(4-(5-cyano-3-(trifluoromethyl)-1H-pyrazol-1-yl)phenyl)-3-fluoroisonicotinamide,
N-(4-(5-acetyl-3-(trifluoromethyl)-1H-pyrazol-1-yl)phenyl)-2-fluorobenzamide,
N-(4-(5-cyano-3-(trifluoromethyl)-1H-pyrazol-1-yl)phenyl)-2-fluoronicotinamide,
N-(4-(5-cyano-3-(trifluoromethyl)-1H-pyrazol-1-yl)phenyl)-2,3,5-trifluorobenzamide,
2-fluoro-N-(4-(5-(2-thienyl)-3-(trifluoromethyl)-1H-pyrazol-1-yl)phenyl)benzamide,
2-fluoro-N-(4-(5-(methylsulfanyl)-3-(trifluoromethyl)-1H-pyrazol-1-yl)phenyl)benzamide,
2-fluoro-N-(4-(5-(3-pyridinyl)-3-(trifluoromethyl)-1H-pyrazol-1-yl)phenyl)benzamide,
3-fluoro-N-(4-(5-(2-thienyl)-3-(trifluoromethyl)-1H-pyrazol-1-yl)phenyl)isonicotinamide,
N-(4-(5-methoxy-3-(trifluoromethyl)-1H-pyrazol-1-yl)phenyl)isonicotinamide,
2-fluoro-N-(4-(5-methoxy-3-(trifluoromethyl)-1H-pyrazol-1-yl)phenyl)benzamide,
N-(4-(5-methoxy-3-(trifluoromethyl)-1H-pyrazol-1-yl)phenyl)-4-methyl-1,2,3-thiadiazole-5-carboxamide,
N-(4-(5-acetyl-3-(trifluoromethyl)-1H-pyrazol-1-yl)phenyl)-2-fluoronicotinamide,
2-fluoro-N-(4-(5-(methylsulfanyl)-3-(trifluoromethyl)-1H-pyrazol-1-yl)phenyl)nicotinamide,
2-fluoro-N-(4-(5-methoxy-3-(trifluoromethyl)-1H-pyrazol-1-yl)phenyl)nicotinamide,
N-(4-(5-ethoxy-3-(trifluoromethyl)-1H-pyrazol-1-yl)phenyl)-2-fluoronicotinamide,
3-fluoro-N-(4-(5-(methylsulfanyl)-3-(trifluoromethyl)-1H-pyrazol-1-yl)phenyl)isonicotinamide,
3-fluoro-N-(4-(5-methoxy-3-(trifluoromethyl)-1H-pyrazol-1-yl)phenyl)isonicotinamide,
N-(4-(5-(difluoromethoxy)-3-(trifluoromethyl)-1H-pyrazol-1-yl)phenyl)isonicotinamide,
N-(4-(5-(difluoromethoxy)-3-(trifluoromethyl)-1H-pyrazol-1-yl)phenyl)-4-methyl-1,2,3-thiadiazole-5-carboxamide,
N-(4-(5-(difluoromethoxy)-3-(trifluoromethyl)-1H-pyrazol-1-yl)phenyl)-2-fluoronicotinamide,
N-(4-(5-chloro-3-(trifluoromethyl)-1H-pyrazol-1-yl)phenyl)-2-fluoronicotinamide,
2-fluoro-N-(4-(5-nitro-3-(trifluoromethyl)-1H-pyrazol-1-yl)phenyl)benzamide,
N-(4-(5-(difluoromethoxy)-3-(trifluoromethyl)-1H-pyrazol-1-yl)phenyl)-3-fluoroisonicotinamide,
N-(4-(5-chloro-3-(trifluoromethyl)-1H-pyrazol-1-yl)phenyl)-3-fluoroisonicotinamide,
N-(4-(5-bromo-3-(trifluoromethyl)-1H-pyrazol-1-yl)phenyl)-3-fluoroisonicotinamide,
3-fluoro-N-(4-(5-nitro-3-(trifluoromethyl)-1H-pyrazol-1-yl)phenyl)isonicotinamide,
N-(4-(5-bromo-3-(trifluoromethyl)-1H-pyrazol-1-yl)phenyl)-4-methyl-1,2,3-thiadiazole-5-carboxamide,
N-(4-(5-chloro-3-(trifluoromethyl)-1H-1,2,4-triazol-1-yl)phenyl)-3-fluoroisonicotinamide,
3-fluoro-N-(4-(5-(1-methyl-1H-pyrrol-3-yl)-3-(trifluoromethyl)-1H-pyrazol-1-yl)phenyl)isonicotinamide,
3-chloro-N-(4-(5-chloro-3-(trifluoromethyl)-1H-pyrazol-1-yl)phenyl)isonicotinamide,
N-(4-(5-bromo-3-(trifluoromethyl)-1H-pyrazol-1-yl)phenyl)-2,3-difluorobenzamide,
N-(4-(5-bromo-3-(trifluoromethyl)-1H-pyrazol-1-yl)phenyl)-3-chloroisonicotinamide,
2-chloro-N-(4-(5-cyano-3-(trifluoromethyl)-1H-pyrazol-1-yl)phenyl)benzamide,
3-chloro-N-(4-(5-cyano-3-(trifluoromethyl)-1H-pyrazol-1-yl)phenyl)isonicotinamide,
N-(4-(5-(difluoromethoxy)-3-(trifluoromethyl)-1H-pyrazol-1-yl)phenyl)-2-fluorobenzamide,
2-chloro-N-(4-(5-(difluoromethoxy)-3-(trifluoromethyl)-1H-pyrazol-1-yl)phenyl)benzamide,
N-(4-(5-(difluoromethoxy)-3-(trifluoromethyl)-1H-pyrazol-1-yl)phenyl)-2,3-difluorobenzamide,
3-fluoro-N-(4-(3-(4-pyridinyl)-5-(trifluoromethyl)-1H-pyrazol-1-yl)phenyl)isonicotinamide,
N-(4-(5-(difluoromethyl) -3-(3-pyridinyl)-1H-pyrazol-1-yl)phenyl)-3-fluoroisonicotinamide,
N-(4-(5-cyano-3-(2-pyridinyl)-1H-pyrazol-1-yl)phenyl)-3-fluoroisonicotinamide,
N-(4-(5-cyano-3-(3-pyridinyl)-1H-pyrazol-1-yl)phenyl)-4-methyl-1,2,3-thiadiazole-5-carboxamide,
3-fluoro-N-(4-(5-nitro-3-(3-pyridinyl)-1H-pyrazol-1-yl)phenyl)isonicotinamide,
4-methyl-N-(4-(5-nitro-3-(3-pyridinyl)-1H-pyrazol-1-yl)phenyl)-1,2,3-thiadiazolecarboxamide,
N-(4-(5-cyano-3-(1,3-thiazol-2-yl)-1H-pyrazol-1-yl)phenyl)-3-fluoroisonicotinamide,
N-(4-(3-(5-bromo-3-pyridinyl)-5-(difluoromethoxy)-1H-pyrazol-1-yl)phenyl)-3-fluoroisonicotinamide,
N-(4-(5-cyano-3-(1,3-thiazol-2-yl)-1H-pyrazol-1-yl)phenyl)-4-methyl-1,2,3-thiadiazole-5-carboxamide,
N-(4-(5-cyano-3-(1,3-thiazol-2-yl)-1H-pyrazol-1-yl)phenyl)-3-fluoroisonicotinamide,
4-methyl-N-(4-(3-(1,3-thiazol-2-yl)-5-(trifluoromethyl)-1H-pyrazol-1-yl)phenyl)-1,2,3-thiadiazole-5-carboxamide,
N-(4-(3-(2,4-dimethyl-1,3-thiazol-5-yl)-5-(trifluoromethyl)-1H-pyrazol-1-yl)phenyl)-3-fluoroisonicotinamide,
3-fluoro-N-(4-(3-tetrahydro-2-furanyl-5-(trifluoromethyl)-1H-pyrazol-1-yl)phenyl)isonicotinamide,
N-(4-(5-chloro-3-(1,3-thiazol-2-yl)-1H-pyrazol-1-yl)phenyl)-3-fluoroisonicotinamide,
N-(4-(5-chloro-3-(3-furyl)-1H-pyrazol-1-yl)phenyl)-4-methyl-1,2,3-thiadiazole-5 -carboxamide,
N-(4-(5-chloro-3-(3-furyl)-1H-pyrazol-1-yl)phenyl)-3-fluoroisonicotinamide,
N-(4-(5-cyano-3-tetrahydro-2-furanyl-1H-pyrazol-1-yl)phenyl)-3-fluoroisonicotinamide,
N-(4-(5-(difluoromethoxy)-3-(1-methyl-1H-pyrrol-3-yl)-1H-pyrazol-1-yl)phenyl)-3-fluoroisonicotinamide,
N-(4-(5-(difluoromethoxy)-3-(3-furyl)-1H-pyrazol-1-yl)phenyl)-3-fluoroisonicotinamide, and
N-(4-(5-(difluoromethoxy)-3-(1-methyl-1H-pyrrol-2-yl)-1H-pyrazol-1-yl)phenyl)-3-fluoroisonicotinamide.

43. A method of inhibiting interleukin-2, interleukin-4, and interleukin-5 production in a mammal comprising adminstering a therapeutically effective amount of a compound of claim 1.

44. A method of treating immunologically-mediated diseases in a mammal comprising administering a therapeutically effective amount of a compound of Formula I 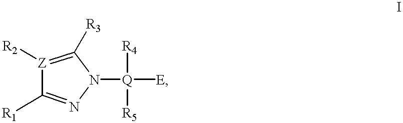
or a pharmaceutically acceptable salt or prodrug thereof, where
R₁ and R₃ are independently selected from
(1) hydrogen,
(2) aryl,
(3) perfluoroalkyl of one to fifteen carbons,
(4) halo,
(5) —CN,
(6) —NO₂,
(7) —OH,
(8) —OG where G is a hydroxyl protecting group,
(9) —CO₂R₆ where R₆ is selected from
(a) hydrogen,
(b) cycloalkyl of three to twelve carbons,
(c) aryl,
(d) aryl substituted with 1, 2, 3, 4, or 5 substituents independently selected from
(i) alkyl of one to fifteen carbons,
(ii) alkoxy of one to fifteen carbons,
(iii) thioalkoxy of one to fifteen carbons,
(iv) halo,
(v) —NO₂, and
(vi) —N₃,
(e) a carboxy protecting group,
(f) alkyl of one to fifteen carbons,
(g) alkyl of one to fifteen carbons substituted with 1, 2, or 3, or 4 substituents independently selected from
(i) alkoxy of one to fifteen carbons,
(ii) thioalkoxy of one to fifteen carbons,
(iii) aryl,
(iv) aryl substituted with 1, 2, 3, 4, or 5 substituents independently selected from alkyl of one to fifteen carbons,
alkoxy of one to fifteen carbons,
thioalkoxy of one to fifteen carbons,
halo,
—NO₂, and
—N₃,
(v) cycloalkyl of three to twelve carbons, and
(vi) halo,
(h) alkenyl of three to fifteen carbons, provided that a carbon of a carbon-carbon double bond is not attached directly to oxygen,
(i) alkynyl of three to fifteen carbons, provided that a carbon of a carbon-carbon triple bond is not attached directly to oxygen, and
(j) cycloalkyl of three to twelve carbons,
(10) —L₁NR₇R₈ where L₁ is selected from
(a) a covalent bond,
(b) —X′C(X)— where X and X′ are independently O or S,
(c) —C(X)—, and
(d) —NR₆— and
R₇ and R₈ are independently selected from
(a) hydrogen,
(b) alkanoyl where the alkyl part is one to fifteen carbons,
(c) alkoxycarbonyl where the alkyl part is one to fifteen carbons,
(d) alkoxycarbonyl where the alkyl part is one to fifteen carbons and is substituted with 1 or 2 substituents selected from the group consisting of aryl,
(e) cycloalkyl of three to twelve carbons,
(f) aryl,
(g) aryl substituted with 1, 2, 3, 4, or 5 substituents independently selected from
(i) alkyl of one to fifteen carbons,
(ii) alkoxy of one to fifteen carbons,
(iii) thioalkoxy of one to fifteen carbons,
(iv) halo,
(v) —NO₂, and
(vi) —N₃,
(h) —OR₆,
provided that only one of R₇ or R₈ is —OR₆,
(i) a nitrogen protecting group,
(j) alkyl of one to fifteen carbons,
(k) alkyl of one to fifteen carbons substituted with 1, 2, or 3, or 4 substituents independently selected from
(i) alkoxy of one to fifteen carbons,
(ii) thioalkoxy of one to fifteen carbons,
(iii) aryl,
(iv) aryl substituted with 1, 2, 3, 4, or 5 substituents independently selected from alkyl of one to fifteen carbons,
alkoxy of one to fifteen carbons,
thioalkoxy of one to fifteen carbons,
halo,
—NO₂, and
—N₃,
(v) cycloalkyl of three to fifteen carbons,
(vi) halo,
(vii) —CO₂R₆, and
(viii) —OH,
(l) alkenyl of three to fifteen carbons,
provided that a carbon of a carbon-carbon double bond is not attached directly to nitrogen,
(m) alkynyl of three to fifteen carbons, provided that a carbon of a carbon-carbon triple bond is not attached directly to nitrogen,
(n) —SO₂-alkyl, and
(o) cycloalkyl of three to twelve carbons, or
R₇ and R₈ together with the nitrogen atom to which they are attached form a ring selected from
(i) aziridine,
(ii) azetidine,
(iii) pyrrolidine,
(iv) piperidine,
(v) piperazine,
(vi) morpholine,
(vii) thiomorpholine, and
(viii) thiomorpholine sulfone
where (i)-(viii) can be optionally substituted with 1, 2, or 3 substituents selected from the group consisting of alkyl of one to fifteen carbons,
(11) —L₂R₉ where L₂ is selected from
(a) —L₁—,
(b) —O—, and
(c) —S(O)ₜ— where t is 0, 1, or 2 and
R₉ is selected from
(a) cycloalkyl of three to twelve carbons,
(b) aryl
(c) aryl substituted with 1, 2, 3, 4, or 5 substituents independently selected from
(i) alkyl of one to fifteen carbons,
(ii) alkoxy of one to fifteen carbons,
(iii) thioalkoxy of one to fifteen carbons,
(iv) halo,
(v) —NO₂, and
(vi) —N₃,
(d) alkyl of one to fifteen carbons,
(e) heterocycle,
(f) alkenyl of two to fifteen carbons, and
(e) alkyl of one to fifteen carbons substituted with 1, 2, or 3, or 4 substituents independently selected from
(i) alkenyl of two to fifteen carbons,
(ii) alkoxy of one to fifteen carbons,
(iii) —CN,
(iv) —CO₂R₆,
(v) —OH,
provided that no two —OH groups are attached to the same carbon,
(vi) thioalkoxy of one to fifteen carbons,
(vii) alkynyl of two to fifteen carbons,
(viii) aryl,
(ix) aryl substituted with 1, 2, 3, 4, or 5 substituents independently selected from alkyl of one to fifteen carbons,
alkoxy of one to fifteen carbons,
thioalkoxy of one to fifteen carbons,
halo,
—NO₂, and
—N₃,
(x) cycloalkyl of three to twelve carbons, and
(xi) halo,
(xii) —NR₇R₈,
(xiii) heterocycle, and
(xiv) heterocycle substituted with 1, 2, or 3, or 4 substituents independently selected from alkyl of one to fifteen carbons,
alkoxy of one to fifteen carbons,
thioalkoxy of one to fifteen carbons,
halo,
—NO₂, and
—N₃,
(12) alkyl of one to fifteen carbons substituted with 1, 2, 3, 4, or 5 halo substituents,
(13) alkyl of one to fifteen carbons,
(14) alkenyl of two to fifteen carbons,
(15) alkynyl of two to fifteen carbons
where (13)-(15) can be optionally substituted with
(a) (═X),
(b) alkanoyloxy where the alkyl part is one to fifteen carbons,
(c) alkoxy of one to fifteen carbons,
(d) alkoxy of one to fifteen carbons substituted with 1, 2, 3, 4, or 5 substituents selected from the group consisting of halo,
(e) thioalkoxy of one to fifteen carbons,
(f) perfluoroalkoxy of one to fifteen carbons,
(g) —N₃,
(h) —NO₂,
(i) —CN,
(j) —OH,
(k) —OG
(l) cycloalkyl of three to twelve carbons,
(m) halo,
(n) —CO₂R₆,
(o) —L₁NR₇R₈, and
(p) —L₂R₉,
(16) —L₂-heterocycle, and
(17) —L₂-heterocycle where the heterocycle is substituted with 1, 2, 3 or 4 substituents independently selected from
(a) alkyl of one to fifteen carbons,
(b) perfluoroalkyl of one to fifteen carbons,
(c) alkoxy of one to fifteen carbons,
(d) thioalkoxy of one to fifteen carbons,
(e) halo, and
(f) —NO₂,
(18) —NR_{X}C(O)NR_{Y}R_{Z} where R_{X}, R_{Y} and R_{Z} are independently selected from
(a) hydrogen and
(b) alkyl of one to fifteen carbons,
(19) —C(═NR_{X})NR_{Y}R_{Z},
(20) —NR_{X}C(═NR_{X′})NR_{Y}R_{Z} where R_{X}, R_{Y} and R_{Z} are defined previously and R_{X′} is selected from
(a) hydrogen and
(b) alkyl of one to fifteen carbons,
(21) —NR_{X}C(O)OR_{W}, where R_{W} is selected from
(a) alkyl of one to fifteen carbons and
(b) alkenyl of three to fifteen carbons,
provided that a carbon of a carbon-carbon double bond is not attached directly to oxygen, and
(22) —OC(O)NR₇R₈;
Z is nitrogen or carbon;
R₂ is absent or is selected from
(1) hydrogen,
(2) —CO₂R₆,
(3) alkyl of one to fifteen carbons,
(4) —C(O)R_{6′} where R_{6′} is selected from
(a) alkyl of one to fifteen carbons,
(b) aryl, and
(c) heterocycle,
(5) —C(O)NR_{7′}R_{8′} where R₇′ and R_{8′} are independently selected from
(a) hydrogen,
(b) alkyl of one to fifteen carbons, or
R_{7′} and R_{8′} together with the nitrogen to which they are attached form a ring selected from
(i) piperidine,
(ii) piperazine,
(iii) morpholine,
(iv) thiomorpholine, and
(v) thiomorpholine sulfone
(6) perfluoroalkyl of one to fifteen carbons,
(7) cycloalkyl of three to ten carbons,
(8) alkyl of one to fifteen carbons substituted with 1, 2, 3, 4, or 5 substituents selected from the group conststing of halo,
(9) alkyl of one to fifteen carbons substituted with
(a) —CN,
(b) —OH,
provided that no two —OH groups are attached to the same carbon,
(c) (═X), and
(d) —CO₂R₆, and
(10) halogen;
provided that when X is nitrogen, R₂ is absent;
Q is aryl or heterocycle where, when Q is phenyl, the phenyl is 2-, 3-, or 4- substituted by E relative to the position of attachment of the pyrazole or 1,2,4-triazole ring to the phenyl ring;
R₄ and R₅ are independently selected from
(1) hydrogen,
(2) alkyl of one to fifteen carbons,
(3) alkyl of one to fifteen carbons substituted with 1, 2, 3, 4, or 5 halo substituents,
(4) alkyl of one to fifteen carbons substituted with
(a) —CN,
(b) —CO₂R₆,
(c) —L₁NR₇R₈, and
(d) —L₂R₉,
(5) perfluoroalkyl of one to fifteen carbons,
(6) —CN,
(7) —CO₂R₆,
(8) —L₁NR₇R₈,
(9) —L₂R₉,
(10) alkoxy of one to fifteen carbons,
(11) thioalkoxy of one to fifteen carbons,
(12) halo,
(13) —C(═NR₆)NR₇R₈,
(14) —NR₁₂(═NR₆)NR₇R₈ where R₆, R₇, and R₈ are defined previously and R₁₂ is selected from
(a) hydrogen,
(b) cycloalkyl of three to twelve carbons,
(c) aryl,
(d) alkyl of one to fifteen carbons, and
(e) alkyl of one to fifteen carbons substituted with 1, 2, or 3, or 4 substituents independently selected from
(i) alkenyl of two to fifteen carbons,
(ii) alkoxy of one to fifteen carbons,
(iii) thioalkoxy of one to fifteen carbons,
(iv) alkynyl of two to fifteen carbons, and
(v) aryl,
(15) —L₂-heterocycle, and
(16) —L₂-heterocycle where the heterocycle is substituted with 1, 2, 3, or 4 substituents independently selected from
(a) alkyl of one to fifteen carbons,
(b) perfluoroalkyl of one to fifteen carbons,
(c) alkoxy of one to fifteen carbons,
(d) thioalkoxy of one to fifteen carbons,
(e) halo,
(f) —N₃, and
(g) —NO₂;
E is
(1) —L₃-B where L₃ is selected from
(a) a covalent bond,
(b) alkenylene of two to six carbons in the Z or E configuration,
(c) alkynylene of two to six carbons,
(d) —C(X)—,
(e) —N═N—,
(f) —NR₇—,
(g) —N(R₇)C(O)N(R₈)—,
(h) —N(R₇)SO₂N(R₈)—,
(i) —X—,
(j) —(CH₂)ₘO—,
(k) —O(CH₂)ₘ—,
(l) —N(R₇)C(X)—,
(m) —C(X)N(R₇)—,
(n) —S(O)ₜ(CH₂)ₘ—,
(o) —(CH₂)ₘS(O)ₜ—,
(p) —NR₇(CH₂)ₘ—,
(q) —(CH₂)ₘNR₇—,
(r) —NR₇S(O)ₜ—,
(s) —S(O)ₜNR₇—,
(t) —N═C(H)—,
(u) —C(H)═N—,
(v) —ON═CH—,
(w) —CH═NO—
where (g)-(w) are drawn with their left ends attached to Q,
(x) —N(R₇)C(O)N(R₁₀)(R₁₁)— where R₁₀ and R₁₁ together with the nitrogen atom to which they are attached form a ring selected from
(i) morpholine,
(ii) thiomorpholine,
(iii) thiomorpholine sulfone, and
(iv) piperidine
where (i)-(iv) are attached to Q through the nitrogen to which is attached R₇ and to B through a carbon in the ring,
(y) —N(R₇)SO₂N(R₁₀)(R₁₁)—, and
(z) —N(R₇)C(O)N(R₁₀)(R₁₁)— and
B is selected from
(a) alkyl of one to fifteen carbons,
(b) alkenyl of three to fifteen carbons in the E or Z configuration, provided that a carbon of a carbon-carbon double bond is not directly attached to L₃ when L₃ is other than a covalent bond,
(c) alkynyl of three to fifteen carbons, provided that a carbon of a carbon-carbon triple bond is not directly attached to L₃ when L₃ is other than a covalent bond
where (a), (b) and (c), can be optionally substituted with 1, 2, 3, or 4 substituents independently selected from
(i) 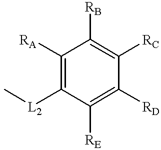
where L₂ is defined previously and RA, RB, RC, RD, and RE are independently selected from hydrogen,
alkanoyl where the alkyl part is one to fifteen carbons,
alkanoyloxy where the alkyl part is one to fifteen carbons,
alkoxy of one to fifteen carbons,
thioalkoxy of one to fifteen carbons,
alkoxy of one to fifteen carbons substituted with 1, 2, 3, 4, or 5 substituents selected from the group consisting of halo,
perfluoroalkyl of one to fifteen carbons,
perfluoroalkoxy of one to fifteen carbons,
—N₃,
—NO₂,
—CN,
—OH,
—OG,
cycloalkyl of three to fifteen carbons,
halo,
—CO₂R₆
—L₁NR₇R₈
—L₂R₉
alkyl of one to fifteen carbons,
alkyl of one to fifteen carbons substituted with 1, 2, 3, 4, or 5 substituents independently selected from (═X), alkanoyloxy where the alkyl part is one to fifteen carbons,
alkoxy of one to fifteen carbons,
thioalkoxy of one to fifteen carbons,
alkoxy of one to fifteen carbons substituted with 1, 2, 3,4, or 5 halo substituents,
perfluoroalkoxy of one to fifteen carbons,
—N₃,
—NO₂,
—CN,
—OH,
provided that no two —OH groups are attached to the same carbon,
—OG,
cycloalkyl of three to fifteen carbons,
halo,
—CO₂R₆,
—L₁NR₇R₈, and
—L₂R₉,
—L₂-heterocycle, and
—L₂-heterocycle where the heterocycle is substituted with 1, 2, 3, or 4 substituents independently selected from alkyl of one to fifteen carbons,
perfluoroalkyl of one to fifteen carbons,
alkoxy of one to fifteen carbons,
thioalkoxy of one to fifteen carbons,
halo,
—NR_{X}C(O)NR_{Y}R_{Z},
—C(═NRX)R_{Y}R_{Z},
—NO₂, and
—N₃,
(ii) (═X)
(iii) alkanoyloxy where the alkyl part is one to fifteen carbons,
(iv) alkoxy of one to fifteen carbons,
(v) alkoxy of one to fifteen carbons substituted with 1, 2, 3, 4, or 5 substituents selected from the group consisting of halo,
(vi) thioalkoxy of one to fifteen carbons,
(vii) perfluoroalkoxy of one to fifteen carbons,
(viii) —N₃,
(ix) —NO₂,
(x) —CN,
(xi) —OH,
provided that no two —OH groups are attached to the same carbon,
(xii) —OG,
(xiii) cycloalkyl of three to fifteen carbons,
(xiv) halo,
(xv) —CO₂R₆,
(xvi) —L₁NR₇R₈,
(xvii) perfluoroalkyl of one to fifteen carbons,
(xviii) —L2-heterocycle, and
(xix) —L2-heterocycle where the heterocycle is substituted with 1, 2, 3, or 4 substituents independently selected from (═X),
alkanoyl where the alkyl part is one to fifteen carbons,
alkanoyloxy where the alkyl part is one to fifteen carbons,
alkoxy of one to fifteen carbons,
alkoxy of one to fifteen carbons substituted with 1, 2, 3, 4, or 5 substituents selected from the group consisting of halo,
thioalkoxy of one to fifteen carbons,
perfluoroalkyl of one to fifteen carbons,
perfluoroalkoxy of one to fifteen carbons,
—N₃,
—NO₂,
—CN,
OH,
provided that no two -OH groups are attached to the same carbon,
—OG,
cycloalkyl of three to fifteen carbons, halo,
CO₂R₆,
—L₁NR₇R₈, and
—L₂R₉,
(d) cycloalkyl of three to twelve carbons,
(e) cycloalkenyl of four to twelve carbons,
provided that a carbon of a carbon-carbon-double bond is not attached directly to L₃ when L₃ is other than a covalent bond
where (d) and (e) can be optionally substituted with 1, 2, 3, 4, or 5 substituents independently selected from
(i) alkyl of one to fifteen carbons,
(ii) aryl,
(iii) alkoxy of one to fifteen carbons,
(iv) thioalkoxy of one to fifteen carbons,
(v) halo,
(vi) —OH,
provided that no two —OH groups are attached to the same carbon,
(vii) oxo,
(viii) perfluoroalkyl,
(ix) heterocycle, and
(x) heterocycle substituted with 1, 2, 3, 4, or 5 substituents independently selected from alkyl of one to fifteen carbons,
perfluoroalkyl of one to fifteen carbons,
alkoxy of one to fifteen carbons,
thioalkoxy of one to fifteen carbons,
halo,
—NO₂, and
—N₃,
(f) 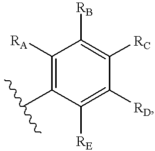
provided that when R₁ and R₃ are both perfluoroalkyl of one carbon, Z is carbon, R₂ is hydrogen, Q is phenyl that is 4-substituted by E relative to the position of attachment of the pyrazole ring to the phenyl group, R₄ and R₅ are hydrogen, E is —L₃-B, L₃ is —N(R₇)C(X)—, R₇ is hydrogen, X is oxygen, and R_{A}, R_{B}, R_{D}, and R_{E} are hydrogen, R_{C} is other than chloro, and
(g) heterocycle where the heterocycle can be optionally substituted with 1, 2, 3, or 4 substituents independently selected from
(i) (═X),
(ii) alkanoyl where the alkyl part is one to fifteen carbons,
(iii) alkanoyloxy where the alkyl part is one to fifteen carbons,
(iv) alkoxy of one to fifteen carbons,
(v) alkoxy of one to fifteen carbons substituted with 1, 2, 3, 4, or 5 substituents selected from the group consisting of halo,
(vi) halo,
(vii) thioalkoxy of one to fifteen carbons,
(viii) perfluoroalkyl of one to fifteen carbons,
(ix) perfluoroalkoxy of one to fifteen carbons,
(x) —N₃,
(xi) —NO₂,
(xii) —CN,
(xiii) —OH,
provided that no two —OH groups are attached to the same carbon,
(xiv) —OG,
(xv) cycloalkyl of three to fifteen carbons,
(xvi) halo,
(xvii) —CO₂R₆,
(xviii) alkyl optionally substituted with —OH,
(xix) —L₁NR₇R₈, and
(xx) —L₂R₉, and
(2) 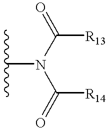
where R₁₃ and R₁₄ are independently selected from
(a) hydrogen,
(b) alkyl of one to fifteen carbons,
(c) alkenyl of three to fifteen carbons in the E or Z configuration, provided that a carbon of a carbon-carbon double bond is not attached directly to the C(═O) group,
(d) alkynyl of three to fifteen carbons,
provided that a a carbon-carbon triple bond is not directly attached to the C(═O) group
where (b), (c), and (d) can be optionally substituted with 1, 2, 3, or 4 substituents independently selected from
(i) 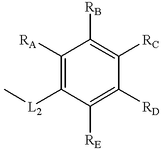
(ii) (═X),
(iii) alkanoyloxy where the alkyl part is one to fifteen carbons,
(iv) alkoxy of one to fifteen carbons,
(v) alkoxy of one to fifteen carbons substituted with 1, 2, 3, 4, or 5 substituents selected from the group consisting of halo,
(vi) thioalkoxy of one to fifteen carbons,
(vii) perfluoroalkoxy of one to fifteen carbons,
(viii) —N₃,
(ix) —NO₂,
(x) —CN,
(xi) —OH,
provided that no two —OH groups are attached to the same carbon,
(xii) —OG,
(xiii) cycloalkyl of three to fifteen carbons,
(xiv) halo,
(xv) —CO₂R₆,
(xvi) —L₁NR₇R₈,
(xvii) perfluoroalkyl of one to fifteen carbons,
(xviii) —L₂-heterocycle, and
(xix) —L₂-heterocycle where the heterocycle is substituted with 1, 2, 3,or 4 substituents independently selected from (═X),
alkanoyl where the alkyl part is one to fifteen carbons,
alkanoyloxy where the alkyl part is one to fifteen carbons,
alkoxy of one to fifteen carbons,
alkoxy of one to fifteen carbons substituted with 1, 2, 3, 4, or 5 substituents selected from the group consisting of halo,
thioalkoxy of one to fifteen carbons,
perfluoroalkyl of one to fifteen carbons,
perfluoroalkoxy of one to fifteen carbons,
—N₃,
—NO₂,
—CN,
—OH,
provided that no two —OH groups are attached to the same carbon,
—OG,
cycloalkyl of three to fifteen carbons,
halo,
—CO₂R₆,
L₁NR₇R₈,
L₂R₉,
(e) cycloalkyl of three to twelve carbons,
(f) cycloalkenyl of four to twelve carbons, provided that a carbon of a carbon-carbon double bond is not attached directly to the C(═O) group
where (e) and (f) can be optionally substituted with 1, 2, 3, 4, or 5 substituents independently selected from
(i) alkyl of one to fifteen carbons,
(ii) aryl,
(iii) alkoxy of one to fifteen carbons,
(iv) thioalkoxy of one to fifteen carbons,
(v) halo,
(vi) —OH,
provided that no two —OH groups are attached to the same carbon,
(vii) heterocycle, and
(viii) heterocycle substituted with 1, 2, 3, 4, or 5 substituents independently selected from alkyl of one to fifteen carbons, perfluoroalkyl of one to fifteen carbons, alkoxy of one to fifteen carbons, thioalkoxy of one to fifteen carbons, halo, —NO₂, and —N₃,
(g) heterocycle, and
(h) heterocycle substituted with 1, 2, 3, or 4 substituents independently selected from
(i) (═X),
(ii) alkanoyl where the alkyl part is one to fifteen carbons,
(iii) alkanoyloxy where the alkyl part is one to fifteen carbons,
(iv) alkoxy of one to fifteen carbons,
(v) alkoxy of one to fifteen carbons substituted with 1, 2, 3, 4, or 5 substituents selected from the group consisting of halo,
(vi) thioalkoxy of one to fifteen carbons,
(vii) perfluoroalkyl of one to fifteen carbons,
(viii) perfluoroalkoxy of one to fifteen carbons,
(ix) —N₃,
(x) —NO₂,
(xi) —CN,
(xii) —OH,
provided that no two —OH groups are attached to the same carbon,
(xiii) —OG,
(xiv) cycloalkyl of three to fifteen carbons,
(xv) halo,
(xvi) —CO₂R₆,
(xvii) —L₁NR₇R₈,
(xviii) —L₂R₉,
provided that at least one of R₁₃ and R₁₄ is other than hydrogen, or R₁₃ and R₁₄ together with the nitrogen to which they are attached form a ring selected from
(a) succinimidyl,
(b) maleimidyl,
(c) glutarimidyl,
(d) phthalimnidyl,
(e) naphthalimidyl,
(f) 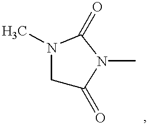
(g) 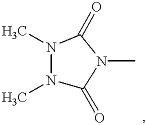
(h) 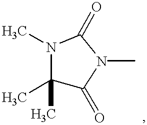
(i) 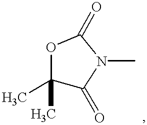
(j) 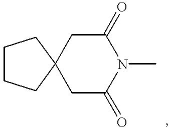
(k) 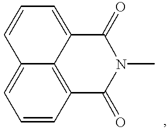
(l) 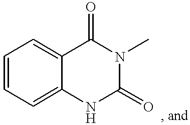
(m) 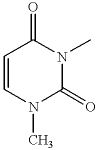
where (a)-(m) can be optionally substituted with 1, 2, 3, 4, or 5 substituents selected from halo and —L₂R₉.
